SEQUENCE LISTING (604 sequences)

SEQ ID NO: 1-99 - skipped (99 entries)

SEQ ID NO: 100 - protein, 125 aa, synthetic construct
QSLVESGGGLVQPGGSLRLSCAASGIDFASSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 101 - protein, 125 aa, synthetic construct
QSLVESGGGLVQPGGSLRLSCAASGIDFSASGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 102 - protein, 125 aa, synthetic construct
QSLVESGGGLVQPGGSLRLSCAASGIDFSSAGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 103 - protein, 125 aa, synthetic construct
QSLVESGGGLVQPGGSLRLSCAASGIDFSSSGYMCWVRQAPGKGLEWVGCIATYSSNTYYAASVKGRFTISKTSSTTVYLQMNSLKTEDTAVYYCARGTYGYTGYTYTMGYFSLWGQGTLVTVSS

SEQ ID NO: 104 - protein, 125 aa, synthetic construct
QSLVESGGGLVQPGGSLRLSCAASGIDFSSSGYMCWVRQAPGKGLEWVGCIYTASSNTYYAASVKGRFTISKTSSTTVYLQMNSLKTEDTAVYYCARGTYGYTGYTYTMGYFSLWGQGTLVTVSS

SEQ ID NO: 105 - protein, 125 aa, synthetic construct
QSLVESGGGLVQPGGSLRLSCAASGIDFSSSGYMCWVRQAPGKGLEWVGCIYTYSSATYYAASVKGRFTISKTSSTTVYLQMNSLKTEDTAVYYCARGTYGYTGYTYTMGYFSLWGQGTLVTVSS

SEQ ID NO: 106 - protein, 125 aa, synthetic construct
QSLVESGGGLVQPGGSLRLSCAASGIDFSSSGYMCWVRQAPGKGLEWVGCIYTYSSNTAYAASVKGRFTISKTSSTTVYLQMNSLKTEDTAVYYCARGTYGYTGYTYTMGYFSLWGQGTLVTVSS

SEQ ID NO: 107 - protein, 125 aa, synthetic construct
QSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGATGYTYTMGYFSLWGQGTLVTVSS

SEQ ID NO: 108 - protein, 125 aa, synthetic construct
QSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGATYTMGYFSLWGQGTLVTVSS

SEQ ID NO: 109 - protein, 125 aa, synthetic construct
QSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYAYTMGYFSLWGQGTLVTVSS

SEQ ID NO: 110 - protein, 125 aa, synthetic construct
QSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTATMGYFSLWGQGTLVTVSS

SEQ ID NO: 111 - protein, 126 aa, synthetic construct
EVQLVESGGGLVQPGGSLRLSCAASGIDFASSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 112 - protein, 126 aa, synthetic construct
EVQLVESGGGLVQPGGSLRLSCAASGIDFDSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 113 - protein, 126 aa, synthetic construct
EVQLVESGGGLVQPGGSLRLSCAASGIDFHSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 114 - protein, 126 aa, synthetic construct
EVQLVESGGGLVQPGGSLRLSCAASGIDFSASGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 115 - protein, 126 aa, synthetic construct
EVQLVESGGGLVQPGGSLRLSCAASGIDFSDSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 116 - protein, 126 aa, synthetic construct
EVQLVESGGGLVQPGGSLRLSCAASGIDFSESGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 117 - protein, 126 aa, synthetic construct
EVQLVESGGGLVQPGGSLRLSCAASGIDFSHSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 118 - protein, 126 aa, synthetic construct
EVQLVESGGGLVQPGGSLRLSCAASGIDFSSAGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 119 - protein, 126 aa, synthetic construct
EVQLVESGGGLVQPGGSLRLSCAASGIDFSSDGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 120 - protein, 126 aa, synthetic construct
EVQLVESGGGLVQPGGSLRLSCAASGIDFSSHGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 121 - protein, 126 aa, synthetic construct
EVQLVESGGGLVQPGGSLRLSCAASGIDFSSSGYMCWVRQAPGKGLEWVGCIATYSSNTYYAASVKGRFTISKTSSTTVYLQMNSLKTEDTAVYYCARGTYGYTGYTYTMGYFSLWGQGTLVTVSS

SEQ ID NO: 122 - protein, 126 aa, synthetic construct
EVQLVESGGGLVQPGGSLRLSCAASGIDFSSSGYMCWVRQAPGKGLEWVGCIDTYSSNTYYAASVKGRFTISKTSSTTVYLQMNSLKTEDTAVYYCARGTYGYTGYTYTMGYFSLWGQGTLVTVSS

SEQ ID NO: 123 - protein, 126 aa, synthetic construct
EVQLVESGGGLVQPGGSLRLSCAASGIDFSSSGYMCWVRQAPGKGLEWVGCIHTYSSNTYYAASVKGRFTISKTSSTTVYLQMNSLKTEDTAVYYCARGTYGYTGYTYTMGYFSLWGQGTLVTVSS

SEQ ID NO: 124 - protein, 126 aa, synthetic construct
EVQLVESGGGLVQPGGSLRLSCAASGIDFSSSGYMCWVRQAPGKGLEWVGCIYTASSNTYYAASVKGRFTISKTSSTTVYLQMNSLKTEDTAVYYCARGTYGYTGYTYTMGYFSLWGQGTLVTVSS

SEQ ID NO: 125 - protein, 126 aa, synthetic construct
EVQLVESGGGLVQPGGSLRLSCAASGIDFSSSGYMCWVRQAPGKGLEWVGCIYTDSSNTYYAASVKGRFTISKTSSTTVYLQMNSLKTEDTAVYYCARGTYGYTGYTYTMGYFSLWGQGTLVTVSS

SEQ ID NO: 126 - protein, 126 aa, synthetic construct
EVQLVESGGGLVQPGGSLRLSCAASGIDFSSSGYMCWVRQAPGKGLEWVGCIYTHSSNTYYAASVKGRFTISKTSSTTVYLQMNSLKTEDTAVYYCARGTYGYTGYTYTMGYFSLWGQGTLVTVSS

SEQ ID NO: 127 - protein, 126 aa, synthetic construct
EVQLVESGGGLVQPGGSLRLSCAASGIDFSSSGYMCWVRQAPGKGLEWVGCIYTYSSATYYAASVKGRFTISKTSSTTVYLQMNSLKTEDTAVYYCARGTYGYTGYTYTMGYFSLWGQGTLVTVSS

SEQ ID NO: 128 - protein, 126 aa, synthetic construct
EVQLVESGGGLVQPGGSLRLSCAASGIDFSSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSS

SEQ ID NO: 129 - protein, 126 aa, synthetic construct
EVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGYTYTMGYFSLWGQGTLVTVSS

SEQ ID NO: 130 - protein, 126 aa, synthetic construct
EVQLVESGGGLVQPGGSLRLSCAASGIDFSSSGYMCWVRQAPGKGLEWVGCIYTYSSHTYYAASVKGRFTISKTSSTTVYLQMNSLKTEDTAVYYCARGTYGYTGYTYTMGYFSLWGQGTLVTVSS

SEQ ID NO: 131 - protein, 126 aa, synthetic construct
EVQLVESGGGLVQPGGSLRLSCAASGIDFSSSGYMCWVRQAPGKGLEWVGCIYTYSSNTAYAASVKGRFTISKTSSTTVYLQMNSLKTEDTAVYYCARGTYGYTGYTYTMGYFSLWGQGTLVTVSS

SEQ ID NO: 132 - protein, 126 aa, synthetic construct
EVQLVESGGGLVQPGGSLRLSCAASGIDFSSSGYMCWVRQAPGKGLEWVGCIYTYSSNTDYAASVKGRFTISKTSSTTVYLQMNSLKTEDTAVYYCARGTYGYTGYTYTMGYFSLWGQGTLVTVSS

SEQ ID NO: 133 - protein, 126 aa, synthetic construct
EVQLVESGGGLVQPGGSLRLSCAASGIDFSSSGYMCWVRQAPGKGLEWVGCIYTYSSNTEYAASVKGRFTISKTSSTTVYLQMNSLKTEDTAVYYCARGTYGYTGYTYTMGYFSLWGQGTLVTVSS

SEQ ID NO: 134 - protein, 126 aa, synthetic construct
EVQLVESGGGLVQPGGSLRLSCAASGIDFSSSGYMCWVRQAPGKGLEWVGCIYTYSSNTHYAASVKGRFTISKTSSTTVYLQMNSLKTEDTAVYYCARGTYGYTGYTYTMGYFSLWGQGTLVTVSS

SEQ ID NO: 135 - protein, 126 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGATGYTYTMGYFSLWGQGTLVTVSS

SEQ ID NO: 136 - protein, 126 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGDTGYTYTMGYFSLWGQGTLVTVSS

SEQ ID NO: 137 - protein, 126 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGHTGYTYTMGYFSLWGQGTLVTVSS

SEQ ID NO: 138 - protein, 126 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGATYTMGYFSLWGQGTLVTVSS

SEQ ID NO: 139 - protein, 126 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGDTYTMGYFSLWGQGTLVTVSS

SEQ ID NO: 140 - protein, 126 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGHTYTMGYFSLWGQGTLVTVSS

SEQ ID NO: 141 - protein, 126 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYAYTMGYFSLWGQGTLVTVSS

SEQ ID NO: 142 - protein, 126 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYDYTMGYFSLWGQGTLVTVSS

SEQ ID NO: 143 - protein, 126 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYHYTMGYFSLWGQGTLVTVSS

SEQ ID NO: 144 - protein, 126 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTATMGYFSLWGQGTLVTVSS

SEQ ID NO: 145 - protein, 126 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTDTMGYFSLWGQGTLVTVSS

SEQ ID NO: 146 - protein, 126 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTHTMGYFSLWGQGTLVTVSS

SEQ ID NO: 147 - protein, 126 aa, synthetic construct
EVQLVESGGGLVQPGGSLRLSCAASGIDFDSSGYMCWVRQAPGKGLEWVGCIYTYSSNTYYAASVKGRFTISKTSSTTVYLQMNSLKTEDTAVYYCARGTYGYTGATYTMGYFSLWGQGTLVTVSS

SEQ ID NO: 148 - protein, 126 aa, synthetic construct
EVQLVESGGGLVQPGGSLRLSCAASGIDFSSSGYMCWVRQAPGKGLEWVGCIYTYSSNTAYAASVKGRFTISKTSSTTVYLQMNSLKTEDTAVYYCARGTYGYTGATYTMGYFSLWGQGTLVTVSS

SEQ ID NO: 149 - protein, 126 aa, synthetic construct
EVQLVESGGGLVQPGGSLRLSCAASGIDFSSSGYMCWVRQAPGKGLEWVGCIYTYSSNTEYAASVKGRFTISKTSSTTVYLQMNSLKTEDTAVYYCARGTYGYTGATYTMGYFSLWGQGTLVTVSS

SEQ ID NO: 150 - protein, 126 aa, synthetic construct
EVQLVESGGGLVQPGGSLRLSCAASGIDFDSSGYMCWVRQAPGKGLEWVGCIYTDSSNTYYAASVKGRFTISKTSSTTVYLQMNSLKTEDTAVYYCARGTYGYTGYTYTMGYFSLWGQGTLVTVSS

SEQ ID NO: 151 - protein, 126 aa, synthetic construct
EVQLVESGGGLVQPGGSLRLSCAASGIDFDSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSS

SEQ ID NO: 152 - protein, 126 aa, synthetic construct
EVQLVESGGGLVQPGGSLRLSCAASGIDFDSSGYMCWVRQAPGKGLEWVGCIYTYSSETYYAASVKGRFTISKTSSTTVYLQMNSLKTEDTAVYYCARGTYGYTGYTYTMGYFSLWGQGTLVTVSS

SEQ ID NO: 153 - protein, 126 aa, synthetic construct
EVQLVESGGGLVQPGGSLRLSCAASGIDFDSSGYMCWVRQAPGKGLEWVGCIYTYSSNTDYAASVKGRFTISKTSSTTVYLQMNSLKTEDTAVYYCARGTYGYTGYTYTMGYFSLWGQGTLVTVSS

SEQ ID NO: 154 - protein, 126 aa, synthetic construct
EVQLVESGGGLVQPGGSLRLSCAASGIDFDSSGYMCWVRQAPGKGLEWVGCIYTYSSNTEYAASVKGRFTISKTSSTTVYLQMNSLKTEDTAVYYCARGTYGYTGYTYTMGYFSLWGQGTLVTVSS

SEQ ID NO: 155 - protein, 126 aa, synthetic construct
EVQLVESGGGLVQPGGSLRLSCAASGIDFDSSGYMCWVRQAPGKGLEWVGCIHTDSSNTYYAASVKGRFTISKTSSTTVYLQMNSLKTEDTAVYYCARGTYGYTGYTYTMGYFSLWGQGTLVTVSS

SEQ ID NO: 156 - protein, 126 aa, synthetic construct
EVQLVESGGGLVQPGGSLRLSCAASGIDFSSSGYMAWVRQAPGKGLEWVGAIYTYSSDTYYAASVKGRFTISKTSSTTVYLQMNSLKTEDTAVYYCARGTYGYTGYTYTMGYFSLWGQGTLVTVSS

SEQ ID NO: 157 - protein, 126 aa, synthetic construct
EVQLVESGGGLVQPGGSLRLSCAASGIDFSSSGYMAWVRQAPGKGLEWVGAIYTYSSNTDYAASVKGRFTISKTSSTTVYLQMNSLKTEDTAVYYCARGTYGYTGYTYTMGYFSLWGQGTLVTVSS

SEQ ID NO: 158 - protein, 126 aa, synthetic construct
EVQLVESGGGLVQPGGSLRLSCAASGIDFSSSGYMCWVRQAPGKGLEWVGCIYTDSSETYYAASVKGRFTISKTSSTTVYLQMNSLKTEDTAVYYCARGTYGYTGYTYTMGYFSLWGQGTLVTVSS

SEQ ID NO: 159 - protein, 126 aa, synthetic construct
EVQLVESGGGLVQPGGSLRLSCAASGIDFSSSGYMCWVRQAPGKGLEWVGCIYTYSSETEYAASVKGRFTISKTSSTTVYLQMNSLKTEDTAVYYCARGTYGYTGYTYTMGYFSLWGQGTLVTVSS

SEQ ID NO: 160 - protein, 126 aa, synthetic construct
EVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGATYTMGYFSLWGQGTLVTVSS

SEQ ID NO: 161-199 - skipped (39 entries)

SEQ ID NO: 200 - protein, 111 aa, synthetic construct
DIQMTQSPSTLSASVGDRVTITCQASQSINSYLAWYQQKPGKAPKLLIYRASSLESGVPSRFSGSGSGTEFTLTISSLQPDDFATYYCQSYYYSGSSNYNAFGGGTKVEIK

SEQ ID NO: 201 - protein, 111 aa, synthetic construct
DIQMTQSPSTLSASVGDRVTITCQASQNISSYLAWYQQKPGKAPKLLIYRASSLESGVPSRFSGSGSGTEFTLTISSLQPDDFATYYCQSYYYSGSSNYNAFGGGTKVEIK

SEQ ID NO: 202 - protein, 111 aa, synthetic construct
DIQMTQSPSTLSASVGDRVTITCQASQNINSYLAWYQQKPGKAPKLLIYDASSLESGVPSRFSGSGSGTEFTLTISSLQPDDFATYYCQSYYYSGSSNYNAFGGGTKVEIK

SEQ ID NO: 203 - protein, 111 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYDSGSSNYNAFGGGTKVEIK

SEQ ID NO: 204 - protein, 111 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYHSGSSNYNAFGGGTKVEIK

SEQ ID NO: 205 - protein, 111 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYSSGSSNYNAFGGGTKVEIK

SEQ ID NO: 206 - protein, 111 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGASNYNAFGGGTKVEIK

SEQ ID NO: 207 - protein, 111 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGDSNYNAFGGGTKVEIK

SEQ ID NO: 208 - protein, 111 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGHSNYNAFGGGTKVEIK

SEQ ID NO: 209 - protein, 111 aa, synthetic construct
DIQMTQSPSTLSASVGDRVTITCQASQSINSYLAWYQQKPGKAPKLLIYDASSLESGVPSRFSGSGSGTEFTLTISSLQPDDFATYYCQSYYYSGSSNYNAFGGGTKVEIK

SEQ ID NO: 210 - protein, 111 aa, synthetic construct
DIQMTQSPSTLSASVGDRVTITCQASQSINSYLAWYQQKPGKAPKLLIYDASSLESGVPSRFSGSGSGTEFTLTISSLQPDDFATYYCQSYYSSGSSNYNAFGGGTKVEIK

SEQ ID NO: 211 - protein, 111 aa, synthetic construct
DIQMTQSPSTLSASVGDRVTITCQASQSINSYLAWYQQKPGKAPKLLIYDASSLESGVPSRFSGSGSGTEFTLTISSLQPDDFATYYCQSYYYSGDSNYNAFGGGTKVEIK

SEQ ID NO: 212 - protein, 111 aa, synthetic construct
DIQMTQSPSTLSASVGDRVTITCQASQSINSYLAWYQQKPGKAPKLLIYRASSLESGVPSRFSGSGSGTEFTLTISSLQPDDFATYYCQSYYDSGSSNYNAFGGGTKVEIK

SEQ ID NO: 213 - protein, 111 aa, synthetic construct
DIQMTQSPSTLSASVGDRVTITCQASQSINSYLAWYQQKPGKAPKLLIYRASSLESGVPSRFSGSGSGTEFTLTISSLQPDDFATYYCQSYYYSGDSNYNAFGGGTKVEIK

SEQ ID NO: 214 - protein, 111 aa, synthetic construct
DIQMTQSPSTLSASVGDRVTITCQASQSINSYLAWYQQKPGKAPKLLIYRASSLESGVPSRFSGSGSGTEFTLTISSLQPDDFATYYCQSYYSSGSSNYNAFGGGTKVEIK

SEQ ID NO: 215 - protein, 111 aa, synthetic construct
DIQMTQSPSTLSASVGDRVTITCQASQNINSYLAWYQQKPGKAPKLLIYDASSLESGVPSRFSGSGSGTEFTLTISSLQPDDFATYYCQSYYYSGDSNYNAFGGGTKVEIK

SEQ ID NO: 216 - protein, 111 aa, synthetic construct
DIQMTQSPSTLSASVGDRVTITCQASQSISSYLAWYQQKPGKAPKLLIYRASSLESGVPSRFSGSGSGTEFTLTISSLQPDDFATYYCQSYYYSGDSNYNAFGGGTKVEIK

SEQ ID NO: 217-299 - skipped (83 entries)

SEQ ID NO: 300 - protein, 125 aa, synthetic construct
QSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSS

SEQ ID NO: 301 - protein, 111 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGGGTKVEIK

SEQ ID NO: 302 - protein, 11 aa, synthetic construct
GIDFSSSGYMC

SEQ ID NO: 303 - protein, 17 aa, synthetic construct
CIYTYSSNTYYAASVKG

SEQ ID NO: 304 - protein, 17 aa, synthetic construct
GTYGYTGYTYTMGYFSL

SEQ ID NO: 305 - protein, 11 aa, synthetic construct
QASQNINSYLA

SEQ ID NO: 306 - protein, 7 aa, synthetic construct
RASSLES

SEQ ID NO: 307 - protein, 13 aa, synthetic construct
QSYYYSGSSNYNA

SEQ ID NO: 308 - protein, 8 aa, synthetic construct
GIDFSSSG

SEQ ID NO: 309 - protein, 4 aa, synthetic construct
TYSS

SEQ ID NO: 310 - protein, 15 aa, synthetic construct
TYGYTGYTYTMGYFS

SEQ ID NO: 311 - protein, 7 aa, synthetic construct
SQNINSY

SEQ ID NO: 312 - protein, 10 aa, synthetic construct
YYYSGSSNYN

SEQ ID NO: 313 - protein, 11 aa, synthetic construct
GIDFASSGYMC

SEQ ID NO: 314 - protein, 11 aa, synthetic construct
GIDFDSSGYMC

SEQ ID NO: 315 - protein, 11 aa, synthetic construct
GIDFHSSGYMC

SEQ ID NO: 316 - protein, 11 aa, synthetic construct
GIDFSASGYMC

SEQ ID NO: 317 - protein, 11 aa, synthetic construct
GIDFSDSGYMC

SEQ ID NO: 318 - protein, 11 aa, synthetic construct
GIDFSESGYMC

SEQ ID NO: 319 - protein, 11 aa, synthetic construct
GIDFSHSGYMC

SEQ ID NO: 320 - protein, 11 aa, synthetic construct
GIDFSSAGYMC

SEQ ID NO: 321 - protein, 11 aa, synthetic construct
GIDFSSDGYMC

SEQ ID NO: 322 - protein, 11 aa, synthetic construct
GIDFSSHGYMC

SEQ ID NO: 323 - protein, 17 aa, synthetic construct
CIATYSSNTYYAASVKG

SEQ ID NO: 324 - protein, 17 aa, synthetic construct
CIDTYSSNTYYAASVKG

SEQ ID NO: 325 - protein, 17 aa, synthetic construct
CIHTYSSNTYYAASVKG

SEQ ID NO: 326 - protein, 17 aa, synthetic construct
CIETYSSNTYYAASVKG

SEQ ID NO: 327 - protein, 17 aa, synthetic construct
CIFTYSSNTYYAASVKG

SEQ ID NO: 328 - protein, 17 aa, synthetic construct
CIYTASSNTYYAASVKG

SEQ ID NO: 329 - protein, 17 aa, synthetic construct
CIYTDSSNTYYAASVKG

SEQ ID NO: 330 - protein, 17 aa, synthetic construct
CIHTDSSNTYYAASVKG

SEQ ID NO: 331 - protein, 17 aa, synthetic construct
CIYTHSSNTYYAASVKG

SEQ ID NO: 332 - protein, 17 aa, synthetic construct
CIYTYSSATYYAASVKG

SEQ ID NO: 333 - protein, 17 aa, synthetic construct
CIYTYSSDTYYAASVKG

SEQ ID NO: 334 - protein, 17 aa, synthetic construct
CIYTYSSETYYAASVKG

SEQ ID NO: 335 - protein, 23 aa, synthetic construct
EPKSCDKTHTCPPCPAPELLGGP

SEQ ID NO: 336 - protein, 13 aa, synthetic construct
EPKSCDKTHTCPP

SEQ ID NO: 337 - protein, 5 aa, synthetic construct
EPKSC

SEQ ID NO: 338 - protein, 20 aa, synthetic construct
ESKYGPPCPPCPAPEFLGGP

SEQ ID NO: 339 - protein, 9 aa, synthetic construct
ESKYGPPCP

SEQ ID NO: 340 - protein, 4 aa, synthetic construct
EPKS

SEQ ID NO: 341 - protein, 6 aa, synthetic construct
EPKSCD

SEQ ID NO: 342 - protein, 7 aa, synthetic construct
EPKSCDK

SEQ ID NO: 343 - protein, 8 aa, synthetic construct
EPKSCDKT

SEQ ID NO: 344 - protein, 9 aa, synthetic construct
EPKSCDKTH

SEQ ID NO: 345 - protein, 17 aa, synthetic construct
CIYTYSSHTYYAASVKG

SEQ ID NO: 346 - protein, 17 aa, synthetic construct
CIYTYSSKTYYAASVKG

SEQ ID NO: 347 - protein, 17 aa, synthetic construct
CIYTYSSRTYYAASVKG

SEQ ID NO: 348 - protein, 17 aa, synthetic construct
CIYTYSSNTAYAASVKG

SEQ ID NO: 349 - protein, 17 aa, synthetic construct
CIYTYSSNTDYAASVKG

SEQ ID NO: 350 - protein, 17 aa, synthetic construct
CIYTYSSNTEYAASVKG

SEQ ID NO: 351 - protein, 17 aa, synthetic construct
CIYTYSSNTHYAASVKG

SEQ ID NO: 352 - protein, 17 aa, synthetic construct
GTYGATGYTYTMGYFSL

SEQ ID NO: 353 - protein, 17 aa, synthetic construct
GTYGDTGYTYTMGYFSL

SEQ ID NO: 354 - protein, 17 aa, synthetic construct
GTYGHTGYTYTMGYFSL

SEQ ID NO: 355 - protein, 17 aa, synthetic construct
GTYGYTGATYTMGYFSL

SEQ ID NO: 356 - protein, 17 aa, synthetic construct
GTYGYTGDTYTMGYFSL

SEQ ID NO: 357 - protein, 17 aa, synthetic construct
GTYGYTGHTYTMGYFSL

SEQ ID NO: 358 - protein, 17 aa, synthetic construct
GTYGYTGYAYTMGYFSL

SEQ ID NO: 359 - protein, 17 aa, synthetic construct
GTYGYTGYDYTMGYFSL

SEQ ID NO: 360 - protein, 17 aa, synthetic construct
GTYGYTGYHYTMGYFSL

SEQ ID NO: 361 - protein, 17 aa, synthetic construct
GTYGYTGYTATMGYFSL

SEQ ID NO: 362 - protein, 17 aa, synthetic construct
GTYGYTGYTDTMGYFSL

SEQ ID NO: 363 - protein, 17 aa, synthetic construct
GTYGYTGYTHTMGYFSL

SEQ ID NO: 364 - protein, 11 aa, synthetic construct
QASQSINSYLA

SEQ ID NO: 365 - protein, 11 aa, synthetic construct
QASQNISSYLA

SEQ ID NO: 366 - protein, 7 aa, synthetic construct
DASSLES

SEQ ID NO: 367 - protein, 13 aa, synthetic construct
QSYYDSGSSNYNA

SEQ ID NO: 368 - protein, 13 aa, synthetic construct
QSYYHSGSSNYNA

SEQ ID NO: 369 - protein, 13 aa, synthetic construct
QSYYSSGSSNYNA

SEQ ID NO: 370 - protein, 13 aa, synthetic construct
QSYYYSGASNYNA

SEQ ID NO: 371 - protein, 13 aa, synthetic construct
QSYYYSGDSNYNA

SEQ ID NO: 372 - protein, 13 aa, synthetic construct
QSYYYSGHSNYNA

SEQ ID NO: 373 - protein, 17 aa, synthetic construct
CIYTDSSETYYAASVKG

SEQ ID NO: 374 - protein, 17 aa, synthetic construct
CIYTYSSETEYAASVKG

SEQ ID NO: 375 - protein, 11 aa, synthetic construct
QASQSISSYLA

SEQ ID NO: 376 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 377 - protein, 760 aa, Macaca fascicularis
MMDQARSAFSNLFGGEPLSYTRFSLARQVDGDNSHVEMKLAVDDEENADNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 378 - protein, 8 aa, synthetic construct
GIDFASSG

SEQ ID NO: 379 - protein, 8 aa, synthetic construct
GIDFDSSG

SEQ ID NO: 380 - protein, 8 aa, synthetic construct
GIDFHSSG

SEQ ID NO: 381 - protein, 8 aa, synthetic construct
GIDFSASG

SEQ ID NO: 382 - protein, 8 aa, synthetic construct
GIDFSDSG

SEQ ID NO: 383 - protein, 8 aa, synthetic construct
GIDFSESG

SEQ ID NO: 384 - protein, 8 aa, synthetic construct
GIDFSHSG

SEQ ID NO: 385 - protein, 8 aa, synthetic construct
GIDFSSAG

SEQ ID NO: 386 - protein, 8 aa, synthetic construct
GIDFSSDG

SEQ ID NO: 387 - protein, 8 aa, synthetic construct
GIDFSSHG

SEQ ID NO: 388 - protein, 4 aa, synthetic construct
TASS

SEQ ID NO: 389 - protein, 4 aa, synthetic construct
TDSS

SEQ ID NO: 390 - protein, 4 aa, synthetic construct
THSS

SEQ ID NO: 391 - protein, 15 aa, synthetic construct
TYGATGYTYTMGYFS

SEQ ID NO: 392 - protein, 15 aa, synthetic construct
TYGDTGYTYTMGYFS

SEQ ID NO: 393 - protein, 15 aa, synthetic construct
TYGHTGYTYTMGYFS

SEQ ID NO: 394 - protein, 15 aa, synthetic construct
TYGYTGATYTMGYFS

SEQ ID NO: 395 - protein, 15 aa, synthetic construct
TYGYTGDTYTMGYFS

SEQ ID NO: 396 - protein, 15 aa, synthetic construct
TYGYTGHTYTMGYFS

SEQ ID NO: 397 - protein, 15 aa, synthetic construct
TYGYTGYAYTMGYFS

SEQ ID NO: 398 - protein, 15 aa, synthetic construct
TYGYTGYDYTMGYFS

SEQ ID NO: 399 - protein, 15 aa, synthetic construct
TYGYTGYHYTMGYFS

SEQ ID NO: 400 - protein, 15 aa, synthetic construct
TYGYTGYTATMGYFS

SEQ ID NO: 401 - protein, 15 aa, synthetic construct
TYGYTGYTDTMGYFS

SEQ ID NO: 402 - protein, 15 aa, synthetic construct
TYGYTGYTHTMGYFS

SEQ ID NO: 403 - protein, 7 aa, synthetic construct
SQSINSY

SEQ ID NO: 404 - protein, 7 aa, synthetic construct
SQNISSY

SEQ ID NO: 405 - protein, 10 aa, synthetic construct
YYDSGSSNYN

SEQ ID NO: 406 - protein, 10 aa, synthetic construct
YYHSGSSNYN

SEQ ID NO: 407 - protein, 11 aa, synthetic construct
YYSSGSSNYNA

SEQ ID NO: 408 - protein, 10 aa, synthetic construct
YYYSGASNYN

SEQ ID NO: 409 - protein, 10 aa, synthetic construct
YYYSGDSNYN

SEQ ID NO: 410 - protein, 10 aa, synthetic construct
YYYSGHSNYN

SEQ ID NO: 411 - protein, 7 aa, synthetic construct
SQSISSY

SEQ ID NO: 412 - protein, 252 aa, synthetic construct
EVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGYTYTMGYFSLWGQGTLVTVSSGGGGSGGGGSGGGGSDIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGGGTKVEIK

SEQ ID NO: 413 - protein, 252 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGGGTKVEIKGGGGSGGGGSGGGGSEVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGYTYTMGYFSLWGQGTLVTVSS

SEQ ID NO: 414 - protein, 257 aa, synthetic construct
EVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGYTYTMGYFSLWGQGTLVTVSSGGGGSGGGGSGGGGSGGGGSDIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGGGTKVEIK

SEQ ID NO: 415 - protein, 257 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGGGTKVEIKGGGGSGGGGSGGGGSGGGGSEVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGYTYTMGYFSLWGQGTLVTVSS

SEQ ID NO: 416 - protein, 252 aa, synthetic construct
EVQLVESGGGLVQPGGSLRLSCAASGIDFSSSGYMCWVRQAPGKCLEWVGCIYTYSSETYYAASVKGRFTISKTSSTTVYLQMNSLKTEDTAVYYCARGTYGYTGYTYTMGYFSLWGQGTLVTVSSGGGGSGGGGSGGGGSDIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGCGTKVEIK

SEQ ID NO: 417 - protein, 252 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGCGTKVEIKGGGGSGGGGSGGGGSEVQLVESGGGLVQPGGSLRLSCAASGIDFSSSGYMCWVRQAPGKCLEWVGCIYTYSSETYYAASVKGRFTISKTSSTTVYLQMNSLKTEDTAVYYCARGTYGYTGYTYTMGYFSLWGQGTLVTVSS

SEQ ID NO: 418 - protein, 252 aa, synthetic construct
DIQMTQSPSTLSASVGDRVTITCQASQNINSYLAWYQQKPGKAPKCLIYRASSLESGVPSRFSGSGSGTEFTLTISSLQPDDFATYYCQSYYYSGSSNYNAFGGGTKVEIKGGGGSGGGGSGGGGSEVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGYTYTMGYFCLWGQGTLVTVSS

SEQ ID NO: 419 - protein, 252 aa, synthetic construct
DIQMTQSPSTLSASVGDRVTITCQASQNINSYLCWYQQKPGKAPKLLIYRASSLESGVPSRFSGSGSGTEFTLTISSLQPDDFATYYCQSYYYSGSSNYNAFGGGTKVEIKGGGGSGGGGSGGGGSEVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGYTYTMGCFSLWGQGTLVTVSS

SEQ ID NO: 420 - protein, 252 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSCYYSGSSNYNAFGGGTKVEIKGGGGSGGGGSGGGGSEVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGYTYTMCYFSLWGQGTLVTVSS

SEQ ID NO: 421 - protein, 257 aa, synthetic construct
EVQLVESGGGLVQPGGSLRLSCAASGIDFSSSGYMCWVRQAPGKCLEWVGCIYTYSSETYYAASVKGRFTISKTSSTTVYLQMNSLKTEDTAVYYCARGTYGYTGYTYTMGYFSLWGQGTLVTVSSGGGGSGGGGSGGGGSGGGGSDIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGCGTKVEIK

SEQ ID NO: 422 - protein, 257 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGCGTKVEIKGGGGSGGGGSGGGGSGGGGSEVQLVESGGGLVQPGGSLRLSCAASGIDFSSSGYMCWVRQAPGKCLEWVGCIYTYSSETYYAASVKGRFTISKTSSTTVYLQMNSLKTEDTAVYYCARGTYGYTGYTYTMGYFSLWGQGTLVTVSS

SEQ ID NO: 423 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 424 - protein, 111 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPP

SEQ ID NO: 425 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 426 - protein, 107 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVESKYGPPCP

SEQ ID NO: 427 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 428 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 429 - protein, 100 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVES

SEQ ID NO: 430 - protein, 223 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLS

SEQ ID NO: 431 - protein, 224 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 432 - protein, 225 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 433 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 434 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 435 - protein, 225 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 436 - protein, 225 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 437 - protein, 10 aa, synthetic construct
EPKSCDKTHT

SEQ ID NO: 438 - protein, 11 aa, synthetic construct
EPKSCDKTHTC

SEQ ID NO: 439 - protein, 12 aa, synthetic construct
EPKSCDKTHTCP

SEQ ID NO: 440 - protein, 14 aa, synthetic construct
EPKSCDKTHTCPPC

SEQ ID NO: 441 - protein, 15 aa, synthetic construct
EPKSCDKTHTCPPCP

SEQ ID NO: 442 - protein, 16 aa, synthetic construct
EPKSCDKTHTCPPCPA

SEQ ID NO: 443 - protein, 17 aa, synthetic construct
EPKSCDKTHTCPPCPAP

SEQ ID NO: 444 - protein, 18 aa, synthetic construct
EPKSCDKTHTCPPCPAPE

SEQ ID NO: 445 - protein, 19 aa, synthetic construct
EPKSCDKTHTCPPCPAPEL

SEQ ID NO: 446 - protein, 20 aa, synthetic construct
EPKSCDKTHTCPPCPAPELL

SEQ ID NO: 447 - protein, 21 aa, synthetic construct
EPKSCDKTHTCPPCPAPELLG

SEQ ID NO: 448 - protein, 22 aa, synthetic construct
EPKSCDKTHTCPPCPAPELLGG

SEQ ID NO: 449 - protein, 4 aa, synthetic construct
ESKY

SEQ ID NO: 450 - protein, 5 aa, synthetic construct
ESKYG

SEQ ID NO: 451 - protein, 6 aa, synthetic construct
ESKYGP

SEQ ID NO: 452 - protein, 7 aa, synthetic construct
ESKYGPP

SEQ ID NO: 453 - protein, 8 aa, synthetic construct
ESKYGPPC

SEQ ID NO: 454 - protein, 10 aa, synthetic construct
ESKYGPPCPP

SEQ ID NO: 455 - protein, 11 aa, synthetic construct
ESKYGPPCPPC

SEQ ID NO: 456 - protein, 12 aa, synthetic construct
ESKYGPPCPPCP

SEQ ID NO: 457 - protein, 13 aa, synthetic construct
ESKYGPPCPPCPA

SEQ ID NO: 458 - protein, 14 aa, synthetic construct
ESKYGPPCPPCPAP

SEQ ID NO: 459 - protein, 15 aa, synthetic construct
ESKYGPPCPPCPAPE

SEQ ID NO: 460 - protein, 16 aa, synthetic construct
ESKYGPPCPPCPAPEF

SEQ ID NO: 461 - protein, 17 aa, synthetic construct
ESKYGPPCPPCPAPEFL

SEQ ID NO: 462 - protein, 18 aa, synthetic construct
ESKYGPPCPPCPAPEFLG

SEQ ID NO: 463 - protein, 19 aa, synthetic construct
ESKYGPPCPPCPAPEFLGG

SEQ ID NO: 464 - protein, 107 aa, synthetic construct [sequence omitted]

SEQ ID NO: 465 - protein, 106 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 466 - protein, 5 aa, synthetic construct
SGGGG

SEQ ID NO: 467 - protein, 10 aa, synthetic construct
SGGGGSGGGG

SEQ ID NO: 468 - protein, 15 aa, synthetic construct
SGGGGSGGGGSGGGG

SEQ ID NO: 469 - protein, 9 aa, synthetic construct
GGGSGGGGG

SEQ ID NO: 470 - protein, 15 aa, synthetic construct
GGGGSGGGGSGGGGS

SEQ ID NO: 471 - protein, 528 aa, synthetic construct
HHHHHHHHGLNDIFEAQKIEWHEGGLYWDDLKRKLSEKLDTTDFTSTIKLLNENLYVPREAGSQKDENLALYIENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTGGSSGLPNIPVQTISRAAAEKLFGNMEGDCPSDWKTDSTCRMVTSESKNVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGL

SEQ ID NO: 472 - protein, 528 aa, synthetic construct
HHHHHHHHGLNDIFEAQKIEWHEGGLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTGGSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGL

SEQ ID NO: 473 - protein, 454 aa, synthetic construct
QSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 474 - protein, 454 aa, synthetic construct
QSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 475 - protein, 455 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 476 - protein, 455 aa, synthetic construct
EVQLVESGGGLVQPGGSLRLSCAASGIDFDSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 477 - protein, 455 aa, synthetic construct
EVQLVESGGGLVQPGGSLRLSCAASGIDFSDSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 478 - protein, 455 aa, synthetic construct
EVQLVESGGGLVQPGGSLRLSCAASGIDFSESGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 479 - protein, 455 aa, synthetic construct
EVQLVESGGGLVQPGGSLRLSCAASGIDFSSAGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 480 - protein, 455 aa, synthetic construct
EVQLVESGGGLVQPGGSLRLSCAASGIDFSSDGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 481 - protein, 455 aa, synthetic construct
EVQLVESGGGLVQPGGSLRLSCAASGIDFSSSGYMCWVRQAPGKGLEWVGCIATYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 482 - protein, 455 aa, synthetic construct
EVQLVESGGGLVQPGGSLRLSCAASGIDFSSSGYMCWVRQAPGKGLEWVGCIDTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 483 - protein, 455 aa, synthetic construct
EVQLVESGGGLVQPGGSLRLSCAASGIDFSSSGYMCWVRQAPGKGLEWVGCIHTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 484 - protein, 455 aa, synthetic construct
EVQLVESGGGLVQPGGSLRLSCAASGIDFSSSGYMCWVRQAPGKGLEWVGCIYTDSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 485 - protein, 455 aa, synthetic construct
EVQLVESGGGLVQPGGSLRLSCAASGIDFSSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 486 - protein, 455 aa, synthetic construct
EVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 487 - protein, 455 aa, synthetic construct
EVQLVESGGGLVQPGGSLRLSCAASGIDFSSSGYMCWVRQAPGKGLEWVGCIYTYSSNTAYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 488 - protein, 455 aa, synthetic construct
EVQLVESGGGLVQPGGSLRLSCAASGIDFSSSGYMCWVRQAPGKGLEWVGCIYTYSSNTDYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 489 - protein, 455 aa, synthetic construct
EVQLVESGGGLVQPGGSLRLSCAASGIDFSSSGYMCWVRQAPGKGLEWVGCIYTYSSNTEYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 490 - protein, 455 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGATGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 491 - protein, 455 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGDTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 492 - protein, 455 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGATYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 493 - protein, 455 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGDTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 494 - protein, 455 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYDYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 495 - protein, 455 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTDTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 496 - protein, 455 aa, synthetic construct
EVQLVESGGGLVQPGGSLRLSCAASGIDFDSSGYMCWVRQAPGKGLEWVGCIYTDSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 497 - protein, 455 aa, synthetic construct
EVQLVESGGGLVQPGGSLRLSCAASGIDFDSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 498 - protein, 455 aa, synthetic construct
EVQLVESGGGLVQPGGSLRLSCAASGIDFDSSGYMCWVRQAPGKGLEWVGCIYTYSSNTDYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 499 - protein, 455 aa, synthetic construct
EVQLVESGGGLVQPGGSLRLSCAASGIDFDSSGYMCWVRQAPGKGLEWVGCIYTYSSNTEYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 500 - protein, 455 aa, synthetic construct
EVQLVESGGGLVQPGGSLRLSCAASGIDFDSSGYMCWVRQAPGKGLEWVGCIHTDSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 501 - protein, 455 aa, synthetic construct
EVQLVESGGGLVQPGGSLRLSCAASGIDFDSSGYMCWVRQAPGKGLEWVGCIYTYSSNTYYAASVKGRFTISKTSSTTVYLQMNSLKTEDTAVYYCARGTYGYTGATYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 502 - protein, 455 aa, synthetic construct
EVQLVESGGGLVQPGGSLRLSCAASGIDFSSSGYMCWVRQAPGKGLEWVGCIYTYSSNTAYAASVKGRFTISKTSSTTVYLQMNSLKTEDTAVYYCARGTYGYTGATYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 503 - protein, 455 aa, synthetic construct
EVQLVESGGGLVQPGGSLRLSCAASGIDFSSSGYMCWVRQAPGKGLEWVGCIYTYSSNTEYAASVKGRFTISKTSSTTVYLQMNSLKTEDTAVYYCARGTYGYTGATYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 504 - protein, 455 aa, synthetic construct
EVQLVESGGGLVQPGGSLRLSCAASGIDFSSSGYMAWVRQAPGKGLEWVGAIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 505 - protein, 455 aa, synthetic construct
EVQLVESGGGLVQPGGSLRLSCAASGIDFSSSGYMAWVRQAPGKGLEWVGAIYTYSSDTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 506 - protein, 455 aa, synthetic construct
EVQLVESGGGLVQPGGSLRLSCAASGIDFSSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 507 - protein, 463 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGQSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 508 - protein, 452 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPQSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 509 - protein, 463 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGQSLVESGGGLVQPGGSLRLSCAASGIDFASSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 510 - protein, 463 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGQSLVESGGGLVQPGGSLRLSCAASGIDFSASGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 511 - protein, 463 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGQSLVESGGGLVQPGGSLRLSCAASGIDFSSAGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 512 - protein, 463 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGQSLVESGGGLVQPGGSLRLSCAASGIDFSSSGYMCWVRQAPGKGLEWVGCIATYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 513 - protein, 463 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGQSLVESGGGLVQPGGSLRLSCAASGIDFSSSGYMCWVRQAPGKGLEWVGCIYTASSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 514 - protein, 463 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGQSLVESGGGLVQPGGSLRLSCAASGIDFSSSGYMCWVRQAPGKGLEWVGCIYTYSSATYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 515 - protein, 463 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGQSLVESGGGLVQPGGSLRLSCAASGIDFSSSGYMCWVRQAPGKGLEWVGCIYTYSSNTAYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 516 - protein, 463 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGQSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGATGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 517 - protein, 463 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGQSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGATYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 518 - protein, 463 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGQSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYAYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 519 - protein, 463 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGQSLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTATMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 520 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 521 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFASSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 522 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFDSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 523 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFHSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 524 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSASGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 525 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSDSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 526 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSHSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 527 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSAGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 528 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSDGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 529 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSHGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 530 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIATYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 531 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIDTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 532 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIHTYSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 533 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTASSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 534 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTDSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 535 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTHSSNTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 536 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSATYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 537 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 538 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSHTYYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 539 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTAYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 540 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTDYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 541 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTHYAASVKGRFTISKTSSTTVYLQMNSLKTEDTAVYYCARGTYGYTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 542 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGATGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 543 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGDTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 544 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGHTGYTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 545 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGATYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 546 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGDTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 547 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGHTYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 548 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYAYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 549 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYDYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 550 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYHYTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 551 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTATMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 552 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTDTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 553 - protein, 464 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGEVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTHTMGYFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 554 - protein, 482 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGEVQLVESGGGLVQPGGSLRLSCAASGIDFSSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSSGGGGSGGGGSGGGGSDIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGGGTKVEIK

SEQ ID NO: 555 - protein, 482 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGEVQLVESGGGLVQPGGSLRLSCAASGIDFSSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSSGGGGSGGGGSGGGGSDIQMTQSPSTLSASVGDRVTITCQASQNINSYLAWYQQKPGKAPKLLIYDASSLESGVPSRFSGSGSGTEFTLTISSLQPDDFATYYCQSYYYSGSSNYNAFGGGTKVEIK

SEQ ID NO: 556 - protein, 482 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGDIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGGGTKVEIKGGGGSGGGGSGGGGSEVQLVESGGGLVQPGGSLRLSCAASGIDFSSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSS

SEQ ID NO: 557 - protein, 482 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGDIQMTQSPSTLSASVGDRVTITCQASQNINSYLAWYQQKPGKAPKLLIYDASSLESGVPSRFSGSGSGTEFTLTISSLQPDDFATYYCQSYYYSGSSNYNAFGGGTKVEIKGGGGSGGGGSGGGGSEVQLVESGGGLVQPGGSLRLSCAASGIDFSSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSS

SEQ ID NO: 558 - protein, 482 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGDIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGGGTKVEIKGGGGSGGGGSGGGGSEVQLVESGGGLVQPGGSLRLSCAASGIDFSSSGYMAWVRQAPGKGLEWVGAIYTYSSDTYYAASVKGRFTISKTSSTTVYLQMNSLKTEDTAVYYCARGTYGYTGYTYTMGYFSLWGQGTLVTVSS

SEQ ID NO: 559 - protein, 482 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGDIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGCGTKVEIKGGGGSGGGGSGGGGSEVQLVESGGGLVQPGGSLRLSCAASGIDFSSSGYMCWVRQAPGKCLEWVGCIYTYSSDTYYAASVKGRFTISKTSSTTVYLQMNSLKTEDTAVYYCARGTYGYTGYTYTMGYFSLWGQGTLVTVSS

SEQ ID NO: 560 - protein, 482 aa, synthetic construct
KTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGDIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGCGTKVEIKGGGGSGGGGSGGGGSEVQLVESGGGLVQPGGSLRLSCAASGIDFSSSGYMAWVRQAPGKCLEWVGAIYTYSSDTYYAASVKGRFTISKTSSTTVYLQMNSLKTEDTAVYYCARGTYGYTGYTYTMGYFSLWGQGTLVTVSS

SEQ ID NO: 561 - protein, 218 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 562 - protein, 229 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTGYPYDVPDYA

SEQ ID NO: 563 - protein, 218 aa, synthetic construct
DIQMTQSPSTLSASVGDRVTITCQASQSINSYLAWYQQKPGKAPKLLIYRASSLESGVPSRFSGSGSGTEFTLTISSLQPDDFATYYCQSYYYSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 564 - protein, 218 aa, synthetic construct
DIQMTQSPSTLSASVGDRVTITCQASQNISSYLAWYQQKPGKAPKLLIYRASSLESGVPSRFSGSGSGTEFTLTISSLQPDDFATYYCQSYYYSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 565 - protein, 218 aa, synthetic construct
DIQMTQSPSTLSASVGDRVTITCQASQNINSYLAWYQQKPGKAPKLLIYDASSLESGVPSRFSGSGSGTEFTLTISSLQPDDFATYYCQSYYYSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 566 - protein, 218 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYDSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 567 - protein, 218 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYHSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 568 - protein, 218 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYSSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 569 - protein, 218 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGASNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 570 - protein, 218 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGDSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 571 - protein, 218 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGHSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 572 - protein, 218 aa, synthetic construct
DIQMTQSPSTLSASVGDRVTITCQASQSINSYLAWYQQKPGKAPKLLIYDASSLESGVPSRFSGSGSGTEFTLTISSLQPDDFATYYCQSYYYSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 573 - protein, 218 aa, synthetic construct
DIQMTQSPSTLSASVGDRVTITCQASQSINSYLAWYQQKPGKAPKLLIYRASSLESGVPSRFSGSGSGTEFTLTISSLQPDDFATYYCQSYYSSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 574 - protein, 218 aa, synthetic construct
DIQMTQSPSTLSASVGDRVTITCQASQSINSYLAWYQQKPGKAPKLLIYRASSLESGVPSRFSGSGSGTEFTLTISSLQPDDFATYYCQSYYDSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 575 - protein, 218 aa, synthetic construct
DIQMTQSPSTLSASVGDRVTITCQASQSINSYLAWYQQKPGKAPKLLIYRASSLESGVPSRFSGSGSGTEFTLTISSLQPDDFATYYCQSYYYSGDSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 576 - protein, 218 aa, synthetic construct
DIQMTQSPSTLSASVGDRVTITCQASQSINSYLAWYQQKPGKAPKLLIYDASSLESGVPSRFSGSGSGTEFTLTISSLQPDDFATYYCQSYYSSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 577 - protein, 218 aa, synthetic construct
DIQMTQSPSTLSASVGDRVTITCQASQSINSYLAWYQQKPGKAPKLLIYDASSLESGVPSRFSGSGSGTEFTLTISSLQPDDFATYYCQSYYYSGDSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 578 - protein, 217 aa, synthetic construct
DIQMTQSPSTLSASVGDRVTITCQASQNINSYLAWYQQKPGKAPKLLIYRASSLESGVPSRFSGSGSGTEFTLTISSLQPDDFATYYCQSYYYSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 579 - protein, 217 aa, synthetic construct
DIQMTQSPSTLSASVGDRVTITCQASQNINSYLAWYQQKPGKAPKLLIYDASSLESGVPSRFSGSGSGTEFTLTISSLQPDDFATYYCQSYYYSGSSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 580 - protein, 452 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGATYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 581 - protein, 452 aa, synthetic construct
EVQLVESGGGLVQPGGSLRLSCAASGIDFSSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 582 - protein, 452 aa, synthetic construct
EVQLVESGGGLVQPGGSLRLSCAASGIDFDSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 583 - protein, 452 aa, synthetic construct
EVQLVESGGGLVQPGGSLRLSCAASGIDFSSSGYMCWVRQAPGKGLEWVGCIYTYSSNTEYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 584 - protein, 452 aa, synthetic construct
EVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 585 - protein, 452 aa, synthetic construct
EVQLVESGGGLVQPGGSLRLSCAASGIDFSSSGYMCWVRQAPGKGLEWVGCIATYSSNTYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 586 - protein, 452 aa, synthetic construct
EVQLVESGGGLVQPGGSLRLSCAASGIDFSSSGYMCWVRQAPGKGLEWVGCIDTYSSNTYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 587 - protein, 452 aa, synthetic construct
EVQLVESGGGLVQPGGSLRLSCAASGIDFSSSGYMCWVRQAPGKGLEWVGCIYTDSSETYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 588 - protein, 452 aa, synthetic construct
EVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGATYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 589 - protein, 452 aa, synthetic construct
EVQLVESGGGLVQPGGSLRLSCAASGIDFSSSGYMCWVRQAPGKGLEWVGCIYTYSSETEYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 590 - protein, 452 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 591 - protein, 452 aa, synthetic construct
EVQLVESGGGLVQPGGSLRLSCAASGIDFSSDGYMCWVRQAPGKGLEWVGCIYTYSSNTYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 592 - protein, 452 aa, synthetic construct
EVQLVESGGGLVQPGGSLRLSCAASGIDFSSSGYMCWVRQAPGKGLEWVGCIYTDSSNTYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 593 - protein, 452 aa, synthetic construct
EVQLVESGGGLVQPGGSLRLSCAASGIDFSSSGYMCWVRQAPGKGLEWVGCIYTYSSNTYYAASVKGRFTISKTSSTTVYLQMNSLKTEDTAVYYCARGTYGYTGATYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 594 - protein, 452 aa, synthetic construct
EVQLVESGGGLVQPGGSLRLSCAASGIDFSSSGYMCWVRQAPGKGLEWVGCIYTYSSDTYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 595 - protein, 452 aa, synthetic construct
EVQLVESGGGLVQPGGSLRLSCAASGIDFSSSGYMCWVRQAPGKGLEWVGCIYTYSSNTEYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 596 - protein, 452 aa, synthetic construct
EVQLVESGGGLVQPGGSLRLSCAASGIDFSSSGYMCWVRQAPGKGLEWVGCIYTYSSETYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 597 - protein, 452 aa, synthetic construct
EVQLVESGGGLVQPGGSLRLSCAASGIDFSSSGYMCWVRQAPGKGLEWVGCIATYSSNTYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 598 - protein, 452 aa, synthetic construct
EVQLVESGGGLVQPGGSLRLSCAASGIDFSSSGYMCWVRQAPGKGLEWVGCIDTYSSNTYYAASVKGRFTISKTSSTTVYLQMNSLKTEDTAVYYCARGTYGYTGYTYTMGYFSLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 599 - protein, 218 aa, synthetic construct
DIQMTQSPSTLSASVGDRVTITCQASQSISSYLAWYQQKPGKAPKLLIYRASSLESGVPSRFSGSGSGTEFTLTISSLQPDDFATYYCQSYYYSGDSNYNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 600 - protein, 227 aa, synthetic construct
SKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 601 - protein, 6 aa, synthetic construct
HHHHHH

SEQ ID NO: 602 - protein, 8 aa, synthetic construct
HHHHHHHH

SEQ ID NO: 603 - protein, 4 aa, synthetic construct
EVQL

SEQ ID NO: 604 - protein, 12 aa, synthetic construct
GAGAGAGAGAGA

(19) United States
(12) Patent Application Publication
(10) Pub. No.: US 2026/0242498 A1
(43) Pub. Date: Aug. 20, 2026

(54) ANTI-TRANSFERRIN RECEPTOR ANTIBODIES AND USES THEREOF

(71) Applicant: Biogen MA Inc., Cambridge, MA (US)

(72) Inventors: Thomas Owen Cameron, Cambridge, MA (US); Karl John Mortley Hanf, Billerica, MA (US); Benjamin Andrew Smith, Belmont, MA (US); Joseph Walter Arndt, Swampscott, MA (US); Caitlin Bryanna Meeks, Arlington, MA (US); Isin Dalkilic-Liddle, Watertown, MA (US); Andreas Lehmann, Belmont, MA (US); Mark Christopher Julian, Winthrop, MA (US)

(21) Appl. No.: 19/162,809

(22) PCT Filed: Mar. 7, 2024
(86) PCT No.: PCT/US2024/018978

§ 371 Date: Sep. 5, 2025

Related U.S. Application Data
(60) Provisional application No. 63/450,906, filed on Mar. 8, 2023.

(51) Int. Cl. C07K 16/28; A61K 39/00; A61K 47/68; A61K 47/69; C07K 16/18

(52) U.S. Cl. CPC C07K 16/2881; A61K 47/6807; A61K 47/6815; A61K 47/6849; A61K 47/6901; A61K 47/6913; A61K 47/6929; C07K 16/18; C12Y 302/01045; A61K 2039/505; C07K 2317/33; C07K 2317/52; C07K 2317/565; C07K 2317/92; C07K 2319/42

(57) ABSTRACT

The present disclosure provides anti-transferrin receptor antibodies, compositions comprising the same and methods of use for delivery of cargo to brain tissue. This disclosure also provides polynucleotides and vectors encoding the anti-transferrin receptor antibodies and cells comprising the same, methods of making the antibodies, and molecules comprising the antibodies.

### CROSS REFERENCE TO RELATED APPLICATIONS

The present application claims the benefit of U.S. Provisional Application No. 63/450,906, filed Mar. 8, 2023, the entire contents of which are hereby incorporated by reference herein.

### TECHNICAL FIELD

This disclosure relates to anti-transferrin receptor antibodies, compositions comprising the same, and methods of use for the delivery of therapeutic cargo to brain tissue. This disclosure also provides related polynucleotides and vectors encoding the anti-transferrin receptor antibodies and cells comprising the same.

### BACKGROUND

The delivery of drugs to the central nervous system has been a challenge in the treatment of neurological diseases such as Alzheimer's disease and Parkinson's disease. For drugs to reach the brain, they first have to penetrate the blood brain barrier, which is a major challenge due to the selectivity of the blood brain barrier. The blood brain barrier acts as a semipermeable membrane, preventing most molecules from entering the nervous system from the blood and allows only low molecular weight (<400 Da) and lipophilic compounds to pass. Most small molecules and large molecules, such as monoclonal antibodies and antisense oligonucleotides, cannot pass through this barrier. Due to this challenging process of drug penetration across the blood brain barrier, a small fraction of therapeutic agents for neurological diseases make it to clinical trials.

There is a need in the art for improved compositions and methods for delivering a therapeutic agent to the central nervous system.

### SUMMARY

This disclosure relates to anti-transferrin receptor antibodies and methods of their use for the delivery of cargo to brain tissue and the treatment of neurological disorders.

In a first aspect, this disclosure features an antibody that binds to human transferrin receptor, comprising a heavy chain variable region (VH) comprising VH complementarity determining region (CDR) 1, VH CDR2, and VH CDR3, and a light chain variable region (VL) comprising VL CDR1, VL CDR2, and VL CDR3,
wherein (a) the VH CDR1, VH CDR2, VH CDR3, VL CDR1, VL CDR2, and VL CDR3 contain collectively one to ten amino acid differences as compared to a parental antibody whose heavy chain CDRs 1, 2, and 3 comprise the amino acid sequences GIDFSSSGYMC (SEQ ID NO: XX), CIYTYSSNTYYAASVKG (SEQ ID NO:XX), and GTYGYTGYTYTMGYFSL (SEQ ID NO: XX), and whose light chain CDRs 1, 2, and 3 comprise the amino acid sequences QASQNINSYLA (SEQ ID NO:XX), RASSLES (SEQ ID NO:XX), and QSYYYSGSSNYNA (SEQ ID NO:XX), or (b) the VH CDR1, VH CDR2, VH CDR3, VL CDR1, VL CDR2, and VL CDR3 contain collectively one to ten amino acid differences as compared to a parental antibody whose heavy chain CDRs 1, 2, and 3 comprise the amino acid sequences GIDFSSSG (SEQ ID NO: XX), TYSS (SEQ ID NO:XX), and TYGYTGYTYTMGYFS (SEQ ID NO:XX), and whose light chain CDRs 1, 2, and 3 comprise the amino acid sequences SQNINSY (SEQ ID NO:XX), RAS (SEQ ID NO:XX), and YYYSGSSNYN (SEQ ID NO:XX), and
wherein the one to ten amino acid differences as compared to the parental antibody are (using AHo numbering) selected from the group consisting of:
VH-S32A, VH-S32D, or VH-S32H;
VH-S33A or VH-S33D;
VH-S33E or VH-S33H;
VH-S38A, VH-S38D, or VH-S38H;
VH-Y59A, VH-Y59D, VH-Y59H, VH-Y59E, or VH-Y59F;
VH-Y61A, VH-Y61D, or VH-Y61H;
VH-N67A, VH-N67D, VH-N67E, VH-N67H, VH-N67K, or VH-N67R;
VH-Y69A, VH-Y69D, VH-Y69E, or VH-Y69H;
VH-Y113A, VH-Y113D, or VH-Y113H;
VH-Y116A, VH-Y116D, or VH-Y116H;
VH-T130A, VH-T130D, or VH-T130H;
VH-Y131A, VH-Y131D, or VH-Y131H;
VL-N30S;
VL-N32S;
VL-R58D;
VL-Y111D, VL-Y111H, or VL-Y111S;
VL-S133A, VL-S133D, or VL-S133H;
VH-Y61D and VH-N67E;
VH-N67E and VH-Y69E; and
VL-N30S and VL-N32S.

In some embodiments, the VH CDR1, VH CDR2, VH CDR3, VL CDR1, VL CDR2, and VL CDR3 contain collectively two amino acid differences as compared to the parental antibody.

In some embodiments, the VH CDR1, VH CDR2, VH CDR3, VL CDR1, VL CDR2, and VL CDR3 contain collectively three amino acid differences as compared to the parental antibody.

In some embodiments, the VH CDR1, VH CDR2, VH CDR3, VL CDR1, VL CDR2, and VL CDR3 contain collectively four amino acid differences as compared to the parental antibody.

In some embodiments, the VH CDR1, VH CDR2, VH CDR3, VL CDR1, VL CDR2, and VL CDR3 contain collectively five amino acid differences as compared to the parental antibody.

In some embodiments, the VH CDR1, VH CDR2, VH CDR3, VL CDR1, VL CDR2, and VL CDR3 contain collectively six amino acid differences as compared to the parental antibody.

In some embodiments:
(a) at least one of VH CDR1, VH CDR2, VH CDR3, VL CDR1, VL CDR2, and/or VL CDR3 are selected from the mutant CDRs depicted in Table 1, and any of VH CDR1, VH CDR2, VH CDR3, VL CDR1, VL CDR2, or VL CDR3 that are not selected from the mutant CDRs depicted in Table 1 are selected from the parental CDRs depicted in Table 1; or
(b) at least one of VH CDR1, VH CDR2, VH CDR3, VL CDR1, VL CDR2, and/or VL CDR3 are selected from the mutant CDRs depicted in Table 2, and any of VH CDR1, VH CDR2, VH CDR3, VL CDR1, VL CDR2, or VL CDR3 that are not selected from the mutant CDRs depicted in Table 2 are selected from the parental CDRs depicted in Table 2.

In some embodiments:
(a) one of VH CDR1, VH CDR2, VH CDR3, VL CDR1, VL CDR2, or VL CDR3 are selected from the mutant CDRs depicted in Table 1 and five of VH CDR1, VH CDR2, VH CDR3, VL CDR1, VL CDR2, and/or VL CDR3 are selected from the parental CDRs depicted in Table 1; or
(b) one of VH CDR1, VH CDR2, VH CDR3, VL CDR1, VL CDR2, or VL CDR3 are selected from the mutant CDRs depicted in Table 2 and five of VH CDR1, VH CDR2, VH CDR3, VL CDR1, VL CDR2, and/or VL CDR3 are selected from the parental CDRs depicted in Table 2.

In some embodiments:
(a) two of VH CDR1, VH CDR2, VH CDR3, VL CDR1, VL CDR2, and/or VL CDR3 are selected from the mutant CDRs depicted in Table 1 and four of VH CDR1, VH CDR2, VH CDR3, VL CDR1, VL CDR2, and/or VL CDR3 are selected from the parental CDRs depicted in Table 1; or
(b) two of VH CDR1, VH CDR2, VH CDR3, VL CDR1, VL CDR2, and/or VL CDR3 are selected from the mutant CDRs depicted in Table 2 and four of VH CDR1, VH CDR2, VH CDR3, VL CDR1, VL CDR2, and/or VL CDR3 are selected from the parental CDRs depicted in Table 2.

In some embodiments:
(a) three of VH CDR1, VH CDR2, VH CDR3, VL CDR1, VL CDR2, and/or VL CDR3 are selected from the mutant CDRs depicted in Table 1 and three of VH CDR1, VH CDR2, VH CDR3, VL CDR1, VL CDR2, and/or VL CDR3 are selected from the parental CDRs depicted in Table 1; or
(b) three of VH CDR1, VH CDR2, VH CDR3, VL CDR1, VL CDR2, and/or VL CDR3 are selected from the mutant CDRs depicted in Table 2 and three of VH CDR1, VH CDR2, VH CDR3, VL CDR1, VL CDR2, and/or VL CDR3 are selected from the parental CDRs depicted in Table 2.

In some embodiments:
(a) four of VH CDR1, VH CDR2, VH CDR3, VL CDR1, VL CDR2, and/or VL CDR3 are selected from the mutant CDRs depicted in Table 1 and two of VH CDR1, VH CDR2, VH CDR3, VL CDR1, VL CDR2, and/or VL CDR3 are selected from the parental CDRs depicted in Table 1; or
(b) four of VH CDR1, VH CDR2, VH CDR3, VL CDR1, VL CDR2, and/or VL CDR3 are selected from the mutant CDRs depicted in Table 2 and two of VH CDR1, VH CDR2, VH CDR3, VL CDR1, VL CDR2, and/or VL CDR3 are selected from the parental CDRs depicted in Table 2.

In some embodiments:
(a) five of VH CDR1, VH CDR2, VH CDR3, VL CDR1, VL CDR2, and/or VL CDR3 are selected from the mutant CDRs depicted in Table 1 and one of VH CDR1, VH CDR2, VH CDR3, VL CDR1, VL CDR2, or VL CDR3 are selected from the parental CDRs depicted in Table 1; or
(b) five of VH CDR1, VH CDR2, VH CDR3, VL CDR1, VL CDR2, and/or VL CDR3 are selected from the mutant CDRs depicted in Table 2 and one of VH CDR1, VH CDR2, VH CDR3, VL CDR1, VL CDR2, or VL CDR3 are selected from the parental CDRs depicted in Table 2.

In some embodiments:
(a) all of VH CDR1, VH CDR2, VH CDR3, VL CDR1, VL CDR2, and VL CDR3 are selected from the mutant CDRs depicted in Table 1; or
(b) all of VH CDR1, VH CDR2, VH CDR3, VL CDR1, VL CDR2, and VL CDR3 are selected from the mutant CDRs depicted in Table 2.

In some embodiments:
the VH CDR1 comprises the amino acid sequence GIDFASSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFDSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFHSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSASGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSDSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSESGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSHSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSAGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSDGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSHGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIATYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIDTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIHTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIETYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIFTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTASSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTDSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIHTDSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTHSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSATYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSETYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSHTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSKTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSRTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTAYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTDYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTEYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTHYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGATGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGDTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGHTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGATYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGDTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGHTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYAYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYDYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYHYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTATMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTDTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTHTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQSINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNISSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence DASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYDSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYHSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYSSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGASNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGDSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGHSNYNA (SEQ ID NO:XX);
the VH CDR1 comprises the amino acid sequence GIDFSSSGYMC (SEQ ID NO:XX); the VH CDR2 comprises the amino acid sequence CIYTDSSETYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSETEYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX); or
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQSISSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX).

In some embodiments:
(a) the VH CDR1 comprises the amino acid sequence GIDFDSSGYMC (SEQ ID NO: XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence DASSLES (SEQ ID NO:XX); and the VL CDR3 comprises the amino acid sequence QSYYYSGSSNYNA (SEQ ID NO:XX);
(b) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence DASSLES (SEQ ID NO:XX); and the VL CDR3 comprises the amino acid sequence QSYYYSGSSNYNA (SEQ ID NO:XX);
(c) the VH CDR1 comprises the amino acid sequence GIDFDSSGYMC (SEQ ID NO: XX); the VH CDR2 comprises the amino acid sequence CIYTDSSNTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(d) the VH CDR1 comprises the amino acid sequence GIDFDSSGYMC (SEQ ID NO: XX); the VH CDR2 comprises the amino acid sequence CIYTYSSETYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(e) the VH CDR1 comprises the amino acid sequence GIDFDSSGYMC (SEQ ID NO: XX); the VH CDR2 comprises the amino acid sequence CIYTYSSNTDYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(f) the VH CDR1 comprises the amino acid sequence GIDFDSSGYMC (SEQ ID NO: XX); the VH CDR2 comprises the amino acid sequence CIYTYSSNTEYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(g) the VH CDR1 comprises the amino acid sequence GIDFDSSGYMC (SEQ ID NO: XX); the VH CDR2 comprises the amino acid sequence CIHTDSSNTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(h) the VH CDR1 comprises the amino acid sequence GIDFDSSGYMC (SEQ ID NO: XX); the VH CDR2 comprises the amino acid sequence CIYTYSSNTYYAASVKG (SEQ ID NO: XX); the VH CDR3 comprises the amino acid sequence GTYGYTGATYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(i) the VH CDR1 comprises the amino acid sequence GIDFSSSGYMC (SEQ ID NO: XX); the VH CDR2 comprises the amino acid sequence CIYTYSSNTAYAASVKG (SEQ ID NO: XX); the VH CDR3 comprises the amino acid sequence GTYGYTGATYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(j) the VH CDR1 comprises the amino acid sequence GIDFSSSGYMC (SEQ ID NO: XX); the VH CDR2 comprises the amino acid sequence CIYTYSSNTEYAASVKG (SEQ ID NO: XX); the VH CDR3 comprises the amino acid sequence GTYGYTGATYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(k) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence DASSLES (SEQ ID NO:XX); and the VL CDR3 comprises the amino acid sequence QSYYYSGSSNYNA (SEQ ID NO:XX);
(l) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence RASSLES (SEQ ID NO:XX); and the VL CDR3 comprises the amino acid sequence QSYYSSGSSNYNA (SEQ ID NO:XX);
(m) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence RASSLES (SEQ ID NO:XX); and the VL CDR3 comprises the amino acid sequence QSYYDSGSSNYNA (SEQ ID NO:XX);
(n) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence RASSLES (SEQ ID NO:XX); and the VL CDR3 comprises the amino acid sequence QSYYYSGDSNYNA (SEQ ID NO:XX);
(o) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence DASSLES (SEQ ID NO:XX); and the VL CDR3 comprises the amino acid sequence QSYYSSGSSNYNA (SEQ ID NO:XX);
(p) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence DASSLES (SEQ ID NO:XX); and the VL CDR3 comprises the amino acid sequence QSYYYSGDSNYNA (SEQ ID NO:XX);
(q) the VH CDR1 comprises the amino acid sequence GIDFDSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(r) the VH CDR1 comprises the amino acid sequence GIDFSSSGYMC (SEQ ID NO: XX); the VH CDR2 comprises the amino acid sequence CIYTYSSETYYAASVKG (SEQ ID NO: XX); the VH CDR3 comprises the amino acid sequence GTYGYTGATYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(s) the VH CDR1 comprises the amino acid sequence GIDFSSSGYMC (SEQ ID NO: XX); the VH CDR2 comprises the amino acid sequence CIYTYSSETYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSISSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(t) the VH CDR1 comprises the amino acid sequence GIDFSSSGYMC (SEQ ID NO: XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSISSYLA (SEQ ID NO: XX); the VL CDR2 comprises the amino acid sequence RASSLES (SEQ ID NO:XX); and the VL CDR3 comprises the amino acid sequence QSYYYSGDSNYNA (SEQ ID NO:XX); or
(u) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSISSYLA (SEQ ID NO: XX); the VL CDR2 comprises the amino acid sequence RASSLES (SEQ ID NO:XX); and the VL CDR3 comprises the amino acid sequence QSYYYSGDSNYNA (SEQ ID NO:XX).

In some embodiments:
(i) the VH is at least 80%, 85%, 90%, 95%, 99%, or 100% identical to any one of SEQ ID NOs: 100-160; and
(ii) the VL is at least 80%, 85%, 90%, 95%, 99%, or 100% identical to any one of SEQ ID NOs: 200-216.

In some embodiments, the VH comprises the amino acid sequence of any one of SEQ ID NOs: 100-160 and the VL comprises the amino acid sequence of any one of SEQ ID NOs: 200-216.

In some embodiments, the antibody is (a) monovalent and has a monovalent affinity (K_{D}) for hTfR1 of >10 nM or is bivalent and has a monovalent affinity (K_{D}) for hTfR1 of >100 nM and/or (b) has an off rate (k_{d}) of >=0.01/s.

In some embodiments:
the VH comprises the amino acid sequence of SEQ ID NO: 100 and the VL comprises the amino acid sequence of SEQ ID NO:301;
the VH comprises the amino acid sequence of SEQ ID NO: 101 and the VL comprises the amino acid sequence of SEQ ID NO:301;
the VH comprises the amino acid sequence of SEQ ID NO: 102 and the VL comprises the amino acid sequence of SEQ ID NO:301;
the VH comprises the amino acid sequence of SEQ ID NO: 103 and the VL comprises the amino acid sequence of SEQ ID NO:301;
the VH comprises the amino acid sequence of SEQ ID NO: 104 and the VL comprises the amino acid sequence of SEQ ID NO:301;
the VH comprises the amino acid sequence of SEQ ID NO: 105 and the VL comprises the amino acid sequence of SEQ ID NO:301;
the VH comprises the amino acid sequence of SEQ ID NO: 106 and the VL comprises the amino acid sequence of SEQ ID NO:301;
the VH comprises the amino acid sequence of SEQ ID NO: 107 and the VL comprises the amino acid sequence of SEQ ID NO:301;
the VH comprises the amino acid sequence of SEQ ID NO: 108 and the VL comprises the amino acid sequence of SEQ ID NO:301;
the VH comprises the amino acid sequence of SEQ ID NO: 109 and the VL comprises the amino acid sequence of SEQ ID NO:301;
the VH comprises the amino acid sequence of SEQ ID NO: 110 and the VL comprises the amino acid sequence of SEQ ID NO:301;
the VH comprises the amino acid sequence of SEQ ID NO:111 and the VL comprises the amino acid sequence of SEQ ID NO:301;
the VH comprises the amino acid sequence of SEQ ID NO:112 and the VL comprises the amino acid sequence of SEQ ID NO:301;
the VH comprises the amino acid sequence of SEQ ID NO: 113 and the VL comprises the amino acid sequence of SEQ ID NO:301;
the VH comprises the amino acid sequence of SEQ ID NO:114 and the VL comprises the amino acid sequence of SEQ ID NO:301;
the VH comprises the amino acid sequence of SEQ ID NO: 115 and the VL comprises the amino acid sequence of SEQ ID NO:301;
the VH comprises the amino acid sequence of SEQ ID NO:116 and the VL comprises the amino acid sequence of SEQ ID NO:301;
the VH comprises the amino acid sequence of SEQ ID NO:117 and the VL comprises the amino acid sequence of SEQ ID NO:301;
the VH comprises the amino acid sequence of SEQ ID NO: 118 and the VL comprises the amino acid sequence of SEQ ID NO:301;
the VH comprises the amino acid sequence of SEQ ID NO: 119 and the VL comprises the amino acid sequence of SEQ ID NO:301;
the VH comprises the amino acid sequence of SEQ ID NO: 120 and the VL comprises the amino acid sequence of SEQ ID NO:301;
the VH comprises the amino acid sequence of SEQ ID NO: 121 and the VL comprises the amino acid sequence of SEQ ID NO:301;
the VH comprises the amino acid sequence of SEQ ID NO: 122 and the VL comprises the amino acid sequence of SEQ ID NO:301;
the VH comprises the amino acid sequence of SEQ ID NO: 123 and the VL comprises the amino acid sequence of SEQ ID NO:301;
the VH comprises the amino acid sequence of SEQ ID NO: 124 and the VL comprises the amino acid sequence of SEQ ID NO:301;
the VH comprises the amino acid sequence of SEQ ID NO: 125 and the VL comprises the amino acid sequence of SEQ ID NO:301;
the VH comprises the amino acid sequence of SEQ ID NO: 126 and the VL comprises the amino acid sequence of SEQ ID NO:301;
the VH comprises the amino acid sequence of SEQ ID NO: 127 and the VL comprises the amino acid sequence of SEQ ID NO:301;
the VH comprises the amino acid sequence of SEQ ID NO: 128 and the VL comprises the amino acid sequence of SEQ ID NO:301;
the VH comprises the amino acid sequence of SEQ ID NO:129 and the VL comprises the amino acid sequence of SEQ ID NO:301;
the VH comprises the amino acid sequence of SEQ ID NO: 130 and the VL comprises the amino acid sequence of SEQ ID NO:301;
the VH comprises the amino acid sequence of SEQ ID NO: 131 and the VL comprises the amino acid sequence of SEQ ID NO:301;
the VH comprises the amino acid sequence of SEQ ID NO: 132 and the VL comprises the amino acid sequence of SEQ ID NO:301;
the VH comprises the amino acid sequence of SEQ ID NO: 133 and the VL comprises the amino acid sequence of SEQ ID NO:301;
the VH comprises the amino acid sequence of SEQ ID NO: 134 and the VL comprises the amino acid sequence of SEQ ID NO:301;
the VH comprises the amino acid sequence of SEQ ID NO: 135 and the VL comprises the amino acid sequence of SEQ ID NO:301;
the VH comprises the amino acid sequence of SEQ ID NO: 136 and the VL comprises the amino acid sequence of SEQ ID NO:301;
the VH comprises the amino acid sequence of SEQ ID NO: 137 and the VL comprises the amino acid sequence of SEQ ID NO:301;
the VH comprises the amino acid sequence of SEQ ID NO: 138 and the VL comprises the amino acid sequence of SEQ ID NO:301;
the VH comprises the amino acid sequence of SEQ ID NO: 139 and the VL comprises the amino acid sequence of SEQ ID NO:301;
the VH comprises the amino acid sequence of SEQ ID NO: 140 and the VL comprises the amino acid sequence of SEQ ID NO:301;
the VH comprises the amino acid sequence of SEQ ID NO: 141 and the VL comprises the amino acid sequence of SEQ ID NO:301;
the VH comprises the amino acid sequence of SEQ ID NO: 142 and the VL comprises the amino acid sequence of SEQ ID NO:301;
the VH comprises the amino acid sequence of SEQ ID NO:143 and the VL comprises the amino acid sequence of SEQ ID NO:301;
the VH comprises the amino acid sequence of SEQ ID NO: 144 and the VL comprises the amino acid sequence of SEQ ID NO:301;
the VH comprises the amino acid sequence of SEQ ID NO: 145 and the VL comprises the amino acid sequence of SEQ ID NO:301;
the VH comprises the amino acid sequence of SEQ ID NO: 146 and the VL comprises the amino acid sequence of SEQ ID NO:301;
the VH comprises the amino acid sequence of SEQ ID NO: 147 and the VL comprises the amino acid sequence of SEQ ID NO:301;
the VH comprises the amino acid sequence of SEQ ID NO: 148 and the VL comprises the amino acid sequence of SEQ ID NO:301;
the VH comprises the amino acid sequence of SEQ ID NO: 149 and the VL comprises the amino acid sequence of SEQ ID NO:301;
the VH comprises the amino acid sequence of SEQ ID NO: 150 and the VL comprises the amino acid sequence of SEQ ID NO:301;
the VH comprises the amino acid sequence of SEQ ID NO:151 and the VL comprises the amino acid sequence of SEQ ID NO:301;
the VH comprises the amino acid sequence of SEQ ID NO:152 and the VL comprises the amino acid sequence of SEQ ID NO:301;
the VH comprises the amino acid sequence of SEQ ID NO: 153 and the VL comprises the amino acid sequence of SEQ ID NO:301;
the VH comprises the amino acid sequence of SEQ ID NO: 154 and the VL comprises the amino acid sequence of SEQ ID NO:301;
the VH comprises the amino acid sequence of SEQ ID NO: 155 and the VL comprises the amino acid sequence of SEQ ID NO:301;
the VH comprises the amino acid sequence of SEQ ID NO: 156 and the VL comprises the amino acid sequence of SEQ ID NO:301;
the VH comprises the amino acid sequence of SEQ ID NO: 157 and the VL comprises the amino acid sequence of SEQ ID NO:301;
the VH comprises the amino acid sequence of SEQ ID NO: 158 and the VL comprises the amino acid sequence of SEQ ID NO:301;
the VH comprises the amino acid sequence of SEQ ID NO: 159 and the VL comprises the amino acid sequence of SEQ ID NO:301;
the VH comprises the amino acid sequence of SEQ ID NO: 160 and the VL comprises the amino acid sequence of SEQ ID NO:301;
the VH comprises the amino acid sequence of SEQ ID NO:300 and the VL comprises the amino acid sequence of SEQ ID NO:200;
the VH comprises the amino acid sequence of SEQ ID NO:300 and the VL comprises the amino acid sequence of SEQ ID NO:201;
the VH comprises the amino acid sequence of SEQ ID NO:300 and the VL comprises the amino acid sequence of SEQ ID NO:202;
the VH comprises the amino acid sequence of SEQ ID NO:300 and the VL comprises the amino acid sequence of SEQ ID NO:203;
the VH comprises the amino acid sequence of SEQ ID NO:300 and the VL comprises the amino acid sequence of SEQ ID NO:204;
the VH comprises the amino acid sequence of SEQ ID NO:300 and the VL comprises the amino acid sequence of SEQ ID NO:205;
the VH comprises the amino acid sequence of SEQ ID NO:300 and the VL comprises the amino acid sequence of SEQ ID NO:206;
the VH comprises the amino acid sequence of SEQ ID NO:300 and the VL comprises the amino acid sequence of SEQ ID NO:207;
the VH comprises the amino acid sequence of SEQ ID NO:300 and the VL comprises the amino acid sequence of SEQ ID NO:208;
the VH comprises the amino acid sequence of SEQ ID NO:300 and the VL comprises the amino acid sequence of SEQ ID NO:209;
the VH comprises the amino acid sequence of SEQ ID NO:300 and the VL comprises the amino acid sequence of SEQ ID NO:210;
the VH comprises the amino acid sequence of SEQ ID NO:300 and the VL comprises the amino acid sequence of SEQ ID NO:211;
the VH comprises the amino acid sequence of SEQ ID NO:300 and the VL comprises the amino acid sequence of SEQ ID NO:212;
the VH comprises the amino acid sequence of SEQ ID NO:300 and the VL comprises the amino acid sequence of SEQ ID NO:213;
the VH comprises the amino acid sequence of SEQ ID NO:300 and the VL comprises the amino acid sequence of SEQ ID NO:214; or
the VH comprises the amino acid sequence of SEQ ID NO:300 and the VL comprises the amino acid sequence of SEQ ID NO:215;
the VH comprises the amino acid sequence of SEQ ID NO:300 and the VL comprises the amino acid sequence of SEQ ID NO:216.

In some embodiments:
(a) the VH comprises the amino acid sequence of SEQ ID NO:112 and the VL comprises the amino acid sequence of SEQ ID NO:202;
(b) the VH comprises the amino acid sequence of SEQ ID NO: 128 and the VL comprises the amino acid sequence of SEQ ID NO:202;
(c) the VH comprises the amino acid sequence of SEQ ID NO: 150 and the VL comprises the amino acid sequence of SEQ ID NO:301;
(d) the VH comprises the amino acid sequence of SEQ ID NO: 152 and the VL comprises the amino acid sequence of SEQ ID NO:301;
(e) the VH comprises the amino acid sequence of SEQ ID NO:153 and the VL comprises the amino acid sequence of SEQ ID NO:301;
(f) the VH comprises the amino acid sequence of SEQ ID NO: 154 and the VL comprises the amino acid sequence of SEQ ID NO:301;
(g) the VH comprises the amino acid sequence of SEQ ID NO:155 and the VL comprises the amino acid sequence of SEQ ID NO:301;
(h) the VH comprises the amino acid sequence of SEQ ID NO: 147 and the VL comprises the amino acid sequence of SEQ ID NO:301;
(i) the VH comprises the amino acid sequence of SEQ ID NO:148 and the VL comprises the amino acid sequence of SEQ ID NO:301;
(j) the VH comprises the amino acid sequence of SEQ ID NO:149 and the VL comprises the amino acid sequence of SEQ ID NO:301;
(k) the VH comprises the amino acid sequence of SEQ ID NO:128 and the VL comprises the amino acid sequence of SEQ ID NO:209;
(l) the VH comprises the amino acid sequence of SEQ ID NO:128 and the VL comprises the amino acid sequence of SEQ ID NO:214;
(m) the VH comprises the amino acid sequence of SEQ ID NO: 128 and the VL comprises the amino acid sequence of SEQ ID NO:212;
(n) the VH comprises the amino acid sequence of SEQ ID NO: 128 and the VL comprises the amino acid sequence of SEQ ID NO:213;
(o) the VH comprises the amino acid sequence of SEQ ID NO: 128 and the VL comprises the amino acid sequence of SEQ ID NO:210;
(p) the VH comprises the amino acid sequence of SEQ ID NO: 128 and the VL comprises the amino acid sequence of SEQ ID NO:211;
(q) the VH comprises the amino acid sequence of SEQ ID NO: 129 and the VL comprises the amino acid sequence of SEQ ID NO:216; or
(r) the VH comprises the amino acid sequence of SEQ ID NO: 128 and the VL comprises the amino acid sequence of SEQ ID NO:216.

In some embodiments, the antibody is a multispecific antibody, bispecific antibody, single chain antibody, an Fab fragment, an F(ab′)₂ fragment, an Fab′ fragment, an Fsc fragment, an Fv fragment, an scFv, an sc(Fv)₂, or a diabody.

In some embodiments, the antibody comprises a constant heavy chain (CH) domain and a constant light chain (CL) domain.

In some embodiments:
(i) the HC comprises an amino acid sequence that is at least 80%, 85%, 90%, 95%, 99%, or 100% identical to the amino acid sequence as set forth in any one of HC-1043 to HC-1094 (as shown in Table 10) and HC-2002 to HC-2020 (as shown in Table 22); and
(ii) the LC comprises an amino acid sequence that is at least 80%, 85%, 90%, 95%, 99%, or 100% identical to the amino acid sequence as set forth in any one of LC-1095 to LC-1114 (as shown in Table 10) and LC-2021 (as shown in Table 22).

In some embodiments, the antibody comprises:
a heavy chain comprising the amino acid sequence set forth in HC-1043, and a light chain comprising the amino acid sequence set forth in LC-1096;
a heavy chain comprising the amino acid sequence set forth in HC-1044, and a light chain comprising the amino acid sequence set forth in LC-1096;
a heavy chain comprising the amino acid sequence set forth in HC-1045, and a light chain comprising the amino acid sequence set forth in LC-1096;
a heavy chain comprising the amino acid sequence set forth in HC-1046, and a light chain comprising the amino acid sequence set forth in LC-1096;
a heavy chain comprising the amino acid sequence set forth in HC-1047, and a light chain comprising the amino acid sequence set forth in LC-1096;
a heavy chain comprising the amino acid sequence set forth in HC-1048, and a light chain comprising the amino acid sequence set forth in LC-1096;
a heavy chain comprising the amino acid sequence set forth in HC-1049, and a light chain comprising the amino acid sequence set forth in LC-1096;
a heavy chain comprising the amino acid sequence set forth in HC-1050, and a light chain comprising the amino acid sequence set forth in LC-1096;
a heavy chain comprising the amino acid sequence set forth in HC-1051, and a light chain comprising the amino acid sequence set forth in LC-1096;
a heavy chain comprising the amino acid sequence set forth in HC-1052, and a light chain comprising the amino acid sequence set forth in LC-1096;
a heavy chain comprising the amino acid sequence set forth in HC-1053, and a light chain comprising the amino acid sequence set forth in LC-1096;
a heavy chain comprising the amino acid sequence set forth in HC-1055, and a light chain comprising the amino acid sequence set forth in LC-1096;
a heavy chain comprising the amino acid sequence set forth in HC-1056, and a light chain comprising the amino acid sequence set forth in LC-1096;
a heavy chain comprising the amino acid sequence set forth in HC-1057, and a light chain comprising the amino acid sequence set forth in LC-1096;
a heavy chain comprising the amino acid sequence set forth in HC-1058, and a light chain comprising the amino acid sequence set forth in LC-1096;
a heavy chain comprising the amino acid sequence set forth in HC-1059, and a light chain comprising the amino acid sequence set forth in LC-1096;
a heavy chain comprising the amino acid sequence set forth in HC-1060, and a light chain comprising the amino acid sequence set forth in LC-1096;
a heavy chain comprising the amino acid sequence set forth in HC-1061, and a light chain comprising the amino acid sequence set forth in LC-1096;
a heavy chain comprising the amino acid sequence set forth in HC-1062, and a light chain comprising the amino acid sequence set forth in LC-1096;
a heavy chain comprising the amino acid sequence set forth in HC-1063, and a light chain comprising the amino acid sequence set forth in LC-1096;
a heavy chain comprising the amino acid sequence set forth in HC-1064, and a light chain comprising the amino acid sequence set forth in LC-1096;
a heavy chain comprising the amino acid sequence set forth in HC-1065, and a light chain comprising the amino acid sequence set forth in LC-1096;
a heavy chain comprising the amino acid sequence set forth in HC-1066, and a light chain comprising the amino acid sequence set forth in LC-1096;
a heavy chain comprising the amino acid sequence set forth in HC-1067, and a light chain comprising the amino acid sequence set forth in LC-1096;
a heavy chain comprising the amino acid sequence set forth in HC-1068, and a light chain comprising the amino acid sequence set forth in LC-1096;
a heavy chain comprising the amino acid sequence set forth in HC-1069, and a light chain comprising the amino acid sequence set forth in LC-1096;
a heavy chain comprising the amino acid sequence set forth in HC-1070, and a light chain comprising the amino acid sequence set forth in LC-1096;
a heavy chain comprising the amino acid sequence set forth in HC-1071, and a light chain comprising the amino acid sequence set forth in LC-1096;
a heavy chain comprising the amino acid sequence set forth in HC-1072, and a light chain comprising the amino acid sequence set forth in LC-1096;
a heavy chain comprising the amino acid sequence set forth in HC-1073, and a light chain comprising the amino acid sequence set forth in LC-1096;
a heavy chain comprising the amino acid sequence set forth in HC-1074, and a light chain comprising the amino acid sequence set forth in LC-1096;
a heavy chain comprising the amino acid sequence set forth in HC-1075, and a light chain comprising the amino acid sequence set forth in LC-1096;
a heavy chain comprising the amino acid sequence set forth in HC-1076, and a light chain comprising the amino acid sequence set forth in LC-1096;
a heavy chain comprising the amino acid sequence set forth in HC-1077, and a light chain comprising the amino acid sequence set forth in LC-1096;
a heavy chain comprising the amino acid sequence set forth in HC-1078, and a light chain comprising the amino acid sequence set forth in LC-1096;
a heavy chain comprising the amino acid sequence set forth in HC-1079, and a light chain comprising the amino acid sequence set forth in LC-1096;
a heavy chain comprising the amino acid sequence set forth in HC-1080, and a light chain comprising the amino acid sequence set forth in LC-1096;
a heavy chain comprising the amino acid sequence set forth in HC-1081, and a light chain comprising the amino acid sequence set forth in LC-1096;
a heavy chain comprising the amino acid sequence set forth in HC-1082, and a light chain comprising the amino acid sequence set forth in LC-1096;
a heavy chain comprising the amino acid sequence set forth in HC-1083, and a light chain comprising the amino acid sequence set forth in LC-1096;
a heavy chain comprising the amino acid sequence set forth in HC-1084, and a light chain comprising the amino acid sequence set forth in LC-1096;
a heavy chain comprising the amino acid sequence set forth in HC-1085, and a light chain comprising the amino acid sequence set forth in LC-1096;
a heavy chain comprising the amino acid sequence set forth in HC-1086, and a light chain comprising the amino acid sequence set forth in LC-1096;
a heavy chain comprising the amino acid sequence set forth in HC-1087, and a light chain comprising the amino acid sequence set forth in LC-1096;
a heavy chain comprising the amino acid sequence set forth in HC-1054, and a light chain comprising the amino acid sequence set forth in LC-1098;
a heavy chain comprising the amino acid sequence set forth in HC-1054, and a light chain comprising the amino acid sequence set forth in LC-1099;
a heavy chain comprising the amino acid sequence set forth in HC-1054, and a light chain comprising the amino acid sequence set forth in LC-1100;
a heavy chain comprising the amino acid sequence set forth in HC-1054, and a light chain comprising the amino acid sequence set forth in LC-1101;
a heavy chain comprising the amino acid sequence set forth in HC-1054, and a light chain comprising the amino acid sequence set forth in LC-1102;
a heavy chain comprising the amino acid sequence set forth in HC-1054, and a light chain comprising the amino acid sequence set forth in LC-1103;
a heavy chain comprising the amino acid sequence set forth in HC-1054, and a light chain comprising the amino acid sequence set forth in LC-1104;
a heavy chain comprising the amino acid sequence set forth in HC-1054, and a light chain comprising the amino acid sequence set forth in LC-1105;
a heavy chain comprising the amino acid sequence set forth in HC-1054, and a light chain comprising the amino acid sequence set forth in LC-1106;
a heavy chain comprising the amino acid sequence set forth in HC-1039, and a light chain comprising the amino acid sequence set forth in LC-1100;
a heavy chain comprising the amino acid sequence set forth in HC-1056, and a light chain comprising the amino acid sequence set forth in LC-1100;
a heavy chain comprising the amino acid sequence set forth in HC-1071, and a light chain comprising the amino acid sequence set forth in LC-1095;
a heavy chain comprising the amino acid sequence set forth in HC-1071, and a light chain comprising the amino acid sequence set forth in LC-1100;
a heavy chain comprising the amino acid sequence set forth in HC-1073, and a light chain comprising the amino acid sequence set forth in LC-1095;
a heavy chain comprising the amino acid sequence set forth in HC-1004, and a light chain comprising the amino acid sequence set forth in LC-1095;
a heavy chain comprising the amino acid sequence set forth in HC-1005, and a light chain comprising the amino acid sequence set forth in LC-1095;
a heavy chain comprising the amino acid sequence set forth in HC-1006, and a light chain comprising the amino acid sequence set forth in LC-1095;
a heavy chain comprising the amino acid sequence set forth in HC-1007, and a light chain comprising the amino acid sequence set forth in LC-1095;
a heavy chain comprising the amino acid sequence set forth in HC-1008, and a light chain comprising the amino acid sequence set forth in LC-1095;
a heavy chain comprising the amino acid sequence set forth in HC-1009, and a light chain comprising the amino acid sequence set forth in LC-1095;
a heavy chain comprising the amino acid sequence set forth in HC-1010, and a light chain comprising the amino acid sequence set forth in LC-1095;
a heavy chain comprising the amino acid sequence set forth in HC-1011, and a light chain comprising the amino acid sequence set forth in LC-1095;
a heavy chain comprising the amino acid sequence set forth in HC-1012, and a light chain comprising the amino acid sequence set forth in LC-1095;
a heavy chain comprising the amino acid sequence set forth in HC-1013, and a light chain comprising the amino acid sequence set forth in LC-1095;
a heavy chain comprising the amino acid sequence set forth in HC-1014, and a light chain comprising the amino acid sequence set forth in LC-1095;
a heavy chain comprising the amino acid sequence set forth in HC-1015, and a light chain comprising the amino acid sequence set forth in LC-1095;
a heavy chain comprising the amino acid sequence set forth in HC-1016, and a light chain comprising the amino acid sequence set forth in LC-1095;
a heavy chain comprising the amino acid sequence set forth in HC-1017, and a light chain comprising the amino acid sequence set forth in LC-1095;
a heavy chain comprising the amino acid sequence set forth in HC-1018, and a light chain comprising the amino acid sequence set forth in LC-1095;
a heavy chain comprising the amino acid sequence set forth in HC-1019, and a light chain comprising the amino acid sequence set forth in LC-1095;
a heavy chain comprising the amino acid sequence set forth in HC-1020, and a light chain comprising the amino acid sequence set forth in LC-1095;
a heavy chain comprising the amino acid sequence set forth in HC-1021, and a light chain comprising the amino acid sequence set forth in LC-1095;
a heavy chain comprising the amino acid sequence set forth in HC-1022, and a light chain comprising the amino acid sequence set forth in LC-1095;
a heavy chain comprising the amino acid sequence set forth in HC-1023, and a light chain comprising the amino acid sequence set forth in LC-1095;
a heavy chain comprising the amino acid sequence set forth in HC-1024, and a light chain comprising the amino acid sequence set forth in LC-1095;
a heavy chain comprising the amino acid sequence set forth in HC-1025, and a light chain comprising the amino acid sequence set forth in LC-1095;
a heavy chain comprising the amino acid sequence set forth in HC-1026, and a light chain comprising the amino acid sequence set forth in LC-1095;
a heavy chain comprising the amino acid sequence set forth in HC-1027, and a light chain comprising the amino acid sequence set forth in LC-1095;
a heavy chain comprising the amino acid sequence set forth in HC-1028, and a light chain comprising the amino acid sequence set forth in LC-1095;
a heavy chain comprising the amino acid sequence set forth in HC-1029, and a light chain comprising the amino acid sequence set forth in LC-1095;
a heavy chain comprising the amino acid sequence set forth in HC-1030, and a light chain comprising the amino acid sequence set forth in LC-1095;
a heavy chain comprising the amino acid sequence set forth in HC-1031, and a light chain comprising the amino acid sequence set forth in LC-1095;
a heavy chain comprising the amino acid sequence set forth in HC-1033, and a light chain comprising the amino acid sequence set forth in LC-1095;
a heavy chain comprising the amino acid sequence set forth in HC-1013, and a light chain comprising the amino acid sequence set forth in LC-1100;
a heavy chain comprising the amino acid sequence set forth in HC-1013, and a light chain comprising the amino acid sequence set forth in LC-1107;
a heavy chain comprising the amino acid sequence set forth in HC-1013, and a light chain comprising the amino acid sequence set forth in LC-1108;
a heavy chain comprising the amino acid sequence set forth in HC-1013, and a light chain comprising the amino acid sequence set forth in LC-1109;
a heavy chain comprising the amino acid sequence set forth in HC-1013, and a light chain comprising the amino acid sequence set forth in LC-1110;
a heavy chain comprising the amino acid sequence set forth in HC-1013, and a light chain comprising the amino acid sequence set forth in LC-1111;
a heavy chain comprising the amino acid sequence set forth in HC-1013, and a light chain comprising the amino acid sequence set forth in LC-1112;
a heavy chain comprising the amino acid sequence set forth in HC-1034, and a light chain comprising the amino acid sequence set forth in LC-1113;
a heavy chain comprising the amino acid sequence set forth in HC-1034, and a light chain comprising the amino acid sequence set forth in LC-1114;
a heavy chain comprising the amino acid sequence set forth in HC-2002, and a light chain comprising the amino acid sequence set forth in LC-1095;
a heavy chain comprising the amino acid sequence set forth in HC-2003, and a light chain comprising the amino acid sequence set forth in LC-1095;
a heavy chain comprising the amino acid sequence set forth in HC-2004, and a light chain comprising the amino acid sequence set forth in LC-1095;
a heavy chain comprising the amino acid sequence set forth in HC-2005, and a light chain comprising the amino acid sequence set forth in LC-1095;
a heavy chain comprising the amino acid sequence set forth in HC-2006, and a light chain comprising the amino acid sequence set forth in LC-1095;
a heavy chain comprising the amino acid sequence set forth in HC-2007, and a light chain comprising the amino acid sequence set forth in LC-1095;
a heavy chain comprising the amino acid sequence set forth in HC-2006, and a light chain comprising the amino acid sequence set forth in LC-2021;
a heavy chain comprising the amino acid sequence set forth in HC-2008, and a light chain comprising the amino acid sequence set forth in LC-1095;
a heavy chain comprising the amino acid sequence set forth in HC-2009, and a light chain comprising the amino acid sequence set forth in LC-1095;
a heavy chain comprising the amino acid sequence set forth in HC-2010, and a light chain comprising the amino acid sequence set forth in LC-1095;
a heavy chain comprising the amino acid sequence set forth in HC-2011, and a light chain comprising the amino acid sequence set forth in LC-1095;
a heavy chain comprising the amino acid sequence set forth in HC-2012, and a light chain comprising the amino acid sequence set forth in LC-2021;
a heavy chain comprising the amino acid sequence set forth in HC-2013, and a light chain comprising the amino acid sequence set forth in LC-1095;
a heavy chain comprising the amino acid sequence set forth in HC-2014, and a light chain comprising the amino acid sequence set forth in LC-1095;
a heavy chain comprising the amino acid sequence set forth in HC-2015, and a light chain comprising the amino acid sequence set forth in LC-1095;
a heavy chain comprising the amino acid sequence set forth in HC-2016, and a light chain comprising the amino acid sequence set forth in LC-1095;
a heavy chain comprising the amino acid sequence set forth in HC-2016, and a light chain comprising the amino acid sequence set forth in LC-2021;
a heavy chain comprising the amino acid sequence set forth in HC-2017, and a light chain comprising the amino acid sequence set forth in LC-1095;
a heavy chain comprising the amino acid sequence set forth in HC-2018, and a light chain comprising the amino acid sequence set forth in LC-1095;
a heavy chain comprising the amino acid sequence set forth in HC-2019, and a light chain comprising the amino acid sequence set forth in LC-1095;
a heavy chain comprising the amino acid sequence set forth in HC-2020, and a light chain comprising the amino acid sequence set forth in LC-1095;
the amino acid sequence set forth in HC-1088;
the amino acid sequence set forth in HC-1089;
the amino acid sequence set forth in HC-1090;
the amino acid sequence set forth in HC-1091;
the amino acid sequence set forth in HC-1092;
the amino acid sequence set forth in HC-1093; or
the amino acid sequence set forth in HC-1094,
wherein the HA tag is optionally excluded from a foregoing amino acid sequence that contains an HA tag.

In some embodiments, the antibody is a monovalent antibody comprising one VH and one VL, or comprising one heavy chain and one light chain.

In some embodiments:
the VH comprises a VH CDR1 comprising the amino acid sequence GIDFSSSGYMC (SEQ ID NO:XX), a VH CDR2 comprising the amino acid sequence CIYTYSSNTYYAASVKG (SEQ ID NO:XX), a VH CDR3 comprising the amino acid sequence GTYGYTGATYTMGYFSL (SEQ ID NO:XX); and wherein the VL comprises a VL CDR1 comprising the amino acid sequence QASQNINSYLA (SEQ ID NO:XX), a VL CDR2 comprising the amino acid sequence RASSLES (SEQ ID NO:XX), and a VL CDR3 comprising the amino acid sequence QSYYYSGSSNYNA (SEQ ID NO:XX);
the VH comprises the amino acid sequence of SEQ ID NO: 138 and the VL comprises the amino acid sequence of SEQ ID NO:301; or the heavy chain comprises the amino acid sequence set forth in HC-2015, and the light chain comprises the amino acid sequence set forth in LC-1095.

In some embodiments, the antibody is a bivalent antibody comprising two VHs and two VLs, or comprising two heavy chains and two light chains.

In some embodiments:
each VH comprises a VH CDR1 comprising the amino acid sequence GIDFSSSGYMC (SEQ ID NO:XX), a VH CDR2 comprising the amino acid sequence CIDTYSSNTYYAASVKG (SEQ ID NO:XX), and a VH CDR3 comprising the amino acid sequence GTYGYTGYTYTMGYFSL (SEQ ID NO:XX); and each VL comprises a VL CDR1 comprising the amino acid sequence QASQNINSYLA (SEQ ID NO:XX); a VL CDR2 comprising the amino acid sequence RASSLES (SEQ ID NO:XX); and a VL CDR3 comprising the amino acid sequence QSYYYSGSSNYNA (SEQ ID NO:XX);
each VH comprises the amino acid sequence of SEQ ID NO: 122 and each VL comprises the amino acid sequence of SEQ ID NO:301; or each heavy chain comprises the amino acid sequence set forth in HC-2008, and each light chain comprises the amino acid sequence set forth in LC-1095.

In another aspect, the disclosure features a nucleic acid or nucleic acids encoding an antibody described herein.

In another aspect, the disclosure features an expression vector or expression vectors comprising a nucleic acid or nucleic acids described herein operably linked to a promoter.

In another aspect, the disclosure features an isolated cell comprising a nucleic acid or nucleic acids described herein or an expression vector or expression vectors described herein.

In another aspect, the disclosure features an isolated cell comprising a first expression vector comprising a first nucleic acid encoding a first polypeptide comprising the VH of an antibody described herein operably linked to a promoter, and a second expression vector comprising a second nucleic acid encoding a second polypeptide comprising the VL of an antibody described herein operably linked to a promoter.

In another aspect, the disclosure features method of making an antibody described herein, comprising culturing a cell described herein and isolating the antibody.

In another aspect, the disclosure features a pharmaceutical composition comprising an antibody described herein and a pharmaceutically acceptable carrier.

In another aspect, the disclosure features a conjugate comprising an antibody described herein and an agent.

In some embodiments, the agent is an antibody, protein, or peptide.

In some embodiments, the agent is an anti-beta amyloid antibody (e.g., aducanumab, bapineuzumab, gantenerumab, solanezumab, donanemab, or lecanemab).

In some embodiments, the agent is an anti-tau antibody, an anti-alpha synuclein antibody, an anti-TDP-43 antibody, an anti-LINGO-1 antibody, an anti-LINGO-2 antibody, an anti-LINGO-3 antibody, an anti-LINGO-4 antibody, an anti-TREM2 antibody, or an anti-C9orf72 dipeptide repeat poly-GA antibody.

In some embodiments, the agent is protein (e.g., progranulin).

In some embodiments, the agent is an enzyme (e.g., glucocerebrosidase).

In some embodiments, the conjugate is a recombinant fusion protein comprising the antibody and the agent.

In some embodiments, the agent is a nucleic acid (e.g., an mRNA, a siRNA, an antisense oligonucleotide, microRNA (miRNA), guide RNA (gRNA), or a phosphoroamidate morpholino oligomer (PMO)). In some embodiments, the nucleic acid is linked to the antibody via a linker.

In some embodiments, the agent is a nanoparticle, liposome, or viral vector.

In another aspect, the disclosure features a method of transporting an agent across the blood brain barrier via transcytosis, the method comprising administering to a human subject a conjugate described herein.

In another aspect, the disclosure features a method of delivering an agent in vivo, the method comprising administering to a human subject a conjugate described herein. In some embodiments, the human subject has a neurological disorder and the method delivers the agent to brain tissue. In some embodiments, the neurological disorder is Alzheimer's disease, Parkinson's disease, frontotemporal dementia, ALS, Huntington's disease, multiple sclerosis, spinal muscular atrophy, muscular dystrophy, spinal cord injury, stroke, an ophthalmological condition, acute or chronic optic neuritis, a psychiatric disorder, Tourette's disease brain injury, a brain tumor, or epilepsy.

In another aspect, the disclosure features a method of treating Alzheimer's disease in a human subject in need thereof, comprising administering to the subject a therapeutically effective amount of a conjugate comprising an antibody described herein and an anti-beta amyloid antibody (e.g., aducanumab, bapineuzumab, gantenerumab, solanezumab, donanemab, or lecanemab).

Other features and advantages of the invention will be apparent from the following detailed description and from the claims.

### BRIEF DESCRIPTION OF THE DRAWINGS

FIGS. 1A-1B are graphs depicting concentration-dependent binding of anti-TfR1 antibodies with single mutations to CHO cells expressing full length human TfR1.

FIGS. 2A-2B are graphs depicting concentration-dependent binding of anti-TfR1 antibodies with multiple mutations to CHO cells expressing full length human TfR1.

FIG. 3 is a graph depicting the relationship between monomeric affinity of anti-TfR1 antibodies and transcytosis mediated by the respective antibodies.

FIG. 4 is a graph depicting concentration-dependent binding of inverted bivalent format mutant anti-TfR1 antibodies to CHO cells expressing full length human TfR1.

FIG. 5 is a series of photographs depicting exposure of mutant anti-TfR1 antibodies in the brain parenchyma across multiple brain regions.

FIG. 6A is a graph depicting concentration-dependent binding of anti-TfR1 mutant bivalent antibodies to CHO cells expressing full length human TfR1 by flow cytometry. FIG. 6B is a graph depicting concentration-dependent binding of anti-TfR1 mutant bivalent antibodies to CHO cells expressing full length cynomolgus TfR1 by flow cytometry.

FIG. 7A is a graph depicting concentration-dependent binding of anti-TfR1 mutant monovalent antibodies to CHO cells expressing full length human (left) or cynomolgus (right) TfR1 by flow cytometry. FIG. 7B is a graph depicting concentration-dependent binding of anti-TfR1 mutant monovalent antibodies to CHO cells expressing full length cynomolgus TfR1 by flow cytometry.

FIG. 8 is a series of graphs depicting time- and concentration-dependent binding of anti-TfR1 mutant monovalent antibodies to recombinant human or cynomolgus TfR1 ectodomain by surface plasmon resonance.

FIG. 9A is a graph depicting in vitro transcytosis of anti-TfR1 mutant bivalent and monovalent antibodies plotted against cell-surface affinity for human TfR1. FIG. 9B is a graph depicting in vitro transcytosis of anti-TfR1 mutant bivalent and monovalent antibodies plotted against monovalent affinity for human TfR1 ectodomain.

FIG. 10A is a graph depicting concentration of mutant anti-TfR1 antibodies in mouse brain lysate one day following IV administration. FIG. 10B is a graph depicting the relationship between uptake of mutant anti-TfR1 antibodies in mouse brain and the cell surface human TfR1 antibody affinity (EC₅₀). FIG. 10C is a graph depicting the relationship between uptake of mutant anti-TfR1 antibodies in mouse brain and the monovalent affinity for recombinant human TfR1 ectodomain (K_{D}).

FIG. 11A is a graph showing the levels of mutant anti-TfR1 antibodies in mouse serum one and seven days after IV administration. FIG. 11B is a graph showing the levels of mutant anti-TfR1 antibodies in mouse brain one and seven days after IV administration.

FIG. 12 is a series of photographs depicting exposure of mutant anti-TfR1 antibodies in mouse brain one day after IV administration.

FIG. 13A depicts the amount of TfR1 present in a human brain endothelial cell line following one day of culture with anti-TfR1 mutant antibodies. FIG. 13B is a graph depicting the relationship between TfR1 level present in a human brain endothelial cell line following one day of culture with anti-TfR1 mutant antibodies and antibody affinity for cell surface TfR1.

FIG. 14A is a graph depicting the reticulocyte counts as a percentage of total blood cells in mice one day following IV administration with mutant anti-TfR1 antibodies. FIG. 14B is a graph depicting the relationship between reticulocyte retention (percent of control antibody treatment) and the antibody affinity for cell surface TfR1 (EC₅₀).

FIG. 15 is a schematic depicting various antibody domains and exemplary antibody formats.

### DETAILED DESCRIPTION

The present disclosure provides antibodies that specifically bind transferrin receptor 1 (TfR1). Related polypeptides, polynucleotides, vectors, cells, compositions and conjugates comprising the antibodies, methods of making the antibodies, and methods of delivering the compositions and conjugates are also provided. The disclosure also provides methods of using the anti-TfR antibodies.

### Definitions

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody” as used herein refers to an immunoglobulin molecule that recognizes and binds a target through at least one antigen-binding site. “Antibody” is used herein in the broadest sense and encompasses various antibody structures, including “antibody fragments” and “antigen-binding fragments.” Thus, the term “antibody” includes, but is not limited to, recombinant antibodies, monoclonal antibodies, chimeric antibodies, humanized antibodies, human antibodies, bispecific antibodies, multispecific antibodies, diabodies, tribodies, tetrabodies, single chain Fv (scFv) antibodies, and antibody fragments as long as they exhibit the desired antigen-binding activity.

The term “intact antibody” or “full-length antibody” refers to an antibody having a structure substantially similar to a native antibody structure. This includes, for example, an antibody comprising two light chains each comprising a variable region and a light chain constant region (CL) and two heavy chains each comprising a variable region and at least heavy chain constant regions CH1, CH2, and CH3 and a hinge region between CH1 and CH2 regions.

The term “antigen-binding fragment”, as used herein refers to a molecule other than an intact antibody that comprises a portion of an antibody and an antigen-binding site. Examples of antibody fragments include, but are not limited to, Fab, Fab′, F(ab′)₂, Fv, single chain antibody molecules (e.g., scFv, sc(Fv)₂), disulfide-linked scFv (dsscFv), diabodies, tribodies, tetrabodies, minibodies, dual variable domain antibodies (DVD), single variable domain antibodies (e.g., camelid antibodies), and multispecific antibodies formed from antibody fragments.

The term “monoclonal antibody” as used herein refers to a substantially homogenous antibody population involved in the highly specific recognition and binding of a single antigenic determinant or epitope. The term “monoclonal antibody” encompasses intact and full-length monoclonal antibodies as well as antibody fragments (e.g., Fab, Fab′, F(ab′)₂, Fv), single chain antibodies (e.g., scFv), fusion proteins comprising an antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The term “chimeric antibody” refers to an antibody in which a portion of the heavy and/or light chain is derived from a first source or species, while the remainder of the heavy and/or light chain is derived from a different source or species.

The term “humanized antibody” as used herein refers to an antibody that comprises a human heavy chain variable region and a light chain variable region wherein the native CDR amino acid residues are replaced by residues from corresponding CDRs from a nonhuman antibody (e.g., mouse, rat, rabbit, or nonhuman primate), wherein the nonhuman antibody has the desired specificity, affinity, and/or activity. In some embodiments, one or more framework region amino acid residues of the human heavy chain or light chain variable regions are replaced by corresponding residues from nonhuman antibody. Furthermore, humanized antibodies can comprise amino acid residues that are not found in the human antibody or in the nonhuman antibody. In some embodiments, these modifications are made to further refine and/or optimize antibody characteristics. In some embodiments, the humanized antibody comprises at least a portion of an immunoglobulin constant region (e.g., CH1, hinge, CH2, CH3, Fc), typically that of a human immunoglobulin.

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, recombinant protein production, and B-cell hybridoma technology.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of software bioinformatic tools available on the internet. X-ray crystallography or electron microscopy (e.g., cryo-electron microscopy) may be used to characterize an epitope on a target protein by analyzing the amino acid residue interactions of an antigen/antibody complex.

The term “specifically binds” or “binds” as used herein refers to an antibody that interacts more frequently, more rapidly, with greater duration, with greater affinity, or with some combination of the above to a particular antigen, epitope, protein, or target molecule than with alternative substances. An antibody that specifically binds an antigen can be identified, for example, by immunoassays, ELISAs, surface plasmon resonance (SPR), or other techniques known to those of skill in the art. In some embodiments, an antibody that specifically binds an antigen (e.g., human TfR1) can bind related antigens (e.g., cyno TfR1). An antibody that specifically binds an antigen can bind the target antigen at a higher affinity than its affinity for a different antigen. The different antigen can be a related antigen. In some embodiments, an antibody that specifically binds an antigen can bind the target antigen with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different antigen. In some embodiments, an antibody that specifically binds a particular antigen binds a different antigen at such a low affinity that binding cannot be detected using an assay described herein or otherwise known in the art. In some embodiments, affinity is measured using SPR technology in a Biacore system as described herein or as known to those of skill in the art.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies, the term “polypeptide” encompasses polypeptides as a single chain and polypeptides of two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 20-40, at least about 40-60 nucleotides or amino acid residues, at least about 60-80 nucleotides or amino acid residues in length or any integral value there between. In some embodiments, identity exists over a longer region than 60-80 nucleotides or amino acid residues, such as at least about 80-100 nucleotides or amino acid residues, and in some embodiments the sequences are substantially identical over the full length of the sequences being compared, for example, (i) the coding region of a nucleotide sequence or (ii) an amino acid sequence.

The phrase “conservative amino acid substitution” as used herein refers to a substitution in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Generally, conservative substitutions in the sequences of polypeptides and/or antibodies do not abrogate the binding of the polypeptide or antibody to the target binding site. Methods of identifying nucleotide and amino acid conservative substitutions that do not eliminate binding are well-known in the art.

The term “vector” as used herein means a construct that is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “isolated” as used herein refers to a polypeptide, soluble protein, antibody, polynucleotide, vector, cell, or composition that is in a form not found in nature. An “isolated” antibody is substantially free of material from the cellular source from which it is derived. In some embodiments, isolated polypeptides, soluble proteins, antibodies, polynucleotides, vectors, cells, or compositions are those that have been purified to a degree that they are no longer in a form in which they are found in nature. In some embodiments, a polypeptide, soluble protein, antibody, polynucleotide, vector, cell, or composition that is isolated is substantially pure. A polypeptide, soluble protein, antibody, polynucleotide, vector, cell, or composition can be isolated from a natural source (e.g., tissue) or from a source such as an engineered cell line. The term “substantially pure” as used herein refers to material that is at least 50% pure (i.e., free from contaminants), at least 90% pure, at least 95% pure, at least 98% pure, or at least 99% pure.

The term “pharmaceutically acceptable” as used herein refers to a substance approved or approvable by a regulatory agency or listed in the U.S. Pharmacopeia, European Pharmacopeia, or other generally recognized pharmacopeia for use in animals, including humans.

The terms “pharmaceutically acceptable excipient, carrier, or adjuvant” as used herein refer to an excipient, carrier, or adjuvant that can be administered to a subject, together with at least one antibody of the disclosure, and that is generally safe, non-toxic, and has no effect on the pharmacological activity of the therapeutic agent. In general, those of skill in the art and the U.S. FDA consider a pharmaceutically acceptable excipient, carrier, or adjuvant to be an inactive ingredient of any formulation.

The term “pharmaceutical composition” as used herein refers to a preparation that is in such form as to permit the biological activity of the antibody to be effective. A pharmaceutical formulation or composition generally comprises additional components, such as a pharmaceutically acceptable excipient, carrier, adjuvant, buffers, etc.

The term “conjugate” as used herein refers to a combination in which two substances are linked by a covalent bond (e.g., an antibody of the disclosure joined to a therapeutic agent). In the conjugate, the two substances may be directly connected or may be connected via a linker. In the present disclosure, one of the two substances is an antibody of the disclosure, and the other is a drug (for example, a physiologically active substance). The linker may be a cleavable linker or a non-cleavable linker.

The term “effective amount” or “therapeutically effective amount” as used herein refers to an amount of an antibody of the disclosure that is required to reach the tissue of interest, or to an amount of a conjugate, a fusion protein or polypeptide, or a complex comprising an antibody of the disclosure and a therapeutic agent that is sufficient to reduce and/or ameliorate the severity and/or duration of (i) a disease, disorder or condition in a subject, and/or (ii) a symptom in a subject. The term also encompasses an amount of a conjugate necessary for the (i) reduction or amelioration of the advancement or progression of a given disease, disorder, or condition, (ii) reduction or amelioration of the recurrence, development, or onset of a given disease, disorder, or condition, and/or (iii) the improvement or enhancement of the prophylactic or therapeutic effect(s) of another agent or therapy (e.g., an agent other than the conjugates provided herein). The term “therapeutic effect” as used herein refers to the effect and/or ability of an agent, e.g., an antibody, a conjugate, a fusion protein or polypeptide, or a complex comprising the antibody of the disclosure to reduce and/or ameliorate the severity and/or duration of (i) a disease, disorder, or condition in a subject, and/or (ii) a symptom in a subject. The term also encompasses the ability of an agent, e.g., a conjugate, to (i) reduce or ameliorate the advancement or progression of a given disease, disorder, or condition, (ii) reduce or ameliorate the recurrence, development, or onset of a given disease, disorder, or condition, and/or (iii) to improve or enhance the prophylactic or therapeutic effect(s) of another agent or therapy (e.g., an agent other than the conjugates provided herein).

As used herein, reference to “about” or “approximately” a value or parameter includes (and describes) embodiments that are directed to that value or parameter. For example, a description referring to “about X” includes description of “X”. “About X” means +/−10% of X. So, “about 10” means a value between 9 to 11.

### TfR1 and Anti-TfR1 Antibodies

Transferrin receptor, also known as CD71, is a transmembrane glycoprotein expressed in various sites of the human body at differing levels, whose function is to mediate cellular uptake of iron from a plasma glycoprotein, transferrin. Iron uptake from transferrin involves the binding of transferrin to the transferrin receptor, internalization of transferrin within an endocytic vesicle by receptor-mediated endocytosis and the release of iron from the protein by a decrease in endosomal pH. Ponka P, Lok C N. Int J Biochem Cell Biol. 1999 October; 31 (10): 1111-37 and Xiaopeng Mo, in Brain Targeted Drug Delivery System, 2019. Apotransferrin (i.e., non-iron conjugate) binds to TfR when bound to two Fe 3+ ions to form holotransferrin (i.e., iron conjugate). The complex of TfR and holotransferrin is translocated into the cell by receptor-mediated endocytosis. CD71 and transferrin dissociate in an endosomal environment, and transferrin moves into the cell while CD71 is recycled to the cell membrane. Thus, transferrin is thought to translocate into cells by proper binding to TfR and proper dissociation. The transferrin receptor system has been exploited for delivery of anticancer drugs and proteins, therapeutic genes into malignant cells, and to deliver other therapeutic agents across the blood brain barrier to the brain.

In humans and cynomolgus monkeys, two transferrin receptors, TfR1 and TfR2 have been characterized. TfR1 is a high affinity ubiquitously expressed receptor while expression of TfR2 is restricted to certain cell types and is unaffected by intracellular iron concentrations. TfR2 binds to transferrin with a 25-30 fold lower affinity than TfR1. The antibodies of the present disclosure bind to TfR1.

The sequences for human TfR1 and cyno TfR1 are as follows:

Human TfR1
(UniProt No. P02786.2; SEQ ID NO: XX)
MMDQARSAFSNLFGGEPLSYTRFSLARQVDGDNSHVEMKLAVDEE
ENADNNTKANVTKPKRCSGSICYGTIAVIVFFLIGFMIGYLGYCK
GVEPKTECERLAGTESPVREEPGEDFPAARRLYWDDLKRKLSEKL
DSTDFTGTIKLLNENSYVPREAGSQKDENLALYVENQFREFKLSK
VWRDQHFVKIQVKDSAQNSVIIVDKNGRLVYLVENPGGYVAYSKA
ATVTGKLVHANFGTKKDFEDLYTPVNGSIVIVRAGKITFAEKVAN
AESLNAIGVLIYMDQTKFPIVNAELSFFGHAHLGTGDPYTPGFPS
FNHTQFPPSRSSGLPNIPVQTISRAAAEKLFGNMEGDCPSDWKTD
STCRMVTSESKNVKLTVSNVLKEIKILNIFGVIKGFVEPDHYVVV
GAQRDAWGPGAAKSGVGTALLLKLAQMFSDMVLKDGFQPSRSIIF
ASWSAGDFGSVGATEWLEGYLSSLHLKAFTYINLDKAVLGTSNFK
VSASPLLYTLIEKTMQNVKHPVTGQFLYQDSNWASKVEKLTLDNA
AFPFLAYSGIPAVSFCFCEDTDYPYLGTTMDTYKELIERIPELNK
VARAAAEVAGQFVIKLTHDVELNLDYERYNSQLLSFVRDLNQYRA
DIKEMGLSLQWLYSARGDFFRATSRLTTDFGNAEKTDRFVMKKLN
DRVMRVEYHFLSPYVSPKESPFRHVFWGSGSHTLPALLENLKLRK
QNNGAFNETLFRNQLALATWTIQGAANALSGDVWDIDNEF
Cyno TfR1
(UniProt No. G8F602; SEQ ID NO: XX)
MMDQARSAFSNLFGGEPLSYTRFSLARQVDGDNSHVEMKLAVDDE
ENADNNTKANGTKPKRCGGNICYGTIAVIIFFLIGFMIGYLGYCK
GVEPKTECERLAGTESPAREEPEEDFPAAPRLYWDDLKRKLSEKL
DTTDFTSTIKLLNENLYVPREAGSQKDENLALYIENQFREFKLSK
VWRDQHFVKIQVKDSAQNSVIIVDKNGGLVYLVENPGGYVAYSKA
ATVTGKLVHANFGTKKDFEDLDSPVNGSIVIVRAGKITFAEKVAN
AESLNAIGVLIYMDQTKFPIVKADLSFFGHAHLGTGDPYTPGFPS
FNHTQFPPSQSSGLPNIPVQTISRAAAEKLFGNMEGDCPSDWKTD
STCKMVTSENKSVKLTVSNVLKETKILNIFGVIKGFVEPDHYVVV
GAQRDAWGPGAAKSSVGTALLLKLAQMFSDMVLKDGFQPSRSIIF
ASWSAGDFGSVGATEWLEGYLSSLHLKAFTYINLDKAVLGTSNFK
VSASPLLYTLIEKTMQDVKHPVTGRSLYQDSNWASKVEKLTLDNA
AFPFLAYSGIPAVSFCFCEDTDYPYLGTTMDTYKELVERIPELNK
VARAAAEVAGQFVIKLTHDTELNLDYERYNSQLLLFLRDLNQYRA
DVKEMGLSLQWLYSARGDFFRATSRLTTDFRNAEKRDKFVMKKLN
DRVMRVEYYFLSPYVSPKESPFRHVFWGSGSHTLSALLESLKLRR
QNNSAFNETLFRNQLALATWTIQGAANALSGDVWDIDNEF

The present disclosure provides antibodies that bind TfR1.

In some embodiments, the anti-TfR1 antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgA, IgD, IgE, IgG, or IgM antibody. In some embodiments, the antibody is an IgG antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody. In some instances, the antibody comprises the human kappa light chain constant region. In other embodiments, the antibody comprises a human lambda light chain constant region. In some embodiments, the antibody is an antibody fragment comprising an antigen-binding site. In some embodiments, the antibody is a scFv. In some embodiments, the antibody is a disulfide-linked scFv. In some embodiments, the antibody is a bispecific antibody or a multispecific antibody. In some embodiments, the antibody is a monovalent antibody. In some embodiments, the antibody is a monospecific antibody. In some embodiments, the antibody is a bivalent antibody. In some embodiments, the antibody has a conventional (right-side up) orientation. In some embodiments, the antibody has an inverted (upside-down) orientation.

In some instances, the antibody is an Fab, Fab′, F(ab)₂, scFv, sc(Fv)₂, diabody, or nanobody. In some embodiments, the interchain disulfide in the antibody or antigen binding fragment (e.g., Fab) is removed. The Fab or Fab′ contains a variable heavy (VH) and a variable light domain (VL).

In some embodiments, the antibody is isolated. In some embodiments, the antibody is substantially pure.

In some embodiments, the anti-TfR1 antibody is a humanized antibody. Various methods for generating humanized antibodies are known in the art. In some embodiments, a humanized antibody comprises one or more amino acid residues that have been introduced into its sequence from a source that is non-human. In some embodiments, humanization is performed by substituting one or more non-human CDR sequences for the corresponding CDR sequences of a human antibody.

The choice of which human heavy chain variable region and/or light chain variable region are used for generating humanized antibodies can be made based on a variety of factors and by a variety of methods known in the art. In some embodiments, the “best-fit” method is used where the sequence of the variable region of a non-human (e.g., rodent) antibody is screened against the entire library of known human variable region sequences. The human sequence that is most similar to that of the non-human (e.g., rodent) sequence is selected as the human variable region framework for the humanized antibody. In some embodiments, a particular variable region framework derived from a consensus sequence of all human antibodies of a particular subgroup of light or heavy chains is selected as the variable region framework. In some embodiments, the variable region framework sequence is derived from the consensus sequences of the most abundant human subclasses. In some embodiments, human germline genes are used as the source of the variable region framework sequences.

Other methods for humanization include, but are not limited to, (i) a method called “superhumanization” that is described as the direct transfer of CDRs to a human germline framework, (ii) a method termed Human String Content (HSC) that is based on a metric of “antibody humanness”, (iii) methods based on generation of large libraries of humanized variants (including phage, ribosomal, and yeast display libraries), and (iv) methods based on framework region shuffling.

In some embodiments, the anti-TfR1 antibody is a “human antibody”. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology may be used to produce human antibodies and antibody fragments in vitro, from immunoglobulin variable region gene repertoires from unimmunized donors. Techniques for the generation and use of antibody phage libraries are well-known in the art. Once antibodies are identified, affinity maturation strategies known in the art, including but not limited to, chain shuffling and site-directed mutagenesis, may be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

In some embodiments, the anti-TfR1 antibody is a bispecific antibody. Bispecific antibodies are capable of recognizing and binding at least two different antigens or epitopes. The different epitopes can either be within the same molecule (e.g., two epitopes on TfR1) or on different molecules (e.g., one epitope on TfR1 and one epitope on a different target). In some embodiments, a bispecific antibody has enhanced potency as compared to an individual antibody or to a combination of more than one antibody. In some embodiments, a bispecific antibody has reduced toxicity as compared to an individual antibody or to a combination of more than one antibody. It is known to those of skill in the art that any therapeutic agent may have unique pharmacokinetics (PK) (e.g., circulating half-life). In some embodiments, a bispecific antibody has the ability to synchronize the PK of two active binding agents wherein the two individual binding agents have different PK profiles. In some embodiments, a bispecific antibody has the ability to concentrate the actions of two agents in a common area (e.g., tissue) in a subject. In some embodiments, a bispecific antibody has the ability to concentrate the actions of two agents to a common target (e.g., a specific cell type). In some embodiments, a bispecific antibody has the ability to target the actions of two agents to more than one biological pathway or function. In some embodiments, a bispecific antibody has the ability to target two different cells and bring them closer together.

In some embodiments, a bispecific antibody has decreased toxicity and/or side effects. In some embodiments, a bispecific antibody has decreased toxicity and/or side effects as compared to a mixture of the two individual antibodies or the antibodies as single agents. In some embodiments, a bispecific antibody has an increased therapeutic index. In some embodiments, a bispecific antibody has an increased therapeutic index as compared to a mixture of the two individual antibodies or the antibodies as single agents.

Several techniques for making bispecific antibodies are known by those skilled in the art. In some embodiments, the bispecific antibodies comprise heavy chain constant regions with modifications in the amino acids that are part of the interface between the two heavy chains. These modifications are made to enhance heterodimer formation and generally reduce or eliminate homodimer formation. In some embodiments, the bispecific antibodies are generated using a knobs-into-holes (KIH) strategy. See, e.g., Ridgway et al. Protein Eng. 1996; 9 (7): 617-21 and Klein et al. MAbs. 2012; 4 (6): 653-663.

In some embodiments, the bispecific antibodies comprise light chain constant regions with modifications in the amino acids that are part of the interface between the two light chains. These modifications are made to reduce or eliminate light chain mispairing. See, e.g., Lewis et al. Nat Biotech 2014; 32 (2): 191-98. In some embodiments, the bispecific antibodies comprise an scFv that covalently links the VH and VL and removes CH1 and CL. In some embodiments, the bispecific antibodies comprise an scFab or Fcab (see, e.g., Wozniak-Knopp et al. PEDS 2010; 23 (4): 289-97), single-domain antibodies (e.g., with VHHs from camelid species or sharks), or Duet Mabs (see, e.g., Mazor et al. Mabs 2015; 7 (2): 377-89).

Bispecific antibodies can be intact antibodies or antibody fragments comprising antigen-binding sites.

Anti-TfR1 antibodies with more than two specificities are contemplated in this disclosure. In some embodiments, trispecific or tetraspecific antibodies are generated. Anti-TfR1 antibodies with more than two valencies are contemplated. In some embodiments, trivalent or tetravalent antibodies are generated.

CDRs of an antibody are defined by those skilled in the art using a variety of methods/systems. These systems and/or definitions have been developed and refined over a number of years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia. Software programs (e.g., abYsis) are available and known to those of skill in the art for analysis of antibody sequence and determination of CDRs.

The CDR sequences described in Table 1 include the union of all positions in the Kabat CDR definitions (Kabat, E. A., Wu, T. T., Perry, H. M., Gottesman, K. S. & Foeller, C. (1991). Sequences of Proteins of Immunological Interest, 5th edit. National Institutes of Health, Bethesda, MD) and the Chothia CDR definitions (Chothia, C. & Lesk, A. M. J. Mol. Biol (1987) 196, 901-917) (Chothia, C. et al. Nature (1989) 342, 877-883) (Al-Lazikani, B., Lesk, A. M. & Chothia, C. J. Mol. Biol (1997) 273, 927-948). This “union” definition of the CDRs is also known as the “Wolfguy” definition by Bujotzek et al. (Bujotzek A1, Dunbar J, Lipsmeier F, Schäfer W, Antes I, Deane C M, Georges G. (2015) “Prediction of VH-VL domain orientation for antibody variable domain modeling.” Proteins Apr; 83 (4): 681-95. doi: 10.1002/prot.24756)). In some embodiments, the CDR definition is based on a combination of Kabat and Chothia definitions (Exemplary system). However, it will be understood that reference to a heavy chain CDR or CDRs and/or a light chain CDR or CDRs of a specific antibody will encompass all CDR definitions as known to those of skill in the art. In one instance, the anti-TfR1 antibody used in the methods described herein comprises the six CDRs of any H2C/L0 mutant disclosed herein based on the Wolfguy or Union definition. In one instance, the anti-TfR1 antibody used in any of the methods described herein comprises the six CDRs of any H2C/L0 mutant disclosed herein based on the Chothia definition. In one instance, the anti-TfR1 antibody used in the methods described herein comprises the six CDRs of any H2C/L0 mutant disclosed herein based on the Kabat definition. In one instance, the anti-TfR1 antibody used in the methods described herein comprises the six CDRs of any H2C/L0 mutant disclosed herein based on the AbM definition. In one instance, the anti-TfR1 antibody used in the methods described herein comprises the six CDRs of any H2C/L0 mutant disclosed herein based on the IMGT definition. In one instance, the anti-TfR1 antibody used in the methods described herein comprises the six CDRs of any H2C/L0 mutant disclosed herein based on the Contact definition.

Table 1 describes the “parental” CDRs (according to the Union definition) of the reference H2C/L0 antibody as well as mutant CDRs present in H2C/L0 mutants described herein. Mutant amino acid positions are identified with AHo numbering (Honegger et al., J Mol Biol, 2001, 309 (3): 657-70). In some embodiments, the anti-TfR1 antibody is an anti-TfR1 antibody that comprises at least one of the CDR mutations described in Table 1 (e.g., 1-10, 1, 2, 3, 4, 5, 6, 7, 8, 9, or 10 of the CDR mutations described in Table 1), but otherwise contains the parental CDR sequences described in Table 1. In some embodiments, an anti-TfR1 antibody comprises (i) one, two, and/or three mutant heavy chain CDRs described in Table 1, and/or (ii) one, two, and/or three mutant light chain CDRs described in Table 1, with all remaining CDRs selected from the parental CDRs depicted in Table 1.

TABLE 1
Union CDRs for H2C/LO and H2C/LO Mutants
Parental CDRs

| Heavy Chain | Heavy Chain | Heavy Chain | Light Chain | Light Chain | Light Chain |
|---|---|---|---|---|---|
| CDR1 | CDR2 | CDR3 | CDR1 | CDR2 | CDR3 |
| GIDFSSSGYMC | CIYTYSSNTYY | GTYGYTGYTY | QASQNINSYLA | RASSLES | QSYYYSGSSNY |
| | AASVKG | TMGYFSL | | | NA |

Mutant CDRs

| Muta- | Heavy Chain | Heavy Chain | Heavy Chain | Light Chain | Light Chain | Light Chain |
|---|---|---|---|---|---|---|
| tion | CDR1 | CDR2 | CDR3 | CDR1 | CDR2 | CDR3 |
| VH- | GIDFASSGYMC | | | | | |
| S32A | | | | | | |
| VH- | GIDFDSSGYMC | | | | | |
| S32D | | | | | | |
| VH- | GIDFHSSGYMC | | | | | |
| S32H | | | | | | |
| VH- | GIDFSASGYMC | | | | | |
| S33A | | | | | | |
| VH- | GIDFSDSGYMC | | | | | |
| S33D | | | | | | |
| VH- | GIDFSESGYMC | | | | | |
| S33E | | | | | | |
| VH- | GIDFSHSGYMC | | | | | |
| S33H | | | | | | |
| VH- | GIDFSSAGYMC | | | | | |
| S38A | | | | | | |
| VH- | GIDFSSDGYMC | | | | | |
| S38D | | | | | | |
| VH- | GIDFSSHGYMC | | | | | |
| S38H | | | | | | |
| VH- | | CIATYSSNTYY | | | | |
| Y59A | | AASVKG | | | | |
| VH- | | CIDTYSSNTYY | | | | |
| Y59D | | AASVKG | | | | |
| VH- | | CIHTYSSNTYY | | | | |
| Y59H | | AASVKG | | | | |
| VH- | | CIETYSSNTYY | | | | |
| Y59E | | AASVKG | | | | |
| VH- | | CIFTYSSNTYY | | | | |
| Y59F | | AASVKG | | | | |
| VH- | | CIYTASSNTYY | | | | |
| Y61A | | AASVKG | | | | |
| VH- | | CIYTDSSNTYY | | | | |
| Y61D | | AASVKG | | | | |
| VH- | | CIHTDSSNTYY | | | | |
| Y59H/ | | AASVKG | | | | |
| Y61D | | | | | | |
| VH- | | CIYTHSSNTYY | | | | |
| Y61H | | AASVKG | | | | |
| VH- | | CIYTYSSATYY | | | | |
| N67A | | AASVKG | | | | |
| VH- | | CIYTYSSDTYY | | | | |
| N67D | | AASVKG | | | | |
| VH- | | CIYTYSSETYY | | | | |
| N67E | | AASVKG | | | | |
| VH- | | CIYTYSSHTYY | | | | |
| N67H | | AASVKG | | | | |
| VH- | | CIYTYSSKTYY | | | | |
| N67K | | AASVKG | | | | |
| VH- | | CIYTYSSRTYY | | | | |
| N67R | | AASVKG | | | | |
| VH- | | CIYTYSSNTAY | | | | |
| Y69A | | AASVKG | | | | |
| VH- | | CIYTYSSNTDY | | | | |
| Y69D | | AASVKG | | | | |
| VH- | | CIYTYSSNTEY | | | | |
| Y69E | | AASVKG | | | | |
| VH- | | CIYTYSSNTHY | | | | |
| Y69H | | AASVKG | | | | |
| VH- | | | GTYGATGYTY | | | |
| Y113A | | | TMGYFSL | | | |
| VH- | | | GTYGDTGYTY | | | |
| Y113D | | | TMGYFSL | | | |
| VH- | | | GTYGHTGYTY | | | |
| Y113H | | | TMGYFSL | | | |
| VH- | | | GTYGYTGATY | | | |
| Y116A | | | TMGYFSL | | | |
| VH- | | | GTYGYTGDTY | | | |
| Y116D | | | TMGYFSL | | | |
| VII- | | | GTYGYTGHTY | | | |
| Y116H | | | TMGYFSL | | | |
| VH- | | | GTYGYTGYAY | | | |
| T130A | | | TMGYFSL | | | |
| VH- | | | GTYGYTGYDY | | | |
| T130D | | | TMGYFSL | | | |
| VH- | | | GTYGYTGYHY | | | |
| T130II | | | TMGYFSL | | | |
| VH- | | | GTYGYTGYTA | | | |
| Y131A | | | TMGYFSL | | | |
| VII- | | | GTYGYTGYTD | | | |
| Y131D | | | TMGYFSL | | | |
| VH- | | | GTYGYTGYTH | | | |
| Y131H | | | TMGYFSL | | | |
| VL- | | | | QASQSINSYLA | | |
| N30S | | | | | | |
| VL- | | | | QASQNISSYLA | | |
| N32S | | | | | | |
| VL- | | | | | DASSLES | |
| R58D | | | | | | |
| VL- | | | | | | QSYYDSGSSNY |
| Y111D | | | | | | NA |
| VL- | | | | | | QSYYHSGSSNY |
| Y111H | | | | | | NA |
| VL- | | | | | | QSYYSSGSSNY |
| Y111S | | | | | | NA |
| VL- | | | | | | QSYYYSGASN |
| S133A | | | | | | YNA |
| VL- | | | | | | QSYYYSGDSN |
| S133D | | | | | | YNA |
| VL- | | | | | | QSYYYSGHSN |
| S133H | | | | | | YNA |
| VH- | | CIYTDSSETYY | | | | |
| Y61D/ | | AASVKG | | | | |
| N67E | | | | | | |
| VH- | | CIYTYSSETEY | | | | |
| N67E/ | | AASVKG | | | | |
| Y69E | | | | | | |
| VL- | | | | QASQSISSYLA | | |
| N30S/ | | | | | | |
| N32S | | | | | | |

Table 2 describes the “parental” CDRs (according to the Chothia definition) of the H2C/L0 antibody as well as mutant CDRs present in H2C/L0 mutants described herein. Mutant amino acid positions are identified with AHo numbering. In some embodiments, the anti-TfR1 antibody is an anti-TfR1 antibody that comprises at least one of the CDR mutations described in Table 2 (e.g., 1-10, 1, 2, 3, 4, 5, 6, 7, 8, 9, or 10 of the CDR mutations described in Table 2), but otherwise contains the parental CDR sequences described in Table 2. In some embodiments, an anti-TfR1 antibody comprises (i) one, two, and/or three mutant heavy chain CDRs described in Table 2, and/or (ii) one, two, and/or three mutant light chain CDRs described in Table 2, with all remaining CDRs selected from the parental CDRs depicted in Table 2.

TABLE 2
Chothia CDRs for H2C/LO and H2C/LO Mutants
Parental CDRs

| Heavy Chain | Heavy Chain | Heavy Chain | Light Chain | Light Chain | Light Chain |
|---|---|---|---|---|---|
| CDR1 | CDR2 | CDR3 | CDR1 | CDR2 | CDR3 |
| GIDFSSSG | TYSS | TYGYTGYTYT | SQNINSY | RAS | YYYSGSSNYN |
| | | MGYFS | | | |

Mutant CDRs

| Muta- | Heavy Chain | Heavy Chain | Heavy Chain | Light Chain | Light Chain | Light Chain |
|---|---|---|---|---|---|---|
| tion | CDR1 | CDR2 | CDR3 | CDR1 | CDR2 | CDR3 |
| VH- | GIDFASSG | | | | | |
| S32A | | | | | | |
| VH- | GIDFDSSG | | | | | |
| S32D | | | | | | |
| VH- | GIDFHSSG | | | | | |
| S32H | | | | | | |
| VH- | GIDFSASG | | | | | |
| S33A | | | | | | |
| VH- | GIDFSDSG | | | | | |
| S33D | | | | | | |
| VH- | GIDFSESG | | | | | |
| S33E | | | | | | |
| VH- | GIDFSHSG | | | | | |
| S33H | | | | | | |
| VH- | GIDFSSAG | | | | | |
| S38A | | | | | | |
| VH- | GIDFSSDG | | | | | |
| S38D | | | | | | |
| VH- | GIDFSSHG | | | | | |
| S38H | | | | | | |
| VH- | | TASS | | | | |
| Y61A | | | | | | |
| VH- | | TDSS | | | | |
| Y61D | | | | | | |
| VH- | | THSS | | | | |
| Y61H | | | | | | |
| VH- | | | TYGATGYTYT | | | |
| Y113A | | | MGYFS | | | |
| VH- | | | TYGDTGYTYT | | | |
| Y113D | | | MGYFS | | | |
| VH- | | | TYGHTGYTYT | | | |
| Y113H | | | MGYFS | | | |
| VH- | | | TYGYTGATYT | | | |
| Y116A | | | MGYFS | | | |
| VH- | | | TYGYTGDTYT | | | |
| Y116D | | | MGYFS | | | |
| VH- | | | TYGYTGHTYT | | | |
| Y116H | | | MGYFS | | | |
| VH- | | | TYGYTGYAYT | | | |
| T130A | | | MGYFS | | | |
| VH- | | | TYGYTGYDYT | | | |
| T130D | | | MGYFS | | | |
| VH- | | | TYGYTGYHYT | | | |
| T130H | | | MGYFS | | | |
| VH- | | | TYGYTGYTAT | | | |
| Y131A | | | MGYFS | | | |
| VH- | | | TYGYTGYTDT | | | |
| Y131D | | | MGYFS | | | |
| VH- | | | TYGYTGYTHT | | | |
| Y131H | | | MGYFS | | | |
| VL- | | | | SQSINSY | | |
| N30S | | | | | | |
| VL- | | | | SQNISSY | | |
| N32S | | | | | | |
| VL- | | | | | DAS | |
| R58D | | | | | | |
| VL- | | | | | | YYDSGSSNYN |
| Y111D | | | | | | |
| VL- | | | | | | YYHSGSSNYN |
| Y111H | | | | | | |
| VL- | | | | | | YYSSGSSNYNA |
| Y111S | | | | | | |
| VL- | | | | | | YYYSGASNYN |
| S133A | | | | | | |
| VL- | | | | | | YYYSGDSNYN |
| S133D | | | | | | |
| VL- | | | | | | YYYSGHSNYN |
| S133II | | | | | | |
| VL- | | | | SQSISSY | | |
| N30S/ | | | | | | |
| N32S | | | | | | |

In some embodiments, an anti-TfR1 antibody comprises a VH comprising VH CDR1, VH CDR2, and VH CDR3, and a VL comprising VL CDR1, VL CDR2, and VL CDR3, wherein (a) the VH CDR1, VH CDR2, VH CDR3, VL CDR1, VL CDR2, and VL CDR3 contain collectively one to ten amino acid differences as compared to a parental antibody whose heavy chain CDRs 1, 2, and 3 comprise the amino acid sequences GIDFSSSGYMC (SEQ ID NO: XX), CIYTYSSNTYYAASVKG (SEQ ID NO:XX), and GTYGYTGYTYTMGYFSL (SEQ ID NO: XX), and whose light chain CDRs 1, 2, and 3 comprise the amino acid sequences QASQNINSYLA (SEQ ID NO:XX), RASSLES (SEQ ID NO:XX), and QSYYYSGSSNYNA (SEQ ID NO:XX), or (b) the VH CDR1, VH CDR2, VH CDR3, VL CDR1, VL CDR2, and VL CDR3 contain collectively one to ten amino acid differences as compared to a parental antibody whose heavy chain CDRs 1, 2, and 3 comprise the amino acid sequences GIDFSSSG (SEQ ID NO: XX), TYSS (SEQ ID NO:XX), and TYGYTGYTYTMGYFS (SEQ ID NO:XX), and whose light chain CDRs 1, 2, and 3 comprise the amino acid sequences SQNINSY (SEQ ID NO:XX), RAS (SEQ ID NO:XX), and YYYSGSSNYN (SEQ ID NO:XX),
wherein the one to ten amino acid differences as compared to the parental antibody occur at one or more of the following residues (using Aho numbering):
VH-S32;
VH-S33;
VH-S33;
VH-S38;
VH-Y59;
VH-Y61;
VH-N67;
VH-Y69;
VH-Y113;
VH-Y116;
VH-T130;
VH-Y131;
VL-N30;
VL-N32;
VL-R58;
VL-Y111;
VL-S133;
VH-Y61 and VH-N67;
VH-N67 and VH-Y69; and/or
VL-N30S and VL-N32S, and
wherein the antibody is monovalent and has a monovalent affinity (K_{D}) for hTfR1 of >10 nM or is bivalent and has a monovalent affinity (K_{D}) for hTfR1 of >100 nM.

In some embodiments, an anti-TfR1 antibody comprises a heavy chain CDR1, CDR2, and CDR3 and/or a light chain variable region CDR1, CDR2, and CDR3 from an H2C/L0 mutant antibody described herein. In some embodiments, an anti-TfR1 antibody comprises a heavy chain CDR1, CDR2, and CDR3 and a light chain CDR1, CDR2, and CDR3 from an H2C/L0 mutant antibody described herein. In some embodiments, an anti-TfR1 antibody comprises a humanized version or humanized variant of an H2C/L0 mutant antibody described herein.

In some embodiments, the anti-TfR1 antibody is a variant of an H2C/L0 mutant antibody described herein which comprises one to thirty conservative amino acid substitutions. In some embodiments, a variant of the anti-TfR1 antibody comprises one to twenty-five conservative amino acid substitutions. In some embodiments, a variant of the anti-TfR1 antibody comprises one to twenty conservative amino acid substitutions. In some embodiments, a variant of the anti-TfR1 antibody comprises one to fifteen conservative amino acid substitutions. In some embodiments, a variant of the anti-TfR1 antibody comprises one to ten conservative amino acid substitution(s). In some embodiments, a variant of the anti-TfR1 antibody comprises one to five conservative amino acid substitution(s). In some embodiments, a variant of the anti-TfR1 antibody comprises one to three conservative amino acid substitution(s). In some embodiments, the conservative amino acid substitution(s) is in a CDR of the antibody. In some embodiments, the conservative amino acid substitution(s) is not in a CDR of the antibody. In some embodiments, the conservative amino acid substitution(s) is in a framework region of the antibody.

Table 3 describes the heavy chain variable region sequences of several H2C/L0 mutants described herein. Table 4 describes the light chain variable region sequences of several H2C/L0 mutants described herein. In both tables, mutant amino acid positions are identified with AHo numbering.

TABLE 3
Heavy Chain Variable Regions of H2C/LO Mutants

| | SEQ ID | |
|---|---|---|
| Mutant Name | NO | Sequence |
| VH-S32A | 100 | QSLVESGGGLVQPGGSLRLSCAASGIDFASSGYMCWVRQAPGKGLEWVGCIYT |
| | | YSSNTYYAASVKGRFTISKTSSTTVYLQMNSLKTEDTAVYYCARGTYGYTGYT |
| | | YTMGYFSLWGQGTLVTVSS |
| VH-S33A | 101 | QSLVESGGGLVQPGGSLRLSCAASGIDFSASGYMCWVRQAPGKGLEWVGCIYT |
| | | YSSNTYYAASVKGRFTISKTSSTTVYLQMNSLKTEDTAVYYCARGTYGYTGYT |
| | | YTMGYFSLWGQGTLVTVSS |
| VH-S38A | 102 | QSLVESGGGLVQPGGSLRLSCAASGIDFSSAGYMCWVRQAPGKGLEWVGCIYT |
| | | YSSNTYYAASVKGRFTISKTSSTTVYLQMNSLKTEDTAVYYCARGTYGYTGYT |
| | | YTMGYFSLWGQGTLVTVSS |
| VH-Y59A | 103 | QSLVESGGGLVQPGGSLRLSCAASGIDFSSSGYMCWVRQAPGKGLEWVGCIAT |
| | | YSSNTYYAASVKGRFTISKTSSTTVYLQMNSLKTEDTAVYYCARGTYGYTGYT |
| | | YTMGYFSLWGQGTLVTVSS |
| VH-Y61A | 104 | QSLVESGGGLVQPGGSLRLSCAASGIDFSSSGYMCWVRQAPGKGLEWVGCIYT |
| | | ASSNTYYAASVKGRFTISKTSSTTVYLQMNSLKTEDTAVYYCARGTYGYTGYT |
| | | YTMGYFSLWGQGTLVTVSS |
| VH-N67A | 105 | QSLVESGGGLVQPGGSLRLSCAASGIDFSSSGYMCWVRQAPGKGLEWVGCIYT |
| | | YSSATYYAASVKGRFTISKTSSTTVYLQMNSLKTEDTAVYYCARGTYGYTGYT |
| | | YTMGYFSLWGQGTLVTVSS |
| VH-Y69A | 106 | QSLVESGGGLVQPGGSLRLSCAASGIDFSSSGYMCWVRQAPGKGLEWVGCIYT |
| | | YSSNTAYAASVKGRFTISKTSSTTVYLQMNSLKTEDTAVYYCARGTYGYTGYT |
| | | YTMGYFSLWGQGTLVTVSS |
| VH-Y113A | 107 | QSLVESGGGLVQPGGSLRLSCAASGIDFSSSGYMCWVRQAPGKGLEWVGCIYT |
| | | YSSNTYYAASVKGRFTISKTSSTTVYLQMNSLKTEDTAVYYCARGTYGATGYT |
| | | YTMGYFSLWGQGTLVTVSS |
| VH-Y116A | 108 | QSLVESGGGLVQPGGSLRLSCAASGIDFSSSGYMCWVRQAPGKGLEWVGCIYT |
| | | YSSNTYYAASVKGRFTISKTSSTTVYLQMNSLKTEDTAVYYCARGTYGYTGAT |
| | | YTMGYFSLWGQGTLVTVSS |
| VH-T130A | 109 | QSLVESGGGLVQPGGSLRLSCAASGIDFSSSGYMCWVRQAPGKGLEWVGCIYT |
| | | YSSNTYYAASVKGRFTISKTSSTTVYLQMNSLKTEDTAVYYCARGTYGYTGYA |
| | | YTMGYFSLWGQGTLVTVSS |
| VH-Y131A | 110 | QSLVESGGGLVQPGGSLRLSCAASGIDFSSSGYMCWVRQAPGKGLEWVGCIYT |
| | | YSSNTYYAASVKGRFTISKTSSTTVYLQMNSLKTEDTAVYYCARGTYGYTGYT |
| | | ATMGYFSLWGQGTLVTVSS |
| VH-EVQL- | 111 | EVQLVESGGGLVQPGGSLRLSCAASGIDFASSGYMCWVRQAPGKGLEWVGCIY |
| S32A | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 112 | EVQLVESGGGLVQPGGSLRLSCAASGIDFDSSGYMCWVRQAPGKGLEWVGCIY |
| S32D | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 113 | EVQLVESGGGLVQPGGSLRLSCAASGIDFHSSGYMCWVRQAPGKGLEWVGCIY |
| S32H | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 114 | EVQLVESGGGLVQPGGSLRLSCAASGIDFSASGYMCWVRQAPGKGLEWVGCIY |
| S33A | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 115 | EVQLVESGGGLVQPGGSLRLSCAASGIDFSDSGYMCWVRQAPGKGLEWVGCIY |
| S33D | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 116 | EVQLVESGGGLVQPGGSLRLSCAASGIDFSESGYMCWVRQAPGKGLEWVGCIY |
| S33E | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 117 | EVQLVESGGGLVQPGGSLRLSCAASGIDFSHSGYMCWVRQAPGKGLEWVGCIY |
| S33H | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 118 | EVQLVESGGGLVQPGGSLRLSCAASGIDFSSAGYMCWVRQAPGKGLEWVGCIY |
| S38A | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 119 | EVQLVESGGGLVQPGGSLRLSCAASGIDFSSDGYMCWVRQAPGKGLEWVGCIY |
| S38D | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 120 | EVQLVESGGGLVQPGGSLRLSCAASGIDFSSHGYMCWVRQAPGKGLEWVGCIY |
| S38H | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 121 | EVQLVESGGGLVQPGGSLRLSCAASGIDFSSSGYMCWVRQAPGKGLEWVGCIA |
| Y59A | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 122 | EVQLVESGGGLVQPGGSLRLSCAASGIDFSSSGYMCWVRQAPGKGLEWVGCID |
| Y59D | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 123 | EVQLVESGGGLVQPGGSLRLSCAASGIDFSSSGYMCWVRQAPGKGLEWVGCIH |
| Y59H | | TYSSNTYYAASVKGRFTISKTSSTTVYLQMNSLKTEDTAVYYCARGTYGYTGYT |
| | | YTMGYFSLWGQGTLVTVSS |
| VH-EVQL- | 124 | EVQLVESGGGLVQPGGSLRLSCAASGIDFSSSGYMCWVRQAPGKGLEWVGCIY |
| Y61A | | TASSNTYYAASVKGRFTISKTSSTTVYLQMNSLKTEDTAVYYCARGTYGYTGYT |
| | | YTMGYFSLWGQGTLVTVSS |
| VH-EVQL- | 125 | EVQLVESGGGLVQPGGSLRLSCAASGIDFSSSGYMCWVRQAPGKGLEWVGCIY |
| Y61D | | TDSSNTYYAASVKGRFTISKTSSTTVYLQMNSLKTEDTAVYYCARGTYGYTGYT |
| | | YTMGYFSLWGQGTLVTVSS |
| VH-EVQL- | 126 | EVQLVESGGGLVQPGGSLRLSCAASGIDFSSSGYMCWVRQAPGKGLEWVGCIY |
| Y61H | | THSSNTYYAASVKGRFTISKTSSTTVYLQMNSLKTEDTAVYYCARGTYGYTGYT |
| | | YTMGYFSLWGQGTLVTVSS |
| VH-EVQL- | 127 | EVQLVESGGGLVQPGGSLRLSCAASGIDFSSSGYMCWVRQAPGKGLEWVGCIY |
| N67A | | TYSSATYYAASVKGRFTISKTSSTTVYLQMNSLKTEDTAVYYCARGTYGYTGYT |
| | | YTMGYFSLWGQGTLVTVSS |
| VH-EVQL- | 128 | EVQLVESGGGLVQPGGSLRLSCAASGIDFSSSGYMCWVRQAPGKGLEWVGCIY |
| N67D | | TYSSDTYYAASVKGRFTISKTSSTTVYLQMNSLKTEDTAVYYCARGTYGYTGYT |
| | | YTMGYFSLWGQGTLVTVSS |
| VH-EVQL- | 129 | EVQLVESGGGLVQPGGSLRLSCAASGIDFSSSGYMCWVRQAPGKGLEWVGCIY |
| N67E | | TYSSETYYAASVKGRFTISKTSSTTVYLQMNSLKTEDTAVYYCARGTYGYTGYT |
| | | YTMGYFSLWGQGTLVTVSS |
| VH-EVQL- | 130 | EVQLVESGGGLVQPGGSLRLSCAASGIDFSSSGYMCWVRQAPGKGLEWVGCIY |
| N67H | | TYSSHTYYAASVKGRFTISKTSSTTVYLQMNSLKTEDTAVYYCARGTYGYTGYT |
| | | YTMGYFSLWGQGTLVTVSS |
| VH-EVQL- | 131 | EVQLVESGGGLVQPGGSLRLSCAASGIDFSSSGYMCWVRQAPGKGLEWVGCIY |
| Y69A | | TYSSNTAYAASVKGRFTISKTSSTTVYLQMNSLKTEDTAVYYCARGTYGYTGYT |
| | | YTMGYFSLWGQGTLVTVSS |
| VH-EVQL- | 132 | EVQLVESGGGLVQPGGSLRLSCAASGIDFSSSGYMCWVRQAPGKGLEWVGCIY |
| Y69D | | TYSSNTDYAASVKGRFTISKTSSTTVYLQMNSLKTEDTAVYYCARGTYGYTGYT |
| | | YTMGYFSLWGQGTLVTVSS |
| VH-EVQL- | 133 | EVQLVESGGGLVQPGGSLRLSCAASGIDFSSSGYMCWVRQAPGKGLEWVGCIY |
| Y69E | | TYSSNTEYAASVKGRFTISKTSSTTVYLQMNSLKTEDTAVYYCARGTYGYTGYT |
| | | YTMGYFSLWGQGTLVTVSS |
| VH-EVQL- | 134 | EVQLVESGGGLVQPGGSLRLSCAASGIDFSSSGYMCWVRQAPGKGLEWVGCIY |
| Y69H | | TYSSNTHYAASVKGRFTISKTSSTTVYLQMNSLKTEDTAVYYCARGTYGYTGYT |
| | | YTMGYFSLWGQGTLVTVSS |
| VH-EVQL- | 135 | EVQLVESGGGLVQPGGSLRLSCAASGIDFSSSGYMCWVRQAPGKGLEWVGCIY |
| Y113A | | TYSSNTYYAASVKGRFTISKTSSTTVYLQMNSLKTEDTAVYYCARGTYGATGYT |
| | | YTMGYFSLWGQGTLVTVSS |
| VH-EVQL- | 136 | EVQLVESGGGLVQPGGSLRLSCAASGIDFSSSGYMCWVRQAPGKGLEWVGCIY |
| Y113D | | TYSSNTYYAASVKGRFTISKTSSTTVYLQMNSLKTEDTAVYYCARGTYGDTGYT |
| | | YTMGYFSLWGQGTLVTVSS |
| VH-EVQL- | 137 | EVQLVESGGGLVQPGGSLRLSCAASGIDFSSSGYMCWVRQAPGKGLEWVGCIY |
| Y113H | | TYSSNTYYAASVKGRFTISKTSSTTVYLQMNSLKTEDTAVYYCARGTYGHTGYT |
| | | YTMGYFSLWGQGTLVTVSS |
| VH-EVQL- | 138 | EVQLVESGGGLVQPGGSLRLSCAASGIDFSSSGYMCWVRQAPGKGLEWVGCIY |
| Y116A | | TYSSNTYYAASVKGRFTISKTSSTTVYLQMNSLKTEDTAVYYCARGTYGYTGAT |
| | | YTMGYFSLWGQGTLVTVSS |
| VH-EVQL- | 139 | EVQLVESGGGLVQPGGSLRLSCAASGIDFSSSGYMCWVRQAPGKGLEWVGCIY |
| Y116D | | TYSSNTYYAASVKGRFTISKTSSTTVYLQMNSLKTEDTAVYYCARGTYGYTGDT |
| | | YTMGYFSLWGQGTLVTVSS |
| VH-EVQL- | 140 | EVQLVESGGGLVQPGGSLRLSCAASGIDFSSSGYMCWVRQAPGKGLEWVGCIY |
| Y116H | | TYSSNTYYAASVKGRFTISKTSSTTVYLQMNSLKTEDTAVYYCARGTYGYTGHT |
| | | YTMGYFSLWGQGTLVTVSS |
| VH-EVQL- | 141 | EVQLVESGGGLVQPGGSLRLSCAASGIDFSSSGYMCWVRQAPGKGLEWVGCIY |
| T130A | | TYSSNTYYAASVKGRFTISKTSSTTVYLQMNSLKTEDTAVYYCARGTYGYTGY |
| | | AYTMGYFSLWGQGTLVTVSS |
| VH-EVQL- | 142 | EVQLVESGGGLVQPGGSLRLSCAASGIDFSSSGYMCWVRQAPGKGLEWVGCIY |
| T130D | | TYSSNTYYAASVKGRFTISKTSSTTVYLQMNSLKTEDTAVYYCARGTYGYTGY |
| | | DYTMGYFSLWGQGTLVTVSS |
| VH-EVQL- | 143 | EVQLVESGGGLVQPGGSLRLSCAASGIDFSSSGYMCWVRQAPGKGLEWVGCIY |
| T130H | | TYSSNTYYAASVKGRFTISKTSSTTVYLQMNSLKTEDTAVYYCARGTYGYTGY |
| | | HYTMGYFSLWGQGTLVTVSS |
| VH-EVQL- | 144 | EVQLVESGGGLVQPGGSLRLSCAASGIDFSSSGYMCWVRQAPGKGLEWVGCIY |
| Y131A | | TYSSNTYYAASVKGRFTISKTSSTTVYLQMNSLKTEDTAVYYCARGTYGYTGYT |
| | | ATMGYFSLWGQGTLVTVSS |
| VH-EVQL- | 145 | EVQLVESGGGLVQPGGSLRLSCAASGIDFSSSGYMCWVRQAPGKGLEWVGCIY |
| Y131D | | TYSSNTYYAASVKGRFTISKTSSTTVYLQMNSLKTEDTAVYYCARGTYGYTGYT |
| | | DTMGYFSLWGQGTLVTVSS |
| VH-EVQL- | 146 | EVQLVESGGGLVQPGGSLRLSCAASGIDFSSSGYMCWVRQAPGKGLEWVGCIY |
| Y131H | | TYSSNTYYAASVKGRFTISKTSSTTVYLQMNSLKTEDTAVYYCARGTYGYTGYT |
| | | HTMGYFSLWGQGTLVTVSS |
| VH-EVQL- | 147 | EVQLVESGGGLVQPGGSLRLSCAASGIDFDSSGYMCWVRQAPGKGLEWVGCIY |
| S32D/Y116A | | TYSSNTYYAASVKGRFTISKTSSTTVYLQMNSLKTEDTAVYYCARGTYGYTGAT |
| | | YTMGYFSLWGQGTLVTVSS |
| VH-EVQL- | 148 | EVQLVESGGGLVQPGGSLRLSCAASGIDFSSSGYMCWVRQAPGKGLEWVGCIY |
| Y69A/Y116A | | TYSSNTAYAASVKGRFTISKTSSTTVYLQMNSLKTEDTAVYYCARGTYGYTGAT |
| | | YTMGYFSLWGQGTLVTVSS |
| VH-EVQL- | 149 | EVQLVESGGGLVQPGGSLRLSCAASGIDFSSSGYMCWVRQAPGKGLEWVGCIY |
| Y69E/Y116A | | TYSSNTEYAASVKGRFTISKTSSTTVYLQMNSLKTEDTAVYYCARGTYGYTGAT |
| | | YTMGYFSLWGQGTLVTVSS |
| VH-EVQL- | 150 | EVQLVESGGGLVQPGGSLRLSCAASGIDFDSSGYMCWVRQAPGKGLEWVGCIY |
| S32D/Y61D | | TDSSNTYYAASVKGRFTISKTSSTTVYLQMNSLKTEDTAVYYCARGTYGYTGYT |
| | | YTMGYFSLWGQGTLVTVSS |
| VH-EVQL- | 151 | EVQLVESGGGLVQPGGSLRLSCAASGIDFDSSGYMCWVRQAPGKGLEWVGCIY |
| S32D/N67D | | TYSSDTYYAASVKGRFTISKTSSTTVYLQMNSLKTEDTAVYYCARGTYGYTGYT |
| | | YTMGYFSLWGQGTLVTVSS |
| VH-EVQL- | 152 | EVQLVESGGGLVQPGGSLRLSCAASGIDFDSSGYMCWVRQAPGKGLEWVGCIY |
| S32D/N67E | | TYSSETYYAASVKGRFTISKTSSTTVYLQMNSLKTEDTAVYYCARGTYGYTGYT |
| | | YTMGYFSLWGQGTLVTVSS |
| VH-EVQL- | 153 | EVQLVESGGGLVQPGGSLRLSCAASGIDFDSSGYMCWVRQAPGKGLEWVGCIY |
| S32D/Y69D | | TYSSNTDYAASVKGRFTISKTSSTTVYLQMNSLKTEDTAVYYCARGTYGYTGYT |
| | | YTMGYFSLWGQGTLVTVSS |
| VH-EVQL- | 154 | EVQLVESGGGLVQPGGSLRLSCAASGIDFDSSGYMCWVRQAPGKGLEWVGCIY |
| S32D/Y69E | | TYSSNTEYAASVKGRFTISKTSSTTVYLQMNSLKTEDTAVYYCARGTYGYTGYT |
| | | YTMGYFSLWGQGTLVTVSS |
| VH-EVQL- | 155 | EVQLVESGGGLVQPGGSLRLSCAASGIDFDSSGYMCWVRQAPGKGLEWVGCIH |
| S32D/Y59H/Y6 | | TDSSNTYYAASVKGRFTISKTSSTTVYLQMNSLKTEDTAVYYCARGTYGYTGYT |
| 1D | | YTMGYFSLWGQGTLVTVSS |
| VH-EVQL- | 156 | EVQLVESGGGLVQPGGSLRLSCAASGIDFSSSGYMAWVRQAPGKGLEWVGAIY |
| CACA/N67D | | TYSSDTYYAASVKGRFTISKTSSTTVYLQMNSLKTEDTAVYYCARGTYGYTGYT |
| | | YTMGYFSLWGQGTLVTVSS |
| VH-EVQL- | 157 | EVQLVESGGGLVQPGGSLRLSCAASGIDFSSSGYMAWVRQAPGKGLEWVGAIY |
| CACA/Y69D | | TYSSNTDYAASVKGRFTISKTSSTTVYLQMNSLKTEDTAVYYCARGTYGYTGYT |
| | | YTMGYFSLWGQGTLVTVSS |
| VH-EVQL- | 158 | EVQLVESGGGLVQPGGSLRLSCAASGIDFSSSGYMCWVRQAPGKGLEWVGCIY |
| Y61D/N67E | | TDSSETYYAASVKGRFTISKTSSTTVYLQMNSLKTEDTAVYYCARGTYGYTGYT |
| | | YTMGYFSLWGQGTLVTVSS |
| VH-EVQL- | 159 | EVQLVESGGGLVQPGGSLRLSCAASGIDFSSSGYMCWVRQAPGKGLEWVGCIY |
| N67E/Y69E | | TYSSETEYAASVKGRFTISKTSSTTVYLQMNSLKTEDTAVYYCARGTYGYTGYT |
| | | YTMGYFSLWGQGTLVTVSS |
| VH-EVQL- | 160 | EVQLVESGGGLVQPGGSLRLSCAASGIDFSSSGYMCWVRQAPGKGLEWVGCIY |
| N67E/Y116A | | TYSSETYYAASVKGRFTISKTSSTTVYLQMNSLKTEDTAVYYCARGTYGYTGAT |
| | | YTMGYFSLWGQGTLVTVSS |

TABLE 4
Light Chain Variable Regions of H2C/LO Mutants

| | SEQ ID | |
|---|---|---|
| Mutant Name | NO | Sequence |
| VL-N30S | 200 | DIQMTQSPSTLSASVGDRVTITCQASQSINSYLAWYQQKPGKAPKLLIYRASSLE |
| | | SGVPSRFSGSGSGTEFTLTISSLQPDDFATYYCQSYYYSGSSNYNAFGGGTKVEIK |
| VL-N32S | 201 | DIQMTQSPSTLSASVGDRVTITCQASQNISSYLAWYQQKPGKAPKLLIYRASSLE |
| | | SGVPSRFSGSGSGTEFTLTISSLQPDDFATYYCQSYYYSGSSNYNAFGGGTKVEIK |
| VL-R58D | 202 | DIQMTQSPSTLSASVGDRVTITCQASQNINSYLAWYQQKPGKAPKLLIYDASSLE |
| | | SGVPSRFSGSGSGTEFTLTISSLQPDDFATYYCQSYYYSGSSNYNAFGGGTKVEIK |
| VL-Y111D | 203 | DIQMTQSPSTLSASVGDRVTITCQASQNINSYLAWYQQKPGKAPKLLIYRASSLE |
| | | SGVPSRFSGSGSGTEFTLTISSLQPDDFATYYCQSYYDSGSSNYNAFGGGTKVEIK |
| VI-Y111H | 204 | DIQMTQSPSTLSASVGDRVTITCQASQNINSYLAWYQQKPGKAPKILIYRASSLE |
| | | SGVPSRFSGSGSGTEFTLTISSLQPDDFATYYCQSYYHSGSSNYNAFGGGTKVEIK |
| VL-Y111S | 205 | DIQMTQSPSTLSASVGDRVTITCQASQNINSYLAWYQQKPGKAPKLLIYRASSLE |
| | | SGVPSRFSGSGSGTEFTLTISSLQPDDFATYYCQSYYSSGSSNYNAFGGGTKVEIK |
| VL-S133A | 206 | DIQMTQSPSTLSASVGDRVTITCQASQNINSYLAWYQQKPGKAPKLLIYRASSLE |
| | | SGVPSRFSGSGSGTEFTLTISSLQPDDFATYYCQSYYYSGASNYNAFGGGTKVEI |
| | | K |
| VL-S133D | 207 | DIQMTQSPSTLSASVGDRVTITCQASQNINSYLAWYQQKPGKAPKLLIYRASSLE |
| | | SGVPSRFSGSGSGTEFTLTISSLQPDDFATYYCQSYYYSGDSNYNAFGGGTKVEI |
| | | K |
| VL-S133H | 208 | DIQMTQSPSTLSASVGDRVTITCQASQNINSYLAWYQQKPGKAPKLLIYRASSLE |
| | | SGVPSRFSGSGSGTEFTLTISSLQPDDFATYYCQSYYYSGHSNYNAFGGGTKVEI |
| | | K |
| VL-N30S/R58D | 209 | DIQMTQSPSTLSASVGDRVTITCQASQSINSYLAWYQQKPGKAPKLLIYDASSLE |
| | | SGVPSRFSGSGSGTEFTLTISSLQPDDFATYYCQSYYYSGSSNYNAFGGGTKVEIK |
| VL- | 210 | DIQMTQSPSTLSASVGDRVTITCQASQSINSYLAWYQQKPGKAPKLLIYDASSLE |
| N30S/R58D/Y11 | | SGVPSRFSGSGSGTEFTLTISSLQPDDFATYYCQSYYSSGSSNYNAFGGGTKVEIK |
| 1S | | |
| VL- | 211 | DIQMTQSPSTLSASVGDRVTITCQASQSINSYLAWYQQKPGKAPKLLIYDASSLE |
| N30S/R58D/S13 | | SGVPSRFSGSGSGTEFTLTISSLQPDDFATYYCQSYYYSGDSNYNAFGGGTKVEI |
| 3D | | K |
| VL- | 212 | DIQMTQSPSTLSASVGDRVTITCQASQSINSYLAWYQQKPGKAPKLLIYRASSLE |
| N30S/Y111D | | SGVPSRFSGSGSGTEFTLTISSLQPDDFATYYCQSYYDSGSSNYNAFGGGTKVEIK |
| VL- | 213 | DIQMTQSPSTLSASVGDRVTITCQASQSINSYLAWYQQKPGKAPKLLIYRASSLE |
| N30S/S133D | | SGVPSRFSGSGSGTEFTLTISSLQPDDFATYYCQSYYYSGDSNYNAFGGGTKVEI |
| | | K |
| VL- | 214 | DIQMTQSPSTLSASVGDRVTITCQASQSINSYLAWYQQKPGKAPKLLIYRASSLE |
| N30S/Y111S | | SGVPSRFSGSGSGTEFTLTISSLQPDDFATYYCQSYYSSGSSNYNAFGGGTKVEIK |
| VL- | 215 | DIQMTQSPSTLSASVGDRVTITCQASQNINSYLAWYQQKPGKAPKLLIYDASSLE |
| R58D/S133D | | SGVPSRFSGSGSGTEFTLTISSLQPDDFATYYCQSYYYSGDSNYNAFGGGTKVEI |
| | | K |
| VL-N30S/N32S/ | 216 | DIQMTQSPSTLSASVGDRVTITCQASQSISSYLAWYQQKPGKAPKLLIYRASSLES |
| S133D | | GVPSRFSGSGSGTEFTLTISSLQPDDFATYYCQSYYYSGDSNYNAFGGGTKVEIK |

In some embodiments, an anti-TfR1 antibody comprises a heavy chain variable region comprising at least 75%, 80%, 85%, 90%, 95%, 96%, 97%, 98%, 99%, or 100% identity to a sequence set forth in Table 3 and a light chain variable region comprising at least 75%, 80%, 85%, 90%, 95%, 96%, 97%, 98%, 99%, or 100% identity to a sequence set forth in Table 4, where the VH and VL is not identical to the parental H2C/L0 VH and VL sequence.

In some embodiments, an anti-TfR1 antibody comprises a heavy chain variable region comprising an amino acid sequence that has the three VH CDRs of any H2C/L0 mutant antibody described herein and which has at least 75%, 80%, 85%, 90%, 95%, 96%, 97%, 98%, 99%, or 100% identity to any one of the VH sequences set forth in Table 3 and a light chain variable region comprising an amino acid sequence that has the three VL CDRs of any H2C/L0 mutant antibody described herein and which has at least 75%, 80%, 85%, 90%, 95%, 96%, 97%, 98%, 99%, or 100% identity to any one of the sequences set forth in Table 4.

In some embodiments of the anti-TfR1 antibody:
the VH CDR1 comprises the amino acid sequence GIDFASSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFDSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFHSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSASGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSDSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSESGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSHSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSAGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSDGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSHGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIATYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIDTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIHTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIETYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIFTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIYTASSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTDSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIHTDSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTHSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSATYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSETYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSHTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSKTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSRTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIYTYSSNTAYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTDYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTEYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTHYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGATGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGDTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGHTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGATYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGDTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGHTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYAYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYDYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYHYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTATMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTDTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTHTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQSINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNISSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence DASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYDSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYHSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYSSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGASNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGDSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGHSNYNA (SEQ ID NO:XX);
the VH CDR1 comprises the amino acid sequence GIDFSSSGYMC (SEQ ID NO:XX); the VH CDR2 comprises the amino acid sequence CIYTDSSETYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSETEYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX); or
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQSISSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX).

In some embodiments of the anti-TfR1 antibody:
(a) the VH CDR1 comprises the amino acid sequence GIDFDSSGYMC (SEQ ID NO: XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence DASSLES (SEQ ID NO:XX); and the VL CDR3 comprises the amino acid sequence QSYYYSGSSNYNA (SEQ ID NO:XX);
(b) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence DASSLES (SEQ ID NO:XX); and
(c) the VH CDR1 comprises the amino acid sequence GIDFDSSGYMC (SEQ ID NO: XX); the VH CDR2 comprises the amino acid sequence CIYTDSSNTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(d) the VH CDR1 comprises the amino acid sequence GIDFDSSGYMC (SEQ ID NO: XX); the VH CDR2 comprises the amino acid sequence CIYTYSSETYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(e) the VH CDR1 comprises the amino acid sequence GIDFDSSGYMC (SEQ ID NO: XX); the VH CDR2 comprises the amino acid sequence CIYTYSSNTDYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(f) the VH CDR1 comprises the amino acid sequence GIDFDSSGYMC (SEQ ID NO: XX); the VH CDR2 comprises the amino acid sequence CIYTYSSNTEYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(g) the VH CDR1 comprises the amino acid sequence GIDFDSSGYMC (SEQ ID NO: XX); the VH CDR2 comprises the amino acid sequence CIHTDSSNTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and
(h) the VH CDR1 comprises the amino acid sequence GIDFDSSGYMC (SEQ ID NO: XX); the VH CDR2 comprises the amino acid sequence CIYTYSSNTYYAASVKG (SEQ ID NO: XX); the VH CDR3 comprises the amino acid sequence GTYGYTGATYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(i) the VH CDR1 comprises the amino acid sequence GIDFSSSGYMC (SEQ ID NO: XX); the VH CDR2 comprises the amino acid sequence CIYTYSSNTAYAASVKG (SEQ ID NO: XX); the VH CDR3 comprises the amino acid sequence GTYGYTGATYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(j) the VH CDR1 comprises the amino acid sequence GIDFSSSGYMC (SEQ ID NO: XX); the VH CDR2 comprises the amino acid sequence CIYTYSSNTEYAASVKG (SEQ ID NO: XX); the VH CDR3 comprises the amino acid sequence GTYGYTGATYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(k) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence DASSLES (SEQ ID NO:XX); and the VL CDR3 comprises the amino acid sequence QSYYYSGSSNYNA (SEQ ID NO:XX);
(l) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence RASSLES (SEQ ID NO:XX); and the VL CDR3 comprises the amino acid sequence QSYYSSGSSNYNA (SEQ ID NO:XX);
(m) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence RASSLES (SEQ ID NO:XX); and the VL CDR3 comprises the amino acid sequence QSYYDSGSSNYNA (SEQ ID NO:XX);
(n) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence RASSLES (SEQ ID NO:XX); and the VL CDR3 comprises the amino acid sequence QSYYYSGDSNYNA (SEQ ID NO:XX);
(o) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence DASSLES (SEQ ID NO:XX); and the VL CDR3 comprises the amino acid sequence QSYYSSGSSNYNA (SEQ ID NO:XX);
(p) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence DASSLES (SEQ ID NO:XX); and the VL CDR3 comprises the amino acid sequence QSYYYSGDSNYNA (SEQ ID NO:XX);
(q) the VH CDR1 comprises the amino acid sequence GIDFDSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and
(r) the VH CDR1 comprises the amino acid sequence GIDFSSSGYMC (SEQ ID NO: XX); the VH CDR2 comprises the amino acid sequence CIYTYSSETYYAASVKG (SEQ ID NO: XX); the VH CDR3 comprises the amino acid sequence GTYGYTGATYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(s) the VH CDR1 comprises the amino acid sequence GIDFSSSGYMC (SEQ ID NO: XX); the VH CDR2 comprises the amino acid sequence CIYTYSSETYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSISSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(t) the VH CDR1 comprises the amino acid sequence GIDFSSSGYMC (SEQ ID NO: XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSISSYLA (SEQ ID NO: XX); the VL CDR2 comprises the amino acid sequence RASSLES (SEQ ID NO:XX); and the VL CDR3 comprises the amino acid sequence QSYYYSGDSNYNA (SEQ ID NO:XX); or
(u) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSISSYLA (SEQ ID NO: XX); the VL CDR2 comprises the amino acid sequence RASSLES (SEQ ID NO:XX); and the VL CDR3 comprises the amino acid sequence QSYYYSGDSNYNA (SEQ ID NO:XX).

In some embodiments of the anti-TfR1 antibody:
the VH comprises the amino acid sequence of SEQ ID NO: 100 and the VL comprises the amino acid sequence of SEQ ID NO:301;
the VH comprises the amino acid sequence of SEQ ID NO: 101 and the VL comprises the amino acid sequence of SEQ ID NO:301;
the VH comprises the amino acid sequence of SEQ ID NO: 102 and the VL comprises the amino acid sequence of SEQ ID NO:301;
the VH comprises the amino acid sequence of SEQ ID NO: 103 and the VL comprises the amino acid sequence of SEQ ID NO:301;
the VH comprises the amino acid sequence of SEQ ID NO: 104 and the VL comprises the amino acid sequence of SEQ ID NO:301;
the VH comprises the amino acid sequence of SEQ ID NO: 105 and the VL comprises the amino acid sequence of SEQ ID NO:301;
the VH comprises the amino acid sequence of SEQ ID NO: 106 and the VL comprises the amino acid sequence of SEQ ID NO:301;
the VH comprises the amino acid sequence of SEQ ID NO:107 and the VL comprises the amino acid sequence of SEQ ID NO:301;
the VH comprises the amino acid sequence of SEQ ID NO: 108 and the VL comprises the amino acid sequence of SEQ ID NO:301;
the VH comprises the amino acid sequence of SEQ ID NO: 109 and the VL comprises the amino acid sequence of SEQ ID NO:301;
the VH comprises the amino acid sequence of SEQ ID NO: 110 and the VL comprises the amino acid sequence of SEQ ID NO:301;
the VH comprises the amino acid sequence of SEQ ID NO:111 and the VL comprises the amino acid sequence of SEQ ID NO:301;
the VH comprises the amino acid sequence of SEQ ID NO:112 and the VL comprises the amino acid sequence of SEQ ID NO:301;
the VH comprises the amino acid sequence of SEQ ID NO: 113 and the VL comprises the amino acid sequence of SEQ ID NO:301;
the VH comprises the amino acid sequence of SEQ ID NO:114 and the VL comprises the amino acid sequence of SEQ ID NO:301;
the VH comprises the amino acid sequence of SEQ ID NO:115 and the VL comprises the amino acid sequence of SEQ ID NO:301;
the VH comprises the amino acid sequence of SEQ ID NO: 116 and the VL comprises the amino acid sequence of SEQ ID NO:301;
the VH comprises the amino acid sequence of SEQ ID NO: 117 and the VL comprises the amino acid sequence of SEQ ID NO:301;
the VH comprises the amino acid sequence of SEQ ID NO:118 and the VL comprises the amino acid sequence of SEQ ID NO:301;
the VH comprises the amino acid sequence of SEQ ID NO: 119 and the VL comprises the amino acid sequence of SEQ ID NO:301;
the VH comprises the amino acid sequence of SEQ ID NO: 120 and the VL comprises the amino acid sequence of SEQ ID NO:301;
the VH comprises the amino acid sequence of SEQ ID NO: 121 and the VL comprises the amino acid sequence of SEQ ID NO:301;
the VH comprises the amino acid sequence of SEQ ID NO: 122 and the VL comprises the amino acid sequence of SEQ ID NO:301;
the VH comprises the amino acid sequence of SEQ ID NO: 123 and the VL comprises the amino acid sequence of SEQ ID NO:301;
the VH comprises the amino acid sequence of SEQ ID NO: 124 and the VL comprises the amino acid sequence of SEQ ID NO:301;
the VH comprises the amino acid sequence of SEQ ID NO:125 and the VL comprises the amino acid sequence of SEQ ID NO:301;
the VH comprises the amino acid sequence of SEQ ID NO: 126 and the VL comprises the amino acid sequence of SEQ ID NO:301;
the VH comprises the amino acid sequence of SEQ ID NO: 127 and the VL comprises the amino acid sequence of SEQ ID NO:301;
the VH comprises the amino acid sequence of SEQ ID NO: 128 and the VL comprises the amino acid sequence of SEQ ID NO:301;
the VH comprises the amino acid sequence of SEQ ID NO: 129 and the VL comprises the amino acid sequence of SEQ ID NO:301;
the VH comprises the amino acid sequence of SEQ ID NO:130 and the VL comprises the amino acid sequence of SEQ ID NO:301;
the VH comprises the amino acid sequence of SEQ ID NO: 131 and the VL comprises the amino acid sequence of SEQ ID NO:301;
the VH comprises the amino acid sequence of SEQ ID NO: 132 and the VL comprises the amino acid sequence of SEQ ID NO:301;
the VH comprises the amino acid sequence of SEQ ID NO: 133 and the VL comprises the amino acid sequence of SEQ ID NO:301;
the VH comprises the amino acid sequence of SEQ ID NO: 134 and the VL comprises the amino acid sequence of SEQ ID NO:301;
the VH comprises the amino acid sequence of SEQ ID NO:135 and the VL comprises the amino acid sequence of SEQ ID NO:301;
the VH comprises the amino acid sequence of SEQ ID NO: 136 and the VL comprises the amino acid sequence of SEQ ID NO:301;
the VH comprises the amino acid sequence of SEQ ID NO:137 and the VL comprises the amino acid sequence of SEQ ID NO:301;
the VH comprises the amino acid sequence of SEQ ID NO:138 and the VL comprises the amino acid sequence of SEQ ID NO:301;
the VH comprises the amino acid sequence of SEQ ID NO: 139 and the VL comprises the amino acid sequence of SEQ ID NO:301;
the VH comprises the amino acid sequence of SEQ ID NO: 140 and the VL comprises the amino acid sequence of SEQ ID NO:301;
the VH comprises the amino acid sequence of SEQ ID NO: 141 and the VL comprises the amino acid sequence of SEQ ID NO:301;
the VH comprises the amino acid sequence of SEQ ID NO: 142 and the VL comprises the amino acid sequence of SEQ ID NO:301;
the VH comprises the amino acid sequence of SEQ ID NO: 143 and the VL comprises the amino acid sequence of SEQ ID NO:301;
the VH comprises the amino acid sequence of SEQ ID NO: 144 and the VL comprises the amino acid sequence of SEQ ID NO:301;
the VH comprises the amino acid sequence of SEQ ID NO:145 and the VL comprises the amino acid sequence of SEQ ID NO:301;
the VH comprises the amino acid sequence of SEQ ID NO:146 and the VL comprises the amino acid sequence of SEQ ID NO:301;
the VH comprises the amino acid sequence of SEQ ID NO: 147 and the VL comprises the amino acid sequence of SEQ ID NO:301;
the VH comprises the amino acid sequence of SEQ ID NO: 148 and the VL comprises the amino acid sequence of SEQ ID NO:301;
the VH comprises the amino acid sequence of SEQ ID NO: 149 and the VL comprises the amino acid sequence of SEQ ID NO:301;
the VH comprises the amino acid sequence of SEQ ID NO:150 and the VL comprises the amino acid sequence of SEQ ID NO:301;
the VH comprises the amino acid sequence of SEQ ID NO: 151 and the VL comprises the amino acid sequence of SEQ ID NO:301;
the VH comprises the amino acid sequence of SEQ ID NO: 152 and the VL comprises the amino acid sequence of SEQ ID NO:301;
the VH comprises the amino acid sequence of SEQ ID NO: 153 and the VL comprises the amino acid sequence of SEQ ID NO:301;
the VH comprises the amino acid sequence of SEQ ID NO: 154 and the VL comprises the amino acid sequence of SEQ ID NO:301;
the VH comprises the amino acid sequence of SEQ ID NO: 155 and the VL comprises the amino acid sequence of SEQ ID NO:301;
the VH comprises the amino acid sequence of SEQ ID NO: 156 and the VL comprises the amino acid sequence of SEQ ID NO:301;
the VH comprises the amino acid sequence of SEQ ID NO: 157 and the VL comprises the amino acid sequence of SEQ ID NO:301;
the VH comprises the amino acid sequence of SEQ ID NO: 158 and the VL comprises the amino acid sequence of SEQ ID NO:301;
the VH comprises the amino acid sequence of SEQ ID NO: 159 and the VL comprises the amino acid sequence of SEQ ID NO:301;
the VH comprises the amino acid sequence of SEQ ID NO:160 and the VL comprises the amino acid sequence of SEQ ID NO:301;
the VH comprises the amino acid sequence of SEQ ID NO:300 and the VL comprises the amino acid sequence of SEQ ID NO:200;
the VH comprises the amino acid sequence of SEQ ID NO:300 and the VL comprises the amino acid sequence of SEQ ID NO:201;
the VH comprises the amino acid sequence of SEQ ID NO:300 and the VL comprises the amino acid sequence of SEQ ID NO:202;
the VH comprises the amino acid sequence of SEQ ID NO:300 and the VL comprises the amino acid sequence of SEQ ID NO:203;
the VH comprises the amino acid sequence of SEQ ID NO:300 and the VL comprises the amino acid sequence of SEQ ID NO:204;
the VH comprises the amino acid sequence of SEQ ID NO:300 and the VL comprises the amino acid sequence of SEQ ID NO:205;
the VH comprises the amino acid sequence of SEQ ID NO:300 and the VL comprises the amino acid sequence of SEQ ID NO:206;
the VH comprises the amino acid sequence of SEQ ID NO:300 and the VL comprises the amino acid sequence of SEQ ID NO:207;
the VH comprises the amino acid sequence of SEQ ID NO:300 and the VL comprises the amino acid sequence of SEQ ID NO:208;
the VH comprises the amino acid sequence of SEQ ID NO:300 and the VL comprises the amino acid sequence of SEQ ID NO:209;
the VH comprises the amino acid sequence of SEQ ID NO:300 and the VL comprises the amino acid sequence of SEQ ID NO:210;
the VH comprises the amino acid sequence of SEQ ID NO:300 and the VL comprises the amino acid sequence of SEQ ID NO:211;
the VH comprises the amino acid sequence of SEQ ID NO:300 and the VL comprises the amino acid sequence of SEQ ID NO:212;
the VH comprises the amino acid sequence of SEQ ID NO:300 and the VL comprises the amino acid sequence of SEQ ID NO:213;
the VH comprises the amino acid sequence of SEQ ID NO:300 and the VL comprises the amino acid sequence of SEQ ID NO:214; or
the VH comprises the amino acid sequence of SEQ ID NO:300 and the VL comprises the amino acid sequence of SEQ ID NO:215;
the VH comprises the amino acid sequence of SEQ ID NO:300 and the VL comprises the amino acid sequence of SEQ ID NO:216.

In some embodiments of the anti-TfR1 antibody:
(a) the VH comprises the amino acid sequence of SEQ ID NO: 112 and the VL comprises the amino acid sequence of SEQ ID NO:202;
(b) the VH comprises the amino acid sequence of SEQ ID NO: 128 and the VL comprises the amino acid sequence of SEQ ID NO:202;
(c) the VH comprises the amino acid sequence of SEQ ID NO:150 and the VL comprises the amino acid sequence of SEQ ID NO:301;
(d) the VH comprises the amino acid sequence of SEQ ID NO: 152 and the VL comprises the amino acid sequence of SEQ ID NO:301;
(e) the VH comprises the amino acid sequence of SEQ ID NO:153 and the VL comprises the amino acid sequence of SEQ ID NO:301;
(f) the VH comprises the amino acid sequence of SEQ ID NO: 154 and the VL comprises the amino acid sequence of SEQ ID NO:301;
(g) the VH comprises the amino acid sequence of SEQ ID NO: 155 and the VL comprises the amino acid sequence of SEQ ID NO:301;
(h) the VH comprises the amino acid sequence of SEQ ID NO: 147 and the VL comprises the amino acid sequence of SEQ ID NO:301;
(i) the VH comprises the amino acid sequence of SEQ ID NO:148 and the VL comprises the amino acid sequence of SEQ ID NO:301;
(j) the VH comprises the amino acid sequence of SEQ ID NO:149 and the VL comprises the amino acid sequence of SEQ ID NO:301;
(k) the VH comprises the amino acid sequence of SEQ ID NO: 128 and the VL comprises the amino acid sequence of SEQ ID NO:209;
(l) the VH comprises the amino acid sequence of SEQ ID NO:128 and the VL comprises the amino acid sequence of SEQ ID NO:214;
(m) the VH comprises the amino acid sequence of SEQ ID NO: 128 and the VL comprises the amino acid sequence of SEQ ID NO:212;
(n) the VH comprises the amino acid sequence of SEQ ID NO: 128 and the VL comprises the amino acid sequence of SEQ ID NO:213;
(o) the VH comprises the amino acid sequence of SEQ ID NO:128 and the VL comprises the amino acid sequence of SEQ ID NO:210;
(p) the VH comprises the amino acid sequence of SEQ ID NO:128 and the VL comprises the amino acid sequence of SEQ ID NO:211;
(q) the VH comprises the amino acid sequence of SEQ ID NO:129 and the VL comprises the amino acid sequence of SEQ ID NO:216; or
(r) the VH comprises the amino acid sequence of SEQ ID NO: 128 and the VL comprises the amino acid sequence of SEQ ID NO:216.

In some embodiments, an anti-TfR1 antibody described herein is an scFv. In some embodiments, an anti-TfR1 scFv comprises a stabilizing mutation. In some embodiments, a stabilizing mutation is or comprises a disulfide bond between the variable heavy chain (VH) and variable light chain (VL). In some embodiments, a stabilizing mutation is or comprises a longer linker relative to another anti-TfR1 scFv. Table 5 describes peptide sequences of exemplary anti-TfR1 scFvs. Exemplary linkers (e.g., linker sequences) are shown in Table 6.

TABLE 5
Exemplary Stabilized scFv Sequences

| Pep- | | Affinity | scFv | |
|---|---|---|---|---|
| tide | Peptide | Red. | Stabilization | |
| Name | Description | Mut. | Muts. | Peptide Sequence |
| scFv- | II2C/LO-EVQL VII- | VII-N67E | none | EVQLVESGGGLVQPGGSLRLSCAA |
| 3001 | 3x-VL scFv VH- | | | SGIDFSSSGYMCWVRQAPGKGLE |
| | N67E | | | WVGCIYTYSSETYYAASVKGRFTI |
| | | | | SKTSSTTVYLQMNSLKTEDTAVYY |
| | | | | CARGTYGYTGYTYTMGYFSLWG |
| | | | | QGTLVTVSSGGGGSGGGGSGGGG |
| | | | | SDIQMTQSPSTLSASVGDRVTITCQ |
| | | | | ASQNINSYLAWYQQKPGKAPKLLI |
| | | | | YRASSLESGVPSRFSGSGSGTEFTL |
| | | | | TISSLQPDDFATYYCQSYYYSGSSN |
| | | | | YNAFGGGTKVEIK |
| scFv- | H2C/LO-EVQL VL- | VH-N67E | none | DIQMTQSPSTLSASVGDRVTITCQA |
| 3002 | 3x-VH scFv VH- | | | SQNINSYLAWYQQKPGKAPKLLIY |
| | N67E | | | RASSLESGVPSRFSGSGSGTEFTLTI |
| | | | | SSLQPDDFATYYCQSYYYSGSSNY |
| | | | | NAFGGGTKVEIKGGGGSGGGGSG |
| | | | | GGGSEVQLVESGGGLVQPGGSLRL |
| | | | | SCAASGIDFSSSGYMCWVRQAPG |
| | | | | KGLEWVGCIYTYSSETYYAASVK |
| | | | | GRFTISKTSSTTVYLQMNSLKTEDT |
| | | | | AVYYCARGTYGYTGYTYTMGYFS |
| | | | | LWGQGTLVTVSS |
| scFv- | H2C/LO-EVQL VH- | VH-N67E | longer linker | EVQLVESGGGLVQPGGSLRLSCAA |
| 3003 | 4x-VL scFv VH- | | | SGIDFSSSGYMCWVRQAPGKGLE |
| | N67E | | | WVGCIYTYSSETYYAASVKGRFTI |
| | | | | SKTSSTTVYLQMNSLKTEDTAVYY |
| | | | | CARGTYGYTGYTYTMGYFSLWG |
| | | | | QGTLVTVSSGGGGSGGGGSGGGG |
| | | | | SGGGGSDIQMTQSPSTLSASVGDR |
| | | | | VTITCQASQNINSYLAWYQQKPGK |
| | | | | APKLLIYRASSLESGVPSRFSGSGS |
| | | | | GTEFTLTISSLQPDDFATYYCQSYY |
| | | | | YSGSSNYNAFGGGTKVEIK |
| scFv- | II2C/LO-EVQL VL- | VII-N67E | longer linker | DIQMTQSPSTLSASVGDRVTITCQA |
| 3004 | 4x-VH scFv VH- | | | SQNINSYLAWYQQKPGKAPKLLIY |
| | N67E | | | RASSLESGVPSRFSGSGSGTEFTITI |
| | | | | SSLQPDDFATYYCQSYYYSGSSNY |
| | | | | NAFGGGTKVEIKGGGGSGGGGSG |
| | | | | GGGSGGGGSEVQLVESGGGLVQP |
| | | | | GGSLRLSCAASGIDFSSSGYMCWV |
| | | | | RQAPGKGLEWVGCIYTYSSETYY |
| | | | | AASVKGRFTISKTSSTTVYLQMNS |
| | | | | LKTEDTAVYYCARGTYGYTGYTY |
| | | | | TMGYFSLWGQGTLVTVSS |
| scFv- | H2C/LO-EVQL VH- | VH-N67E | VH-VL DSB | EVQLVESGGGLVQPGGSLRLSCAA |
| 3005 | 3x-VL scFv VH- | | | SGIDFSSSGYMCWVRQAPGKCLE |
| | G51C/N67E VL- | | | WVGCIYTYSSETYYAASVKGRFTI |
| | G141C | | | SKTSSTTVYLQMNSLKTEDTAVYY |
| | | | | CARGTYGYTGYTYTMGYFSLWG |
| | | | | QGTLVTVSSGGGGSGGGGSGGGG |
| | | | | SDIQMTQSPSTLSASVGDRVTITCQ |
| | | | | ASQNINSYLAWYQQKPGKAPKLLI |
| | | | | YRASSLESGVPSRFSGSGSGTEFTL |
| | | | | TISSLQPDDFATYYCQSYYYSGSSN |
| | | | | YNAFGCGTKVEIK |
| scFv- | H2C/LO-EVQL VL- | VH-N67E | VH-VL DSB | DIQMTQSPSTLSASVGDRVTITCQA |
| 3006 | 3x-VH scFv VL- | | | SQNINSYLAWYQQKPGKAPKLLIY |
| | G141C VH- | | | RASSLESGVPSRFSGSGSGTEFTLTI |
| | G51C/N67E | | | SSLQPDDFATYYCQSYYYSGSSNY |
| | | | | NAFGCGTKVEIKGGGGSGGGGSG |
| | | | | GGGSEVQLVESGGGIVQPGGSLRI. |
| | | | | SCAASGIDFSSSGYMCWVRQAPG |
| | | | | KCLEWVGCIYTYSSETYYAASVK |
| | | | | GRFTISKTSSTTVYLQMNSLKTEDT |
| | | | | AVYYCARGTYGYTGYTYTMGYFS |
| | | | | LWGQGTLVTVSS |
| scFv- | H2C/LO-EVQL VL- | VH-N67E | VH-VL DSB | DIQMTQSPSTLSASVGDRVTITCQA |
| 3007 | 3x-VH scFv VL- | | | SQNINSYLAWYQQKPGKAPKCLIY |
| | L54C VH- | | | RASSLESGVPSRFSGSGSGTEFTLTI |
| | N67E/S137C | | | SSLQPDDFATYYCQSYYYSGSSNY |
| | | | | NAFGGGTKVEIKGGGGSGGGGSG |
| | | | | GGGSEVQLVESGGGLVQPGGSLRL |
| | | | | SCAASGIDFSSSGYMCWVRQAPG |
| | | | | KGLEWVGCIYTYSSETYYAASVK |
| | | | | GRFTISKTSSTTVYLQMNSLKTEDT |
| | | | | AVYYCARGTYGYTGYTYTMGYF |
| | | | | CLWGQGTLVTVSS |
| scFv- | H2C/LO-EVQL VL- | VH-N67E | VH-VL DSB | DIQMTQSPSTLSASVGDRVTITCQA |
| 3008 | 3x-VH scFv VL- | | | SQNINSYLCWYQQKPGKAPKLLIY |
| | A42C VH- | | | RASSLESGVPSRFSGSGSGTEFTLTI |
| | N67E/Y135C | | | SSLQPDDFATYYCQSYYYSGSSNY |
| | | | | NAFGGGTKVEIKGGGGSGGGGSG |
| | | | | GGGSEVQLVESGGGLVQPGGSLRL |
| | | | | SCAASGIDFSSSGYMCWVRQAPG |
| | | | | KGLEWVGCIYTYSSETYYAASVK |
| | | | | GRFTISKTSSTTVYLQMNSLKTEDT |
| | | | | AVYYCARGTYGYTGYTYTMGCFS |
| | | | | I.WGQGTLVTVSS |
| scFv- | II2C/LO-EVQL VL- | VII-N67E | VII-VL DSB | DIQMTQSPSTLSASVGDRVTITCQA |
| 3009 | 3x-VH scFv VL- | | | SQNINSYLAWYQQKPGKAPKLLIY |
| | Y109C VH- | | | RASSLESGVPSRFSGSGSGTEFTITI |
| | N67E/G134C | | | SSLQPDDFATYYCQSCYYSGSSNY |
| | | | | NAFGGGTKVEIKGGGGSGGGGSG |
| | | | | GGGSEVQLVESGGGLVQPGGSLRL |
| | | | | SCAASGIDFSSSGYMCWVRQAPG |
| | | | | KGLEWVGCIYTYSSETYYAASVK |
| | | | | GRFTISKTSSTTVYLQMNSLKTEDT |
| | | | | AVYYCARGTYGYTGYTYTMCYFS |
| | | | | LWGQGTLVTVSS |
| scFv- | H2C/LO-EVQL VH- | VH-N67E | VH-VL DSB, | EVQLVESGGGLVQPGGSLRLSCAA |
| 3010 | 4x-VL scFv VH- | | longer linker | SGIDFSSSGYMCWVRQAPGKCLE |
| | G51C/N67E VL- | | | WVGCIYTYSSETYYAASVKGRFTI |
| | G141C | | | SKTSSTTVYLQMNSLKTEDTAVYY |
| | | | | CARGTYGYTGYTYTMGYFSLWG |
| | | | | QGTLVTVSSGGGGSGGGGSGGGG |
| | | | | SGGGGSDIQMTQSPSTLSASVGDR |
| | | | | VTITCQASQNINSYLAWYQQKPGK |
| | | | | APKLLIYRASSLESGVPSRFSGSGS |
| | | | | GTEFTLTISSLQPDDFATYYCQSYY |
| | | | | YSGSSNYNAFGCGTKVEIK |
| scFv- | H2C/LO-EVQL VL- | VH-N67E | VH-VL DSB, | DIQMTQSPSTLSASVGDRVTITCQA |
| 3011 | 4x-VH scFv VL- | | longer linker | SQNINSYLAWYQQKPGKAPKLLIY |
| | G141C VH- | | | RASSLESGVPSRFSGSGSGTEFTLTI |
| | G51C/N67E | | | SSLQPDDFATYYCQSYYYSGSSNY |
| | | | | NAFGCGTKVEIKGGGGSGGGGSG |
| | | | | GGGSGGGGSEVQLVESGGGIVQP |
| | | | | GGSLRLSCAASGIDFSSSGYMCWV |
| | | | | RQAPGKCLEWVGCIYTYSSETYYA |
| | | | | ASVKGRFTISKTSSTTVYLQMNSL |
| | | | | KTEDTAVYYCARGTYGYTGYTYT |
| | | | | MGYFSLWGQGTLVTVSS |

### Constant Regions of Anti-TfR1 Antibodies

In some embodiments, the variable region of an anti-TfR1 antibody described herein is fused to a constant region. A constant region has a constant heavy chain (CH) domain (e.g., CH1, hinge, CH2, and/or CH3 domain(s) or any combination thereof) and a constant light chain (CL) domain. In some embodiments, the CH domain is from an IgG1 molecule or an IgG4 molecule. In some embodiments, the CH domain is from an IgG2 molecule, an IgG3 molecule, or an IgG molecule. The VH of an anti-TfR1 antibody described herein can be fused to any one of the following constant heavy chain (CH) constructs as shown in Table 6 below. The VL of the anti-TfR1 antibody described herein can be fused to any one of the following constant light chain (CL) constructs as shown in Table 6 below. In some embodiments, the hinge region is any hinge region known in the art. In some embodiments, the hinge region is naturally occurring, e.g., from a naturally occurring IgG1, IgG2, IgG3, or IgG4 molecule. In other embodiments, the hinge region contains modification(s) relative to a naturally occurring hinge.

In some embodiments, an anti-TfR1 antibody of the disclosure is one in which at least one or more of the constant regions has been modified or deleted. In some embodiments, an antibody may comprise one or more modifications to the heavy chain constant domain (CH1, CH2 or CH3) and/or to the light chain constant region (CL). In some embodiments, the heavy chain constant region of the modified antibody comprises at least one human constant region. In some embodiments, the heavy chain constant region of the modified antibody comprises more than one human constant region. In some embodiments, the VH is fused to any CH1 construct known in the art, and the VL is fused to any CL known in the art. In some embodiments, the constant light chain (CL) of the constructs is a naturally occurring human Kappa constant region. In some embodiments, modifications to the constant region comprise additions, deletions, or substitutions of one or more amino acids in one or more regions. In some embodiments, one or more regions are partially or entirely deleted from the constant regions of a modified antibody. In some embodiments, the entire CH2 and CH3 domains have been removed from an antibody. In some embodiments, a deleted constant region is replaced by a short amino acid spacer that provides some of the molecular flexibility typically imparted by the absent constant region. In some embodiments, a modified antibody comprises a CH1 domain directly fused to the hinge region of the antibody. In some embodiments, a modified antibody comprises an Fab fused to the bottom of an Fc.

In some embodiments, an anti-TfR1 antibody of the disclosure contains a linker (e.g., a linker as shown in Table 6 below). In some embodiments, a linker is positioned between the Fc region and the Fab region of an anti-TfR1 antibody of the disclosure.

TABLE 6
Constant Region, Hinge Sequences, and Linker Sequences

| | |
|---|---|
| Domain | Sequence |

IgG1 or IgG4 CH1

| | |
|---|---|
| IgG1 CH1 (no hinge) | ASTKGPSVFPLAPSSKSTSGGTAALGCLVKDYFPEPVTVSWNSGAL |
| (SEQ ID NO: XX) | TSGVHTFPAVLQSSGLYSLSSVVTVPSSSLGTQTYICNVNHKPSNT |
| | KVDKKV |
| IgG1 CH1 (with hinge underlined) | ASTKGPSVFPLAPSSKSTSGGTAALGCLVKDYFPEPVTVSWNSGA |
| (SEQ ID NO: XX) | LTSGVHTFPAVLQSSGLYSLSSVVTVPSSSLGTQTYICNVNHKPSN |
| | TKVDKKVEPKSCDKTHTCPP |
| IgG1 CH1 (no hinge) | ASTKGPSVFPLAPSSKSTSGGTAALGCLVKDYFPEPVTVSWNSGA |
| (SEQ ID NO: XX) | LTSGVHTFPAVLQSSGLYSLSSVVTVPSSSLGTQTYICNVNHKPSN |
| | TKVDKRV |
| IgG1 CH1 (with hinge underlined) | ASTKGPSVFPLAPSSKSTSGGTAALGCLVKDYFPEPVTVSWNSGA |
| (SEQ ID NO: XX) | LTSGVHTFPAVLQSSGLYSLSSVVTVPSSSLGTQTYICNVNHKPSN |
| | TKVDKRVESKYGPPCP |
| IgG1 CH1 (no hinge) | ASTKGPSVFPLAPSSKSTSGGTAALGCLVKDYFPEPVTVSWNSGA |
| (SEQ ID NO: XX) | LTSGVHTFPAVLQSSGLYSLSSVVTVPSSSLGTQTYICNVNHKPSN |
| | TKVDKKV |
| IgGI CH1 (with hinge underlined) | ASTKGPSVFPLAPSSKSTSGGTAALGCLVKDYFPEPVTVSWNSGA |
| (SEQ ID NO: XX) | LTSGVHTFPAVLQSSGLYSLSSVVTVPSSSLGTQTYICNVNHKPSN |
| | TKVDKKVEPKSC |
| IgG4 CH1 (no hinge) | ASTKGPSVFPLAPCSRSTSESTAALGCLVKDYFPEPVTVSWNSGAL |
| (SEQ ID NO: XX) | TSGVHTFPAVLQSSGLYSLSSVVTVPSSSLGTKTYTCNVDHKPSNT |
| | KVDKRV |
| IgG4 CH1 (with hinge underlined) | ASTKGPSVFPLAPCSRSTSESTAALGCLVKDYFPEPVTVSWNSGAL |
| (SEQ ID NO: XX) | TSGVHTFPAVLQSSGLYSLSSVVTVPSSSLGTKTYTCNVDHKPSNT |
| | KVDKRVES |
| Fc hIgG1.agly DelPG | KTHTCPPCPAPELLGGPSVFLFPPKPKDTLMISRTPEVTCVVVDVS |
| (SEQ ID NO: XX) | HEDPEVKFNWYVDGVEVHNAKTKPREEQYQSTYRVVSVLTVLHQ |
| | DWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLS |
| Fc hIgG1.agly DelG | KTHTCPPCPAPELLGGPSVFLFPPKPKDTLMISRTPEVTCVVVDVS |
| (SEQ ID NO: XX) | HEDPEVKFNWYVDGVEVHNAKTKPREEQYQSTYRVVSVLTVLHQ |
| | DWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSP |
| Fc hIgG1.agly | KTHTCPPCPAPELLGGPSVFLFPPKPKDTIMISRTPEVTCVVVDVS |
| (SEQ ID NO: XX) | HEDPEVKFNWYVDGVEVHNAKTKPREEQYQSTYRVVSVLTVLHQ |
| | DWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| HingeFc hIgG1.agly | DKTHTCPPCPAPELLGGPSVFLFPPKPKDTLMISRTPEVTCVVVDV |
| (SEQ ID NO: XX) | SHEDPEVKFNWYVDGVEVHNAKTKPREEQYQSTYRVVSVLTVLH |
| | QDWLNGKEYKCKVSNKALPAPIEKTISKAKGQPREPQVYTLPPSR |
| | DELTKNQVSLTCLVKGFYPSDIAVEWESNGQPENNYKTTPPVLDS |
| | DGSFFLYSKLTVDKSRWQQGNVFSCSVMHEALHNHYTQKSLSLSP |
| | G |
| HingeFc hIgG1.agly (KnobS) | DKTHTCPPCPAPELLGGPSVFLFPPKPKDTLMISRTPEVTCVVVDV |
| (SEQ ID NO: XX) | SHEDPEVKFNWYVDGVEVHNAKTKPREEQYQSTYRVVSVLTVLH |
| | QDWLNGKEYKCKVSNKALPAPIEKTISKAKGQPREPQVYTLPPCR |
| | DELTKNQVSLWCLVKGFYPSDIAVEWESNGQPENNYKTTPPVLDS |
| | DGSFFLYSKLTVDKSRWQQGNVFSCSVMHEALHNHYTQKSLSLSP |
| | G |
| Fc hIgG1.agly(KnobS) | KTHTCPPCPAPELLGGPSVFLFPPKPKDTLMISRTPEVTCVVVDVS |
| (SEQ ID NO: XX) | HEDPEVKFNWYVDGVEVHNAKTKPREEQYQSTYRVVSVLTVLHQ |
| | DWLNGKEYKCKVSNKALPAPIEKTISKAKGQPREPQVYTLPPCRD |
| | ELTKNQVSLWCLVKGFYPSDIAVEWESNGQPENNYKTTPPVLDSD |
| | GSFFLYSKLTVDKSRWQQGNVFSCSVMHEALHNHYTQKSLSLSPG |
| HingeFc hIgG1.agly (HoleS) | KTHTCPPCPAPELLGGPSVFLFPPKPKDTLMISRTPEVTCVVVDVS |
| (SEQ ID NO: XX) | HEDPEVKFNWYVDGVEVHNAKTKPREEQYQSTYRVVSVLTVLHQ |
| | DWLNGKEYKCKVSNKALPAPIEKTISKAKGQPREPQVCTLPPSRD |
| | ELTKNQVSLSCAVKGFYPSDIAVEWESNGQPENNYKTTPPVLDSD |
| | GSFFLVSKLTVDKSRWQQGNVFSCSVMHEALHNHYTQKSLSLSPG |

Hinge region

| | |
|---|---|
| IgG1 hinge A (SEQ ID NO: XX) | EPKSCDKTHTCPPCPAPELLGGP |
| IgG1 hinge B (SEQ ID NO: XX) | EPKSCDKTHTCPP |
| IgG1 hinge C (SEQ ID NO: XX) | EPKSC |
| IgG4 hinge D (SEQ ID NO: XX) | ESKYGPPCPPCPAPEFLGGP |
| IgG4 hinge E (SEQ ID NO: XX) | ESKYGPPCP |
| IgG4 hinge F | ES |
| IgG1 hinge G (SEQ ID NO: XX) | EPKS |
| IgG1 hinge II (SEQ ID NO: XX) | EPKSCD |
| IgG1 hinge I (SEQ ID NO: XX) | EPKSCDK |
| IgG1 hinge J (SEQ ID NO: XX) | EPKSCDKT |
| IgG1 hinge K (SEQ ID NO: XX) | EPKSCDKTH |
| IgG1 hinge L (SEQ ID NO: XX) | EPKSCDKTHT |
| IgG1 hinge M (SEQ ID NO: XX) | EPKSCDKTHTC |
| IgG1 hinge N (SEQ ID NO: XX) | EPKSCDKTHTCP |
| IgG1 hinge O (SEQ ID NO: XX) | EPKSCDKTHTCPPC |
| IgG1 hinge P (SEQ ID NO: XX) | EPKSCDKTHTCPPCP |
| IgG1 hinge Q (SEQ ID NO: XX) | EPKSCDKTHTCPPCPA |
| IgGI hinge R (SEQ ID NO: XX) | EPKSCDKTHTCPPCPAP |
| IgGI hinge S (SEQ ID NO: XX) | EPKSCDKTHTCPPCPAPE |
| IgG1 hinge T (SEQ ID NO: XX) | EPKSCDKTHTCPPCPAPEL |
| IgGI hinge U (SEQ ID NO: XX) | EPKSCDKTHTCPPCPAPELL |
| IgG1 hinge V (SEQ ID NO: XX) | EPKSCDKTHTCPPCPAPELLG |
| IgG1 hinge W (SEQ ID NO: XX) | EPKSCDKTHTCPPCPAPELLGG |
| IgG4 hinge X | ESK |
| IgG4 hinge Y (SEQ ID NO: XX) | ESKY |
| IgG4 hinge Z (SEQ ID NO: XX) | ESKYG |
| IgG4 hinge ZA (SEQ ID NO: XX) | ESKYGP |
| IgG4 hinge ZB (SEQ ID NO: XX) | ESKYGPP |
| IgG4 hinge ZC (SEQ ID NO: XX) | ESKYGPPC |
| IgG4 hinge ZD (SEQ ID NO: XX) | ESKYGPPCPP |
| IgG4 hinge ZE (SEQ ID NO: XX) | ESKYGPPCPPC |
| IgG4 hinge 7.F (SEQ ID NO: XX) | ESKYGPPCPPCP |
| IgG4 hinge ZG (SEQ ID NO: XX) | ESKYGPPCPPCPA |
| IgG4 hinge ZH (SEQ ID NO: XX) | ESKYGPPCPPCPAP |
| IgG4 hinge ZI (SEQ ID NO: XX) | ESKYGPPCPPCPAPE |
| IgG4 hinge ZJ (SEQ ID NO: XX) | ESKYGPPCPPCPAPEF |
| IgG4 hinge ZK (SEQ ID NO: XX) | ESKYGPPCPPCPAPEFL |
| IgG4 hinge ZL (SEQ ID NO: XX) | ESKYGPPCPPCPAPEFLG |
| IgG4 hinge ZM (SEQ ID NO: XX) | ESKYGPPCPPCPAPEFLGG |
| IgG4 hinge ZN (SEQ ID NO: XX) | ESKYGPPCPPCPAPEFLGGP |

CL domain

| | |
|---|---|
| Human kappa CL (SEQ ID NO: | RTVAAPSVFIFPPSDEQLKSGTASVVCLLNNFYPREAKVQWKVDN |
| XX) | ALQSGNSQESVTEQDSKDSTYSLSSTLTLSKADYEKHKVYACEVT |
| | HQGLSSPVTKSFNRGEC |
| Human kappa CL (A C214) | RTVAAPSVFIFPPSDEQLKSGTASVVCLLNNFYPREAKVQWKVDN |
| (SEQ ID NO: XX) | ALQSGNSQESVTEQDSKDSTYSLSSTLTLSKADYEKHKVYACEVT |
| | HQGLSSPVTKSFNRGE |

Linker

| | |
|---|---|
| LinkerS | S |
| LinkerSG2 | SGG |
| LinkgerSG4 (SEQ ID NO: XX) | SGGGG |
| LinkerSG4SG4 (SEQ ID NO: XX) | SGGGGSGGGG |
| LinkerSG4SG4SG4 (SEQ ID NO: | SGGGGSGGGGSGGGG |
| XX) | |
| LinkerG3SG5 (SEQ ID NO: XX) | GGGSGGGGG |
| LinkerG4SG4SG4S (SEQ ID NO: | GGGGSGGGGSGGGGS |
| XX) | |
| LinkerTG | TG |

Other exemplary constant regions, e.g., hinge regions, that can be combined with the antibody variable regions described herein include but are not limited to the hinge regions described in Peters S J, et al. J Biol Chem. 2012 Jul. 13; 287 (29): 24525-33; and Heads J T, et al. Protein Sci. 2012 September; 21 (9): 1315-22; incorporated herein by reference in their entirety.

In some embodiments, an anti-TfR1 antibody comprises:
a heavy chain comprising the amino acid sequence set forth in HC-1043, and a light chain comprising the amino acid sequence set forth in LC-1096;
a heavy chain comprising the amino acid sequence set forth in HC-1044, and a light chain comprising the amino acid sequence set forth in LC-1096;
a heavy chain comprising the amino acid sequence set forth in HC-1045, and a light chain comprising the amino acid sequence set forth in LC-1096;
a heavy chain comprising the amino acid sequence set forth in HC-1046, and a light chain comprising the amino acid sequence set forth in LC-1096;
a heavy chain comprising the amino acid sequence set forth in HC-1047, and a light chain comprising the amino acid sequence set forth in LC-1096;
a heavy chain comprising the amino acid sequence set forth in HC-1048, and a light chain comprising the amino acid sequence set forth in LC-1096;
a heavy chain comprising the amino acid sequence set forth in HC-1049, and a light chain comprising the amino acid sequence set forth in LC-1096;
a heavy chain comprising the amino acid sequence set forth in HC-1050, and a light chain comprising the amino acid sequence set forth in LC-1096;
a heavy chain comprising the amino acid sequence set forth in HC-1051, and a light chain comprising the amino acid sequence set forth in LC-1096;
a heavy chain comprising the amino acid sequence set forth in HC-1052, and a light chain comprising the amino acid sequence set forth in LC-1096;
a heavy chain comprising the amino acid sequence set forth in HC-1053, and a light chain comprising the amino acid sequence set forth in LC-1096;
a heavy chain comprising the amino acid sequence set forth in HC-1055, and a light chain comprising the amino acid sequence set forth in LC-1096;
a heavy chain comprising the amino acid sequence set forth in HC-1056, and a light chain comprising the amino acid sequence set forth in LC-1096;
a heavy chain comprising the amino acid sequence set forth in HC-1057, and a light chain comprising the amino acid sequence set forth in LC-1096;
a heavy chain comprising the amino acid sequence set forth in HC-1058, and a light chain comprising the amino acid sequence set forth in LC-1096;
a heavy chain comprising the amino acid sequence set forth in HC-1059, and a light chain comprising the amino acid sequence set forth in LC-1096;
a heavy chain comprising the amino acid sequence set forth in HC-1060, and a light chain comprising the amino acid sequence set forth in LC-1096;
a heavy chain comprising the amino acid sequence set forth in HC-1061, and a light chain comprising the amino acid sequence set forth in LC-1096;
a heavy chain comprising the amino acid sequence set forth in HC-1062, and a light chain comprising the amino acid sequence set forth in LC-1096;
a heavy chain comprising the amino acid sequence set forth in HC-1063, and a light chain comprising the amino acid sequence set forth in LC-1096;
a heavy chain comprising the amino acid sequence set forth in HC-1064, and a light chain comprising the amino acid sequence set forth in LC-1096;
a heavy chain comprising the amino acid sequence set forth in HC-1065, and a light chain comprising the amino acid sequence set forth in LC-1096;
a heavy chain comprising the amino acid sequence set forth in HC-1066, and a light chain comprising the amino acid sequence set forth in LC-1096;
a heavy chain comprising the amino acid sequence set forth in HC-1067, and a light chain comprising the amino acid sequence set forth in LC-1096;
a heavy chain comprising the amino acid sequence set forth in HC-1068, and a light chain comprising the amino acid sequence set forth in LC-1096;
a heavy chain comprising the amino acid sequence set forth in HC-1069, and a light chain comprising the amino acid sequence set forth in LC-1096;
a heavy chain comprising the amino acid sequence set forth in HC-1070, and a light chain comprising the amino acid sequence set forth in LC-1096;
a heavy chain comprising the amino acid sequence set forth in HC-1071, and a light chain comprising the amino acid sequence set forth in LC-1096;
a heavy chain comprising the amino acid sequence set forth in HC-1072, and a light chain comprising the amino acid sequence set forth in LC-1096;
a heavy chain comprising the amino acid sequence set forth in HC-1073, and a light chain comprising the amino acid sequence set forth in LC-1096;
a heavy chain comprising the amino acid sequence set forth in HC-1074, and a light chain comprising the amino acid sequence set forth in LC-1096;
a heavy chain comprising the amino acid sequence set forth in HC-1075, and a light chain comprising the amino acid sequence set forth in LC-1096;
a heavy chain comprising the amino acid sequence set forth in HC-1076, and a light chain comprising the amino acid sequence set forth in LC-1096;
a heavy chain comprising the amino acid sequence set forth in HC-1077, and a light chain comprising the amino acid sequence set forth in LC-1096;
a heavy chain comprising the amino acid sequence set forth in HC-1078, and a light chain comprising the amino acid sequence set forth in LC-1096;
a heavy chain comprising the amino acid sequence set forth in HC-1079, and a light chain comprising the amino acid sequence set forth in LC-1096;
a heavy chain comprising the amino acid sequence set forth in HC-1080, and a light chain comprising the amino acid sequence set forth in LC-1096;
a heavy chain comprising the amino acid sequence set forth in HC-1081, and a light chain comprising the amino acid sequence set forth in LC-1096;
a heavy chain comprising the amino acid sequence set forth in HC-1082, and a light chain comprising the amino acid sequence set forth in LC-1096;
a heavy chain comprising the amino acid sequence set forth in HC-1083, and a light chain comprising the amino acid sequence set forth in LC-1096;
a heavy chain comprising the amino acid sequence set forth in HC-1084, and a light chain comprising the amino acid sequence set forth in LC-1096;
a heavy chain comprising the amino acid sequence set forth in HC-1085, and a light chain comprising the amino acid sequence set forth in LC-1096;
a heavy chain comprising the amino acid sequence set forth in HC-1086, and a light chain comprising the amino acid sequence set forth in LC-1096;
a heavy chain comprising the amino acid sequence set forth in HC-1087, and a light chain comprising the amino acid sequence set forth in LC-1096;
a heavy chain comprising the amino acid sequence set forth in HC-1054, and a light chain comprising the amino acid sequence set forth in LC-1098;
a heavy chain comprising the amino acid sequence set forth in HC-1054, and a light chain comprising the amino acid sequence set forth in LC-1099;
a heavy chain comprising the amino acid sequence set forth in HC-1054, and a light chain comprising the amino acid sequence set forth in LC-1100;
a heavy chain comprising the amino acid sequence set forth in HC-1054, and a light chain comprising the amino acid sequence set forth in LC-1101;
a heavy chain comprising the amino acid sequence set forth in HC-1054, and a light chain comprising the amino acid sequence set forth in LC-1102;
a heavy chain comprising the amino acid sequence set forth in HC-1054, and a light chain comprising the amino acid sequence set forth in LC-1103;
a heavy chain comprising the amino acid sequence set forth in HC-1054, and a light chain comprising the amino acid sequence set forth in LC-1104;
a heavy chain comprising the amino acid sequence set forth in HC-1054, and a light chain comprising the amino acid sequence set forth in LC-1105;
a heavy chain comprising the amino acid sequence set forth in HC-1054, and a light chain comprising the amino acid sequence set forth in LC-1106;
a heavy chain comprising the amino acid sequence set forth in HC-1039, and a light chain comprising the amino acid sequence set forth in LC-1100;
a heavy chain comprising the amino acid sequence set forth in HC-1056, and a light chain comprising the amino acid sequence set forth in LC-1100;
a heavy chain comprising the amino acid sequence set forth in HC-1071, and a light chain comprising the amino acid sequence set forth in LC-1095;
a heavy chain comprising the amino acid sequence set forth in HC-1071, and a light chain comprising the amino acid sequence set forth in LC-1100;
a heavy chain comprising the amino acid sequence set forth in HC-1073, and a light chain comprising the amino acid sequence set forth in LC-1095;
a heavy chain comprising the amino acid sequence set forth in HC-1004, and a light chain comprising the amino acid sequence set forth in LC-1095;
a heavy chain comprising the amino acid sequence set forth in HC-1005, and a light chain comprising the amino acid sequence set forth in LC-1095;
a heavy chain comprising the amino acid sequence set forth in HC-1006, and a light chain comprising the amino acid sequence set forth in LC-1095;
a heavy chain comprising the amino acid sequence set forth in HC-1007, and a light chain comprising the amino acid sequence set forth in LC-1095;
a heavy chain comprising the amino acid sequence set forth in HC-1008, and a light chain comprising the amino acid sequence set forth in LC-1095;
a heavy chain comprising the amino acid sequence set forth in HC-1009, and a light chain comprising the amino acid sequence set forth in LC-1095;
a heavy chain comprising the amino acid sequence set forth in HC-1010, and a light chain comprising the amino acid sequence set forth in LC-1095;
a heavy chain comprising the amino acid sequence set forth in HC-1011, and a light chain comprising the amino acid sequence set forth in LC-1095;
a heavy chain comprising the amino acid sequence set forth in HC-1012, and a light chain comprising the amino acid sequence set forth in LC-1095;
a heavy chain comprising the amino acid sequence set forth in HC-1013, and a light chain comprising the amino acid sequence set forth in LC-1095;
a heavy chain comprising the amino acid sequence set forth in HC-1014, and a light chain comprising the amino acid sequence set forth in LC-1095;
a heavy chain comprising the amino acid sequence set forth in HC-1015, and a light chain comprising the amino acid sequence set forth in LC-1095;
a heavy chain comprising the amino acid sequence set forth in HC-1016, and a light chain comprising the amino acid sequence set forth in LC-1095;
a heavy chain comprising the amino acid sequence set forth in HC-1017, and a light chain comprising the amino acid sequence set forth in LC-1095;
a heavy chain comprising the amino acid sequence set forth in HC-1018, and a light chain comprising the amino acid sequence set forth in LC-1095;
a heavy chain comprising the amino acid sequence set forth in HC-1019, and a light chain comprising the amino acid sequence set forth in LC-1095;
a heavy chain comprising the amino acid sequence set forth in HC-1020, and a light chain comprising the amino acid sequence set forth in LC-1095;
a heavy chain comprising the amino acid sequence set forth in HC-1021, and a light chain comprising the amino acid sequence set forth in LC-1095;
a heavy chain comprising the amino acid sequence set forth in HC-1022, and a light chain comprising the amino acid sequence set forth in LC-1095;
a heavy chain comprising the amino acid sequence set forth in HC-1023, and a light chain comprising the amino acid sequence set forth in LC-1095;
a heavy chain comprising the amino acid sequence set forth in HC-1024, and a light chain comprising the amino acid sequence set forth in LC-1095;
a heavy chain comprising the amino acid sequence set forth in HC-1025, and a light chain comprising the amino acid sequence set forth in LC-1095;
a heavy chain comprising the amino acid sequence set forth in HC-1026, and a light chain comprising the amino acid sequence set forth in LC-1095;
a heavy chain comprising the amino acid sequence set forth in HC-1027, and a light chain comprising the amino acid sequence set forth in LC-1095;
a heavy chain comprising the amino acid sequence set forth in HC-1028, and a light chain comprising the amino acid sequence set forth in LC-1095;
a heavy chain comprising the amino acid sequence set forth in HC-1029, and a light chain comprising the amino acid sequence set forth in LC-1095;
a heavy chain comprising the amino acid sequence set forth in HC-1030, and a light chain comprising the amino acid sequence set forth in LC-1095;
a heavy chain comprising the amino acid sequence set forth in HC-1031, and a light chain comprising the amino acid sequence set forth in LC-1095;
a heavy chain comprising the amino acid sequence set forth in HC-1033, and a light chain comprising the amino acid sequence set forth in LC-1095;
a heavy chain comprising the amino acid sequence set forth in HC-1013, and a light chain comprising the amino acid sequence set forth in LC-1100;
a heavy chain comprising the amino acid sequence set forth in HC-1013, and a light chain comprising the amino acid sequence set forth in LC-1107;
a heavy chain comprising the amino acid sequence set forth in HC-1013, and a light chain comprising the amino acid sequence set forth in LC-1108;
a heavy chain comprising the amino acid sequence set forth in HC-1013, and a light chain comprising the amino acid sequence set forth in LC-1109;
a heavy chain comprising the amino acid sequence set forth in HC-1013, and a light chain comprising the amino acid sequence set forth in LC-1110;
a heavy chain comprising the amino acid sequence set forth in HC-1013, and a light chain comprising the amino acid sequence set forth in LC-1111;
a heavy chain comprising the amino acid sequence set forth in HC-1013, and a light chain comprising the amino acid sequence set forth in LC-1112;
a heavy chain comprising the amino acid sequence set forth in HC-1034, and a light chain comprising the amino acid sequence set forth in LC-1113;
a heavy chain comprising the amino acid sequence set forth in HC-1034, and a light chain comprising the amino acid sequence set forth in LC-1114;
a heavy chain comprising the amino acid sequence set forth in HC-2002, and a light chain comprising the amino acid sequence set forth in LC-1095;
a heavy chain comprising the amino acid sequence set forth in HC-2003, and a light chain comprising the amino acid sequence set forth in LC-1095;
a heavy chain comprising the amino acid sequence set forth in HC-2004, and a light chain comprising the amino acid sequence set forth in LC-1095;
a heavy chain comprising the amino acid sequence set forth in HC-2005, and a light chain comprising the amino acid sequence set forth in LC-1095;
a heavy chain comprising the amino acid sequence set forth in HC-2006, and a light chain comprising the amino acid sequence set forth in LC-1095;
a heavy chain comprising the amino acid sequence set forth in HC-2007, and a light chain comprising the amino acid sequence set forth in LC-1095;
a heavy chain comprising the amino acid sequence set forth in HC-2006, and a light chain comprising the amino acid sequence set forth in LC-2021;
a heavy chain comprising the amino acid sequence set forth in HC-2008, and a light chain comprising the amino acid sequence set forth in LC-1095;
a heavy chain comprising the amino acid sequence set forth in HC-2009, and a light chain comprising the amino acid sequence set forth in LC-1095;
a heavy chain comprising the amino acid sequence set forth in HC-2010, and a light chain comprising the amino acid sequence set forth in LC-1095;
a heavy chain comprising the amino acid sequence set forth in HC-2011, and a light chain comprising the amino acid sequence set forth in LC-1095;
a heavy chain comprising the amino acid sequence set forth in HC-2012, and a light chain comprising the amino acid sequence set forth in LC-2021;
a heavy chain comprising the amino acid sequence set forth in HC-2013, and a light chain comprising the amino acid sequence set forth in LC-1095;
a heavy chain comprising the amino acid sequence set forth in HC-2014, and a light chain comprising the amino acid sequence set forth in LC-1095;
a heavy chain comprising the amino acid sequence set forth in HC-2015, and a light chain comprising the amino acid sequence set forth in LC-1095;
a heavy chain comprising the amino acid sequence set forth in HC-2016, and a light chain comprising the amino acid sequence set forth in LC-1095;
a heavy chain comprising the amino acid sequence set forth in HC-2016, and a light chain comprising the amino acid sequence set forth in LC-2021;
a heavy chain comprising the amino acid sequence set forth in HC-2017, and a light chain comprising the amino acid sequence set forth in LC-1095;
a heavy chain comprising the amino acid sequence set forth in HC-2018, and a light chain comprising the amino acid sequence set forth in LC-1095;
a heavy chain comprising the amino acid sequence set forth in HC-2019, and a light chain comprising the amino acid sequence set forth in LC-1095;
a heavy chain comprising the amino acid sequence set forth in HC-2020, and a light chain comprising the amino acid sequence set forth in LC-1095;
the amino acid sequence set forth in HC-1088;
the amino acid sequence set forth in HC-1089;
the amino acid sequence set forth in HC-1090;
the amino acid sequence set forth in HC-1091;
the amino acid sequence set forth in HC-1092;
the amino acid sequence set forth in HC-1093; or
the amino acid sequence set forth in HC-1094,
wherein the HA tag is optionally excluded from any foregoing amino acid sequence that contains an HA tag.

In some embodiments, an anti-TfR1 antibody comprises a heavy chain having at least 80%, at least 85%, at least 90%, at least 95%, at least 99%, or 100% sequence identity to a heavy chain sequence identified herein, wherein the anti-TfR1 antibody does not have a VH and VL identical to the VH and VL of H2C/L0. In some embodiments, an anti-TfR1 antibody comprises a light chain having at least 80%, at least 85%, at least 90%, at least 95%, at least 99%, or 100% sequence identity to a light chain sequence identified herein, wherein the anti-TfR1 antibody does not have a VH and VL identical to the VH and VL of H2C/L0. In some embodiments, an anti-TfR1 antibody comprises a heavy chain having at least 80%, at least 85%, at least 90%, at least 95%, at least 99%, or 100% sequence identity to a heavy chain sequence identified herein and a light chain having at least 80%, at least 85%, at least 90%, at least 95%, at least 99%, or 100% sequence identity to a light chain sequence identified herein, wherein the anti-TfR1 antibody does not have a VH and VL identical to the VH and VL of H2C/L0.

In some embodiments, an anti-TfR1 antibody comprises a heavy chain comprising an amino acid sequence that is at least 80%, 85%, 90%, 95%, 99%, or 100% identical to the amino acid sequence as set forth in any one of HC-1043 to HC-1094 (as shown in Table 10) and HC-2002 to HC-2020 (as shown in Table 22); and/or comprises a light chain comprising an amino acid sequence that is at least 80%, 85%, 90%, 95%, 99%, or 100% identical to the amino acid sequence as set forth in any one of LC-1095 to LC-1114 (as shown in Table 10) and LC-2021 (as shown in Table 22).

In some embodiments, an anti-TfR1 antibody comprises a heavy chain having an amino acid sequence that has one, two, or three, or more modifications (e.g., substitutions, deletions, or insertions) to a heavy chain sequence identified herein, wherein the anti-TfR1 antibody does not have a VH and VL identical to the VH and VL of H2C/L0. In some embodiments, an anti-TfR1 antibody comprises a light chain having an amino acid sequence that has one, two, or three, or more modifications (e.g., substitutions, deletions, or insertions) to a light chain sequence identified herein, wherein the anti-TfR1 antibody does not have a VH and VL identical to the VH and VL of H2C/L0. In some embodiments, an anti-TfR1 antibody comprises a heavy chain having an amino acid sequence that has one, two, or three, or more modifications (e.g., substitutions, deletions, or insertions) to a heavy chain sequence identified herein and a light chain having an amino acid sequence that has one, two, or three, or more modifications (e.g., substitutions, deletions, or insertions) to a light chain sequence identified herein, wherein the anti-TfR1 antibody does not have a VH and VL identical to the VH and VL of H2C/L0.

The present disclosure further embraces additional variants and equivalents that are substantially homologous to the recombinant, monoclonal, chimeric, humanized, and human antibodies, or antibody fragments thereof, described herein. In some embodiments, it is desirable to modulate the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

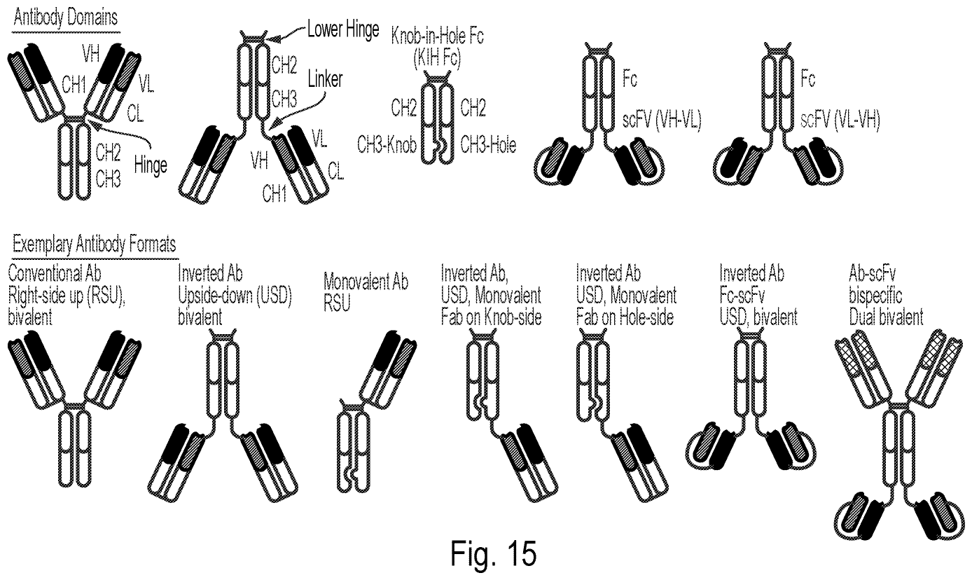

Effector function of antibodies can be modulated by amino acid mutations and/or domain substitutions (e.g., including but not limited to those described in Dumet et al. MABS 2019; 11 (8): 1341-50). Additional characteristics such as pharmacokinetics (e.g., Dall'acqua et al J of Immunology 2002; 169 (9) 5171-80), glycosylation, immunogenicity, solubility, and stability can be engineered by modification of Fc by mutations or substitutions. In addition, novel antigen specificity can be engineered into constant domains to create new paratopes (e.g., Wozniak-Knopp et al. PEDS 2010; 23 (4): 289-97). The affinity or avidity of a Fab may be modulated by changing the linkages between domains of antibodies such as removing the Fab from the top portion of the antibody and linking the Fab to the Fc C-terminus by a linker of any length from zero to 40 amino acids and fusing into the N-terminus of either the VH or VL domain of the Fab creating an “upside-down” or “inverted” antibody with potentially modulated affinity or avidity for binding to antigen, and modulated effector function (e.g., Weber et al. Cell Reports 2018; 22:149-62). Additional exemplary antibody formats are depicted in FIG. 15.

Variations may be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions may optionally be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant may be determined by systematically making insertions, deletions, or substitutions in the sequence and testing the resulting variant proteins for activity as compared to the parent protein.

In some embodiments, variants may include addition of amino acid residues at the amino- and/or carboxyl-terminal end of the antibody or polypeptide. The length of additional amino acids residues may range from one residue to a hundred or more residues. In some embodiments, a variant comprises an N-terminal methionyl residue. In some embodiments, the variant comprises an additional polypeptide/protein (e.g., Fc region) to create a fusion protein. In some embodiments, a variant is engineered to be detectable and may comprise a detectable label and/or protein (e.g., a fluorescent tag or an enzyme).

In some embodiments, a cysteine residue not involved in maintaining the proper conformation of an antibody is substituted or deleted to modulate the antibody's characteristics, for example, to improve oxidative stability and/or prevent aberrant disulfide crosslinking. Conversely, in some embodiments, one or more cysteine residues are added to create disulfide bond(s) to improve stability.

In some embodiments, an antibody of the present disclosure comprises variant hinge regions incapable of forming disulfide linkages between identical heavy chains (e.g., reduce homodimer formation). In some embodiments, the antibodies comprise heavy chains with changes in amino acids that result in altered electrostatic interactions. In some embodiments, the antibodies comprise heavy chains with changes in amino acids that result in altered hydrophobic/hydrophilic interactions.

In some embodiments, an antibody of the present disclosure is “deimmunized”. The deimmunization of antibodies generally consists of introducing specific amino acid mutations (e.g., substitutions, deletions, additions) that result in removal of predicted T-cell epitopes without significantly reducing the binding affinity or other desired characteristics of the antibody.

The variant antibodies or polypeptides described herein may be generated using methods known in the art, including but not limited to, site-directed mutagenesis, alanine scanning mutagenesis, and PCR mutagenesis.

In some embodiments an anti-TfR1 antibody described herein is chemically modified. In some embodiments, the anti-TfR1 antibody has been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications may be carried out by known techniques.

Generally speaking, antigen-antibody interactions are non-covalent and reversible, formed by a combination of hydrogen bonds, hydrophobic interactions, electrostatic and van der Waals forces. When describing the strength of an antigen-antibody complex, the terms affinity and/or avidity are often used. The binding of an antibody to its antigen is a reversible process, and the affinity of the binding is typically reported as an equilibrium dissociation constant (K_{D}). K_{D} is the ratio of an antibody dissociation rate (koff, also referred to herein as k_{d}) (how quickly it dissociates from its antigen) to the antibody association rate (kon) (how quickly it binds to its antigen). In some embodiments, K_{D} values are determined by measuring the kon and koff rates of a specific antibody/antigen interaction and then using a ratio of these values to calculate the K_{D} value. In some embodiments, K_{D} values are used to evaluate and rank the strength of individual antibody/antigen interactions. The lower the K_{D} of an antibody, the higher the affinity of the antibody for its target. In some embodiments, affinity is measured using SPR technology in a Biacore system. Avidity gives a measure of the overall strength of an antibody-antigen complex. It is dependent on three major parameters: (i) affinity of the antibody for the target, (ii) valency of both the antibody and antigen, and (iii) structural arrangement of the parts that interact.

In some embodiments, an anti-TfR1 antibody described herein has a monovalent affinity (K_{D}) for hTfR1 of >10 nM (e.g., >25 nM, >50 nM, >100 nM, >500 nM, >600 nM, >700 nM, >800 nM, >900 nM, 1000 nM, >10 nM to 100 nM, >10 nM to 1000 nM, >10 nM to 1 uM, >20 nM to 1 uM, 20 nM to 1 uM, 100 nM to 1 uM, 100 nM to 500 nM, 500 nM to 1 uM, 50 nM to 1 uM, 100 nM to 1 uM, 500 nM to 10 uM, or 1 uM to 10 uM) and/or an off rate (k_{d}) of >=0.01/s. In some embodiments, the anti-TfR1 antibody is monovalent or multivalent (e.g., bivalent). In some embodiments, an anti-TfR1 antibody described herein is a multivalent (e.g., bivalent) antibody and has a monovalent affinity of >100 nM. In some embodiments, an anti-TfR1 antibody described herein is a multivalent (e.g., bivalent) antibody and has a monovalent affinity of >500 nM. In some embodiments, an anti-TfR1 antibody described herein is a multivalent (e.g., bivalent) antibody and has a monovalent affinity of >1000 nM. In some embodiments, an anti-TfR1 antibody described herein is a multivalent (e.g., bivalent) antibody and has a monovalent affinity of ≥2000 nM. In some embodiments, an anti-TfR1 antibody described herein is a multivalent (e.g., bivalent) antibody and has a monovalent affinity of >3000 nM. In some embodiments, an anti-TfR1 antibody described herein is a multivalent (e.g., bivalent) antibody and has a monovalent affinity of >4000 nM. In some embodiments, an anti-TfR1 antibody described herein is a multivalent (e.g., bivalent) antibody and has a monovalent affinity of 1000 nM to 5000 nM. In some embodiments, an anti-TfR1 antibody described herein is a multivalent (e.g., bivalent) antibody and has a bivalent affinity for cell surface TfR1 of about 5 nM to about 500 nM, e.g., about 5 nM to about 250 nm, e.g., about 10 nM to about 200 nM, e.g., about 10 nM to about 150 nM, e.g., about 10 nM to about 100 nM. In some embodiments, an anti-TfR1 antibody described herein is a monovalent antibody and has a monovalent affinity for hTfR1 of >10 nM. In some embodiments, an anti-TfR1 antibody described herein is a monovalent antibody and has a monovalent affinity for hTfR1 of 20 nM to 1000 nM. In some embodiments, an anti-TfR1 antibody described herein is a monovalent antibody and has a monovalent affinity for hTfR1 of 50 nM to 1000 nM. In some embodiments, an anti-TfR1 antibody described herein is a monovalent antibody and has a monovalent affinity for hTfR1 of 100 nM to 1000 nM. In some embodiments, an anti-TfR1 antibody described herein is a monovalent antibody and has a monovalent affinity for hTfR1 of 500 nM to 1000 nM.

### Methods of Making Anti-TfR1 Antibodies

The anti-TfR1 antibodies described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional variants thereof. In some embodiments, a DNA sequence encoding a polypeptide of interest is constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), a polynucleotide sequence encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In some embodiments, a recombinant expression vector is used to amplify and express DNA encoding an antibody against human TfR1. For example, a recombinant expression vector can be a replicable DNA construct that includes synthetic or cDNA-derived DNA fragments encoding a polypeptide chain of an anti-TfR1 antibody operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence that is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can also be included. DNA regions are “operatively linked” when they are functionally related to each other. For example, DNA for a signal peptide (secretory leader) is operatively linked to DNA for a polypeptide if it is expressed as a precursor that participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide may include an N-terminal methionine residue. This residue can optionally be subsequently cleaved from the expressed recombinant protein to provide a final product.

The choice of an expression control sequence and an expression vector generally depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

In some embodiments, an anti-TfR1 antibody of the present disclosure is expressed from one or more vectors. In some embodiments, a heavy chain polypeptide is expressed by one vector and a light chain polypeptide is expressed by a second vector. In some embodiments, a heavy chain polypeptide and a light chain polypeptide are expressed by one vector. Thus, the present disclosure provides vectors encoding an anti-TfR1 antibody described herein. In one embodiment, the vector encodes a heavy chain polypeptide of an anti-TfR1 antibody described herein. In one embodiment, the vector encodes a light chain polypeptide of an anti-TfR1 antibody described herein. In one embodiment, the vector encodes a heavy chain polypeptide and a light chain polypeptide of an anti-TfR1 antibody described herein.

Suitable host cells for expression of an anti-TfR1 antibody or a TfR1 protein or fragment thereof to use as an antigen or immunogen include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described herein. Cell-free translation systems may also be employed. Appropriate cloning and expression vectors for use with bacterial, fungal, yeast, and mammalian cellular hosts, as well as methods of protein production, including antibody production are well-known in the art.

Various mammalian culture systems may be used to express recombinant polypeptides. Expression of recombinant proteins in mammalian cells may be desirable because these proteins are generally correctly folded, appropriately modified, and biologically functional. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present disclosure provides cells comprising the anti-TfR1 antibody described herein. The present disclosure also provides cells comprising one or more polynucleotides encoding an anti-TfR1 antibody described herein or one or more vectors encoding anti-TfR1 antibody described herein. In one embodiment, the cell comprises a polynucleotide encoding an anti-TfR1 antibody described herein. In one embodiment, the cell comprises a first polynucleotide encoding a heavy chain of an anti-TfR1 antibody described herein and a second polynucleotide encoding a light chain of an anti-TfR1 antibody described herein. In one embodiment, the cell comprises a polynucleotide encoding a heavy chain and a light chain of an anti-TfR1 antibody described herein. In one embodiment, the cell comprises a vector encoding a an anti-TfR1 antibody described herein. In one embodiment, the cell comprises a first vector encoding a heavy chain of an anti-TfR1 antibody described herein and a second vector encoding a light chain of an anti-TfR1 antibody described herein. In one embodiment, the cell comprises a vector encoding a heavy chain and a light chain of an anti-TfR1 antibody described herein. In some embodiments, the cells produce the anti-TfR1 antibodies described herein. In some embodiments, the cells produce an antibody. In some embodiments, the cells produce an antibody that binds human TfR1. In some embodiments, the cells produce an antibody that binds cyno TfR1. In some embodiments, the cells produce an antibody that binds human TfR1 and cyno TfR1. In some embodiments, the cell is a prokaryotic cell (e.g., E. coli). In some embodiments, the cell is a eukaryotic cell. In some embodiments, the cell is a mammalian cell. In some embodiments, the cell is a hybridoma cell.

Proteins produced by a host cell can be purified according to any suitable method. Standard methods include chromatography (e.g., ion exchange, affinity, and sizing column chromatography), centrifugation, differential solubility, or by any other standard technique for protein purification. Affinity tags such as hexa-histidine (SEQ ID NO:XX), maltose binding domain, influenza coat sequence, and glutathione-S-transferase can be attached to the protein to allow easy purification by passage over an appropriate affinity column. Affinity chromatography used for purifying immunoglobulins include, but are not limited to, Protein A, Protein G, and Protein L chromatography. Isolated proteins can be physically characterized using techniques known to those of skill in the art, including but not limited to, proteolysis, size exclusion chromatography (SEC), mass spectrometry (MS), nuclear magnetic resonance (NMR), isoelectric focusing (IEF), high performance liquid chromatography (HPLC), and x-ray crystallography. The purity of isolated proteins can be determined using techniques known to those of skill in the art, including but not limited to, SDS-PAGE, SEC, capillary gel electrophoresis, IEF, and capillary isoelectric focusing (cIEF).

In some embodiments, supernatants from expression systems that secrete recombinant protein into culture media are first concentrated using a commercially available protein concentration filter, for example, an Amicon® or Millipore Pellicon® ultrafiltration unit.

Following the concentration step, the concentrate can be applied to a suitable purification matrix.

In some embodiments, an anion exchange resin is employed, for example, a matrix or substrate having pendant diethylaminoethyl (DEAE) groups. The matrices can be acrylamide, agarose, dextran, cellulose, or other types commonly employed in protein purification. In some embodiments, a cation exchange step is employed. Suitable cation exchangers include various insoluble matrices comprising sulfopropyl or carboxymethyl groups. In some embodiments, a hydroxyapatite media is employed, including but not limited to, ceramic hydroxyapatite (CHT).

In some embodiments, one or more reverse-phase HPLC steps employing hydrophobic RP-HPLC media, e.g., silica gel having pendant methyl or other aliphatic groups, are employed to further purify a recombinant protein. In some embodiments, hydrophobic interaction chromatography (HIC) is used to separate recombinant proteins based on their hydrophobicity. HIC is a useful separation technique for purifying proteins while maintaining biological activity due to the use of conditions and matrices that operate under less denaturing conditions than some other techniques. Some or all of the foregoing purification steps, in various combinations, can be employed to provide a homogeneous recombinant protein.

In some embodiments, the antibody of the this disclosure is an Fab that can be generated by first making a full monoclonal Ab followed by digesting the monoclonal antibody by chemical or enzymatic cleavage (e.g., pepsin, papain, or ficin digestion) to yield a F(ab′)₂ fragment, followed by reduction of those fragments to yield Fab fragments. Such techniques are known in the art. See, e.g., Victor C-G et al., Biosensors and Bioelectronics, 2016 (85): 32-45. Alternatively, the antibody of this disclosure is made by recombinant synthesis of F(ab′)₂ antibody fragments, followed by chemical reduction of these fragments to yield Fab units.

### Polynucleotides

In some embodiments, the disclosure encompasses polynucleotides comprising polynucleotides that encode a polypeptide (e.g., an anti-TfR1 antibody) described herein. The term “polynucleotides that encode a polypeptide” encompasses a polynucleotide that includes only coding sequences for the polypeptide as well as a polynucleotide that includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide comprises a polynucleotide (e.g., a nucleotide sequence) encoding a heavy chain of an anti-TfR1 antibody described herein. In some embodiments, the polynucleotide comprises a polynucleotide (e.g., a nucleotide sequence) encoding a light chain of an anti-TfR1 antibody described herein. In some embodiments, the polynucleotide comprises a polynucleotide (e.g., a nucleotide sequence) encoding a heavy chain of an anti-TfR1 antibody described herein and a polynucleotide (e.g., a nucleotide sequence) encoding a light chain of an anti-TfR1 antibody.

In some embodiments, the polynucleotide comprises a polynucleotide (e.g., a nucleotide sequence) encoding a polypeptide comprising a VH amino acid sequence depicted in Table 3. In some embodiments, the polynucleotide comprises a polynucleotide (e.g., a nucleotide sequence) encoding a polypeptide comprising a VL amino acid sequence depicted in Table 4. In some embodiments, the polynucleotide comprises a polynucleotide (e.g., a nucleotide sequence) encoding a polypeptide comprising a VH amino acid sequence depicted in Table 3 and a polypeptide comprising a VL amino acid sequence depicted in Table 4. In some embodiments, the polynucleotide comprises a polynucleotide (e.g., a nucleotide sequence) encoding a polypeptide comprising a heavy chain amino acid sequence depicted in Table 10. In some embodiments, the polynucleotide comprises a polynucleotide (e.g., a nucleotide sequence) encoding a polypeptide comprising a light chain amino acid sequence depicted in Table 10. In some embodiments, the polynucleotide comprises a polynucleotide (e.g., a nucleotide sequence) encoding a polypeptide comprising a heavy chain amino acid sequence depicted in Table 10 and a polypeptide comprising a light chain amino acid sequence depicted in Table 10.

The polynucleotide variants can contain alterations in the coding regions, non-coding regions, or both. In some embodiments, a polynucleotide variant contains alterations that produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). In some embodiments, a polynucleotide variant comprises one or more mutated codons comprising one or more (e.g., 1, 2, or 3) substitutions to the codon that change the amino acid encoded by that codon. Methods for introducing one or more substitutions into a codon are known in the art, such as, e.g., PCR mutagenesis and site-directed mutagenesis. Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, a polynucleotide comprises the coding sequence for a polypeptide (e.g., an antibody) fused in the same reading frame to a polynucleotide that aids in expression and secretion of a polypeptide from a host cell (e.g., a leader sequence that functions as a secretory sequence for controlling transport of a polypeptide). The polypeptide can have the leader sequence cleaved by the host cell to form a “mature” form of the polypeptide.

In some embodiments, a polynucleotide comprises the coding sequence for a polypeptide (e.g., an antibody) fused in the same reading frame to a marker or tag sequence. For example, in some embodiments, a marker sequence is a hexa-histidine (SEQ ID NO:XX) tag (HIS-tag) that allows for efficient purification of the polypeptide fused to the marker. In some embodiments, a marker sequence is a hemagglutinin (HA) tag derived from the influenza hemagglutinin protein when a mammalian host (e.g., COS-7 cells) is used. In some embodiments, the marker sequence is a FLAG™ tag. In some embodiments, a marker is used in conjunction with other markers or tags.

In some embodiments, the polynucleotides are isolated. In some embodiments, the polynucleotides are substantially pure.

### Vectors and Cells

Vectors and cells comprising each and every one of the polynucleotides described herein are also provided. In some embodiments, an expression vector comprises a polynucleotide molecule encoding an anti-TfR1 antibody described herein. In some embodiments, an expression vector comprises a polynucleotide molecule encoding a polypeptide that is part of a an anti-TfR1 antibody described herein. In some embodiments, an expression vector comprises a polynucleotide molecule encoding a heavy chain polypeptide of an anti-TfR1 antibody described herein. In some embodiments, an expression vector comprises a polynucleotide molecule encoding a light chain polypeptide of an anti-TfR1 antibody described herein. In some embodiments, an expression vector comprises a polynucleotide molecule encoding a heavy chain polypeptide and a light chain polypeptide of anti-TfR1 antibody described herein. In some embodiments, a host cell comprises an expression vector comprising the polynucleotide molecule encoding an anti-TfR1 antibody described herein. In some embodiments, a host cell comprises an expression vector comprising the polynucleotide molecule encoding a polypeptide that is part of an anti-TfR1 antibody described herein. In some embodiments, a host cell comprises a polynucleotide molecule encoding an anti-TfR1 antibody described herein. In some embodiments, a host cell comprises an expression vector comprising a polynucleotide molecule encoding a heavy chain polypeptide of an anti-TfR1 antibody described herein. In some embodiments, a host cell comprises an expression vector comprises a polynucleotide molecule encoding a light chain polypeptide of an anti-TfR1 antibody described herein. In some embodiments, a host cell comprises an expression vector comprises a first polynucleotide encoding a heavy chain polypeptide and a second polynucleotide light chain polypeptide of an anti-TfR1 antibody described herein. In some embodiments, a host cell comprises: (ii) a first expression vector comprising a polynucleotide molecule encoding a heavy chain polypeptide of an anti-TfR1 antibody described herein, and (ii) a second expression vector comprising a polynucleotide molecule encoding a light chain polypeptide of the anti-TfR1 antibody.

### Analysis of Physical/Chemical Properties of Anti-TfR1 Antibodies

Anti-TfR1 antibodies of the present disclosure may be analyzed for their physical/chemical properties and/or biological activities by various methods known in the art. In some embodiments, an anti-TfR1 antibody is tested for its ability to bind TfR1 (e.g., human TfR1 and/or cyno TfR1). Binding assays include, but are not limited to, SPR (e.g., Biacore), ELISA, and flow cytometry. In some embodiments, an anti-TfR1 antibody is tested for its ability to inhibit, reduce, or block binding of transferrin to its TfR1 receptor. In some embodiments, an anti-TfR1 antibody is tested for its ability to inhibit, reduce, or block TfR1 activity. In some embodiments, an anti-TfR1 antibody is tested for its ability to internalize with TFR1 and induce increased internalization of TfR1. In addition, antibodies may be evaluated for solubility, stability, thermostability, viscosity, expression levels, expression quality, and/or purification efficiency.

In some embodiments, assays are provided for identifying an anti-TfR1 antibody that affects TfR1 activity. In some embodiments, SPR, ELISA, or FACS assays are used to assess the ability of an anti-TfR1 antibody to block binding of TfR1 to Tf. In some embodiments, cytotoxicity assays are used to assess the ability of an anti-TfR1 antibody to affect natural killer (NK) cell activity. In some embodiments, proliferation assays are used to assess the ability of an anti-TfR1 antibody to affect T-cell activity.

In some embodiments, an anti-TfR1 antibody described herein is an antagonist of human TfR1. In some instances, the terms “inhibiting”, “inducing”, “reducing”, “increasing”, “enhancing” are relative to levels/activity in the absence of treatment with a conjugate comprising the anti-TfR1 antibody. In some instances, the terms “inhibiting”, “inducing”, “reducing”, “increasing”, “enhancing” are relative to levels/activity prior to treatment with a conjugate comprising the anti-TfR1 antibody.

### Anti-TfR1 Antibody Conjugates and Complexes

The present disclosure also provides conjugates comprising an anti-TfR1 antibody described herein conjugated to a second molecule. In some embodiments, the second molecule comprises any agent, e.g., therapeutic agent, described herein.

Conjugates comprising an anti-TfR1 antibody described herein may be made using any suitable method known in the art. In some embodiments, the components of the conjugate are linked by covalent interactions. In some embodiments, conjugates are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, an anti-TfR1 antibody described herein is conjugated to a detectable substance or molecule that allows the agent to be used for diagnosis and/or detection. A detectable substance can include, but is not limited to, enzymes, such as horseradish peroxidase, alkaline phosphatase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³In, ¹¹²In, ¹¹¹In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Re, ¹⁰⁵Rh, ⁹⁷Ru, ³⁵S, ⁴⁷Sc, ⁷⁵Se, ¹⁵³Sm, ¹¹³Sn, ¹¹⁷Sn, ⁸⁵Sr, ^{99m}Tc, ²⁰¹Ti, ¹³³Xe, ⁹⁰Y, ⁶⁹Yb, ¹⁷⁵Yb, ⁶⁵Zn; positron emitting metals; and magnetic metal ions.

An anti-TfR1 antibody described herein can also be conjugated to a second antibody to form an antibody heteroconjugate.

In some embodiments, the anti-TfR1 antibody of the present disclosure can be conjugated to a molecule or drug, such as a nucleic acid, e.g., an antisense oligonucleotide, a short interfering RNA (siRNA), an RNA such as messenger RNA (mRNA), microRNA (miRNA), guide RNA (gRNA), a phosphoroamidate morpholino oligomer or an aptamer, etc. In some embodiments, the anti-TfR1 antibody is conjugated to a particle, e.g., lipid particle or nanoparticle, which can contain a therapeutic agent such as one described herein. In some embodiments, the anti-TfR1 antibody is conjugated to a viral particle, e.g., a viral particle comprising a therapeutic nucleic acid and/or protein, e.g., a viral particle for gene therapy (e.g., an adeno-associated virus or a lentivirus). The anti-TfR1 antibody may be linked to the drug by a linker. In some embodiments, the anti-TfR1 antibody is conjugated to a small molecule such as a cytotoxic agent (e.g., maitansine). In some embodiments, the anti-TfR1 antibody is conjugated to an anti-inflammatory agent (e.g., a glucocorticoid). In some embodiments, the anti-TfR1 antibody is conjugated to a half-life extension moiety (e.g., polyethylene glycol). Methods of preparing antibody-nucleic acid conjugates, such as the conjugates contemplated in this disclosure are well-known in the art. See, e.g., US Patent Application Publication No. US20190240346, and U.S. Pat. Nos. 10,881,743 and 10,550,188, and International Patent Application Publication No. WO1991004753, the disclosures of which are incorporated by reference herein in their entirety.

In some embodiments, the conjugate is a fusion protein. Fusion proteins comprising an anti-TfR1 antibody described herein can be made using any suitable method known in the art. Such a fusion protein can include a fusion of an anti-TfR1 antibody of the disclosure (including bispecific, multispecific, or multivalent anti-TfR1 antibodies) with a therapeutic polypeptide or antibody. In one instance, an anti-TfR1 antibody described herein is conjugated via a fusion protein to an anti-beta amyloid antibody (e.g., aducanumab). In one instance, an anti-TfR1 antibody described herein is conjugated via a fusion protein to rituximab. In one instance, an anti-TfR1 antibody described herein is conjugated via a fusion protein to an enzyme (e.g., iduronate 2-sulfatase, glucocerebrosidase, alpha-L-iduronidase, or sulfamidase). In one instance, avidin can be added to the C-terminus of the heavy chain to produce a fusion protein as described in Candelaria P V et al. Front Immunol. 2021; 12:607692. The fusion protein may be further conjugated or complexed to a second molecule or drug, such as a biotinylated drug, as described in Daniels T R, et al. Biochim Biophys Acta. 2012; 1820 (3): 291-317.

In some embodiments, complexes comprising an anti-TfR1 antibody described herein can be made using any suitable method known in the art. In some embodiments, the components of the complex are linked by non-covalent interactions. Such compounds comprise an anti-TfR1 antibody complexed with another agent, e.g., therapeutic agent, or complexed with a lipid or nanoparticle which has a therapeutic polypeptide or protein.

### Tissue Targeting and Use of Anti-TfR1 Antibodies

In some embodiments, an anti-TfR1 antibody described can be used to target brain tissue and transport an agent across the blood brain barrier for the treatment of a neurological disorder. Exemplary neurological disorders include Alzheimer's disease, Parkinson's disease, frontotemporal dementia, ALS, Huntington's disease, multiple sclerosis, spinal muscular atrophy, muscular dystrophy, spinal cord injury, stroke, ophthalmological conditions, acute or chronic optic neuritis, psychiatric disorders, Tourette's disease brain injury, brain tumors, and epilepsy.

Exemplary therapeutic agents for the treatment of Alzheimer's disease include caprylic triglyceride, anti-tau antibody, anti-beta amyloid antibody, anti-DKK1 antibody, APOE antagonist antibody, donepezil, quinidine, a serotonin 6 receptor antagonist, a beta-secretase inhibitor, a RAGE antagonist, a BACE inhibitor, an amyloid beta-protein inhibitor, a phosphodiesterase 9A inhibitor, bisnorcymserine, bryostatin-1, an alpha-7 potentiator, a purinoceptor P2Y6 agonist, a tau protein aggregation/TDP-43 aggregation inhibitor, N3pG-Aß mAb, an mGlu2 agonist, quinazolinone, a mitochondrial protein stimulant, an amyloid precursor protein secretase inhibitor, a 5HT6 antagonist, R-phenserine, an amyloid beta/tau protein inhibitor, a MAO-B inhibitor, an Lp-PLA2 inhibitor, a 5-HT6 receptor antagonist, a BET protein inhibitor, an anti-protofibrillar AB mAb, nomethiazole, a histamine H3 receptor antagonist, a PPAR-delta/gamma agonist, abeotaxane, and a p38 mitogen-activated protein kinase inhibitor.

Exemplary therapeutic agents for the treatment of ALS include an anti-SOD1 antibody, anti-DR6 antibody, anti-DPR antibody, dexpramipexole, arimoclomal, GM6, ibudilast, a macrophage modulator, a NOGO-A inhibitor, and a troponin complex stimulant.

Exemplary therapeutic agents for the treatment of brain injury include apomorphine, a cytokine inhibitor/neuropeptide receptor modulator, and a progesterone receptor agonist.

Exemplary therapeutic agents for the treatment of brain tumors include an IDH1 inhibitor, doxorubicin, paclitaxel, an anti-EGFRvIII antibody-drug conjugate, bevacizumab, a FGF-R kinase inhibitor, a PI3K inhibitor, cabozantinib, iodine I 131 derlotuximab biotin, a PDGFR inhibitor, carboxyamidotriazole orotate, a non-neurotoxic derivative of penclomidine, golvatinib, dexanabinol, a TGF-beta 1 kinase inhibitor, afatinib, an IDO inhibitor, cabazitaxel, a Src kinase/pre-tubulin inhibitor, a SMO protein inhibitor, an endothelin A/B receptor antagonist, a proteasome inhibitor, a T-type calcium channel antagonist, a thapsigargin analogue, irinotecan, nivolumab, a CSF-1R inhibitor, pelareorep, an EGFR antagonist, an exportin-1 protein inhibitor/nuclear protein inhibitor, a BIRC5 protein inhibitor, evofosfamide, abeotaxane, ENG protein inhibitor, trans-sodium crocetinate, an N7-alkylating agent, a targeted anti-angiogenic agent, and veliparib.

Exemplary therapeutic agents for the treatment of epilepsy include everolimus, eslicarbazepine acetate, alprazolam, brivaracetam, carbamazepine, cannabidiol, a 4-aminobutyrate transaminase inhibitor, perampanel, a GABA-A receptor agonist, synthetic huperzine, pregabalin, clobazam, diazepam, a GABAA synaptic and extra-synaptic receptor modulator, topiramate IV, lacosamide, and a serotonin receptor agonist.

Exemplary therapeutic agents for the treatment of genetic disorders (e.g., Friedrich's ataxia, late infantile neuronal ceroid, spinal and bulbar muscular atrophy, ataxia telangiectasia, pantothenate kinase-associated neurodegeneration, spinal muscular atrophy, familial amyloid polyneuropathy, Rett syndrome, Leigh syndrome, Wilson's disease) include a NF/E2 related factor 2 stimulant, interferon gamma-1b, rhTPP1 enzyme replacement therapy, vatiquinone, deferiprone, nusinersen, ISIS-TTRRX, a serotonin 1A receptor agonist, cytokine inhibitors/neuropeptide receptor modulator, an siRNA inhibitor targeting TTR, phosphopantothenate replacement, DcpS inhibitor, cysteamine bitartrate, indolepropionic acid, a transthyretin dissociation inhibitor, and bis-choline tetrathiomolybdate.

Exemplary therapeutic agents for the treatment of headache include an anti-CGRP mAb, a CGRP receptor antagonist mAb, sumatriptan, dextromethorphan/quinidine, onabotulinumtoxinA, a serotonin-1F receptor agonist, a nNOS inhibitor/5HT, dihydroergotamine, cyclobenzaprine, and aspirin/sumatriptan combination.

Exemplary therapeutic agents for the treatment of Huntington's disease include laquinimod, a PDE10 inhibitor, pridopidine, cysteamine bitartrate, and aVMAT2 inhibitor.

Exemplary therapeutic agents for the treatment of multiple sclerosis include natalizumab, monomethyl fumarate prodrug, anti-LINGO-1 antibody, a Nck protein modulator, a SIPR-1/5 receptor agonist, fingolimod, an anti-CD52 mAb, idebenone, a PPAR-gamma agonist/modulator, laquinimod, a tyrosine kinase inhibitor, an anti-CD19 mAb, ibudilast, guanabenz, an anti-CD20 mAb, interferon beta-1b, an IL-7 receptor inhibitor, a SIP1 receptor agonist, a myelin protein stimulant, estriol succinate, imilecleucel-T, an anti-VLA 2 mAb, a BAFF-R modulator, a CD100 antigen inhibitor, an anti-DR6 antibody, and an NF-kappa B inhibitor.

Exemplary therapeutic agents for the treatment of muscular dystrophy include a myostatin inhibitor, drisapersen, eteplirsen, halofuginone, idebenone, ISIS-DMPKRY, a (steroid receptor agonist, a GAPDH inhibitor, a genetic transcription inhibitor, tadalafil, ataluren, and a glucocorticoid receptor agonist.

Exemplary therapeutic agents for the treatment of pain include a neublastin, P2X3 purinoreceptor antagonist, a SNARE protein antagonist, oxycodone-naltrexone core (abuse resistant), amitriptyline/ketamine, rintatolimod, a cannabinoid receptor CB2 agonist, a non-eryhropoietic peptide, a PPAR-gamma agonist, a glycogen phosphorylase inhibitor, a NMDA receptor antagonist, zoledronic acid, an early growth response protein 1 inhibitor, a (histamine-3 receptor antagonist, buprenorphine, a cytokine inhibitor, cebranopadol, celecoxib, an arachidonic acid analog, a synthetic capsaicin, a Nav1.7 sodium channel inhibitor, an opioid kappa receptor agonist, duloxetine, a nerve growth factor stimulant, dexmedetomidine, a voltage-gated sodium channel inhibitor, bupivacaine, an angiotensin type 2 receptor antagonist, a nerve growth factor inhibitor, a p38 inhibitor, rapastinel, levorphanol, a CGRP mAb, pregabalin, an mGlu2/3 receptor agonist, a CACNA2D1 protein modulator, a bone resorption factor inhibitor, neublastin, a mu-opioid analgesic, an nNOS inhibitor, O-desmethyltramadol, palmitoylethanolamide, a GABA A agonist, a TRPV-1 receptor agonist, nabiximols, a cyclo-oxygenase 2 inhibitor, a nerve growth factor modulator, cyclobenzaprine, flurbiprofen, a fatty acid amide hydrolase inhibitor, and ibuprofen/phosphatidylcholine.

Exemplary therapeutic agents for the treatment of Parkinson's disease include amantadine, apomorphine, an alpha7 nicotine acetylcholine receptor partial agonist, an anti-alpha-synuclein antibody, alpha-synuclein inhibitor, levodopa, a D1 potentiator, dipraglurant, a serotonin 1A/1B partial agonist, fipamezole, GM6, a retinoid X receptor agonist, istradefylline, rotigotine, pramipexole/rasagiline, R-phenserine, a serotonin 2A/6 receptor antagonist, an adenosine A2A receptor antagonist, safinamide, and a dopamine receptor agonist.

Exemplary therapeutic agents for the treatment of spasticity include baclofen, onabotulinumtoxinA, abobotulinumtoxinA, arbaclofen, nabiximols, and incobotulinumtoxinA.

Exemplary therapeutic agents for the treatment of spinal cord injury include an anti-Lingo-1 antibody, anti-NgR1 antibody, neublastin, a nervous system modulator, a Rho GTP-binding protein-inhibitor, and fibroblast growth factor receptor.

Exemplary therapeutic agents for the treatment of stroke include natalizumab, recombinant mutant form of human wild-type activated protein C, ticagrelor, dalfampridine, aspirin, nimodipine microparticles, GM6, a PARP inhibitor, a PDZ domain inhibitor, a beta amyloid inhibitor, dabigatran, and sodium nitrite.

Exemplary therapeutic agents for the treatment of Tourette's Syndrome include a histamine-3 receptor antagonist, a 4-aminobutyrate transaminase inhibitor, abobotulinumtoxinA, ecopipam, a VMAT2 inhibitor, acamprosate, and vigabatrin.

Other exemplary therapeutic agents for the treatment of other neurological disorders include a myostatin inhibitor, NF/E2 related factor 2 stimulant, anti-tau antibody, a myeloperoxidase inhibitor, a mitochondrial permeability transition pore inhibitor, belimumab, type II-B activin receptor modulator mAb, a C1 esterase inhibitor, ferric carboxymaltose, amifampridine, fingolimod, a monoamine oxidase B inhibitor, a neurotransmitter modulator, a dopamine receptor agonist, an anti-CD19 mAb, a VMAT2 inhibitor, a CD20 mAb, thymosin beta-4, an anti-IL-6 receptor mAb, eculizumab, an AMPA receptor modulator, a steroid hydroxylase inhibitor, pyridoxal phosphate, abeotaxane, aceneuramic acid, and sodium oxybate.

In some embodiments, the conjugate is a fusion polypeptide comprising an anti-TfR1 antibody described herein and a whole antibody or antibody fragment (the therapeutic agent). In certain embodiments, the whole antibody or antibody fragment is an anti-beta amyloid antibody, an anti-tau antibody, an anti-alpha synuclein antibody, an anti-TDP-43 antibody, an anti-LINGO-1 antibody, an anti-LINGO-2 antibody, an anti-LINGO-3 antibody, an anti-LINGO-4 antibody, an anti-TREM2 antibody, an anti-C9orf72 dipeptide repeat poly-GA antibody (i.e., antibody capable of binding a dipeptide repeat (DPR) of poly-glycine-alanine (GA) having at least 6 repeats (GA) 6 as translated from the chromosome 9 open reading frame 72 (C9orf72) gene, an anti-TWEAK antibody, or an anti-TWEAK-R antibody.

In some embodiments, the conjugate is a fusion polypeptide comprising an anti-TfR1 antibody described herein and an antisense oligonucleotide (e.g., nusinersen).

### Pharmaceutical Compositions

The present disclosure provides compositions comprising an anti-TfR1 antibody described herein. The present disclosure also provides pharmaceutical compositions comprising an anti-TfR1 antibody described herein and a pharmaceutically acceptable vehicle.

Formulations are prepared for storage and/or use by combining an anti-TfR1 antibody of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22^{nd} Edition, 2012, Pharmaceutical Press, London). In some embodiments, the formulation is in the form of an aqueous solution. In some embodiments, the formulation is lyophilized or in an alternative dried form.

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present disclosure, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol, and cellulose acetate.

The binding agents of the present disclosure may be formulated in any suitable form for delivery to a target cell/tissue. In some embodiments, an anti-TfR1 antibody can be formulated as a liposome, microparticle, microcapsule, albumin microsphere, microemulsion, nano-particle, nanocapsule, or macroemulsion. In some embodiments, the pharmaceutical formulation includes an anti-TfR1 antibody of the present disclosure complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE).

In some embodiments, an anti-TfR1 antibody is formulated as a sustained-release preparation. Suitable examples of sustained-release preparations include semi-permeable matrices of solid hydrophobic polymers containing an agent, where the matrices are in the form of shaped articles (e.g., films or microcapsules). Sustained-release matrices include but are not limited to polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions or formulations of the present disclosure can be administered in any number of ways for either local or systemic treatment. In some embodiments, administration is topical by epidermal or transdermal patches, ointments, lotions, creams, gels, drops, suppositories, sprays, liquids and powders. In some embodiments, administration is pulmonary by inhalation or insufflation of powders or aerosols, including by nebulizer, intratracheal, and intranasal. In some embodiments, administration is oral. In some embodiments, administration is parenteral including intravenous, intraarterial, intratumoral, subcutaneous, intraperitoneal, intramuscular (e.g., injection or infusion), or intracranial (e.g., intrathecal or intraventricular). In some embodiments, administration is by intravenous injection or intravenous infusion. In some embodiments, administration is by intramuscular injection.

Various delivery systems are known and can be used to administer an anti-TfR1 antibody described herein. In some embodiments, an anti-TfR1 antibody or a composition described herein is delivered in a controlled release or sustained release system. In some embodiments, a pump is used to achieve controlled or sustained release. In some embodiments, polymeric materials are used to achieve controlled or sustained release of the anti-TfR1 antibody herein. Examples of polymers used in sustained release formulations include, but are not limited to, poly 2-hydroxy ethyl methacrylate, polymethyl methacrylate, polyacrylic acid, polyethylene-co-vinyl acetate, polymethacrylic acid, polyglycolides (PLG), polyanhydrides, poly N-vinyl pyrrolidone, polyvinyl alcohol (PVA), polyacrylamide, polyethylene glycol (PEG), polylactides (PLA), polylactide-co-glycolides (PLGA), and polyorthoesters. Any polymer used in a sustained release formulation should be inert, free of leachable impurities, stable on storage, sterile, and biodegradable.

Additional delivery systems can be used to administer an anti-TfR1 antibody described herein including, but not limited to, injectable drug delivery devices and osmotic pumps. Injectable drug delivery devices include, for example, hand-held devices (e.g., autoinjectors) or wearable devices. Different types of osmotic pump systems may include single compartment systems, dual compartment systems, and multiple compartment systems.

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### EXAMPLES

### Example 1: Generation of Anti-TfR1 Antibodies with Reduced Affinity

Without wishing to be bound by any theory, it is believed that anti-TfR1 antibodies can transport agents across brain endothelial cells via transcytosis, and that this process can be especially efficient and productive with antibodies having a weaker monovalent affinity and/or a faster off-rate, e.g., a K_{D} of >100 nM and/or a k_{d} of >=0.01/s. Monovalent antibody fragments, by virtue of reduced avidity of binding, can generally transport across the blood brain barrier with tighter monovalent affinities than bivalent antibodies. Thus, monovalent antibody fragments with monovalent affinity in the range K_{D}>10 nM can also transport across the blood-brain barrier, whereas for bivalent antibodies, having monovalent affinity of K_{D}>100 nM is preferred. In an effort to produce anti-TfR1 antibodies suitable for transport of therapeutic cargo across the brain endothelial cells, variants of a reference anti-TfR1 antibody (H2C/L0) were prepared in order to produce anti-TfR1 antibodies with reduced affinity for TfR1 and enhanced ability to mediate transcytosis. H2C/L0 is a humanized version of a rabbit anti-TfR1 antibody; H2C/L0 binds to human TfR1 and cynomolgus TfR with high affinity (e.g., K_{D} of <=10 nM). A brief description of the generation of the reference antibody, H2C/L0, is described in Example 9.

Proteins were expressed by transient transfection of suspension CHO-S cells in serum-free medium. Conditioned supernatant was collected by centrifugation and filtration. Protein concentration was estimated using biolayer interferometry (ForteBio Octet, ProA tips) by extrapolation of initial binding rates to a standard curve. CHO supernatants containing protein were diluted accordingly for further experiments.

For flow cytometry cell binding studies, proteins were bound to CHO cells expressing full length human TfR for 1-2 hours on ice, cells were washed twice with isotonic buffer, incubated with fluorescent secondary reagent (PE conjugated) that binds human IgG, washed twice, fixed by paraformaldehyde and analyzed on a flow cytometer. Mean fluorescence intensity (MFI) of the PE fluorophore was calculated and represents binding of antibody to cells.

For surface plasmon resonance binding studies, binding affinity and kinetics measurements were performed with a Biacore T200 instrument (Cytiva). A CM5 sensor chip was coated with anti-hIgG capture reagent, according to the manufacturer's protocol (Human Antibody Capture Kit; Cytiva), then equilibrated in running buffer: 10 mM HEPES pH 7.4, 150 mM NaCl, 3 mM EDTA, 0.05% bovine serum albumin, 0.005% surfactant P20. Anti-TfR1 H2C/L0 variant antibodies in CHO supernatant, normalized at 15 mcg/ml according to titers measured by anti-hIgG bilayer interferometry (ForteBio), were diluted in running buffer to 1 mg/ml, and captured on the sensor chip surface at 10-100 pg/mm² from injections of 10 mcL/min for 1 minute. Monomeric protein with human TfR1 apical domain (His8-BirApep-Cyno minTfR ECD huApi, amino acid sequence shown below), or with cynomolgus TfR1 apical domain (His8-BirApep-Cyno minTfR ECD, amino acid sequence shown below) was injected in single-cycle kinetics mode at increasing concentrations of 3.125, 12.5, 50, 200 and 800 nM at 30 mcL/minute for 3 minutes each and dissociation was monitored for 7 minutes.

His8-BirApep-Cyno minTfR ECD huApi
(SEQ ID NO: XX)
HHHHHHHHGLNDIFEAQKIEWHEGGLYWDDLKRKLSEKLDTTDFT
STIKLLNENLYVPREAGSQKDENLALYIENQFREFKLSKVWRDQH
FVKIQVKDSAQNSVIIVDKNGRLVYLVENPGGYVAYSKAATVTGK
LVHANFGTKKDFEDLYTPVNGSIVIVRAGKITFAEKVANAESLNA
IGVLIYMDQTKFPIVNAELSFFGHAHLGTGDPYTGGSSGLPNIPV
QTISRAAAEKLFGNMEGDCPSDWKTDSTCRMVTSESKNVKLTVSN
VLKETKILNIFGVIKGFVEPDHYVVVGAQRDAWGPGAAKSSVGTA
LLLKLAQMFSDMVLKDGFQPSRSIIFASWSAGDFGSVGATEWLEG
YLSSLHLKAFTYINLDKAVLGTSNFKVSASPLLYTLIEKTMQDVK
HPVTGRSLYQDSNWASKVEKLTLDNAAFPFLAYSGIPAVSFCFCE
DTDYPYLGTTMDTYKELVERIPELNKVARAAAEVAGQFVIKLTHD
TELNLDYERYNSQLLLFLRDLNQYRADVKEMGL
His8-BirApep-Cyno minTfR ECD
(SEQ ID NO: XX)
HHHHHHHHGLNDIFEAQKIEWHEGGLYWDDLKRKLSEKLDTTDFT
STIKLLNENLYVPREAGSQKDENLALYIENQFREFKLSKVW
RDQHFVKIQVKDSAQNSVIIVDKNGGLVYLVENPGGYVAYSKAAT
VTGKLVHANFGTKKDFEDLDSPVNGSIVIVRAGKITFAEKV
ANAESLNAIGVLIYMDQTKFPIVKADLSFFGHAHLGTGDPYTGGS
SGLPNIPVQTISRAAAEKLFGNMEGDCPSDWKTDSTCKMVTSENK
SVKLTVSNVLKETKILNIFGVIKGFVEPDHYVVVGAQRDAWGPGA
AKSSVGTALLLKLAQMFSDMVLKDGFQPSRSIIFASWSAGDFGSV
GATEWLEGYLSSLHLKAFTYINLDKAVLGTSNFKVSASPLLYTLI
EKTMQDVKHPVTGRSLYQDSNWASKVEKLTLDNAAFPFLAY
SGIPAVSFCFCEDTDYPYLGTTMDTYKELVERIPELNKVARAAAE
VAGQFVIKLTHDTELNLDYERYNSQLLLFLRDLNQYRADVKEMGL

Following each cycle of injections, the anti-hIgG capture surface was regenerated with 3M MgCl₂ for 2×1 minute. Affinity and kinetic parameters were analyzed by fitting sensogram data with a 1:1 binding model using the Biacore T200 Evaluation Software (Cytiva). In cases where the kinetics were determined to be too fast to be accurately determined (dissociation rate constant k_{d}>0.05/s), stead-state affinity analysis was used to estimate the equilibrium dissociation constant, K_{D}. In cases where the binding response at 800 nM TfR was not sufficient (<50% of the saturation binding response expected base on antibody capture levels) no affinity was reported (K_{D}>800 nM). Some antibodies were retested for binding monomeric TfR up to 4000 nM of antigen with human apical domain and up to 8000 nM for antigen with cynomolgus apical domain.

Positions of the H2C/L0 paratope to be mutated were identified by examining the primary antibody sequence and the three-dimensional structure of H2C/L0 with its target, TfR. The amino acids alanine, histidine and aspartate were chosen as residues with diverse structures and properties that are often well tolerated in antibodies. In some cases, mutation to serine was tested (e.g., VL-N30S, VL-N32S, and VL-Y111S) to increase human-ness and thereby reduce immunogenicity. Overall, when either introduced to H2C/L0 in the conventional (right-side up) or inverted (upside-down) orientation, binding data show that many of these mutations reduce binding to human and/or cynomolgus TfR either on cells or in solution or both.

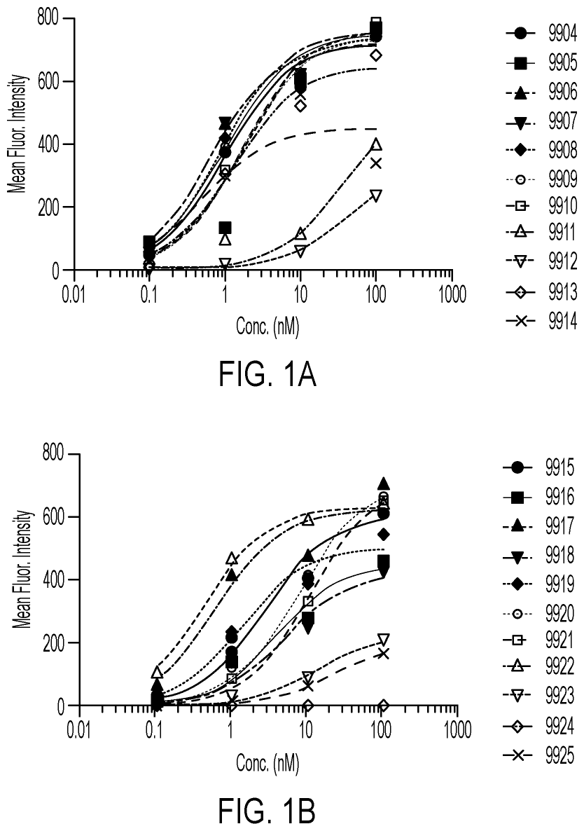

Tables 7 and 8A summarize affinity changes at each position in H2C/L0 mutants by the amino acid used for mutation. Table 8B identifies the protein IDs used in Table 8A. The amino acid sequences of the antibody chains corresponding to the protein IDs used in these experiments are detailed in Tables 9 and 10. Concentration-dependent binding of H2C/L0 mutant antibodies to CHO cells is shown in FIGS. 1A-1B. Different amino acids were differentially impactful at different positions. For example, at VH-Y59, mutation to histidine was tolerated but to either alanine or aspartic acid resulted in significant binding loss, whereas at VH-T130, mutation to alanine was well tolerated but mutation to either histidine or aspartic acid resulted in significant loss of affinity.

TABLE 7
H2C/L0 and H2C/L0 Mutants Binding to TfR

| | | | | SPR (monovalent binding) | | | |
|---|---|---|---|---|---|---|---|
| | | Flow Cytometry | | Human TfR Apical | | Cyno TfR Apical | |
| Protein | | huTfR-CHO binding | | Domain binding | | Domain binding | |
| ID | Description | EC₅₀ (nM) | Max MFI | k_{d} (/s) | K_{D} (nM) | k_{d} (/s) | K_{D} (nM) |
| 9904 | H2C/L0 hIgG1.agly | 1.0 | 721.4 | 0.00059 | 4.7 | 0.0056 | 28 |
| 9905 | H2C/L0-EVQL hIgG1.agly | 0.7 | 748.4 | 0.00081 | 7.5 | 0.0112 | 43 |
| 9906 | H2C/L0-EVQL VH-S32D hIgG1.agly | 0.9 | 760.6 | 0.00098 | 5.6 | 0.0069 | 29 |
| 9907 | H2C/L0-EVQL VH-S33D hIgG1.agly | 0.7 | 715.8 | 0.0008 | 5.5 | 0.0082 | 37 |
| 9908 | H2C/L0-EVQL VH-S33E hIgG1.agly | 0.9 | 733.5 | 0.00068 | 4.5 | 0.007 | 34 |
| 9909 | H2C/L0-EVQL VH-S38A hIgG1.agly | 1.0 | 750.8 | 0.00354 | 38.8 | 0.0304 | 326 |
| 9910 | H2C/L0-EVQL VH-S38D hIgG1.agly | 1.6 | 745.6 | 0.00344 | 29.8 | 0.0087 | 202 |
| 9911 | H2C/L0-EVQL VH-Y59A hIgG1.agly | 32.9 | 525.7 | >0.05 | 1240 | >0.05 | 4000 |
| 9912 | H2C/L0-EVQL VH-Y59D hIgG1.agly | 43.3 | 343.8 | >0.05 | ~5700 | >0.05 | >8000 |
| 9913 | H2C/L0-EVQL VH-Y59H hIgG1.agly | 1.2 | 647.2 | 0.0148 | 379 | >0.05 | 491 |
| 9914 | H2C/L0-EVQL VH-Y61D hIgG1.agly | 0.5 | 451.3 | 0.00984 | 138 | >0.05 | 663 |
| 9915 | H2C/L0-EVQL VH-N67D hIgG1.agly | 2.3 | 587 | >0.05 | 423 | >0.05 | 524 |
| 9916 | H2C/L0-EVQL VH-N67E hIgG1.agly | 4.0 | 456.3 | >0.05 | 1005 | >0.05 | 495 |
| 9917 | H2C/L0-EVQL VH-Y69A hIgG1.agly | 0.6 | 625.3 | 0.04 | 194 | >0.05 | 708 |
| 9918 | H2C/L0-EVQL VH-Y69D hIgG1.agly | 4.6 | 424.9 | >0.05 | 1365 | >0.05 | ~2990 |
| 9919 | H2C/L0-EVQL VH-Y69E hIgG1.agly | 1.4 | 507.6 | >0.05 | 742 | >0.05 | 2164 |
| 9920 | H2C/L0-EVQL VH-Y113A hIgG1.agly | 7.4 | 710 | 0.0101 | 738 | >0.05 | >800 |
| 9921 | H2C/L0-EVQL VH-Y113D hIgG1.agly | 11.3 | 724.9 | >0.05 | >800 | >0.05 | >800 |
| 9922 | H2C/L0-EVQL VH-Y116A hIgG1.agly | 0.4 | 634.8 | 0.0092 | 144 | >0.05 | 340 |
| 9923 | H2C/L0-EVQL VH-Y116D hIgG1.agly | 13.3 | 233.2 | >0.05 | 4320 | >0.05 | >8000 |
| 9924 | H2C/L0-EVQL VH-T130D hIgG1.agly | 0.2 | 0.7 | >0.05 | >800 | >0.05 | >800 |
| 9925 | H2C/L0-EVQL VH-Y131D hIgG1.agly | 22.6 | 205.7 | >0.05 | >4000 | >0.05 | >8000 |

TABLE 8A
H2C/L0 and H2C/L0 Mutants Binding
to TfR (EC50, nM; bivalent)

| | QSL VH-Nterm | | EVQL VH-Nterm | | | | |
|---|---|---|---|---|---|---|---|
| | wt | A | wt | A | H | D | S |
| parent | 0.2 | | 0.3 | | | | |
| VH-S32 | | 0.2 | | 0.2 | 0.3 | 0.8 | |
| VH-S33 | | 0.4 | | 0.2 | 0.2 | 0.3 | |
| VH-S38 | | 0.4 | | 0.4 | 0.8 | 0.9 | |
| VH-Y59 | | 8.1 | | 9.6 | 0.4 | 45.8 | |
| VH-Y61 | | 0.2 | | 0.3 | 0.4 | 0.6 | |
| VH-N67 | | 0.3 | | 0.4 | 0.4 | 1.0 | |
| VH-Y69 | | 0.2 | | 0.2 | 0.3 | 1.8 | |
| VH-Y113 | | 2.3 | | 1.8 | 0.4 | 4.0 | |
| VH-Y116 | | 0.2 | | 0.2 | 0.2 | 10.8 | |
| VH-T130 | | 0.3 | | 0.4 | 33.9 | 40.1 | |
| VH-Y131 | | 0.2 | | 0.2 | 0.6 | 23.6 | |
| VL-N30 | | | | | | | 0.2 |
| VL-N32 | | | | | | | 1.1 |
| VL-R58 | | | | | | 0.7 | |
| VL-Y111 | | | | | 0.3 | 10.8 | 0.5 |
| VL-S133 | | | | 0.2 | 0.4 | 0.5 | |

TABLE 8B
Protein IDs for H2C/L0 and H2C/L0 Mutants in Table 8A

| | QSL VH-Nterm | | EVQL VH-Nterm | | | | |
|---|---|---|---|---|---|---|---|
| | wt | A | wt | A | H | D | S |
| parent | 10026 | | 10051 | | | | |
| VH-S32 | | 10040 | | 10052 | 10054 | 10053 | |
| VH-S33 | | 10041 | | 10055 | 10057 | 10056 | |
| VH-S38 | | 10042 | | 10058 | 10060 | 10059 | |
| VH-Y59 | | 10043 | | 10061 | 10063 | 10062 | |
| VH-Y61 | | 10044 | | 10064 | 10066 | 10065 | |
| VH-N67 | | 10045 | | 10067 | 10069 | 10068 | |
| VH-Y69 | | 10046 | | 10070 | 10072 | 10071 | |
| VH-Y113 | | 10047 | | 10073 | 10075 | 10074 | |
| VH-Y116 | | 10048 | | 10076 | 10078 | 10077 | |
| VH-T130 | | 10049 | | 10079 | 10081 | 10080 | |
| VH-Y131 | | 10050 | | 10082 | 10084 | 10083 | |
| VL-N30 | | | | | | | 10085 |
| VL-N32 | | | | | | | 10086 |
| VL-R58 | | | | | | 9404 | |
| VL-Y111 | | | | | 10088 | 10087 | 10089 |
| VL-S133 | | | | 10090 | 10092 | 10091 | |

TABLE 9
Protein Descriptions of H2C/LO Mutants and Other Antibodies

| Pro- | Pep- | Pep- | Pep- | | | | | | |
|---|---|---|---|---|---|---|---|---|---|
| tein | tide | tide | tide | | Antibody | | | | |
| ID | 21 | 2 | 3 | Protein Name | Format | Mut1 | Mut2 | Mut3 | Mut4 |
| 10026 | HC-1037 | LC-1096 | | hIgG1.agly-(SG4)2-H2C/L0 | Inverted Ab | | | | |
| | | | | VH-CH1 LC-HA | | | | | |
| 10040 | HC-1043 | LC-1096 | | hIgG1.agly-(SG4)2-H2C/L0 | Inverted Ab | VH- | | | |
| | | | | VH-CH1 VH-S32A LC-HA | | S32A | | | |
| 10041 | HC-1044 | LC-1096 | | hIgG1.agly-(SG4)2-H2C/L0 | Inverted Ab | VH- | | | |
| | | | | VH-CH1 VH-S33A LC-HA | | S33A | | | |
| 10042 | HC-1045 | LC-1096 | | hIgG1.agly-(SG4)2-H2C/L0 | Inverted Ab | VH- | | | |
| | | | | VH-CH1 VH-S38A LC-HA | | S38A | | | |
| 10043 | HC-1046 | LC-1096 | | hIgG1.agly-(SG4)2-H2C/L0 | Inverted Ab | VH- | | | |
| | | | | VH-CH1 VH-Y59A LC-HA | | Y59A | | | |
| 10044 | HC-1047 | LC-1096 | | hIgG1.agly-(SG4)2-H2C/L0 | Inverted Ab | VH- | | | |
| | | | | VH-CH1 VH-Y61A LC-HA | | Y61A | | | |
| 10045 | HC-1048 | LC-1096 | | hIgG1.agly-(SG4)2-H2C/L0 | Inverted Ab | VH- | | | |
| | | | | VH-CH1 VH-N67A LC-HA | | N67A | | | |
| 10046 | HC-1049 | LC-1096 | | hIgG1.agly-(SG4)2-H2C/L0 | Inverted Ab | VH- | | | |
| | | | | VH-CH1 VH-Y69A LC-HA | | Y69A | | | |
| 10047 | HC-1050 | LC-1096 | | hIgG1.agly-(SG4)2-H2C/L0 | Inverted Ab | VH- | | | |
| | | | | VH-CH1 VH-Y113A LC-HA | | Y113A | | | |
| 10048 | HC-1051 | LC-1096 | | hIgG1.agly-(SG4)2-H2C/L0 | Inverted Ab | VH- | | | |
| | | | | VH-CH1 VH-Y116A LC-HA | | Y116A | | | |
| 10049 | HC-1052 | LC-1096 | | hIgG1.agly-(SG4)2-H2C/L0 | Inverted Ab | VH- | | | |
| | | | | VH-CH1 VH-T130A LC-HA | | T130A | | | |
| 10050 | HC-1053 | LC-1096 | | hIgG1.agly-(SG4)2-H2C/L0 | Inverted Ab | VH- | | | |
| | | | | VH-CH1 VH-Y131A LC-HA | | Y131A | | | |
| 10051 | HC-1054 | LC-1096 | | hIgG1.agly-(SG4)2-H2C/L0- | Inverted Ab | | | | |
| | | | | EVQL VH-CH1 LC-HA | | | | | |
| 10052 | HC-1055 | LC-1096 | | hIgG1.agly-(SG4)2-H2C/L0- | Inverted Ab | VH- | | | |
| | | | | EVQL VH-CH1 VH-S32A LC- | | S32A | | | |
| | | | | HA | | | | | |
| 10053 | HC-1056 | LC-1096 | | hIgG1.agly-(SG4)2-H2C/L0- | Inverted Ab | VH- | | | |
| | | | | EVQL VH-CH1 VH-S32D LC- | | S32D | | | |
| | | | | HA | | | | | |
| 10054 | HC-1057 | LC-1096 | | hIgG1.agly-(SG4)2-H2C/L0- | Inverted Ab | VH- | | | |
| | | | | EVQL VH-CH1 VH-S32H LC- | | S32H | | | |
| | | | | HA | | | | | |
| 10055 | HC-1058 | LC-1096 | | hIgG1.agly-(SG4)2-H2C/L0- | Inverted Ab | VH- | | | |
| | | | | EVQL VH-CH1 VH-S33A LC- | | S33A | | | |
| | | | | HA | | | | | |
| 10056 | HC-1059 | LC-1096 | | hIgG1.agly-(SG4)2-H2C/L0- | Inverted Ab | VH- | | | |
| | | | | EVQL VH-CH1 VH-S33D LC- | | S33D | | | |
| | | | | HA | | | | | |
| 10057 | HC-1060 | LC-1096 | | hIgG1.agly-(SG4)2-H2C/L0- | Inverted Ab | VH- | | | |
| | | | | EVQL VH-CH1 VH-S33H LC- | | S33H | | | |
| | | | | HA | | | | | |
| 10058 | HC-1061 | LC-1096 | | hIgG1.agly-(SG4)2-H2C/L0- | Inverted Ab | VH- | | | |
| | | | | EVQL VH-CH1 VH-S38A LC- | | S38A | | | |
| | | | | HA | | | | | |
| 10059 | HC-1062 | LC-1096 | | hIgG1.agly-(SG4)2-H2C/L0- | Inverted Ab | VH- | | | |
| | | | | EVQL VH-CH1 VH-S38D LC- | | S38D | | | |
| | | | | HA | | | | | |
| 10060 | HC-1063 | LC-1096 | | hIgG1.agly-(SG4)2-H2C/L0- | Inverted Ab | VH- | | | |
| | | | | EVQL VH-CH1 VH-S38H LC- | | S38H | | | |
| | | | | HA | | | | | |
| 10061 | HC-1064 | LC-1096 | | hIgG1.agly-(SG4)2-H2C/L0- | Inverted Ab | VH- | | | |
| | | | | EVQL VH-CH1 VH-Y59A LC- | | Y59A | | | |
| | | | | HA | | | | | |
| 10062 | HC-1065 | LC-1096 | | hIgG1.agly-(SG4)2-H2C/L0- | Inverted Ab | VH- | | | |
| | | | | EVQL VH-CH1 VH-Y59D LC- | | Y59D | | | |
| | | | | HA | | | | | |
| 10063 | HC-1066 | LC-1096 | | hIgG1.agly-(SG4)2-H2C/L0- | Inverted Ab | VH- | | | |
| | | | | EVQL VH-CH1 VH-Y59H LC- | | Y59H | | | |
| | | | | HA | | | | | |
| 10064 | HC-1067 | LC-1096 | | hIgG1.agly-(SG4)2-H2C/L0- | Inverted Ab | VH- | | | |
| | | | | EVQL VH-CH1 VH-Y61A LC- | | Y61A | | | |
| | | | | HA | | | | | |
| 10065 | HC-1068 | LC-1096 | | hIgG1.agly-(SG4)2-H2C/L0- | Inverted Ab | VH- | | | |
| | | | | EVQL VH-CH1 VH-Y61D LC- | | Y61D | | | |
| | | | | HA | | | | | |
| 10066 | HC-1069 | LC-1096 | | hIgG1.agly-(SG4)2-H2C/L0- | Inverted Ab | VH- | | | |
| | | | | EVQL VH-CH1 VH-Y61H LC- | | Y61H | | | |
| | | | | HA | | | | | |
| 10067 | HC-1070 | LC-1096 | | hIgG1.agly-(SG4)2-H2C/L0- | Inverted Ab | VH- | | | |
| | | | | EVQL VH-CH1 VH-N67A LC- | | N67A | | | |
| | | | | HA | | | | | |
| 10068 | HC-1071 | LC-1096 | | hIgG1.agly-(SG4)2-H2C/L0- | Inverted Ab | VH- | | | |
| | | | | EVQL VH-CH1 VH-N67D LC- | | N67D | | | |
| | | | | HA | | | | | |
| 10069 | HC-1072 | LC-1096 | | hIgG1.agly-(SG4)2-H2C/L0- | Inverted Ab | VH- | | | |
| | | | | EVQL VH-CH1 VH-N67H LC- | | N67H | | | |
| | | | | HA | | | | | |
| 10070 | HC-1073 | LC-1096 | | hIgG1.agly-(SG4)2-H2C/L0- | Inverted Ab | VH- | | | |
| | | | | EVQL VH-CH1 VH-Y69A LC- | | Y69A | | | |
| | | | | HA | | | | | |
| 10071 | HC-1074 | LC-1096 | | hIgG1.agly-(SG4)2-H2C/L0- | Inverted Ab | VH- | | | |
| | | | | EVQL VH-CH1 VH-Y69D LC- | | Y69D | | | |
| | | | | HA | | | | | |
| 10072 | HC-1075 | LC-1096 | | hIgG1.agly-(SG4)2-H2C/L0- | Inverted Ab | VH- | | | |
| | | | | EVQL VH-CH1 VH-Y69H LC- | | Y69H | | | |
| | | | | HA | | | | | |
| 10073 | HC-1076 | LC-1096 | | hIgG1.agly-(SG4)2-H2C/L0- | Inverted Ab | VH- | | | |
| | | | | EVQL VH-CH1 VH-Y113A | | Y113A | | | |
| | | | | LC-HA | | | | | |
| 10074 | HC-1077 | LC-1096 | | hIgG1.agly-(SG4)2-H2C/L0- | Inverted Ab | VH- | | | |
| | | | | EVQL VH-CH1 VH-Y113D | | Y113D | | | |
| | | | | LC-HA | | | | | |
| 10075 | HC-1078 | LC-1096 | | hIgG1.agly-(SG4)2-H2C/L0- | Inverted Ab | VH- | | | |
| | | | | EVQL VH-CH1 VH-Y113H | | Y113H | | | |
| | | | | LC-HA | | | | | |
| 10076 | HC-1079 | LC-1096 | | hIgG1.agly-(SG4)2-H2C/L0- | Inverted Ab | VH- | | | |
| | | | | EVQL VH-CH1 VH-Y116A | | Y116A | | | |
| | | | | LC-HA | | | | | |
| 10077 | HC-1080 | LC-1096 | | hIgG1.agly-(SG4)2-H2C/L0- | Inverted Ab | VH- | | | |
| | | | | EVQL VH-CH1 VH-Y116D | | Y116D | | | |
| | | | | LC-HA | | | | | |
| 10078 | HC-1081 | LC-1096 | | hIgG1.agly-(SG4)2-H2C/L0- | Inverted Ab | VH- | | | |
| | | | | EVQL VH-CH1 VH-Y116H | | Y116H | | | |
| | | | | LC-HA | | | | | |
| 10079 | HC-1082 | LC-1096 | | hIgG1.agly-(SG4)2-H2C/L0- | Inverted Ab | VH- | | | |
| | | | | EVQL VH-CH1 VH-T130A | | T130A | | | |
| | | | | LC-HA | | | | | |
| 10080 | HC-1083 | LC-1096 | | hIgG1.agly-(SG4)2-H2C/L0- | Inverted Ab | VH- | | | |
| | | | | EVQL VH-CH1 VH-T130D | | T130D | | | |
| | | | | LC-HA | | | | | |
| 10081 | HC-1084 | LC-1096 | | hIgG1.agly-(SG4)2-H2C/L0- | Inverted Ab | VH- | | | |
| | | | | EVQL VH-CH1 VH-T130H | | T130H | | | |
| | | | | LC-HA | | | | | |
| 10082 | HC-1085 | LC-1096 | | hIgG1.agly-(SG4)2-H2C/L0- | Inverted Ab | VH- | | | |
| | | | | EVQL VH-CH1 VH-Y131A | | Y131A | | | |
| | | | | LC-HA | | | | | |
| 10083 | HC-1086 | LC-1096 | | hIgG1.agly-(SG4)2-H2C/L0- | Inverted Ab | VH- | | | |
| | | | | EVQL VH-CH1 VH-Y131D | | Y131D | | | |
| | | | | LC-HA | | | | | |
| 10084 | HC-1087 | LC-1096 | | hIgG1.agly-(SG4)2-H2C/L0- | Inverted Ab | VH- | | | |
| | | | | EVQL VH-CH1 VH-Y131H | | Y131H | | | |
| | | | | LC-HA | | | | | |
| 9393 | HC-1054 | LC-1095 | | hIgG1.agly-(SG4)2-H2C/L0- | Inverted Ab | | | | |
| | | | | EVQL VH-CH1 LC | | | | | |
| 10085 | HC-1054 | LC-1098 | | hIgG1.agly-(SG4)2-H2C/L0- | Inverted Ab | VL- | | | |
| | | | | EVQL VH-CH1 LC VL-N30S | | N30S | | | |
| 10086 | HC-1054 | LC-1099 | | hIgG1.agly-(SG4)2-H2C/L0- | Inverted Ab | VL- | | | |
| | | | | EVQL VH-CH1 LC VL-N32S | | N32S | | | |
| 9404 | HC-1054 | LC-1100 | | hIgG1.agly-(SG4)2-H2C/L0- | Inverted Ab | VL- | | | |
| | | | | EVQL VH-CH1 LC VL-R58D | | R58D | | | |
| 10087 | HC-1054 | LC-1101 | | hIgG1.agly-(SG4)2-H2C/L0- | Inverted Ab | VL- | | | |
| | | | | EVQL VH-CH1 LC VL-Y111D | | Y111D | | | |
| 10088 | HC-1054 | LC-1102 | | hIgG1.agly-(SG4)2-H2C/L0- | Inverted Ab | VL- | | | |
| | | | | EVQL VH-CH1 LC VL-Y111H | | Y111H | | | |
| 10089 | HC-1054 | LC-1103 | | hIgG1.agly-(SG4)2-H2C/L0- | Inverted Ab | VL- | | | |
| | | | | EVQL VH-CH1 LC VL-Y111S | | Y111S | | | |
| 10090 | HC-1054 | LC-1104 | | hIgG1.agly-(SG4)2-H2C/L0- | Inverted Ab | VL- | | | |
| | | | | EVQL VH-CH1 LC VL-S133A | | S133A | | | |
| 10091 | HC-1054 | LC-1105 | | hIgG1.agly-(SG4)2-H2C/L0- | Inverted Ab | VL- | | | |
| | | | | EVQL VH-CH1 LC VL-S133D | | S133D | | | |
| 10092 | HC-1054 | LC-1106 | | hIgG1.agly-(SG4)2-H2C/L0- | Inverted Ab | VL- | | | |
| | | | | EVQL VH-CH1 LC VL-S133H | | S133H | | | |
| 9395 | HC-1039 | LC-1100 | | hIgG1.agly-DelG-H2C/LO VH- | Inverted Ab | VL- | | | |
| | | | | CH1 LC VL-R58D | | R58D | | | |
| 9398 | HC-1056 | LC-1100 | | hIgG1.agly-(SG4)2-H2C/L0- | Inverted Ab | VH- | VL- | | |
| | | | | EVQL VH-CH1 VH-S32D LC | | S32D | R58D | | |
| | | | | VL-R58D | | | | | |
| 9400 | HC-1071 | LC-1095 | | hIgG1.agly-(SG4)2-H2C/L0- | Inverted Ab | VH- | | | |
| | | | | EVQL VH-CH1 VH-N67D LC | | N67D | | | |
| 9401 | HC-1071 | LC-1100 | | hIgG1.agly-(SG4)2-H2C/L0- | Inverted Ab | VH- | VL- | | |
| | | | | EVQL VH-CH1 VH-N67D LC | | N67D | R58D | | |
| | | | | VL-R58D | | | | | |
| 9402 | HC-1073 | LC-1095 | | hIgG1.agly-(SG4)2-H2C/L0- | Inverted Ab | VH- | | | |
| | | | | EVQL VH-CH1 VH-Y69A LC | | Y69A | | | |
| 9122 | HC-1001 | LC-1095 | | huH2C/LO bivalent RSU | Conventional | | | | |
| | | | | hIgG1.agly LC hKappa | Ab | | | | |
| 9124 | HC-1037 | LC-1095 | | Fc hIgG1.agly H2C/LO Fab | Inverted Ab | | | | |
| | | | | bivalent USD LC (wt) | | | | | |
| 9904 | HC-1001 | LC-1095 | | H2C/L0 hIgG1.agly | Conventional | | | | |
| | | | | | Ab | | | | |
| 9905 | HC-1003 | LC-1095 | | H2C/LO-EVQL hIgG1.agly | Conventional | | | | |
| | | | | | Ab | | | | |
| 9906 | HC-1004 | LC-1095 | | H2C/LO-EVQL VH-S32D | Conventional | VH- | | | |
| | | | | hIgG1.agly | Ab | S32D | | | |
| 9907 | HC-1005 | LC-1095 | | H2C/LO-EVQL VH-S33D | Conventional | VH- | | | |
| | | | | hIgG1.agly | Ab | S33D | | | |
| 9908 | HC-1006 | LC-1095 | | H2C/LO-EVQL VH-S33E | Conventional | VH- | | | |
| | | | | hIgG1.agly | Ab | S33E | | | |
| 9909 | HC-1007 | LC-1095 | | H2C/LO-EVQL VH-S38A | Conventional | VH- | | | |
| | | | | hIgG1.agly | Ab | S38A | | | |
| 9910 | HC-1008 | LC-1095 | | H2C/LO-EVQL VH-S38D | Conventional | VH- | | | |
| | | | | hIgG1.agly | Ab | S38D | | | |
| 9911 | HC-1009 | LC-1095 | | H2C/LO-EVQL VH-Y59A | Conventional | VH- | | | |
| | | | | hIgG1.agly | Ab | Y59A | | | |
| 9912 | HC-1010 | LC-1095 | | H2C/LO-EVQL VH-Y59D | Conventional | VH- | | | |
| | | | | hIgG1.agly | Ab | Y59D | | | |
| 9913 | HC-1011 | LC-1095 | | H2C/LO-EVQL VH-Y59H | Conventional | VH- | | | |
| | | | | hIgG1.agly | Ab | Y59H | | | |
| 9914 | HC-1012 | LC-1095 | | H2C/LO-EVQL VH-Y61D | Conventional | VH- | | | |
| | | | | hIgG1.agly | Ab | Y61D | | | |
| 9915 | HC-1013 | LC-1095 | | H2C/LO-EVQL VH-N67D | Conventional | VH- | | | |
| | | | | hIgG1.agly | Ab | N67D | | | |
| 9916 | HC-1014 | LC-1095 | | H2C/LO-EVQL VH-N67E | Conventional | VH- | | | |
| | | | | hIgG1.agly | Ab | N67E | | | |
| 9917 | HC-1015 | LC-1095 | | H2C/LO-EVQL VH-Y69A | Conventional | VH- | | | |
| | | | | hIgG1.agly | Ab | Y69A | | | |
| 9918 | HC-1016 | LC-1095 | | H2C/LO-EVQL VH-Y69D | Conventional | VH- | | | |
| | | | | hIgG1.agly | Ab | Y69D | | | |
| 9919 | HC-1017 | LC-1095 | | H2C/LO-EVQL VH-Y69E | Conventional | VH- | | | |
| | | | | hIgG1.agly | Ab | Y69E | | | |
| 9920 | HC-1018 | LC-1095 | | H2C/LO-EVQL VH-Y113A | Conventional | VH- | | | |
| | | | | hIgG1.agly | Ab | Y113A | | | |
| 9921 | HC-1019 | LC-1095 | | H2C/LO-EVQL VH-Y113D | Conventional | VH- | | | |
| | | | | hIgG1.agly | Ab | Y113D | | | |
| 9922 | HC-1020 | LC-1095 | | H2C/LO-EVQL VH-Y116A | Conventional | VH- | | | |
| | | | | hIgG1.agly | Ab | Y116A | | | |
| 9923 | HC-1021 | LC-1095 | | H2C/LO-EVQL VH-Y116D | Conventional | VH- | | | |
| | | | | hIgG1.agly | Ab | Y116D | | | |
| 9924 | HC-1022 | LC-1095 | | H2C/LO-EVQL VH-T130D | Conventional | VH- | | | |
| | | | | hIgG1.agly | Ab | T130D | | | |
| 9925 | HC-1023 | LC-1095 | | H2C/LO-EVQL VH-Y131D | Conventional | VH- | | | |
| | | | | hIgG1.agly | Ab | Y131D | | | |
| 9926 | HC-1024 | LC-1095 | | H2C/LO-EVQL VH-S32D/Y61D | Conventional | VH- | VH- | | |
| | | | | hIgG1.agly | Ab | S32D | Y61D | | |
| 9927 | HC-1025 | LC-1095 | | H2C/LO-EVQL VH-S32D/N67E | Conventional | VH- | VH- | | |
| | | | | hIgG1.agly | Ab | S32D | N67E | | |
| 9928 | HC-1026 | LC-1095 | | H2C/LO-EVQL VH-S32D/Y69D | Conventional | VH- | VH- | | |
| | | | | hIgG1.agly | Ab | S32D | Y69D | | |
| 9929 | HC-1027 | LC-1095 | | H2C/LO-EVQL VH-S32D/Y69E | Conventional | VH- | VH- | | |
| | | | | hIgG1.agly | Ab | S32D | Y69E | | |
| 9930 | HC-1028 | LC-1095 | | H2C/LO-EVQL VH- | Conventional | VH- | VH- | VH- | |
| | | | | S32D/Y59H/Y61D hIgG1.agly | Ab | S32D | Y59H | Y61D | |
| 9931 | HC-1029 | LC-1095 | | H2C/LO-EVQL VH- | Conventional | VH- | VH- | | |
| | | | | S32D/Y116A hIgG1.agly | Ab | S32D | Y116A | | |
| 9932 | HC-1030 | LC-1095 | | H2C/LO-EVQL VH- | Conventional | VH- | VH- | | |
| | | | | Y69A/Y116A hIgG1.agly | Ab | Y69A | Y116A | | |
| 9933 | HC-1031 | LC-1095 | | H2C/LO-EVQL VH- | Conventional | VH- | VH- | | |
| | | | | Y69E/Y116A hIgG1.agly | Ab | Y69E | Y116A | | |
| 9934 | HC-1032 | LC-1095 | | H2C/LO-EVQL VH-CACA | Conventional | | | | |
| | | | | hIgG1.agly | Ab | | | | |
| 9935 | HC-1033 | LC-1095 | | H2C/LO-EVQL VH- | Conventional | VH- | | | |
| | | | | CACA/N67D hIgG1.agly | Ab | N67D | | | |
| 9936 | HC-1013 | LC-1100 | | H2C/LO-EVQL VH-N67D VL- | Conventional | VH- | VL- | | |
| | | | | R58D hIgG1.agly | Ab | N67D | R58D | | |
| 9937 | HC-1013 | LC-1107 | | H2C/LO-EVQL VH-N67D VL- | Conventional | VH- | VL- | VL- | |
| | | | | N30S/R58D hIgG1.agly | Ab | N67D | N30S | R58D | |
| 9938 | HC-1013 | LC-1108 | | H2C/LO-EVQL VH-N67D VL- | Conventional | VH- | VL- | VL- | |
| | | | | N30S/Y111S hIgG1.agly | Ab | N67D | N30S | Y111S | |
| 9939 | HC-1013 | LC-1109 | | H2C/LO-EVQL VH-N67D VL- | Conventional | VH- | VL- | VL- | |
| | | | | N30S/Y111D hIgG1.agly | Ab | N67D | N30S | Y111D | |
| 9940 | HC-1013 | LC-1110 | | H2C/LO-EVQL VH-N67D VL- | Conventional | VH- | VL- | VL- | |
| | | | | N30S/S133D hIgG1.agly | Ab | N67D | N30S | S133D | |
| 9941 | HC-1013 | LC-1111 | | H2C/LO-EVQL VH-N67D VL- | Conventional | VH- | VL- | VL- | VL- |
| | | | | N30S/R58D/Y111S hIgG1.agly | Ab | N67D | N30S | R58D | Y111S |
| 9942 | HC-1013 | LC-1112 | | H2C/LO-EVQL VH-N67D VL- | Conventional | VH- | VL- | VL- | VL- |
| | | | | N30S/R58D/S133D hIgG1.agly | Ab | N67D | N30S | R58D | S133D |
| 9943 | HC-1034 | LC-1113 | | H2C/LO-EVQL VH-N67D LC- | Conventional | VH- | | | |
| | | | | DelC214 HC-C220S hIgG1. | Ab | N67D | | | |
| | | | | agly | | | | | |
| 9944 | HC-1034 | LC-1114 | | H2C/LO-EVQL VH-N67D VL- | Conventional | VH- | VL- | | |
| | | | | R58D LC-DelC214 HC-C220S | Ab | N67D | R58D | | |
| | | | | hIgG1.agly | | | | | |
| 9945 | HC-1088 | | | hGlag Fc H2C/LO VH-VL scFv | Fc-scFv | VH- | | | |
| | | | | VH-N67D | Inverted Ab | N67D | | | |
| 9946 | HC-1089 | | | hGlag Fc H2C/LO VH-VL scFv | Fc-scFv | VH- | VL- | | |
| | | | | VH-N67D VL-R58D | Inverted Ab | N67D | R58D | | |
| 9947 | HC-1090 | | | hGlag Fc H2C/LO VL-VH scFv | Fc-scFv | VH- | | | |
| | | | | VH-N67D | Inverted Ab | N67D | | | |
| 9948 | HC-1091 | | | hGlag Fc H2C/LO VL-VH scFv | Fc-scFv | VH- | VL- | | |
| | | | | VL-R58D VH-N67D | Inverted Ab | N67D | R58D | | |
| 9949 | HC-1092 | | | hGlag Fc H2C/LO VL-VH scFv | Fc-scFv | VH- | | | |
| | | | | VH-N67D CACA | Inverted Ab | N67D | | | |
| 9950 | HC-1093 | | | hGlag Fc H2C/LO VL-VH scFv | Fc-scFv | VH- | | | |
| | | | | VH-N67D 51-141dsb | Inverted Ab | N67D | | | |
| 9951 | HC-1094 | | | hGlag Fc H2C/LO VL-VH scFv | Fc-scFv | VH- | | | |
| | | | | VH-N67D CACA 51-141dsb | Inverted Ab | N67D | | | |
| 9956 | | | | Ab1-hIgG1.LALAPG scFv | Ab-scFv | VH- | | | |
| | | | | H2C/LO VL-VH N67D | bispecific | N67D | | | |
| 8849 | | | | Ab2 | Conventional | | | | |
| | | | | | hIgG1 Ab | | | | |

TABLE 10
Peptide Sequences for Antibody Components

| Peptide | Peptide | SEQ ID | |
|---|---|---|---|
| Name | Description | NO | Peptide Sequence |
| HC-1001 | HC hIgG1.agly | | QSLVESGGGLVQPGGSLRLSCAASGIDFSSSGYMCWVRQAPGKGLEWVGCIYTYS |
| | | | SNTYYAASVKGRFTISKTSSTTVYLQMNSLKTEDTAVYYCARGTYGYTGYTYTM |
| | | | GYFSLWGQGTLVTVSSASTKGPSVFPLAPSSKSTSGGTAALGCLVKDYFPEPVTVS |
| | | | WNSGALTSGVHTFPAVLQSSGLYSLSSVVTVPSSSLGTQTYICNVNHKPSNTKVDK |
| | | | KVEPKSCDKTHTCPPCPAPELLGGPSVFLFPPKPKDTLMISRTPEVTCVVVDVSHED |
| | | | PEVKFNWYVDGVEVHNAKTKPREEQYQSTYRVVSVLTVLHQDWLNGKEYKCKV |
| | | | SNKALPAPIEKTISKAKGQPREPQVYTLPPSRDELTKNQVSLTCLVKGFYPSDIAVE |
| | | | WESNGQPENNYKTTPPVLDSDGSFFLYSKLTVDKSRWQQGNVFSCSVMHEALHN |
| | | | HYTQKSLSLSPG |
| HC-1002 | HC hIgG1.agly | | QSLVESGGGLVQPGGSLRLSCAASGIDFSSSGYMCWVRQAPGKGLEWVGCIYTYS |
| | (knob + cys) | | SNTYYAASVKGRFTISKTSSTTVYLQMNSLKTEDTAVYYCARGTYGYTGYTYTM |
| | | | GYFSLWGQGTLVTVSSASTKGPSVFPLAPSSKSTSGGTAALGCLVKDYFPEPVTVS |
| | | | WNSGALTSGVHTFPAVLQSSGLYSLSSVVTVPSSSLGTQTYICNVNHKPSNTKVDK |
| | | | KVEPKSCDKTHTCPPCPAPELLGGPSVFLFPPKPKDTLMISRTPEVTCVVVDVSHED |
| | | | PEVKFNWYVDGVEVHNAKTKPREEQYQSTYRVVSVLTVLHQDWLNGKEYKCKV |
| | | | SNKALPAPIEKTISKAKGQPREPQVYTLPPCRDELTKNQVSLWCLVKGFYPSDIAV |
| | | | EWESNGQPENNYKTTPPVLDSDGSFFLYSKLTVDKSRWQQGNVFSCSVMHEALH |
| | | | NHYTQKSLSLSPG |
| HC-1003 | EVQL | | EVQLVESGGGLVQPGGSLRLSCAASGIDFSSSGYMCWVRQAPGKGLEWVGCIYT |
| | IgG1.agly HC | | YSSNTYYAASVKGRFTISKTSSTTVYLQMNSLKTEDTAVYYCARGTYGYTGYTYT |
| | | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1004 | EVQL | | EVQLVESGGGLVQPGGSLRLSCAASGIDFDSSGYMCWVRQAPGKGLEWVGCIYT |
| | hIgG1.agly | | YSSNTYYAASVKGRFTISKTSSTTVYLQMNSLKTEDTAVYYCARGTYGYTGYTYT |
| | VH-S32D | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1005 | EVQL | | EVQLVESGGGLVQPGGSLRLSCAASGIDFSDSGYMCWVRQAPGKGLEWVGCIYT |
| | hIgG1.agly | | YSSNTYYAASVKGRFTISKTSSTTVYLQMNSLKTEDTAVYYCARGTYGYTGYTYT |
| | VH-S33D | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1006 | EVQL | | EVQLVESGGGLVQPGGSLRLSCAASGIDFSESGYMCWVRQAPGKGLEWVGCIYT |
| | hIgG1.agly | | YSSNTYYAASVKGRFTISKTSSTTVYLQMNSLKTEDTAVYYCARGTYGYTGYTYT |
| | VH-S33E | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1007 | EVQL | | EVQLVESGGGLVQPGGSLRLSCAASGIDFSSAGYMCWVRQAPGKGLEWVGCIYT |
| | hIgG1.agly | | YSSNTYYAASVKGRFTISKTSSTTVYLQMNSLKTEDTAVYYCARGTYGYTGYTYT |
| | VH-S38A | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1008 | EVQL | | EVQLVESGGGLVQPGGSLRLSCAASGIDFSSDGYMCWVRQAPGKGLEWVGCIYT |
| | hIgG1.agly | | YSSNTYYAASVKGRFTISKTSSTTVYLQMNSLKTEDTAVYYCARGTYGYTGYTYT |
| | VH-S38D | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1009 | EVQL | | EVQLVESGGGLVQPGGSLRLSCAASGIDFSSSGYMCWVRQAPGKGLEWVGCIAT |
| | hIgG1.agly | | YSSNTYYAASVKGRFTISKTSSTTVYLQMNSLKTEDTAVYYCARGTYGYTGYTYT |
| | VH-Y59A | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1010 | EVQL | | EVQLVESGGGLVQPGGSLRLSCAASGIDFSSSGYMCWVRQAPGKGLEWVGCIDT |
| | hIgG1.agly | | YSSNTYYAASVKGRFTISKTSSTTVYLQMNSLKTEDTAVYYCARGTYGYTGYTYT |
| | VH-Y59D | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1011 | EVQL | | EVQLVESGGGLVQPGGSLRLSCAASGIDFSSSGYMCWVRQAPGKGLEWVGCIHT |
| | hIgG1.agly | | YSSNTYYAASVKGRFTISKTSSTTVYLQMNSLKTEDTAVYYCARGTYGYTGYTYT |
| | VH-Y59H | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1012 | EVQL | | EVQLVESGGGLVQPGGSLRLSCAASGIDFSSSGYMCWVRQAPGKGLEWVGCIYT |
| | hIgG1.agly | | DSSNTYYAASVKGRFTISKTSSTTVYLQMNSLKTEDTAVYYCARGTYGYTGYTYT |
| | VH-Y61D | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1013 | EVQL | | EVQLVESGGGLVQPGGSLRLSCAASGIDFSSSGYMCWVRQAPGKGLEWVGCIYT |
| | hIgG1.agly | | YSSDTYYAASVKGRFTISKTSSTTVYLQMNSLKTEDTAVYYCARGTYGYTGYTYT |
| | VH-N67D | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1014 | EVQL | | EVQLVESGGGLVQPGGSLRLSCAASGIDFSSSGYMCWVRQAPGKGLEWVGCIYT |
| | hIgG1.agly | | YSSETYYAASVKGRFTISKTSSTTVYLQMNSLKTEDTAVYYCARGTYGYTGYTYT |
| | VH-N67E | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1015 | EVQL | | EVQLVESGGGLVQPGGSLRLSCAASGIDFSSSGYMCWVRQAPGKGLEWVGCIYT |
| | hIgG1.agly | | YSSNTAYAASVKGRFTISKTSSTTVYLQMNSLKTEDTAVYYCARGTYGYTGYTYT |
| | VH-Y69A | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG* |
| HC-1016 | EVQL | | EVQLVESGGGLVQPGGSLRLSCAASGIDFSSSGYMCWVRQAPGKGLEWVGCIYT |
| | hIgG1.agly | | YSSNTDYAASVKGRFTISKTSSTTVYLQMNSLKTEDTAVYYCARGTYGYTGYTYT |
| | VH-Y69D | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG* |
| HC-1017 | EVQL | | EVQLVESGGGLVQPGGSLRLSCAASGIDFSSSGYMCWVRQAPGKGLEWVGCIYT |
| | hIgG1.agly | | YSSNTEYAASVKGRFTISKTSSTTVYLQMNSLKTEDTAVYYCARGTYGYTGYTYT |
| | VH-Y69E | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG* |
| HC-1018 | EVQL | | EVQLVESGGGLVQPGGSLRLSCAASGIDFSSSGYMCWVRQAPGKGLEWVGCIYT |
| | hIgG1.agly | | YSSNTYYAASVKGRFTISKTSSTTVYLQMNSLKTEDTAVYYCARGTYGATGYTYT |
| | VH-Y113A | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG* |
| HC-1019 | EVQL | | EVQLVESGGGLVQPGGSLRLSCAASGIDFSSSGYMCWVRQAPGKGLEWVGCIYT |
| | hIgG1.agly | | YSSNTYYAASVKGRFTISKTSSTTVYLQMNSLKTEDTAVYYCARGTYGDTGYTYT |
| | VH-Y113D | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG* |
| HC-1020 | EVQL | | EVQLVESGGGLVQPGGSLRLSCAASGIDFSSSGYMCWVRQAPGKGLEWVGCIYT |
| | hIgG1.agly | | YSSNTYYAASVKGRFTISKTSSTTVYLQMNSLKTEDTAVYYCARGTYGYTGATYT |
| | VH-Y116A | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1021 | EVQL | | EVQLVESGGGLVQPGGSLRLSCAASGIDFSSSGYMCWVRQAPGKGLEWVGCIYT |
| | hIgG1.agly | | YSSNTYYAASVKGRFTISKTSSTTVYLQMNSLKTEDTAVYYCARGTYGYTGDTYT |
| | VH-Y116D | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1022 | EVQL | | EVQLVESGGGLVQPGGSLRLSCAASGIDFSSSGYMCWVRQAPGKGLEWVGCIYT |
| | hIgG1.agly | | YSSNTYYAASVKGRFTISKTSSTTVYLQMNSLKTEDTAVYYCARGTYGYTGYDYT |
| | VH-T130D | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1023 | EVQL | | EVQLVESGGGLVQPGGSLRLSCAASGIDFSSSGYMCWVRQAPGKGLEWVGCIYT |
| | hIgG1.agly | | YSSNTYYAASVKGRFTISKTSSTTVYLQMNSLKTEDTAVYYCARGTYGYTGYTDT |
| | VH-Y131D | | MGYFSLWGQGTLVTVSSASTKGPSVFPLAPSSKSTSGGTAALGCLVKDYFPEPVT |
| | | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1024 | EVQL | | EVQLVESGGGLVQPGGSLRLSCAASGIDFDSSGYMCWVRQAPGKGLEWVGCIYT |
| | hIgG1.agly | | DSSNTYYAASVKGRFTISKTSSTTVYLQMNSLKTEDTAVYYCARGTYGYTGYTYT |
| | VH- | | MGYFSLWGQGTLVTVSSASTKGPSVFPLAPSSKSTSGGTAALGCLVKDYFPEPVT |
| | S32D/Y61D | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1025 | EVQL | | EVQLVESGGGLVQPGGSLRLSCAASGIDFDSSGYMCWVRQAPGKGLEWVGCIYT |
| | hIgG1.agly | | YSSDTYYAASVKGRFTISKTSSTTVYLQMNSLKTEDTAVYYCARGTYGYTGYTYT |
| | VH- | | MGYFSLWGQGTLVTVSSASTKGPSVFPLAPSSKSTSGGTAALGCLVKDYFPEPVT |
| | S32D/N67D | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1026 | EVQL | | EVQLVESGGGLVQPGGSLRLSCAASGIDFDSSGYMCWVRQAPGKGLEWVGCIYT |
| | hIgG1.agly | | YSSNTDYAASVKGRFTISKTSSTTVYLQMNSLKTEDTAVYYCARGTYGYTGYTYT |
| | VH- | | MGYFSLWGQGTLVTVSSASTKGPSVFPLAPSSKSTSGGTAALGCLVKDYFPEPVT |
| | S32D/Y69D | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1027 | EVQL | | EVQLVESGGGLVQPGGSLRLSCAASGIDFDSSGYMCWVRQAPGKGLEWVGCIYT |
| | hIgG1.agly | | YSSNTEYAASVKGRFTISKTSSTTVYLQMNSLKTEDTAVYYCARGTYGYTGYTYT |
| | VH- | | MGYFSLWGQGTLVTVSSASTKGPSVFPLAPSSKSTSGGTAALGCLVKDYFPEPVT |
| | S32D/Y69E | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1028 | EVQL | | EVQLVESGGGLVQPGGSLRLSCAASGIDFDSSGYMCWVRQAPGKGLEWVGCIHT |
| | hIgG1.agly | | DSSNTYYAASVKGRFTISKTSSTTVYLQMNSLKTEDTAVYYCARGTYGYTGYTYT |
| | VH- | | MGYFSLWGQGTLVTVSSASTKGPSVFPLAPSSKSTSGGTAALGCLVKDYFPEPVT |
| | S32D/Y59H/Y | | VSWNSGALTSGVHTFPAVLQSSGLYSLSSVVTVPSSSLGTQTYICNVNHKPSNTKV |
| | 61D | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1029 | EVQL | | EVQLVESGGGLVQPGGSLRLSCAASGIDFDSSGYMCWVRQAPGKGLEWVGCIYT |
| | hIgG1.agly | | YSSNTYYAASVKGRFTISKTSSTTVYLQMNSLKTEDTAVYYCARGTYGYTGATYT |
| | VH- | | MGYFSLWGQGTLVTVSSASTKGPSVFPLAPSSKSTSGGTAALGCLVKDYFPEPVT |
| | S32D/Y116A | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1030 | EVQL | | EVQLVESGGGLVQPGGSLRLSCAASGIDFSSSGYMCWVRQAPGKGLEWVGCIYT |
| | hIgG1.agly | | YSSNTAYAASVKGRFTISKTSSTTVYLQMNSLKTEDTAVYYCARGTYGYTGATYT |
| | VH- | | MGYFSLWGQGTLVTVSSASTKGPSVFPLAPSSKSTSGGTAALGCLVKDYFPEPVT |
| | Y69A/Y116A | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1031 | EVQL | | EVQLVESGGGLVQPGGSLRLSCAASGIDFSSSGYMCWVRQAPGKGLEWVGCIYT |
| | hIgG1.agly | | YSSNTEYAASVKGRFTISKTSSTTVYLQMNSLKTEDTAVYYCARGTYGYTGATYT |
| | VH- | | MGYFSLWGQGTLVTVSSASTKGPSVFPLAPSSKSTSGGTAALGCLVKDYFPEPVT |
| | Y69E/Y116A | | VSWNSGALTSGVHTFPAVLQSSGLYSLSSVVTVPSSSLGTQTYICNVNHKPSNTKV |
| | | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1032 | EVQL | | EVQLVESGGGLVQPGGSLRLSCAASGIDFSSSGYMAWVRQAPGKGLEWVGAIYT |
| | hIgG1.agly | | YSSNTYYAASVKGRFTISKTSSTTVYLQMNSLKTEDTAVYYCARGTYGYTGYTYT |
| | | | MGYFSLWGQGTLVTVSSASTKGPSVFPLAPSSKSTSGGTAALGCLVKDYFPEPVT |
| | VH- | | VSWNSGALTSGVHTFPAVLQSSGLYSLSSVVTVPSSSLGTQTYICNVNHKPSNTKV |
| | C42A/C57A | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1033 | EVQL | | EVQLVESGGGLVQPGGSLRLSCAASGIDFSSSGYMAWVRQAPGKGLEWVGAIYT |
| | hIgG1.agly | | YSSDTYYAASVKGRFTISKTSSTTVYLQMNSLKTEDTAVYYCARGTYGYTGYTYT |
| | VH- | | MGYFSLWGQGTLVTVSSASTKGPSVFPLAPSSKSTSGGTAALGCLVKDYFPEPVT |
| | C42A/C57A/N | | VSWNSGALTSGVHTFPAVLQSSGLYSLSSVVTVPSSSLGTQTYICNVNHKPSNTKV |
| | 67D | | DKKVEPKSCDKTHTCPPCPAPELLGGPSVFLFPPKPKDTLMISRTPEVTCVVVDVS |
| | | | HEDPEVKFNWYVDGVEVHNAKTKPREEQYQSTYRVVSVLTVLHQDWLNGKEYK |
| | | | CKVSNKALPAPIEKTISKAKGQPREPQVYTLPPSRDELTKNQVSLTCLVKGFYPSDI |
| | | | AVEWESNGQPENNYKTTPPVLDSDGSFFLYSKLTVDKSRWQQGNVFSCSVMHEA |
| | | | LHNHYTQKSLSLSPG |
| HC-1034 | EVQL | | EVQLVESGGGLVQPGGSLRLSCAASGIDFSSSGYMCWVRQAPGKGLEWVGCIYT |
| | hIgG1.agly | | YSSDTYYAASVKGRFTISKTSSTTVYLQMNSLKTEDTAVYYCARGTYGYTGYTYT |
| | VH- | | MGYFSLWGQGTLVTVSSASTKGPSVFPLAPSSKSTSGGTAALGCLVKDYFPEPVT |
| | C220S/N67D | | VSWNSGALTSGVHTFPAVLQSSGLYSLSSVVTVPSSSLGTQTYICNVNHKPSNTKV |
| | | | DKKVEPKSSDKTHTCPPCPAPELLGGPSVFLFPPKPKDTLMISRTPEVTCVVVDVSH |
| | | | EDPEVKFNWYVDGVEVHNAKTKPREEQYQSTYRVVSVLTVLHQDWLNGKEYKC |
| | | | KVSNKALPAPIEKTISKAKGQPREPQVYTLPPSRDELTKNQVSLTCLVKGFYPSDIA |
| | | | VEWESNGQPENNYKTTPPVLDSDGSFFLYSKLTVDKSRWQQGNVFSCSVMHEAL |
| | | | HNHYTQKSLSLSPG |
| HC-1036 | Fc | | KTHTCPPCPAPELLGGPSVFLFPPKPKDTLMISRTPEVTCVVVDVSHEDPEVKFNW |
| | hIgG1.agly(hol | | YVDGVEVHNAKTKPREEQYQSTYRVVSVLTVLHQDWLNGKEYKCKVSNKALPA |
| | e + cys) | | PIEKTISKAKGQPREPQVCTLPPSRDELTKNQVSLSCAVKGFYPSDIAVEWESNGQP |
| | | | ENNYKTTPPVLDSDGSFFLVSKLTVDKSRWQQGNVFSCSVMHEALHNHYTQKSL |
| | | | SLSPG |
| HC-1037 | hIgG1.agly- | | KTHTCPPCPAPELLGGPSVFLFPPKPKDTLMISRTPEVTCVVVDVSHEDPEVKFNW |
| | SG4SG4-VH- | | YVDGVEVHNAKTKPREEQYQSTYRVVSVLTVLHQDWLNGKEYKCKVSNKALPA |
| | CH1 | | PIEKTISKAKGQPREPQVYTLPPSRDELTKNQVSLTCLVKGFYPSDIAVEWESNGQP |
| | | | ENNYKTTPPVLDSDGSFFLYSKLTVDKSRWQQGNVFSCSVMHEALHNHYTQKSL |
| | | | SLSPGSGGGGSGGGGQSLVESGGGLVQPGGSLRLSCAASGIDFSSSGYMCWVRQA |
| | | | PGKGLEWVGCIYTYSSNTYYAASVKGRFTISKTSSTTVYLQMNSLKTEDTAVYYC |
| | | | ARGTYGYTGYTYTMGYFSLWGQGTLVTVSSASTKGPSVFPLAPSSKSTSGGTAAL |
| | | | GCLVKDYFPEPVTVSWNSGALTSGVHTFPAVLQSSGLYSLSSVVTVPSSSLGTQTY |
| | | | ICNVNHKPSNTKVDKKVEPKSC |
| HC-1039 | hIgG1.agly- | | KTHTCPPCPAPELLGGPSVFLFPPKPKDTLMISRTPEVTCVVVDVSHEDPEVKFNW |
| | DelG-VH- | | YVDGVEVHNAKTKPREEQYQSTYRVVSVLTVLHQDWLNGKEYKCKVSNKALPA |
| | CH1 | | PIEKTISKAKGQPREPQVYTLPPSRDELTKNQVSLTCLVKGFYPSDIAVEWESNGQP |
| | | | ENNYKTTPPVLDSDGSFFLYSKLTVDKSRWQQGNVFSCSVMHEALHNHYTQKSL |
| | | | SLSPQSLVESGGGLVQPGGSLRLSCAASGIDFSSSGYMCWVRQAPGKGLEWVGCI |
| | | | YTYSSNTYYAASVKGRFTISKTSSTTVYLQMNSLKTEDTAVYYCARGTYGYTGYT |
| | | | YTMGYFSLWGQGTLVTVSSASTKGPSVFPLAPSSKSTSGGTAALGCLVKDYFPEP |
| | | | VTVSWNSGALTSGVHTFPAVLQSSGLYSLSSVVTVPSSSLGTQTYICNVNHKPSNT |
| | | | KVDKKVEPKSC |
| HC-1043 | hIgG1.agly- | | KTHTCPPCPAPELLGGPSVFLFPPKPKDTLMISRTPEVTCVVVDVSHEDPEVKFNW |
| | SG4SG4-VH- | | YVDGVEVHNAKTKPREEQYQSTYRVVSVLTVLHQDWLNGKEYKCKVSNKALPA |
| | CH1 VH-S32A | | PIEKTISKAKGQPREPQVYTLPPSRDELTKNQVSLTCLVKGFYPSDIAVEWESNGQP |
| | | | ENNYKTTPPVLDSDGSFFLYSKLTVDKSRWQQGNVFSCSVMHEALHNHYTQKSL |
| | | | SLSPGSGGGGSGGGGQSLVESGGGLVQPGGSLRLSCAASGIDFASSGYMCWVRQA |
| | | | PGKGLEWVGCIYTYSSNTYYAASVKGRFTISKTSSTTVYLQMNSLKTEDTAVYYC |
| | | | ARGTYGYTGYTYTMGYFSLWGQGTLVTVSSASTKGPSVFPLAPSSKSTSGGTAAL |
| | | | GCLVKDYFPEPVTVSWNSGALTSGVHTFPAVLQSSGLYSLSSVVTVPSSSLGTQTY |
| | | | ICNVNHKPSNTKVDKKVEPKSC |
| HC-1044 | hIgG1.agly- | | KTHTCPPCPAPELLGGPSVFLFPPKPKDTLMISRTPEVTCVVVDVSHEDPEVKFNW |
| | SG4SG4-VH- | | YVDGVEVHNAKTKPREEQYQSTYRVVSVLTVLHQDWLNGKEYKCKVSNKALPA |
| | CH1 VH-S33A | | PIEKTISKAKGQPREPQVYTLPPSRDELTKNQVSLTCLVKGFYPSDIAVEWESNGQP |
| | | | ENNYKTTPPVLDSDGSFFLYSKLTVDKSRWQQGNVFSCSVMHEALHNHYTQKSL |
| | | | SLSPGSGGGGSGGGGQSLVESGGGLVQPGGSLRLSCAASGIDFSASGYMCWVRQA |
| | | | PGKGLEWVGCIYTYSSNTYYAASVKGRFTISKTSSTTVYLQMNSLKTEDTAVYYC |
| | | | ARGTYGYTGYTYTMGYFSLWGQGTLVTVSSASTKGPSVFPLAPSSKSTSGGTAAL |
| | | | GCLVKDYFPEPVTVSWNSGALTSGVHTFPAVLQSSGLYSLSSVVTVPSSSLGTQTY |
| | | | ICNVNHKPSNTKVDKKVEPKSC |
| HC-1045 | hIgG1.agly- | | KTHTCPPCPAPELLGGPSVFLFPPKPKDTLMISRTPEVTCVVVDVSHEDPEVKFNW |
| | SG4SG4-VH- | | YVDGVEVHNAKTKPREEQYQSTYRVVSVLTVLHQDWLNGKEYKCKVSNKALPA |
| | CH1 VH-S38A | | PIEKTISKAKGQPREPQVYTLPPSRDELTKNQVSLTCLVKGFYPSDIAVEWESNGQP |
| | | | ENNYKTTPPVLDSDGSFFLYSKLTVDKSRWQQGNVFSCSVMHEALHNHYTQKSL |
| | | | SLSPGSGGGGSGGGGQSLVESGGGLVQPGGSLRLSCAASGIDFSSAGYMCWVRQA |
| | | | PGKGLEWVGCIYTYSSNTYYAASVKGRFTISKTSSTTVYLQMNSLKTEDTAVYYC |
| | | | ARGTYGYTGYTYTMGYFSLWGQGTLVTVSSASTKGPSVFPLAPSSKSTSGGTAAL |
| | | | GCLVKDYFPEPVTVSWNSGALTSGVHTFPAVLQSSGLYSLSSVVTVPSSSLGTQTY |
| | | | ICNVNHKPSNTKVDKKVEPKSC |
| HC-1046 | hIgG1.agly- | | KTHTCPPCPAPELLGGPSVFLFPPKPKDTLMISRTPEVTCVVVDVSHEDPEVKFNW |
| | SG4SG4-VH- | | YVDGVEVHNAKTKPREEQYQSTYRVVSVLTVLHQDWLNGKEYKCKVSNKALPA |
| | CH1 VH- | | PIEKTISKAKGQPREPQVYTLPPSRDELTKNQVSLTCLVKGFYPSDIAVEWESNGQP |
| | Y59A | | ENNYKTTPPVLDSDGSFFLYSKLTVDKSRWQQGNVFSCSVMHEALHNHYTQKSL |
| | | | SLSPGSGGGGSGGGGQSLVESGGGLVQPGGSLRLSCAASGIDFSSSGYMCWVRQA |
| | | | PGKGLEWVGCIATYSSNTYYAASVKGRFTISKTSSTTVYLQMNSLKTEDTAVYYC |
| | | | ARGTYGYTGYTYTMGYFSLWGQGTLVTVSSASTKGPSVFPLAPSSKSTSGGTAAL |
| | | | GCLVKDYFPEPVTVSWNSGALTSGVHTFPAVLQSSGLYSLSSVVTVPSSSLGTQTY |
| | | | ICNVNHKPSNTKVDKKVEPKSC |
| HC-1047 | hIgG1.agly- | | KTHTCPPCPAPELLGGPSVFLFPPKPKDTLMISRTPEVTCVVVDVSHEDPEVKFNW |
| | SG4SG4-VH- | | YVDGVEVHNAKTKPREEQYQSTYRVVSVLTVLHQDWLNGKEYKCKVSNKALPA |
| | CH1 VH- | | PIEKTISKAKGQPREPQVYTLPPSRDELTKNQVSLTCLVKGFYPSDIAVEWESNGQP |
| | Y61A | | ENNYKTTPPVLDSDGSFFLYSKLTVDKSRWQQGNVFSCSVMHEALHNHYTQKSL |
| | | | SLSPGSGGGGSGGGGQSLVESGGGLVQPGGSLRLSCAASGIDFSSSGYMCWVRQA |
| | | | PGKGLEWVGCIYTASSNTYYAASVKGRFTISKTSSTTVYLQMNSLKTEDTAVYYC |
| | | | ARGTYGYTGYTYTMGYFSLWGQGTLVTVSSASTKGPSVFPLAPSSKSTSGGTAAL |
| | | | GCLVKDYFPEPVTVSWNSGALTSGVHTFPAVLQSSGLYSLSSVVTVPSSSLGTQTY |
| | | | ICNVNHKPSNTKVDKKVEPKSC |
| HC-1048 | hIgG1.agly- | | KTHTCPPCPAPELLGGPSVFLFPPKPKDTLMISRTPEVTCVVVDVSHEDPEVKFNW |
| | SG4SG4-VH- | | YVDGVEVHNAKTKPREEQYQSTYRVVSVLTVLHQDWLNGKEYKCKVSNKALPA |
| | CH1 VH- | | PIEKTISKAKGQPREPQVYTLPPSRDELTKNQVSLTCLVKGFYPSDIAVEWESNGQP |
| | N67A | | ENNYKTTPPVLDSDGSFFLYSKLTVDKSRWQQGNVFSCSVMHEALHNHYTQKSL |
| | | | SLSPGSGGGGSGGGGQSLVESGGGLVQPGGSLRLSCAASGIDFSSSGYMCWVRQA |
| | | | PGKGLEWVGCIYTYSSATYYAASVKGRFTISKTSSTTVYLQMNSLKTEDTAVYYC |
| | | | ARGTYGYTGYTYTMGYFSLWGQGTLVTVSSASTKGPSVFPLAPSSKSTSGGTAAL |
| | | | GCLVKDYFPEPVTVSWNSGALTSGVHTFPAVLQSSGLYSLSSVVTVPSSSLGTQTY |
| | | | ICNVNHKPSNTKVDKKVEPKSC |
| HC-1049 | hIgG1.agly- | | KTHTCPPCPAPELLGGPSVFLFPPKPKDTLMISRTPEVTCVVVDVSHEDPEVKFNW |
| | SG4SG4-VH- | | YVDGVEVHNAKTKPREEQYQSTYRVVSVLTVLHQDWLNGKEYKCKVSNKALPA |
| | CH1 VH- | | PIEKTISKAKGQPREPQVYTLPPSRDELTKNQVSLTCLVKGFYPSDIAVEWESNGQP |
| | Y69A | | ENNYKTTPPVLDSDGSFFLYSKLTVDKSRWQQGNVFSCSVMHEALHNHYTQKSL |
| | | | SLSPGSGGGGSGGGGQSLVESGGGLVQPGGSLRLSCAASGIDFSSSGYMCWVRQA |
| | | | PGKGLEWVGCIYTYSSNTAYAASVKGRFTISKTSSTTVYLQMNSLKTEDTAVYYC |
| | | | ARGTYGYTGYTYTMGYFSLWGQGTLVTVSSASTKGPSVFPLAPSSKSTSGGTAAL |
| | | | GCLVKDYFPEPVTVSWNSGALTSGVHTFPAVLQSSGLYSLSSVVTVPSSSLGTQTY |
| | | | ICNVNHKPSNTKVDKKVEPKSC |
| HC-1050 | hIgG1.agly- | | KTHTCPPCPAPELLGGPSVFLFPPKPKDTLMISRTPEVTCVVVDVSHEDPEVKFNW |
| | SG4SG4-VH- | | YVDGVEVHNAKTKPREEQYQSTYRVVSVLTVLHQDWLNGKEYKCKVSNKALPA |
| | CH1 VH- | | PIEKTISKAKGQPREPQVYTLPPSRDELTKNQVSLTCLVKGFYPSDIAVEWESNGQP |
| | Y113A | | ENNYKTTPPVLDSDGSFFLYSKLTVDKSRWQQGNVFSCSVMHEALHNHYTQKSL |
| | | | SLSPGSGGGGSGGGGQSLVESGGGLVQPGGSLRLSCAASGIDFSSSGYMCWVRQA |
| | | | PGKGLEWVGCIYTYSSNTYYAASVKGRFTISKTSSTTVYLQMNSLKTEDTAVYYC |
| | | | ARGTYGATGYTYTMGYFSLWGQGTLVTVSSASTKGPSVFPLAPSSKSTSGGTAAL |
| | | | GCLVKDYFPEPVTVSWNSGALTSGVHTFPAVLQSSGLYSLSSVVTVPSSSLGTQTY |
| | | | ICNVNHKPSNTKVDKKVEPKSC |
| HC-1051 | hIgG1.agly- | | KTHTCPPCPAPELLGGPSVFLFPPKPKDTLMISRTPEVTCVVVDVSHEDPEVKFNW |
| | SG4SG4-VH- | | YVDGVEVHNAKTKPREEQYQSTYRVVSVLTVLHQDWLNGKEYKCKVSNKALPA |
| | CH1 VH- | | PIEKTISKAKGQPREPQVYTLPPSRDELTKNQVSLTCLVKGFYPSDIAVEWESNGQP |
| | Y116A | | ENNYKTTPPVLDSDGSFFLYSKLTVDKSRWQQGNVFSCSVMHEALHNHYTQKSL |
| | | | SLSPGSGGGGSGGGGQSLVESGGGLVQPGGSLRLSCAASGIDFSSSGYMCWVRQA |
| | | | PGKGLEWVGCIYTYSSNTYYAASVKGRFTISKTSSTTVYLQMNSLKTEDTAVYYC |
| | | | ARGTYGYTGATYTMGYFSLWGQGTLVTVSSASTKGPSVFPLAPSSKSTSGGTAAL |
| | | | GCLVKDYFPEPVTVSWNSGALTSGVHTFPAVLQSSGLYSLSSVVTVPSSSLGTQTY |
| | | | ICNVNHKPSNTKVDKKVEPKSC |
| HC-1052 | hIgG1.agly- | | KTHTCPPCPAPELLGGPSVFLFPPKPKDTLMISRTPEVTCVVVDVSHEDPEVKFNW |
| | SG4SG4-VH- | | YVDGVEVHNAKTKPREEQYQSTYRVVSVLTVLHQDWLNGKEYKCKVSNKALPA |
| | CH1 VH- | | PIEKTISKAKGQPREPQVYTLPPSRDELTKNQVSLTCLVKGFYPSDIAVEWESNGQP |
| | T130A | | ENNYKTTPPVLDSDGSFFLYSKLTVDKSRWQQGNVFSCSVMHEALHNHYTQKSL |
| | | | SLSPGSGGGGSGGGGQSLVESGGGLVQPGGSLRLSCAASGIDFSSSGYMCWVRQA |
| | | | PGKGLEWVGCIYTYSSNTYYAASVKGRFTISKTSSTTVYLQMNSLKTEDTAVYYC |
| | | | ARGTYGYTGYAYTMGYFSLWGQGTLVTVSSASTKGPSVFPLAPSSKSTSGGTAAL |
| | | | GCLVKDYFPEPVTVSWNSGALTSGVHTFPAVLQSSGLYSLSSVVTVPSSSLGTQTY |
| | | | ICNVNHKPSNTKVDKKVEPKSC |
| HC-1053 | hIgG1.agly- | | KTHTCPPCPAPELLGGPSVFLFPPKPKDTLMISRTPEVTCVVVDVSHEDPEVKFNW |
| | SG4SG4-VH- | | YVDGVEVHNAKTKPREEQYQSTYRVVSVLTVLHQDWLNGKEYKCKVSNKALPA |
| | CH1 VH- | | PIEKTISKAKGQPREPQVYTLPPSRDELTKNQVSLTCLVKGFYPSDIAVEWESNGQP |
| | Y131A | | ENNYKTTPPVLDSDGSFFLYSKLTVDKSRWQQGNVFSCSVMHEALHNHYTQKSL |
| | | | SLSPGSGGGGSGGGGQSLVESGGGLVQPGGSLRLSCAASGIDFSSSGYMCWVRQA |
| | | | PGKGLEWVGCIYTYSSNTYYAASVKGRFTISKTSSTTVYLQMNSLKTEDTAVYYC |
| | | | ARGTYGYTGYTATMGYFSLWGQGTLVTVSSASTKGPSVFPLAPSSKSTSGGTAAL |
| | | | GCLVKDYFPEPVTVSWNSGALTSGVHTFPAVLQSSGLYSLSSVVTVPSSSLGTQTY |
| | | | ICNVNHKPSNTKVDKKVEPKSC |
| HC-1054 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 | | ENNYKTTPPVLDSDGSFFLYSKLTVDKSRWQQGNVFSCSVMHEALHNHYTQKSL |
| | | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1055 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH-S32A | | ENNYKTTPPVLDSDGSFFLYSKLTVDKSRWQQGNVFSCSVMHEALHNHYTQKSL |
| | | | SLSPGSGGGGSGGGGEVQLVESGGGLVQPGGSLRLSCAASGIDFASSGYMCWVRQ |
| | | | APGKGLEWVGCIYTYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1056 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH-S32D | | ENNYKTTPPVLDSDGSFFLYSKLTVDKSRWQQGNVFSCSVMHEALHNHYTQKSL |
| | | | SLSPGSGGGGSGGGGEVQLVESGGGLVQPGGSLRLSCAASGIDFDSSGYMCWVRQ |
| | | | APGKGLEWVGCIYTYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1057 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH-S32H | | ENNYKTTPPVLDSDGSFFLYSKLTVDKSRWQQGNVFSCSVMHEALHNHYTQKSL |
| | | | SLSPGSGGGGSGGGGEVQLVESGGGLVQPGGSLRLSCAASGIDFHSSGYMCWVRQ |
| | | | APGKGLEWVGCIYTYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1058 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH-S33A | | ENNYKTTPPVLDSDGSFFLYSKLTVDKSRWQQGNVFSCSVMHEALHNHYTQKSL |
| | | | SLSPGSGGGGSGGGGEVQLVESGGGLVQPGGSLRLSCAASGIDFSASGYMCWVRQ |
| | | | APGKGLEWVGCIYTYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1059 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH-S33D | | ENNYKTTPPVLDSDGSFFLYSKLTVDKSRWQQGNVFSCSVMHEALHNHYTQKSL |
| | | | SLSPGSGGGGSGGGGEVQLVESGGGLVQPGGSLRLSCAASGIDFSDSGYMCWVRQ |
| | | | APGKGLEWVGCIYTYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1060 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH-S33H | | ENNYKTTPPVLDSDGSFFLYSKLTVDKSRWQQGNVFSCSVMHEALHNHYTQKSL |
| | | | SLSPGSGGGGSGGGGEVQLVESGGGLVQPGGSLRLSCAASGIDFSHSGYMCWVRQ |
| | | | APGKGLEWVGCIYTYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1061 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CHI VH-S38A | | ENNYKTTPPVLDSDGSFFLYSKLTVDKSRWQQGNVFSCSVMHEALHNHYTQKSL |
| | | | SLSPGSGGGGSGGGGEVQLVESGGGLVQPGGSLRLSCAASGIDFSSAGYMCWVRQ |
| | | | APGKGLEWVGCIYTYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1062 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH-S38D | | ENNYKTTPPVLDSDGSFFLYSKLTVDKSRWQQGNVFSCSVMHEALHNHYTQKSL |
| | | | SLSPGSGGGGSGGGGEVQLVESGGGLVQPGGSLRLSCAASGIDFSSDGYMCWVRQ |
| | | | APGKGLEWVGCIYTYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1063 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH-S38H | | ENNYKTTPPVLDSDGSFFLYSKLTVDKSRWQQGNVFSCSVMHEALHNHYTQKSL |
| | | | SLSPGSGGGGSGGGGEVQLVESGGGLVQPGGSLRLSCAASGIDFSSHGYMCWVRQ |
| | | | APGKGLEWVGCIYTYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1064 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y59A | | SLSPGSGGGGSGGGGEVQLVESGGGLVQPGGSLRLSCAASGIDFSSSGYMCWVRQ |
| | | | APGKGLEWVGCIATYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1065 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y59D | | SLSPGSGGGGSGGGGEVQLVESGGGLVQPGGSLRLSCAASGIDFSSSGYMCWVRQ |
| | | | APGKGLEWVGCIDTYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1066 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CHI VH- | | ENNYKTTPPVLDSDGSFFLYSKLTVDKSRWQQGNVFSCSVMHEALHNHYTQKSL |
| | Y59H | | SLSPGSGGGGSGGGGEVQLVESGGGLVQPGGSLRLSCAASGIDFSSSGYMCWVRQ |
| | | | APGKGLEWVGCIHTYSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1067 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y61A | | SLSPGSGGGGSGGGGEVQLVESGGGLVQPGGSLRLSCAASGIDFSSSGYMCWVRQ |
| | | | APGKGLEWVGCIYTASSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1068 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y61D | | SLSPGSGGGGSGGGGEVQLVESGGGLVQPGGSLRLSCAASGIDFSSSGYMCWVRQ |
| | | | APGKGLEWVGCIYTDSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1069 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y61H | | SLSPGSGGGGSGGGGEVQLVESGGGLVQPGGSLRLSCAASGIDFSSSGYMCWVRQ |
| | | | APGKGLEWVGCIYTHSSNTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1070 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | N67A | | SLSPGSGGGGSGGGGEVQLVESGGGLVQPGGSLRLSCAASGIDFSSSGYMCWVRQ |
| | | | APGKGLEWVGCIYTYSSATYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1071 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | N67D | | SLSPGSGGGGSGGGGEVQLVESGGGLVQPGGSLRLSCAASGIDFSSSGYMCWVRQ |
| | | | APGKGLEWVGCIYTYSSDTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1072 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | N67H | | SLSPGSGGGGSGGGGEVQLVESGGGLVQPGGSLRLSCAASGIDFSSSGYMCWVRQ |
| | | | APGKGLEWVGCIYTYSSHTYYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1073 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y69A | | SLSPGSGGGGSGGGGEVQLVESGGGLVQPGGSLRLSCAASGIDFSSSGYMCWVRQ |
| | | | APGKGLEWVGCIYTYSSNTAYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1074 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y69D | | SLSPGSGGGGSGGGGEVQLVESGGGLVQPGGSLRLSCAASGIDFSSSGYMCWVRQ |
| | | | APGKGLEWVGCIYTYSSNTDYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1075 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y69H | | SLSPGSGGGGSGGGGEVQLVESGGGLVQPGGSLRLSCAASGIDFSSSGYMCWVRQ |
| | | | APGKGLEWVGCIYTYSSNTHYAASVKGRFTISKTSSTTVYLQMNSLKTEDTAVYY |
| | | | CARGTYGYTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1076 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y113A | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGATGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1077 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y113D | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGDTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1078 | hIgG1. agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y113H | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGHTGYTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1079 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y116A | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGYTGATYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1080 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y116D | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGYTGDTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1081 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y116H | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGYTGHTYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1082 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | T130A | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGYTGYAYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1083 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | T130D | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGYTGYDYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1084 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | T130H | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGYTGYHYTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1085 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y131A | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGYTGYTATMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1086 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y131D | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGYTGYTDTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1087 | hIgG1.agly- | | KTHTCPPCPAPELLGGPSVFLFPPKPKDTLMISRTPEVTCVVVDVSHEDPEVKFNW |
| | SG4SG4- | | YVDGVEVHNAKTKPREEQYQSTYRVVSVLTVLHQDWLNGKEYKCKVSNKALPA |
| | EVQL VH- | | PIEKTISKAKGQPREPQVYTLPPSRDELTKNQVSLTCLVKGFYPSDIAVEWESNGQP |
| | CH1 VH- | | ENNYKTTPPVLDSDGSFFLYSKLTVDKSRWQQGNVFSCSVMHEALHNHYTQKSL |
| | Y131H | | SLSPGSGGGGSGGGGEVQLVESGGGLVQPGGSLRLSCAASGIDFSSSGYMCWVRQ |
| | | | APGKGLEWVGCIYTYSSNTYYAASVKGRFTISKTSSTTVYLQMNSLKTEDTAVYY |
| | | | CARGTYGYTGYTHTMGYFSLWGQGTLVTVSSASTKGPSVFPLAPSSKSTSGGTAA |
| | | | LGCLVKDYFPEPVTVSWNSGALTSGVHTFPAVLQSSGLYSLSSVVTVPSSSLGTQT |
| | | | YICNVNHKPSNTKVDKKVEPKSC |
| HC-1088 | hGlag Fc | | KTHTCPPCPAPELLGGPSVFLFPPKPKDTLMISRTPEVTCVVVDVSHEDPEVKFNW |
| | VH-VL scFv | | YVDGVEVHNAKTKPREEQYQSTYRVVSVLTVLHQDWLNGKEYKCKVSNKALPA |
| | VH-N67D | | PIEKTISKAKGQPREPQVYTLPPSRDELTKNQVSLTCLVKGFYPSDIAVEWESNGQP |
| | | | ENNYKTTPPVLDSDGSFFLYSKLTVDKSRWQQGNVFSCSVMHEALHNHYTQKSL |
| | | | SLSPGSGGGGEVQLVESGGGLVQPGGSLRLSCAASGIDFSSSGYMCWVRQAPGKG |
| | | | LEWVGCIYTYSSDTYYAASVKGRFTISKTSSTTVYLQMNSLKTEDTAVYYCARGT |
| | | | YGYTGYTYTMGYFSLWGQGTLVTVSSGGGGSGGGGSGGGGSDIQMTQSPSTLSA |
| | | | SVGDRVTITCQASQNINSYLAWYQQKPGKAPKLLIYRASSLESGVPSRFSGSGSGT |
| | | | EFTLTISSLQPDDFATYYCQSYYYSGSSNYNAFGGGTKVEIK |
| HC-1089 | hGlag Fc | | KTHTCPPCPAPELLGGPSVFLFPPKPKDTLMISRTPEVTCVVVDVSHEDPEVKFNW |
| | VH-VL scFv | | YVDGVEVHNAKTKPREEQYQSTYRVVSVLTVLHQDWLNGKEYKCKVSNKALPA |
| | VH-N67D VL- | | PIEKTISKAKGQPREPQVYTLPPSRDELTKNQVSLTCLVKGFYPSDIAVEWESNGQP |
| | R58D | | ENNYKTTPPVLDSDGSFFLYSKLTVDKSRWQQGNVFSCSVMHEALHNHYTQKSL |
| | | | SLSPGSGGGGEVQLVESGGGLVQPGGSLRLSCAASGIDFSSSGYMCWVRQAPGKG |
| | | | LEWVGCIYTYSSDTYYAASVKGRFTISKTSSTTVYLQMNSLKTEDTAVYYCARGT |
| | | | YGYTGYTYTMGYFSLWGQGTLVTVSSGGGGSGGGGSGGGGSDIQMTQSPSTLSA |
| | | | SVGDRVTITCQASQNINSYLAWYQQKPGKAPKLLIYDASSLESGVPSRFSGSGSGT |
| | | | EFTLTISSLQPDDFATYYCQSYYYSGSSNYNAFGGGTKVEIK |
| HC-1090 | hGlag Fc | | KTHTCPPCPAPELLGGPSVFLFPPKPKDTLMISRTPEVTCVVVDVSHEDPEVKFNW |
| | VL-VH scFv | | YVDGVEVHNAKTKPREEQYQSTYRVVSVLTVLHQDWLNGKEYKCKVSNKALPA |
| | VH-N67D | | PIEKTISKAKGQPREPQVYTLPPSRDELTKNQVSLTCLVKGFYPSDIAVEWESNGQP |
| | | | ENNYKTTPPVLDSDGSFFLYSKLTVDKSRWQQGNVFSCSVMHEALHNHYTQKSL |
| | | | SLSPGSGGGGDIQMTQSPSTLSASVGDRVTITCQASQNINSYLAWYQQKPGKAPKL |
| | | | LIYRASSLESGVPSRFSGSGSGTEFTLTISSLQPDDFATYYCQSYYYSGSSNYNAFG |
| | | | GGTKVEIKGGGGSGGGGSGGGGSEVQLVESGGGLVQPGGSLRLSCAASGIDFSSS |
| | | | GYMCWVRQAPGKGLEWVGCIYTYSSDTYYAASVKGRFTISKTSSTTVYLQMNSL |
| | | | KTEDTAVYYCARGTYGYTGYTYTMGYFSLWGQGTLVTVSS |
| HC-1091 | hGlag Fc | | KTHTCPPCPAPELLGGPSVFLFPPKPKDTLMISRTPEVTCVVVDVSHEDPEVKFNW |
| | VL-VH scFv | | YVDGVEVHNAKTKPREEQYQSTYRVVSVLTVLHQDWLNGKEYKCKVSNKALPA |
| | VL-R58D VH- | | PIEKTISKAKGQPREPQVYTLPPSRDELTKNQVSLTCLVKGFYPSDIAVEWESNGQP |
| | N67D | | ENNYKTTPPVLDSDGSFFLYSKLTVDKSRWQQGNVFSCSVMHEALHNHYTQKSL |
| | | | SLSPGSGGGGDIQMTQSPSTLSASVGDRVTITCQASQNINSYLAWYQQKPGKAPKL |
| | | | LIYDASSLESGVPSRFSGSGSGTEFTLTISSLQPDDFATYYCQSYYYSGSSNYNAFG |
| | | | GGTKVEIKGGGGSGGGGSGGGGSEVQLVESGGGLVQPGGSLRLSCAASGIDFSSS |
| | | | GYMCWVRQAPGKGLEWVGCIYTYSSDTYYAASVKGRFTISKTSSTTVYLQMNSL |
| | | | KTEDTAVYYCARGTYGYTGYTYTMGYFSLWGQGTLVTVSS |
| HC-1092 | hGlag Fc | | KTHTCPPCPAPELLGGPSVFLFPPKPKDTLMISRTPEVTCVVVDVSHEDPEVKFNW |
| | VL-VH scFv | | YVDGVEVHNAKTKPREEQYQSTYRVVSVLTVLHQDWLNGKEYKCKVSNKALPA |
| | VH-N67D | | PIEKTISKAKGQPREPQVYTLPPSRDELTKNQVSLTCLVKGFYPSDIAVEWESNGQP |
| | CACA | | ENNYKTTPPVLDSDGSFFLYSKLTVDKSRWQQGNVFSCSVMHEALHNHYTQKSL |
| | | | SLSPGSGGGGDIQMTQSPSTLSASVGDRVTITCQASQNINSYLAWYQQKPGKAPKL |
| | | | LIYRASSLESGVPSRFSGSGSGTEFTLTISSLQPDDFATYYCQSYYYSGSSNYNAFG |
| | | | GGTKVEIKGGGGSGGGGSGGGGSEVQLVESGGGLVQPGGSLRLSCAASGIDFSSS |
| | | | GYMAWVRQAPGKGLEWVGAIYTYSSDTYYAASVKGRFTISKTSSTTVYLQMNSL |
| | | | KTEDTAVYYCARGTYGYTGYTYTMGYFSLWGQGTLVTVSS |
| HC-1093 | hGlag Fc | | KTHTCPPCPAPELLGGPSVFLFPPKPKDTLMISRTPEVTCVVVDVSHEDPEVKFNW |
| | VL-VH scFv | | YVDGVEVHNAKTKPREEQYQSTYRVVSVLTVLHQDWLNGKEYKCKVSNKALPA |
| | VH-N67D 51- | | PIEKTISKAKGQPREPQVYTLPPSRDELTKNQVSLTCLVKGFYPSDIAVEWESNGQP |
| | 141dsb | | ENNYKTTPPVLDSDGSFFLYSKLTVDKSRWQQGNVFSCSVMHEALHNHYTQKSL |
| | | | SLSPGSGGGGDIQMTQSPSTLSASVGDRVTITCQASQNINSYLAWYQQKPGKAPKL |
| | | | LIYRASSLESGVPSRFSGSGSGTEFTLTISSLQPDDFATYYCQSYYYSGSSNYNAFG |
| | | | CGTKVEIKGGGGSGGGGSGGGGSEVQLVESGGGLVQPGGSLRLSCAASGIDFSSS |
| | | | GYMCWVRQAPGKCLEWVGCIYTYSSDTYYAASVKGRFTISKTSSTTVYLQMNSL |
| | | | KTEDTAVYYCARGTYGYTGYTYTMGYFSLWGQGTLVTVSS |
| HC-1094 | hGlag Fc | | KTHTCPPCPAPELLGGPSVFLFPPKPKDTLMISRTPEVTCVVVDVSHEDPEVKFNW |
| | VL-VH scFv | | YVDGVEVHNAKTKPREEQYQSTYRVVSVLTVLHQDWLNGKEYKCKVSNKALPA |
| | VH-N67D | | PIEKTISKAKGQPREPQVYTLPPSRDELTKNQVSLTCLVKGFYPSDIAVEWESNGQP |
| | CACA 51- | | ENNYKTTPPVLDSDGSFFLYSKLTVDKSRWQQGNVFSCSVMHEALHNHYTQKSL |
| | 141dsb | | SLSPGSGGGGDIQMTQSPSTLSASVGDRVTITCQASQNINSYLAWYQQKPGKAPKL |
| | | | LIYRASSLESGVPSRFSGSGSGTEFTLTISSLQPDDFATYYCQSYYYSGSSNYNAFG |
| | | | CGTKVEIKGGGGSGGGGSGGGGSEVQLVESGGGLVQPGGSLRLSCAASGIDFSSS |
| | | | GYMAWVRQAPGKCLEWVGAIYTYSSDTYYAASVKGRFTISKTSSTTVYLQMNSL |
| | | | KTEDTAVYYCARGTYGYTGYTYTMGYFSLWGQGTLVTVSS |
| LC-1095 | LC hKappa | | DIQMTQSPSTLSASVGDRVTITCQASQNINSYLAWYQQKPGKAPKLLIYRASSLES |
| | | | GVPSRFSGSGSGTEFTLTISSLQPDDFATYYCQSYYYSGSSNYNAFGGGTKVEIKRT |
| | | | VAAPSVFIFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRGEC |
| LC-1096 | LC hKappa- | | DIQMTQSPSTLSASVGDRVTITCQASQNINSYLAWYQQKPGKAPKLLIYRASSLES |
| | HA | | GVPSRFSGSGSGTEFTLTISSLQPDDFATYYCQSYYYSGSSNYNAFGGGTKVEIKRT |
| | | | VAAPSVFIFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRGECTGYPY |
| | | | DVPDYA |
| LC-1098 | LC VL-N30S | | DIQMTQSPSTLSASVGDRVTITCQASQSINSYLAWYQQKPGKAPKLLIYRASSLESG |
| | | | VPSRFSGSGSGTEFTLTISSLQPDDFATYYCQSYYYSGSSNYNAFGGGTKVEIKRTV |
| | | | AAPSVFIFPPSDEQLKSGTASVVCLLNNFYPREAKVQWKVDNALQSGNSQESVTE |
| | | | QDSKDSTYSLSSTLTLSKADYEKHKVYACEVTHQGLSSPVTKSFNRGEC |
| LC-1099 | LC VL-N32S | | DIQMTQSPSTLSASVGDRVTITCQASQNISSYLAWYQQKPGKAPKLLIYRASSLESG |
| | | | VPSRFSGSGSGTEFTLTISSLQPDDFATYYCQSYYYSGSSNYNAFGGGTKVEIKRTV |
| | | | AAPSVFIFPPSDEQLKSGTASVVCLLNNFYPREAKVQWKVDNALQSGNSQESVTE |
| | | | QDSKDSTYSLSSTLTLSKADYEKHKVYACEVTHQGLSSPVTKSFNRGEC |
| LC-1100 | LC VL-R58D | | DIQMTQSPSTLSASVGDRVTITCQASQNINSYLAWYQQKPGKAPKLLIYDASSLES |
| | | | GVPSRFSGSGSGTEFTLTISSLQPDDFATYYCQSYYYSGSSNYNAFGGGTKVEIKRT |
| | | | VAAPSVFIFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRGEC |
| LC-1101 | LC VL- | | DIQMTQSPSTLSASVGDRVTITCQASQNINSYLAWYQQKPGKAPKLLIYRASSLES |
| | Y111D | | GVPSRFSGSGSGTEFTLTISSLQPDDFATYYCQSYYDSGSSNYNAFGGGTKVEIKRT |
| | | | VAAPSVFIFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRGEC |
| LC-1102 | LC VL- | | DIQMTQSPSTLSASVGDRVTITCQASQNINSYLAWYQQKPGKAPKLLIYRASSLES |
| | Y111H | | GVPSRFSGSGSGTEFTLTISSLQPDDFATYYCQSYYHSGSSNYNAFGGGTKVEIKRT |
| | | | VAAPSVFIFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRGEC |
| LC-1103 | LC VL-Y111S | | DIQMTQSPSTLSASVGDRVTITCQASQNINSYLAWYQQKPGKAPKLLIYRASSLES |
| | | | GVPSRFSGSGSGTEFTLTISSLQPDDFATYYCQSYYSSGSSNYNAFGGGTKVEIKRT |
| | | | VAAPSVFIFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRGEC |
| LC-1104 | LC VL-S133A | | DIQMTQSPSTLSASVGDRVTITCQASQNINSYLAWYQQKPGKAPKLLIYRASSLES |
| | | | GVPSRFSGSGSGTEFTLTISSLQPDDFATYYCQSYYYSGASNYNAFGGGTKVEIKR |
| | | | TVAAPSVFIFPPSDEQLKSGTASVVCLLNNFYPREAKVQWKVDNALQSGNSQESV |
| | | | TEQDSKDSTYSLSSTLTLSKADYEKHKVYACEVTHQGLSSPVTKSFNRGEC |
| LC-1105 | LC VL-S133D | | DIQMTQSPSTLSASVGDRVTITCQASQNINSYLAWYQQKPGKAPKLLIYRASSLES |
| | | | GVPSRFSGSGSGTEFTLTISSLQPDDFATYYCQSYYYSGDSNYNAFGGGTKVEIKR |
| | | | TVAAPSVFIFPPSDEQLKSGTASVVCLLNNFYPREAKVQWKVDNALQSGNSQESV |
| | | | TEQDSKDSTYSLSSTLTLSKADYEKHKVYACEVTHQGLSSPVTKSFNRGEC |
| LC-1106 | LC VL-S133H | | DIQMTQSPSTLSASVGDRVTITCQASQNINSYLAWYQQKPGKAPKLLIYRASSLES |
| | | | GVPSRFSGSGSGTEFTLTISSLQPDDFATYYCQSYYYSGHSNYNAFGGGTKVEIKR |
| | | | TVAAPSVFIFPPSDEQLKSGTASVVCLLNNFYPREAKVQWKVDNALQSGNSQESV |
| | | | TEQDSKDSTYSLSSTLTLSKADYEKHKVYACEVTHQGLSSPVTKSFNRGEC |
| LC-1107 | LC VL- | | DIQMTQSPSTLSASVGDRVTITCQASQSINSYLAWYQQKPGKAPKLLIYDASSLES |
| | N30S/R58D | | GVPSRFSGSGSGTEFTLTISSLQPDDFATYYCQSYYYSGSSNYNAFGGGTKVEIKRT |
| | | | VAAPSVFIFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRGEC |
| LC-1108 | LC VL- | | DIQMTQSPSTLSASVGDRVTITCQASQSINSYLAWYQQKPGKAPKLLIYRASSLESG |
| | N30S/Y111S | | VPSRFSGSGSGTEFTLTISSLQPDDFATYYCQSYYSSGSSNYNAFGGGTKVEIKRTV |
| | | | AAPSVFIFPPSDEQLKSGTASVVCLLNNFYPREAKVQWKVDNALQSGNSQESVTE |
| | | | QDSKDSTYSLSSTLTLSKADYEKHKVYACEVTHQGLSSPVTKSFNRGEC |
| LC-1109 | LC VL- | | DIQMTQSPSTLSASVGDRVTITCQASQSINSYLAWYQQKPGKAPKLLIYRASSLESG |
| | N30S/Y111D | | VPSRFSGSGSGTEFTLTISSLQPDDFATYYCQSYYDSGSSNYNAFGGGTKVEIKRTV |
| | | | AAPSVFIFPPSDEQLKSGTASVVCLLNNFYPREAKVQWKVDNALQSGNSQESVTE |
| | | | QDSKDSTYSLSSTLTLSKADYEKHKVYACEVTHQGLSSPVTKSFNRGEC |
| LC-1110 | LC VL- | | DIQMTQSPSTLSASVGDRVTITCQASQSINSYLAWYQQKPGKAPKLLIYRASSLESG |
| | N30S/S133D | | VPSRFSGSGSGTEFTLTISSLQPDDFATYYCQSYYYSGDSNYNAFGGGTKVEIKRT |
| | | | VAAPSVFIFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRGEC |
| LC-1111 | LC VL- | | DIQMTQSPSTLSASVGDRVTITCQASQSINSYLAWYQQKPGKAPKLLIYDASSLES |
| | N30S/R58D/R | | GVPSRFSGSGSGTEFTLTISSLQPDDFATYYCQSYYSSGSSNYNAFGGGTKVEIKRT |
| | 58D | | VAAPSVFIFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRGEC |
| LC-1112 | LC VL- | | DIQMTQSPSTLSASVGDRVTITCQASQSINSYLAWYQQKPGKAPKLLIYDASSLES |
| | N30S/R58D/S | | GVPSRFSGSGSGTEFTLTISSLQPDDFATYYCQSYYYSGDSNYNAFGGGTKVEIKR |
| | 133D | | TVAAPSVFIFPPSDEQLKSGTASVVCLLNNFYPREAKVQWKVDNALQSGNSQESV |
| | | | TEQDSKDSTYSLSSTLTLSKADYEKHKVYACEVTHQGLSSPVTKSFNRGEC |
| LC-1113 | LC-DelC214 | | DIQMTQSPSTLSASVGDRVTITCQASQNINSYLAWYQQKPGKAPKLLIYRASSLES |
| | | | GVPSRFSGSGSGTEFTLTISSLQPDDFATYYCQSYYYSGSSNYNAFGGGTKVEIKRT |
| | | | VAAPSVFIFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRGE |
| LC-1114 | LC-DelC214 | | DIQMTQSPSTLSASVGDRVTITCQASQNINSYLAWYQQKPGKAPKLLIYDASSLES |
| | VL-R58D | | GVPSRFSGSGSGTEFTLTISSLQPDDFATYYCQSYYYSGSSNYNAFGGGTKVEIKRT |
| | | | VAAPSVFIFPPSDEQLKSGTASVVCLLNNFYPREAKVQWKVDNALQSGNSQESVT |
| | | | EQDSKDSTYSLSSTLTLSKADYEKHKVYACEVTHQGLSSPVTKSFNRGE |

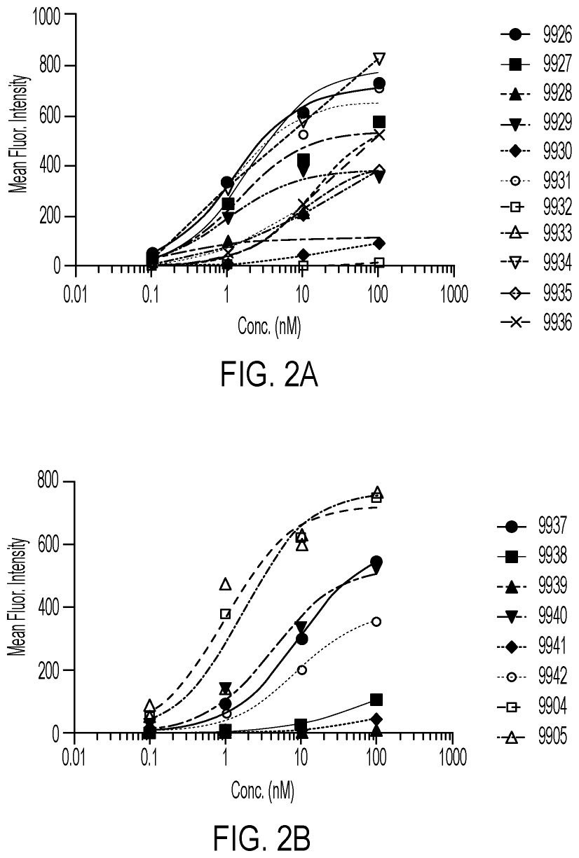

Point mutations were combined to create further engineered variants of H2C/L0 with finely-tuned binding properties. In some cases, mutations that reduce binding (e.g., VH-N67D and VL-Y111S) were combined with mutations that increase human-ness (e.g., VL-N30S, VL-Y111S, and mutation of VH-Nterm from QSL to EVQL) to create a version of H2C/L0 with both reduced binding to target and increased human-ness. Table 11 summarizes affinity changes at each position in H2C/L0 combination mutants by the amino acids used for mutation. Mutation of the VH N-terminus from QSL to EVQL did not significantly change the mutants' affinity to TfR. Concentration-dependent binding of mutant antibodies to CHO cells is shown in FIGS. 2A-2B.

TABLE 11
H2C/L0 and H2C/L0 Combination Mutants Binding to TfR

| | | Flow Cytometry | | SPR (monovalent binding) | | | |
|---|---|---|---|---|---|---|---|
| | | huTfR-CHO binding | | Human TfR Apical | | Cyno TfR Apical | |
| Protein | | EC50 | | Domain binding | | Domain binding | |
| ID | Description | (nM) | Max MFI | kd (/s) | KD (nM) | kd (/s) | KD (nM) |
| 9904 | H2C/L0 hIgG1.agly | 1.0 | 721 | 0.00059 | 4.7 | 0.0056 | 28 |
| 9905 | H2C/L0-EVQL hIgG1.agly | 0.7 | 748 | 0.00081 | 7.5 | 0.0112 | 43 |
| 9926 | H2C/L0-EVQL VH-S32D/Y61D hIgG1.agly | 1.2 | 712 | 0.038 | 150 | >0.05 | 398 |
| 9927 | H2C/L0-EVQL VH-S32D/N67E hIgG1.agly | 1.4 | 541 | 0.064 | 480 | >0.05 | 601 |
| 9928 | H2C/L0-EVQL VH-S32D/Y69D hIgG1.agly | 0.3 | 116 | >0.05 | 2080 | >0.05 | ~5070 |
| 9929 | H2C/L0-EVQL VH-S32D/Y69E hIgG1.agly | 1.0 | 381 | >0.05 | 755 | >0.05 | 2070 |
| 9930 | H2C/L0-EVQL VH-S32D/Y59H/Y61D hIgG1.agly | 12.4 | 101 | >0.05 | >800 | >0.05 | >800 |
| 9931 | H2C/L0-EVQL VH-S32D/Y116A hIgG1.agly | 1.1 | 658 | 0.0095 | 304 | >0.05 | 395 |
| 9932 | H2C/L0-EVQL VH-Y69A/Y116A hIgG1.agly | 54.6 | 20 | >0.05 | >800 | >0.05 | >800 |
| 9933 | H2C/L0-EVQL VH-Y69E/Y116A hIgG1.agly | 34.1 | 9 | >0.05 | >800 | >0.05 | >800 |
| 9934 | H2C/L0-EVQL VH-CACA hIgG1.agly | 2.0 | 788 | 0.0035 | 55 | 0.008 | 200 |
| 9935 | H2C/L0-EVQL VH-CACA/N67D hIgG1.agly | 8.6 | 418 | >0.05 | 473 | >0.05 | >800 |
| 9936 | H2C/L0-EVQL VH-N67D VL-R58D hIgG1.agly | 13.8 | 597 | >0.05 | 462 | >0.05 | 1809 |
| 9937 | H2C/L0-EVQL VH-N67D VL-N30S/R58D hIgG1.agly | 8.8 | 583 | >0.05 | >800 | >0.05 | >800 |
| 9938 | H2C/L0-EVQL VH-N67D VL-N30S/Y111S hIgG1.agly | 42.5 | 147 | >0.05 | >800 | >0.05 | >800 |
| 9939 | H2C/L0-EVQL VH-N67D VL-N30S/Y111D hIgG1.agly | 38.3 | 6 | ~0.05 | 377 | >0.05 | 735 |
| 9940 | H2C/L0-EVQL VH-N67D VL-N30S/S133D hIgG1.agly | 4.2 | 525 | >0.05 | 890 | >0.05 | 1540 |
| | H2C/L0-EVQL VH-N67D VL-N30S/R58D/Y111S | | | | | | |
| 9941 | hIgG1.agly | 72.1 | 74 | >0.05 | >800 | >0.05 | >800 |
| | H2C/L0-EVQL VH-N67D VL-N30S/R58D/S133D | | | | | | |
| 9942 | hIgG1.agly | 8.8 | 384 | >0.05 | 2305 | >0.05 | >8000 |

### Example 2: Transcytosis Mediated by Affinity Dematured H2C/L0 Variants

MDCK II cells (ECACC 00062107) were stably transduced with VSV-G pseudotyped lentiviral particles encoding a human TfR gene under a human EF1a promoter with an Ires-Puromycin resistance cassette. Cells were selected for puromycin resistance to produce a MDCKII/hTfR cell line. Expression of human TfR was validated using anti-TfR antibodies in flow cytometry.

MDCKII/hTfR cells were plated onto Corning 0.4 micron pore transwell inserts with 25,000 cells per insert in complete medium, 0.25 ml in the insert and 1 ml in the bottom chamber. On day 4 the bottom media was fully replaced and half of the insert media is replaced with fresh media. On day 5, test article was added to the top well at 100 nM. On day 7 samples were collected from top and bottom chambers and analyzed for concentration of the test article. Quantitation of test article was performed using commonly available anti-human IgG reagents in a mesoscale discovery assay system, with results extrapolated from a titrated standard curve of the same test article.

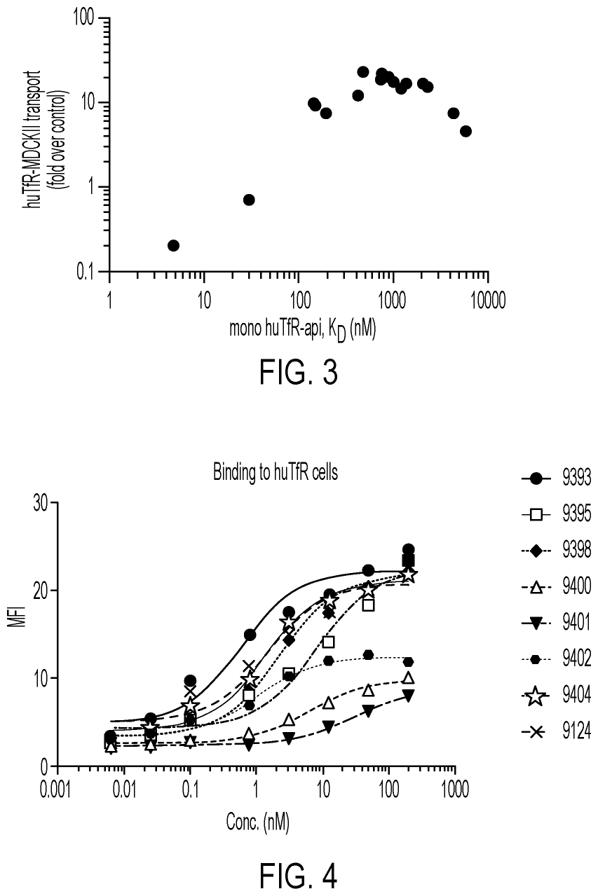

A panel of affinity de-matured H2C/L0 variants, primarily in the conventional antibody (upright, right-side up) bivalent format, were tested for transcytosis across an MDCK II monolayer where the MDCK II cells expressed human TfR. After 2 days, media from the bottom well of the transwell system was removed and concentration of antibody quantified to determine degree of transcytosis. Data are shown in the Table 12, ranked by transcytosis efficiency. Notably, the parental high-affinity H2C/L0 antibody (Protein ID 9122) demonstrated notably less transcytosis than the isotype control non-targeted antibody Ab2 (Protein ID 8849). 20 of 21 tested H2C/L0 variants demonstrated significantly more transcytosis than the non-targeted antibody (threshold 2-fold higher) with the top 3 being more than 20-fold higher than control. A variety of mutations were capable of eliciting this phenomenon. The relationship between monomeric affinity and transcytosis is depicted in FIG. 3.

One antibody evaluated consisted of an inverted Fc-scFv formatted H2C/L0 with the VH-N67D affinity-reducing mutation (Protein ID 9947) which transcytosed nearly identically to the non-scFv equivalent protein (Protein ID 9400). Another protein was a full antibody (Ab1) with a C-terminally fused H2C/L0 scFv bearing affinity reducing variant VH-N67D (Protein ID 9956), and the transcytosis of this protein demonstrates the capacity of these TfR binding shuttles to carry cargo across the barrier.

TABLE 12
Transcytosis Mediated by Affinity Dematured H2C/LO Variants

| | | | Fold | huTfR | CHO | huTfR | cyTfR | | | | | | | | | | | | | | | | |
|---|---|---|---|---|---|---|---|---|---|---|---|---|---|---|---|---|---|---|---|---|---|---|---|
| Pro- | | | Over | CHO | Max | Api | Api | | | | | | | | | | | | | | | | |
| tein | | Accum. | Con- | EC50 | huTfR | KD | KD | VH- | VH- | VH- | VH- | VH- | VH- | VH- | VH- | VH- | VH- | VH- | VH- | VH- | VL- | VL- | VL- |
| ID | Antibody | Fmol | trol | (nM) | MFI | (nM) | (nM) | S32D | S38D | Y59A | Y59D | Y61D | N67D | N67E | Y69A | Y69D | Y69E | Y116A | Y116D | Y131D | N30S | R58D | S133D |
| 8849 | Ab2 hlgG1.ag | 6.2 | 1.0 | | | | | | | | | | | | | | | | | | | | |
| 9122 | huH2C/LO bivalent RSU | 1.5 | 0.2 | 0.2 | 750 | 4.8 | 26 | | | | | | | | | | | | | | | | |
| | hlgG1.agly LC hKappa | | | | | | | | | | | | | | | | | | | | | | |
| 9910 | H2C/LO-EVQL | 4.1 | 0.7 | 1.6 | 746 | 30 | 202 | | Y | | | | | | | | | | | | | | |
| | VH-S38D hlgG1.agly | | | | | | | | | | | | | | | | | | | | | | |
| 9912 | H2C/LO-EVQL | 28.9 | 4.6 | 43.3 | 344 | ~5700 | >8000 | | | | Y | | | | | | | | | | | | |
| | VH-Y59D hlgG1.agly | | | | | | | | | | | | | | | | | | | | | | |
| 9401 | hlgG1.agly-(SG4)2- | 41.5 | 6.6 | n.d. | n.d. | n.d. | n.d. | | | | | | Y | | | | | | | | | Y | |
| | H2C/LO-EVQL VH- | | | | | | | | | | | | | | | | | | | | | | |
| | CH1 VH-N67D | | | | | | | | | | | | | | | | | | | | | | |
| | LC VL-R58D | | | | | | | | | | | | | | | | | | | | | | |
| 9925 | H2C/LO-EVQL | 43.1 | 6.9 | 22.6 | 541 | >4000 | >8000 | | | | | | | | | | | | | Y | | | |
| | VH-Y131D hlgG1.agly | | | | | | | | | | | | | | | | | | | | | | |
| 9956 | Ab1 hlgG1.LALAPG- | 46.1 | 7.4 | n.d. | n.d. | 1450 | ~3100 | | | | | | Y | | | | | | | | | | |
| | (SG4)-H2C/LO VL- | | | | | | | | | | | | | | | | | | | | | | |
| | VH scFv VH-N67D | | | | | | | | | | | | | | | | | | | | | | |
| 9923 | H2C/LO-EVQL | 46.5 | 7.5 | 13.3 | 233 | ~4300 | >8000 | | | | | | | | | | | | Y | | | | |
| | VH-Y116D hlgG1.agly | | | | | | | | | | | | | | | | | | | | | | |
| 9917 | H2C/LO-EVQL | 47.6 | 7.6 | 0.6 | 625 | 194 | 710 | | | | | | | | Y | | | | | | | | |
| | VH-Y69A hlgG1.agly | | | | | | | | | | | | | | | | | | | | | | |
| 9926 | H2C/LO-EVQL | 59.6 | 9.5 | 1.2 | 712 | 150 | 400 | Y | | | | Y | | | | | | | | | | | |
| | VH-S32D/Y61D | | | | | | | | | | | | | | | | | | | | | | |
| | hlgG1.agly | | | | | | | | | | | | | | | | | | | | | | |
| 9922 | H2C/LO-EVQL | 60.8 | 9.7 | 0.4 | 635 | 144 | 340 | | | | | | | | | | | | Y | | | | |
| | VH-Y116A hlgG1.agly | | | | | | | | | | | | | | | | | | | | | | |
| 9400 | hlgG1.agly-(SG4)2- | 69.1 | 11.1 | 1.1 | 199 | 1190 | 2250 | | | | | | Y | | | | | | | | | | |
| | H2C/LO-EVQL VH- | | | | | | | | | | | | | | | | | | | | | | |
| | CH1 VH-N67D LC | | | | | | | | | | | | | | | | | | | | | | |
| 9947 | hlgG1.agly-(SG4)- | 70.7 | 11.4 | n.d. | n.d. | 1920 | ~3900 | | | | | | Y | | | | | | | | | | |
| | H2C/LO VL-VH scFv | | | | | | | | | | | | | | | | | | | | | | |
| | VH-N67D | | | | | | | | | | | | | | | | | | | | | | |
| 9915 | H2C/LO-EVQL | 76.6 | 12.3 | 2.3 | 587 | 420 | 520 | | | | | | Y | | | | | | | | | | |
| | VH-N67D hlgG1.agly | | | | | | | | | | | | | | | | | | | | | | |
| 9911 | H2C/LO-EVOL | 94.0 | 15.1 | 32.9 | 526 | 1240 | 4000 | | | Y | | | | | | | | | | | | | |
| | VH-Y59A hlgG1.agly | | | | | | | | | | | | | | | | | | | | | | |
| 9942 | H2C/LO-EVQL | 96.7 | 15.5 | 8.8 | 384 | 2300 | >8000 | | | | | | Y | | | | | | | | Y | Y | Y |
| | VH-N67D VL- | | | | | | | | | | | | | | | | | | | | | | |
| | N30S/R58D/S133D | | | | | | | | | | | | | | | | | | | | | | |
| | hlgG1.agly | | | | | | | | | | | | | | | | | | | | | | |
| 9928 | H2C/LO-EVQL | 105.7 | 16.9 | 0.3 | 116 | 2080 | ~5100 | Y | | | | | | | | Y | | | | | | | |
| | VH-S32D/Y69D | | | | | | | | | | | | | | | | | | | | | | |
| | hlgG1.agly | | | | | | | | | | | | | | | | | | | | | | |
| 9918 | H2C/LO-EVQL | 107.2 | 17.2 | 4.6 | 425 | 1360 | ~3000 | | | | | | | | | Y | | | | | | | |
| | VH-Y69D hlgG1.agly | | | | | | | | | | | | | | | | | | | | | | |
| 9916 | H2C/LO-EVQL | 109.2 | 17.5 | 4.0 | 456 | 1000 | 500 | | | | | | | Y | | | | | | | | | |
| | VH-N67E hlgG1.agly | | | | | | | | | | | | | | | | | | | | | | |
| 9919 | H2C/LO-EVQL | 118.7 | 19.0 | 1.4 | 508 | 740 | 2160 | | | | | | | | | | Y | | | | | | |
| | VH-Y69E hlgG1.agly | | | | | | | | | | | | | | | | | | | | | | |
| 9940 | H2C/LO-EVQL | 128.5 | 20.6 | 4.2 | 525 | 890 | 1540 | | | | | | Y | | | | | | | | Y | | Y |
| | VH-N67D VL- | | | | | | | | | | | | | | | | | | | | | | |
| | N30S/S133D hlgG1.agly | | | | | | | | | | | | | | | | | | | | | | |
| 9929 | H2C/LO-EVQL | 137.3 | 22.0 | 1.0 | 381 | 760 | 2070 | Y | | | | | | | | | Y | | | | | | |
| | VH-S32D/Y69E | | | | | | | | | | | | | | | | | | | | | | |
| | hlgG1.agly | | | | | | | | | | | | | | | | | | | | | | |
| 9927 | H2C/LO-EVQL | 148.9 | 23.8 | 1.4 | 541 | 480 | 600 | Y | | | | | | Y | | | | | | | | | |
| | VH-S32D/N67E | | | | | | | | | | | | | | | | | | | | | | |
| | hlgG1.agly | | | | | | | | | | | | | | | | | | | | | | |

### Example 3: Transcytosis Mediated by Affinity Dematured H2C/L0 Inverted Format Variants

Caco2 cells (ECACC #86010202) were seeded in 24 well transwells (CLS3379) at 25,000 cells/well in media (DMEM with 10% FBS, 1% Sodium pyruvate, 1% Glutamine, 1% NEAA, 1% Pen/Strep) for 21 days until trans-endothelial electrical resistance reached 1200-2500 Ohm cm².

Test antibodies were added to the top well at 100 nM and 48 hours later samples were collected from the bottom well.

Human IgG levels were analyzed in transwell samples by MSD immunoassay. Samples were incubated on MSD plates (MSD, Cat #L15XB-3/L11XB-3) coated with anti-hIgG Fc capture reagent (Jackson ImmunoResearch, Cat #709-006-098), detected with sulfo-tagged anti-hIgG (MSD, Cat #R32AJ-1), and quantified by interpolation on a standard curve generated for each test antibody.

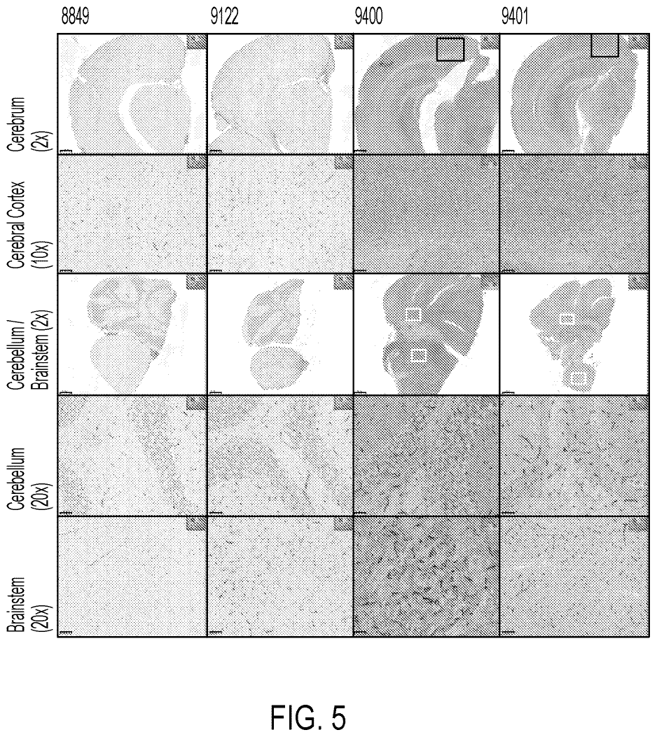

A small panel of affinity de-matured H2C/L0 variants in the inverted (upside-down) bivalent format, were tested for transcytosis across CaCo-2 monolayer. After 2 days, media from the bottom well of the transwell system was removed and concentration of antibody quantified to determine degree of transcytosis. Data are shown in Table 13, ranked by transcytosis efficiency. Notably, the parental high-affinity H2C/L0 antibodies (Protein IDs 9124 and 9393) demonstrated notably less transcytosis than the isotype control non-targeted antibody Ab2 (Protein ID 8849). However, 3 of 6 tested H2C/L0 variants demonstrated significantly more transcytosis than the non-targeted antibody (threshold 2-fold higher). Concentration-dependent binding of the tested antibodies to CHO cells is shown in FIG. 4.

TABLE 13
Transcytosis Mediated by Inverted Format Affinity Dematured H2C/LO Variants

| Pro- | | Transcytosis | | Flow Cytometry | | | | | |
|---|---|---|---|---|---|---|---|---|---|
| tein | | Accum. | Fold Over | huTfR CHO | huTfR CHO | VH- | VH- | VH- | VL- |
| ID | Protein description | Fmol | Control | EC50 (nM) | Max MFI | S32D | N67D | Y69A | R58D |
| 8849 | Ab2 hlgG1.ag | 2.21 | 1 | | | | | | |
| 9124 | hlgG1.agly-(SG4)2-H2C/LO VH-CH1 LC | 0.21 | 0.09 | 1.5 | 16 | | | | |
| 9393 | hlgG1.agly-(SG4)2-H2C/LO-EVQL VH-CH1 LC | 0.52 | 0.23 | 0.6 | 17 | | | | |
| 9395 | hlgG1.agly-DelG-H2C/LO VH-CH1 LC VL-R58D | 0.71 | 0.32 | 8 | 18 | | | | |
| 9398 | hlgG1.agly-(SG4)2-H2C/LO-EVQL VH-CH1 | 0.73 | 0.33 | 2.2 | 18 | Y | | | Y |
| | VH-S32D LC VL-R58D | | | | | | | | Y |
| 9404 | hlgG1.agly-(SG4)2-H2C/LO-EVQL VH-CH1 | 0.82 | 0.37 | 1.4 | 17 | | | | Y |
| | LC VL-R58D | | | | | | | | |
| 9401 | hlgG1.agly-(SG4)2-H2C/LO-EVQL VH-CH1 | 4.75 | 2.15 | 27.3 | 6 | | | | Y |
| | VH-N67D LC VL-R58D | | | | | | Y | | |
| 9400 | hlgG1.agly-(SG4)2-H2C/LO-EVQL VH-CH1 | 13.24 | 5.98 | 5.9 | 7 | | Y | | |
| | VH-N67D LC | | | | | | | | |
| 9402 | hlgG1.agly-(SG4)2-H2C/LO-EVQL VH-CH1 | 14.33 | 6.47 | 1 | 9 | | | Y | |
| | VH-Y69A LC | | | | | | | | |

### Example 4: Several Affinity Dematured Variants Demonstrate Increased Brain Exposure and Minimal Reticulocyte Depletion in TIR Knock-In Mice

To demonstrate the impact of reduced affinity on brain biodistribution of anti-TfR H2C/L0 variants, several exemplary variants were administered to mice expressing an engineered TfR (containing the H2C/L0 epitope), at 20 mg/kg (or molar IgG equivalent) IV by tail-vein injection (four mice per test article group). The following day (24 hours following administration) mice were anesthetized with ketamine/xylazine (100/10 mg/kg i.p.). Blood samples were collected via cardiac puncture. Then mice were perfused, through the left ventrical, with ice-cold PBS/Heparin (1u/mL) at 2 ml/minute for 10 minutes to clear the vasculature of blood. Brains were then removed and hemisected, with one hemisphere flash frozen in liquid nitrogen and the other hemisphere fixed in 10% neutral buffered formalin for 24 hours. Blood samples were analyzed for complete blood cell count, including reticulocytes, with 24 hours of collection (IDEXX). Additionally, serum was generated by allowing blood to clot for 15-30 minutes at room temperature, centrifugation at 2000 g for 10 min, freezing supernatants for further analysis.

Frozen brain hemispheres were homogenized in lysis buffer (50 mM Tris pH 7.5, 150 mM NaCl, 0.25% Na deoxycholate, 1 mM EDTA, 1% NP40, complete protease inhibitors) with zirconium oxide beads (ZROB05 and ZROB10) in a tissue homogenizer (NextAdvance Bullet Blender) for 10 minutes, then incubated at 4 degC, rotating for 1 hour. Lysates were then cleared of debris by centrifugation at 20,000 g for 20 minutes.

Human IgG levels were analyzed in serum and brain lysate by MSD immunoassay. Samples were incubated on MSD plates (MSD, Cat #L15XB-3/L11XB-3) coated with anti-hIgG Fc capture reagent (Jackson ImmunoResearch, Cat #709-006-098), detected with sulfo-tagged anti-hIgG (MSD, Cat #R32AJ-1), and quantified by interpolation on a standard curve generated for each test antibody.

After 24 hours in 10% neutral buffered formalin, fixed brain hemispheres were transferred into phosphate buffered saline, paraffin embedded, separated into six 5 mm corronal segments, sectioned 3-5 mm thick, proteinase K treated for antigen retrieval, and stained for human IgG (Southern Biotech 6145-01, 2 mg/ml, then Leica Refine HRP polymer and DAB chromogen).

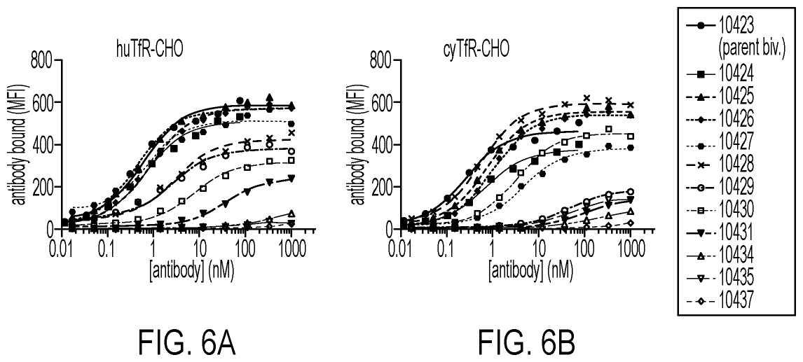

Two mutant inverted bivalent antibodies along with parental H2C/L0 as comparator, were chosen to be studied in vivo using a transgenic mouse model expressing a TfR that contains the H2C/L0 epitope. At 24 hours after dosing, the parental high-affinity bivalent H2C/L0 (Protein ID 9122) showed brain levels at 24-hours comparable to the non-targeted control antibody (Protein ID 8849), but two affinity-reduced bivalent inverted H2C/L0 variants (Protein ID 9400 and 9401) showed significantly increased brain levels, up to 4.9-fold higher than control. Enhanced exposure of the two affinity-reduced bivalent inverted H2C/L0 variants in the brain parenchyma, across multiple brain regions, was observed by hIgG immunohistochemistry and is shown in FIG. 5. The two affinity-reduced inverted variants (9400 and 9401) also demonstrated minimal or no reticulocyte depletion, suggesting favorable safety features. See Table 14.

TABLE 14
Brain Uptake and Reticulocyte Depletion by H2C/L0 and H2C/L0 Mutants

| | | Serum | Brain | Brain hIgG | |
|---|---|---|---|---|---|
| Protein | | hIgG | hIgG | fold over | Reticulocytes |
| ID | Protein description | (nM) | (nM) | control | (%) |
| 8849 | Ab2 hIgG1.ag | 2171 | 2.2 | 1 | 3.05 |
| 9122 | huH2C/L0 bivalent RSU hIgG1.agly LC hKappa | 1306 | 1.5 | 0.7 | 0.55 |
| 9400 | hIgG1.agly-(SG4)2-H2C/L0-EVQL VH-CH1 VH-N67D LC | 1215 | 10.7 | 4.9 | 2.47 |
| 9401 | hIgG1.agly-(SG4)2-H2C/L0-EVQL VH-CH1 VH-N67D LC | 1366 | 10.2 | 4.6 | 3.17 |
| | VL-R58D | | | | |

### Example 5: VH-VL or VL-VH scFv Formatting does not Significantly Change Affinity to TfR

Proteins were expressed by transient transfection of suspension CHO-S cells in serum-free medium. Protein 9915 is H2C/L0-EVQL VH-N67D hIgG1.agly (conventional RSU Ab with EVQL N-terminus of VH and bearing VH-N67D affinity reducing mutation). Protein 9945 is hG1ag Fc H2C/L0 VH-VL scFv VH-N67D (Fc-scFv inverted Ab (VH-VL format) with EVQL N-terminus of VH and bearing VH-N67D affinity reducing mutation). Conditioned supernatant was collected by centrifugation and filtration. Protein concentration was estimated using ForteBio ProA tips by extrapolation initial binding rates to a standard curve. CHO supernatants containing protein were diluted accordingly for further experiments.

For flow cytometry cell binding studies, proteins were bound to CHO cells expressing full length human TfR for 1-2 hours on ice, cells were washed twice with isotonic buffer, incubated with fluorescent secondary reagent (PE conjugated) that binds human IgG, washed twice, fixed by paraformaldehyde and analyzed on a flow cytometer. Mean fluorescence intensity (MFI) of the PE fluorophore was calculated and represents binding of antibody to cells. Due to differential recognition of the fluorescent secondary between the Inverted Ab format and the Fc-scFv Inverted Ab format (which lacks CH1 and CL domains), the MFI was normalized to the max signal of each antibody before plotting for a better comparison.

The affinity-reducing mutation VH-N67D incorporated into H2C/L0 in an scFv format resulted in minimal alteration of affinity to TfR on cells. See Table 15.

TABLE 15
H2C/L0 Mutants Binding to TfR in Different Antibody Formats

| [Ab] | ProtID | ProtID |
|---|---|---|
| (nM) | 9915 | 9945 |

Mean fluorescence intensity

| | | |
|---|---|---|
| 100 | 617 | 356 |
| 10 | 415 | 255 |
| 1 | 221 | 76 |
| 0.1 | 37 | 15 |

Normalized to max signal

| | | |
|---|---|---|
| 100 | 100 | 100 |
| 10 | 67 | 72 |
| 1 | 36 | 21 |
| 0.1 | 6 | 4 |

### Example 6: VH-VL or VL-VH scFv Formatting with or without Additional Stabilizing Disulfide does not Significantly Change Affinity to TfR

Proteins were expressed by transient transfection of suspension CHO-S cells in serum-free medium (see Protein IDs and descriptions below). Conditioned supernatant was collected by centrifugation and filtration. Protein concentration was estimated using ForteBio ProA tips by extrapolation initial binding rates to a standard curve. CHO supernatants containing protein were diluted accordingly for further experiments.

| Protein ID | Protein Description | Format / Comments |
|---|---|---|
| 9915 | EVQL VH-N67D hIgG1.agly | Conventional (upright) Ab with EVQL N-term of VH and |
| | | bearing VH-N67D affinity reducing mutation |
| 9936 | EVQL VH-N67D VL-R58D hIgG1.agly | Conventional (upright) Ab with EVQL N-term of VH and |
| | | bearing VH-N67D and VL-R58D affinity reducing |
| | | mutations |
| 9945 | hG1ag Fc VH-VL scFv VH-N67D | Fc-scFv Inverted Ab (VH-VL format) with EVQL N-term |
| | | of VH and bearing VH-N67D affinity reducing mutation |
| 9946 | hG1ag Fc VH-VL scFv VH-N67D VL- | Fc-scFv Inverted Ab (VH-VL format) with EVQL N-term |
| | R58D | of VH and bearing VH-N67D/VL-R58D affinity reducing |
| | | mutations |
| 9947 | hG1ag Fc VL-VH scFv VH-N67D | Fc-scFv Inverted Ab (VL-VH format) with EVQL N-term |
| | | of VH and bearing VH-N67D affinity reducing mutation |
| 9948 | hG1ag Fc VL-VH scFv VL-R58D VH- | Fc-scFv Inverted Ab (VL-VH format) with EVQL N-term |
| | N67D | of VH and bearing VH-N67D/VL-R58D affinity reducing |
| | | mutations |
| 9949 | hG1ag Fc VL-VH scFv VH-N67D CACA | scFv in format with CH3-linker-VL-linker-VH with affinity |
| | | reducing mutations and with vestigal rabbit intra-VH |
| | | disulfide removed by mutation (C42A/C57A; CACA) |
| 9950 | hG1ag Fc VL-VH scFv VH-N67D 51- | scFv in format with CH3-linker-VL-linker-VH with affinity |
| | 141dsb | reducing mutations and with disulfide bond from VH |
| | | AHo-51 to VL AHo-141 |
| 9951 | hG1ag Fc VL-VH scFv VH-N67D CACA | scFv in format with CH3-linker-VL-linker-VH with affinity |
| | 51-141dsb | reducing mutations and with disulfide bond from VH |
| | | AHo-51 to VL AHo-141 and with vestigal rabbit intra-VH |
| | | disulfide removed by mutation (C42A/C57A; CACA) |

For flow cytometry cell binding studies, proteins were bound to CHO cells expressing full length human TfR or full length cyno TfR for 1-2 hours on ice, cells were washed twice with isotonic buffer, incubated with fluorescent secondary reagent (PE conjugated) that binds human IgG, washed twice, fixed by paraformaldehyde and analyzed on a flow cytometer. Mean fluorescence intensity (MFI) of the PE fluorophore was calculated and represents binding of antibody to cells. Due to differential recognition of the fluorescent secondary between the Inverted Ab format and the Fc-scFv Inverted Ab format (which lacks CH1 and CL domains) and the fact that these reduced-affinity variants do not reach saturation on target at the highest concentration tested (100 nM), the signal observed at 10 nM is shown as a percent of signal observed for the same antibody at 100 nM.

For surface plasmon resonance binding studies, binding affinity and kinetics measurements were performed with a Biacore T200 instrument (Cytiva). A CM5 sensor chip was coated with anti-hIgG capture reagent, according to the manufacturer's protocol (Human Antibody Capture Kit; Cytiva), then equilibrated in running buffer: 10 mM HEPES pH 7.4, 150 mM NaCl, 3 mM EDTA, 0.05% bovine serum albumin, 0.005% surfactant P20. Anti-TfR1 H2C/L0 variant antibodies in CHO supernatant, normalized at 15 mcg/ml according to titers measured by anti-hIgG bilayer interferometry (ForteBio), were diluted in running buffer to 1 mcg/ml, and captured on the sensor chip surface at 10-100 pg/mm² from injections of 10 mcL/min for 1 min. Monomeric protein with human TfR1 apical domain (His8-BirApep-Cyno minTfR ECD huApi), was injected in single-cycle kinetics mode at increasing concentrations of 3.125, 12.5, 50, 200 and 800 nM at 30 mcL/min for 3 min each and dissociation was monitored for 7 min. Following each cycle of injections, the anti-hIgG capture surface was regenerated with 3 M MgCl₂ for 2×1 min. Affinity and kinetic parameters were analyzed by fitting sensogram data with a 1:1 binding model using the Biacore T200 Evaluation Software (Cytiva). In cases where the kinetics were determined to be too fast to be accurately determined (dissociation rate constant k_{d}>0.05/s), stead-state affinity analysis was used to estimate the equilibrium dissociation constant, K_{D}. In cases where the binding response at 800 nM TfR was not sufficient (<50% of the saturation binding response expected base on antibody capture levels) no affinity was reported (K_{D}>800 nM). Some antibodies were retested for binding monomeric TfR up to 4000 nM of antigen with human apical domain and up to 8000 nM for antigen with cynomolgus apical domain.

The affinity-reducing mutation VH-N67D or double-mutation VH-N67D/VL-R58D was incorporated into H2C/L0 in an scFv in either VH-VL or VL-VH format with minimal alteration of affinity to TfR on cells. See Tables 16 and 17. Further, the vestigial rabbit intra VH disulfide between Aho positions 42 and 57 was removed and the scFv still bound to target, and a stabilizing VH-VL disulfide was introduced between Aho positions VH-51 and VL-141 and the scFv still bound to target. See Tables 16 and 17.

TABLE 16
Monovalent Affinity Binding Measures (SPR)

| ProtID | ka (/M /s) | kd (/s) | KD (nM) |
|---|---|---|---|
| 9915 | | >0.05 | 423 |
| 9915 (repeat) | | ~0.05 | 216 |
| 9936 | | >0.05 | 462 |
| 9945 | | >0.05 | 829 |
| 9946 | | >0.05 | >800 |
| 9947 | | >0.05 | 222 |
| 9948 | | >0.05 | 449 |
| 9949 | | >0.05 | >800 |
| 9950 | 20400 | 0.017 | 834 |
| 9951 | | >0.05 | 864 |

TABLE 17
Cell Binding Measures (Flow Cytometry)

| ProtID | 10 nM | 10 nM | |
|---|---|---|---|
| 9915 | 87 | 64 | Conventional Ab |
| 9915 (repeat) | 91 | 82 | Conventional Ab |
| 9936 | 77 | 59 | Conventional Ab |
| 9945 | 79 | 65 | Inverted Fc-scFv |
| 9946 | 37 | 42 | Inverted Fc-scFv |
| 9947 | 51 | 43 | Inverted Fc-scFv |
| 9948 | 40 | 38 | Inverted Fc-scFv |
| 9949 | 52 | 47 | Inverted Fc-scFv |
| 9950 | 69 | 45 | Inverted Fc-scFv |

### Example 7: Removal of Disulfide Between CL and CH1-Upperhinge does not Significantly Change Affinity to TfR

Proteins were expressed by transient transfection of suspension CHO-S cells in serum-free medium (see Protein IDs and descriptions below). Conditioned supernatant was collected by centrifugation and filtration. Protein concentration was estimated using ForteBio ProA tips by extrapolation initial binding rates to a standard curve. CHO supernatants containing protein were diluted accordingly for further experiments.

| Protein ID | Protein Description | Format / Comments |
|---|---|---|
| 9915 | EVQL VH-N67D hIgG1.agly | Conventional Ab with EVQL N-term of VH and bearing VH- |
| | | N67D affinity reducing mutation |
| 9936 | EVQL VH-N67D VL-R58D | Conventional Ab with EVQL N-term of VH and bearing VH- |
| | hIgG1.agly | N67D and VL-R58D affinity reducing mutations |
| 9943 | EVQL VH-N67D LC-DelC214 | Conventional Ab with EVQL N-term of VH, without disulfide |
| | HC-C220S hIgG1.agly | between CL and CH1-upperhinge, and bearing VH-N67D |
| | | affinity reducing mutation |
| 9944 | EVQL VH-N67D VL-R58D LC- | Conventional Ab with EVQL N-term of VH, without disulfide |
| | DelC214 HC-C220S hIgG1.agly | between CL and CH1-upperhinge, and bearing VH-N67D and |
| | | VL-R58D affinity reducing mutaions |

For flow cytometry cell binding studies, proteins were bound to CHO cells expressing full length human TfR for 1-2 hours on ice, cells were washed twice with isotonic buffer, incubated with fluorescent secondary reagent (PE conjugated) that binds human IgG, washed twice, fixed by paraformaldehyde and analyzed on a flow cytometer. Mean fluorescence intensity of the PE fluorophore was calculated and represents binding of antibody to cells.

For surface plasmon resonance binding studies, binding affinity and kinetics measurements were performed with a Biacore T200 instrument (Cytiva). A CM5 sensor chip was coated with anti-hIgG capture reagent, according to the manufacturer's protocol (Human Antibody Capture Kit; Cytiva), then equilibrated in running buffer: 10 mM HEPES pH 7.4, 150 mM NaCl, 3 mM EDTA, 0.05% bovine serum albumin, 0.005% surfactant P20. Anti-TfR1 H2C/L0 variant antibodies in CHO supernatant, normalized at 15 mcg/ml according to titers measured by anti-hIgG bilayer interferometry (ForteBio), were diluted in running buffer to 1 mcg/ml, and captured on the sensor chip surface at 10-100 pg/mm² from injections of 10 mcL/min for 1 min. Monomeric protein with human TfR1 apical domain (His8-BirApep-Cyno minTfR ECD huApi), was injected in single-cycle kinetics mode at increasing concentrations of 3.125, 12.5, 50, 200 and 800 nM at 30 mcL/min for 3 min each and dissociation was monitored for 7 min. Following each cycle of injections, the anti-hIgG capture surface was regenerated with 3 M MgCl₂ for 2×1 min. Affinity and kinetic parameters were analyzed by fitting sensogram data with a 1:1 binding model using the Biacore T200 Evaluation Software (Cytiva). In cases where the kinetics were determined to be too fast to be accurately determined (dissociation rate constant k_{d}>0.05/s), stead-state affinity analysis was used to estimate the equilibrium dissociation constant, K_{D}. In cases where the binding response at 800 nM TfR was not sufficient (<50% of the saturation binding response expected base on antibody capture levels) no affinity was reported (K_{D}>800 nM). Some antibodies were retested for binding monomeric TfR up to 4000 nM of antigen with human apical domain and up to 8000 nM for antigen with cynomolgus apical domain. The disulfide bond between the light chain CL domain and the upper hinge region of the heavy chain was removed in a full antibody format and in the presence of affinity reducing mutations VH-N67D with or without VL-R58D and the resultant antibody still bound to target human TfR on cells. See Tables 18 and 19.

TABLE 18
Cell Binding Measures (Flow Cytometry)

| [Ab] | ProtID | ProtID | ProtID | ProtID |
|---|---|---|---|---|
| (nM) | 9915 | 9936 | 9943 | 9944 |
| 100 | 617 | 528 | n/d* | 486 |
| 10 | 415 | 246 | 393 | 297 |
| 1 | 221 | 67 | 162 | 94 |
| 0.1 | 37 | 13 | 30 | 18 |

TABLE 19
SPR affinity measures (monovalent)

| | | | |
|---|---|---|---|
| ProtID | ka (/M /s) | kd (/s) | KD (nM) |

| | | |
|---|---|---|
| 9915 | >0.05 | 423 |
| 9915 (repeat) | ~0.05 | 216 |
| 9936 | >0.05 | 462 |
| 9943 | >0.05 | >800 |
| 9944 | >0.05 | 300 |

### Example 8: Both Upright (Conventional) and Inverted (Upside-Down) Orientation of Fab Relative to Fc can Bind Well to TfR

Proteins were expressed by transient transfection of suspension CHO-S cells in serum-free medium (see Protein IDs and descriptions below). Conditioned supernatant was collected by centrifugation and filtration. For Protein ID 9915, the protein concentration in CHO supernatant was estimated using ForteBio ProA tips by extrapolation initial binding rates to a standard curve. CHO supernatants containing protein were diluted to create a concentration series. For Protein ID 9400, antibody were purified by immunoaffinity chromatography using Mab-select and monomeric non-aggregated material isolated on size-exclusion chromatography.

| Protein ID | Protein Description | Format / Comments |
|---|---|---|
| 9915 | EVQL VH-N67D hIgG1.agly | Conventional Ab (RSU) with EVQL N-term of VH and |
| | | bearing VH-N67D affinity reducing mutation |
| 9400 | hIgG1.ag Fc-EVQL-N67D bivalent | Inverted Ab (USD) with EVQL N-term of VH and bearing |
| | USD (pur.) | VH-N67D affinity reducing mutation |

For flow cytometry cell binding studies, roteins were bound to CHO cells expressing full length human TfR for 1-2 hours on ice, cells were washed twice with isotonic buffer, incubated with fluorescent secondary reagent (PE conjugated) that binds human IgG, washed twice, fixed by paraformaldehyde and analyzed on a flow cytometer. Mean fluorescence intensity (MFI) of the PE fluorophore was calculated and represents binding of antibody to cells. Due to differential recognition of the fluorescent secondary between the Inverted Ab format and the Fc-scFv Inverted Ab format (which lacks CH1 and CL domains) the MFI was normalized to the max signal of each antibody before plotting for a better comparison.

Antibodies bearing the affinity-reducing mutation N67D formatted into either a conventional (upright, right-side up) antibody format or an inverted (upside-down) antibody format bound to human TfR express on cells. See Tables 20 and 21.

TABLE 20
Flow Cytometry Cell Binding

| [Ab] | ProtID | ProtID |
|---|---|---|
| (nM) | 9915 | 9400 |
| 100 | 617 | 193 |
| 10 | 415 | 189 |
| 1 | 221 | 96 |
| 0.1 | 37.4 | 17 |

TABLE 21
Normalized Flow Cytometry Cell Binding

| [Ab] | ProtID | ProtID |
|---|---|---|
| (nM) | 9915 | 9400 |
| 100 | 100 | 100 |
| 10 | 67 | 98 |
| 1 | 36 | 50 |
| 0.1 | 6 | 9 |

### Example 9: Generation of Reference Antibody H2C/L0

Rabbit monoclonal anti-TfR1 antibodies were generated by immunization of a White New Zealand rabbit with cynomolgus transferrin receptor as the immunogen and then boosted with human transferrin receptor. Serum reactivity against human and cynomolgus transferrin receptor was confirmed and peripheral blood mononuclear cells (PBMCs) were isolated from whole blood from the rabbit. For monoclonal antibody generation, cynomolgus transferrin receptor was incubated with the PBMCs and used to capture B-cells with the correct specificity. The adherent B-cells were cultured and supernatants from individual wells were evaluated for human and cynomolgus transferrin receptor reactivity using fluorescence activated cell sorting (FACS) and enzyme-linked immunosorbent assay (ELISA). RNA was isolated from the B-cells and used for cDNA synthesis. The heavy and light (Vkappa) chains were PCR-amplified and cloned into pCR4 vector by TOPO/TA cloning. Cloned products were transformed into E. coli, and resistant colonies were sequenced using Sanger sequencing.

A chimeric rabbit/human antibody was generated using consensus rabbit VH and VL sequences combined with a human IgG1 framework. Binding to human and cynomolgus TfR1 were confirmed and affinities were determined using SPR analysis to recombinant extracellular domains of human or cynomolgus TfR1. The chimeric rabbit antibody had single digit nM monovalent affinity to human TfR1, cross reactivity to cynomolgus TfR1, less than 10× affinity difference between cynomolgus and human TfR1, no cross reactivity to TfR2, and no epitope overlap with transferrin binding region of TfR1.

The chimeric rabbit antibody was humanized using methods described in U.S. Pat. No. 8,961,976, incorporated by reference herein in its entirety. Further methods to minimize risk of immunogenicity were used as described in U.S. Pat. No. 8,349,324, incorporated by reference herein in its entirety. It has been established that this reference antibody anti-TfR1 H2C/L0, with an apical domain epitope on TfR1, does not compete with holo-transferrin for binding TfR1 (see, e.g., U.S. Pat. No. 11,827,702).

A humanized version of the chimeric rabbit antibody, designated H2C/L0, exhibited strong monovalent affinity (K_{D}<=10 nM) to both human TfR1 and cynomolgus TfR1. The VH and VL regions of H2C/L0 are shown below, with the CDRs underlined (according to the Union definition).

H2C/L0 Heavy Chain Variable Region H2C (VH):
(SEQ ID NO: 300)
QSLVESGGGLVQPGGSLRLSCAASGIDFSSSGYMCWVRQAPGKGLEWVGCIYTYSSNT
YYAASVKGRFTISKTSSTTVYLQMNSLKTEDTAVYYCARGTYGYTGYTYTMGYFSLWG
QGTLVTVSS
H2C/L0 Light Chain Variable Region L0 (VL):
(SEQ ID NO: 301)
DIQMTQSPSTLSASVGDRVTITCQASQNINSYLAWYQQKPGKAPKLLIYRASSLESGVPS
RFSGSGSGTEFTLTISSLQPDDFATYYCQSYYYSGSSNYNAFGGGTKVEIK

### Example 10: Refined Mutagenesis of H2C/L0 for Modulation of TfR Affinity and Improved Species Cross-Reactivity

To maximize the utility of anti-TfR1 H2C/L0 variants for brain delivery of therapeutic agents, a second round of mutagenesis of H2C/L0 was designed based on results in examples 1-4, aimed at generating variants with a wide range of affinities for TfR1, well matched affinity for human and cynomolgus monkey TfR1, and in both bivalent and monovalent antibody formats. Exemplary mutations and combinations of mutations used for affinity modulation of H2C/L0 in bivalent or monovalent format are listed in Table 22 and the sequences of peptide chains comprising these antibodies are listed in Table 23. As used herein, Protein ID 10423 is a bivalent antibody that includes a Peptide 1 and Peptide 2 sequence without any of the defined mutations. As used herein, Protein ID 10441 is a monovalent antibody that includes a Peptide 1 and Peptide 2 sequence without any of the defined mutations and additionally includes Fc-2022 as a Peptide 3. For minimal impact of antibody Fcγ receptor engagement, a low effector function human IgG scaffold with CH1 and CH2 domains from hIgG4, N297Q mutation preventing Fc glycosylation, and S228P mutation for improved stability was used. In addition, monovalent H2C/L0 variants were generated using the knob-in-hole Fc mutations to form Fab-Fc (knob)+Fc (hole) heterodimers, with the Fab-Fc chain further mutated to prevent Protein A binding (H435R/Y436F).

Antibodies were expressed by transient transfection of suspension CHO-S cells in serum-free medium. Conditioned supernatant was collected by centrifugation and filtration. Proteins were purified by loading supernatants on a HiTrap Mab Select SuRe column (Cytiva), eluting with 25 mM sodium phosphate, 100 mM NaCl, pH 2.8, then neutralizing with 1:60 (v:v) 500 mM sodium phosphate pH 8.6. Bivalent antibodies were >95% pure and free of aggregate by SDS microfluidic electrophoresis and analytical SEC following this single step of purification. Monovalent antibody MabSelect eluates contained excess Fc fragments and aggregate. Monovalent antibodies were further purified using CaptureSelect CH1-XL (ThermoFisher) affinity chromatography (eluted with 50 mM sodium acetate pH 4.0, then neutralized to pH 6.0-6.5 with ~1:30 1M HEPES pH7) and HiLoad Superdex 200 pg (Cytiva) size-exclusion chromatography to >95% purity, confirmed by non-reduced intact mass spectrometry.

Binding of purified bivalent and monovalent H2C/L0 affinity variants was assessed by both flow cytometry, characterizing the affinity and avidity for cell-surface TfR1, and by surface plasmon resonance, characterizing the monovalent affinity and kinetics for binding recombinant TfR1 ectodomain.

TABLE 22
Protein Descriptions of H2C/L0 Mutants and Other Antibodies

| Protein | | | | | | | | | |
|---|---|---|---|---|---|---|---|---|---|
| ID | Peptide 1 | Peptide 2 | Peptide 3 | Protein Name | Valency | Mut1 | Mut2 | Mut3 | Mut4 |
| 10424 | HC-2002 | LC-1095 | none | H2C/L0-EVQL hIgG4P.ag/G1 | Bivalent | VH- | | | |
| | | | | VH-Y116A | | Y116A | | | |
| 10425 | HC-2003 | LC-1095 | none | H2C/L0-EVQL hIgG4P.ag/G1 | Bivalent | VH- | | | |
| | | | | VH-N67D | | N67D | | | |
| 10426 | HC-2004 | LC-1095 | none | H2C/L0-EVQL hIgG4P.ag/G1 | Bivalent | VH- | VH- | | |
| | | | | VH-S32D/N67D | | S32D | N67D | | |
| 10427 | HC-2005 | LC-1095 | none | H2C/L0-EVQL hIgG4P.ag/G1 | Bivalent | VH- | | | |
| | | | | VH-Y69E | | Y69E | | | |
| 10428 | HC-2006 | LC-1095 | none | H2C/L0-EVQL hIgG4P.ag/G1 | Bivalent | VH- | | | |
| | | | | VH-N67E | | N67E | | | |
| 10429 | HC-2007 | LC-1095 | none | H2C/L0-EVQL hIgG4P.ag/G1 | Bivalent | VH- | | | |
| | | | | VH-Y59A | | Y59A | | | |
| 10430 | HC-2006 | LC-2021 | none | H2C/L0-EVQL hIgG4P.ag/G1 | Bivalent | VH- | VL- | VL- | VL- |
| | | | | VH-N67E VL-N30S/N32S/S133D | | N67E | N30S | N32S | S133D |
| 10431 | HC-2008 | LC-1095 | none | H2C/L0-EVQL hIgG4P.ag/G1 | Bivalent | VH- | | | |
| | | | | VH-Y59D | | Y59D | | | |
| 10434 | HC-2009 | LC-1095 | none | H2C/L0-EVQL hIgG4P.ag/G1 | Bivalent | VH- | VH- | | |
| | | | | VH-Y61D/N67E | | Y61D | N67E | | |
| 10435 | HC-2010 | LC-1095 | none | H2C/L0-EVQL hIgG4P.ag/G1 | Bivalent | VH- | VH- | | |
| | | | | VH-N67E/Y116A | | N67E | Y116A | | |
| 10437 | HC-2011 | LC-1095 | none | H2C/L0-EVQL hIgG4P.ag/G1 | Bivalent | VH- | VH- | | |
| | | | | VH-N67E/Y69E | | N67E | Y69E | | |
| 10442 | HC-2012 | LC-2021 | Fc-2022 | H2C/L0-EVQL hIgG4P.ag/G1 | Monovalent | VL- | VL- | VL- | |
| | | | | one-armed VL-N30S/N32S/S133D | | N30S | N32S | S133D | |
| 10443 | HC-2013 | LC-1095 | Fc-2022 | H2C/L0-EVQL hIgG4P.ag/G1 | Monovalent | VH- | | | |
| | | | | one-armed VH-S38D | | S38D | | | |
| 10444 | HC-2014 | LC-1095 | Fc-2022 | H2C/L0-EVQL hIgG4P.ag/G1 | Monovalent | VH- | | | |
| | | | | one-armed VH-Y61D | | Y61D | | | |
| 10445 | HC-2015 | LC-1095 | Fc-2022 | H2C/L0-EVQL hIgG4P.ag/G1 | Monovalent | VH- | | | |
| | | | | one-armed VH-Y116A | | Y116A | | | |
| 10446 | HC-2016 | LC-1095 | Fc-2022 | H2C/L0-EVQL hIgG4P.ag/G1 | Monovalent | VH- | | | |
| | | | | one-armed VH-N67D | | N67D | | | |
| 10447 | HC-2016 | LC-2021 | Fc-2022 | H2C/L0-EVQL hIgG4P.ag/G1 | Monovalent | VH- | VL- | VL- | VL- |
| | | | | one-armed VH-N67D VL- | | N67D | N30S | N32S | S133D |
| | | | | N30S/N32S/S133D | | | | | |
| 10448 | HC-2017 | LC-1095 | Fc-2022 | H2C/L0-EVQL hIgG4P.ag/G1 | Monovalent | VH- | | | |
| | | | | one-armed VH-Y69E | | Y69E | | | |
| 10449 | HC-2018 | LC-1095 | Fc-2022 | H2C/L0-EVQL hIgG4P.ag/G1 | Monovalent | VH- | | | |
| | | | | one-armed VH-N67E | | N67E | | | |
| 10450 | HC-2019 | LC-1095 | Fc-2022 | H2C/L0-EVQL hIgG4P.ag/G1 | Monovalent | VH- | | | |
| | | | | one-armed VH-Y59A | | Y59A | | | |
| 10451 | HC-2020 | LC-1095 | Fc-2022 | H2C/L0-EVQL hIgG4P.ag/G1 | Monovalent | VH- | | | |
| | | | | one-armed VH-Y59D | | Y59D | | | |

TABLE 23
Peptide Sequences for Antibody Components

| | | SEQ | |
|---|---|---|---|
| Peptide | Peptide | ID | |
| Name | Description | NO | Peptide Sequence |
| HC- | EVQL | | EVQLVESGGGLVQPGGSLRLSCAASGIDFSSSGYMCWVRQAPGKGLEWVGCIYTYSSNTYY |
| 2002 | hIgG4P.ag/G1 | | AASVKGRFTISKTSSTTVYLQMNSLKTEDTAVYYCARGTYGYTGATYTMGYFSLWGQGTLV |
| | VH-Y116A | | TVSSASTKGPSVFPLAPCSRSTSESTAALGCLVKDYFPEPVTVSWNSGALTSGVHTFPAVLQSS |
| | | | GLYSLSSVVTVPSSSLGTKTYTCNVDHKPSNTKVDKRVESKYGPPCPPCPAPEFLGGPSVFLFP |
| | | | PKPKDTLMISRTPEVTCVVVDVSQEDPEVQFNWYVDGVEVHNAKTKPREEQFQSTYRVVSV |
| | | | LTVLHQDWLNGKEYKCKVSNKGLPSSIEKTISKAKGQPREPQVYTLPPSRDELTKNQVSLTCL |
| | | | VKGFYPSDIAVEWESNGQPENNYKTTPPVLDSDGSFFLYSKLTVDKSRWQQGNVFSCSVMH |
| | | | EALHNHYTQKSLSLSPG |
| HC- | EVQL | | EVQLVESGGGLVQPGGSLRLSCAASGIDFSSSGYMCWVRQAPGKGLEWVGCIYTYSSDTYY |
| 2003 | hIgG4P.ag/G1 | | AASVKGRFTISKTSSTTVYLQMNSLKTEDTAVYYCARGTYGYTGYTYTMGYFSLWGQGTLV |
| | VH-N67D | | TVSSASTKGPSVFPLAPCSRSTSESTAALGCLVKDYFPEPVTVSWNSGALTSGVHTFPAVLQSS |
| | | | GLYSLSSVVTVPSSSLGTKTYTCNVDHKPSNTKVDKRVESKYGPPCPPCPAPEFLGGPSVFLFP |
| | | | PKPKDTLMISRTPEVTCVVVDVSQEDPEVQFNWYVDGVEVHNAKTKPREEQFQSTYRVVSV |
| | | | LTVLHQDWLNGKEYKCKVSNKGLPSSIEKTISKAKGQPREPQVYTLPPSRDELTKNQVSLTCL |
| | | | VKGFYPSDIAVEWESNGQPENNYKTTPPVLDSDGSFFLYSKLTVDKSRWQQGNVFSCSVMH |
| | | | EALHNHYTQKSLSLSPG |
| HC- | EVQL | | EVQLVESGGGLVQPGGSLRLSCAASGIDFDSSGYMCWVRQAPGKGLEWVGCIYTYSSDTYY |
| 2004 | hIgG4P.ag/G1 | | AASVKGRFTISKTSSTTVYLQMNSLKTEDTAVYYCARGTYGYTGYTYTMGYFSLWGQGTLV |
| | VH- | | TVSSASTKGPSVFPLAPCSRSTSESTAALGCLVKDYFPEPVTVSWNSGALTSGVHTFPAVLQSS |
| | S32D/N67D | | GLYSLSSVVTVPSSSLGTKTYTCNVDHKPSNTKVDKRVESKYGPPCPPCPAPEFLGGPSVFLFP |
| | | | PKPKDTLMISRTPEVTCVVVDVSQEDPEVQFNWYVDGVEVHNAKTKPREEQFQSTYRVVSV |
| | | | LTVLHQDWLNGKEYKCKVSNKGLPSSIEKTISKAKGQPREPQVYTLPPSRDELTKNQVSLTCL |
| | | | VKGFYPSDIAVEWESNGQPENNYKTTPPVLDSDGSFFLYSKLTVDKSRWQQGNVFSCSVMH |
| | | | EALHNHYTQKSLSLSPG |
| HC- | EVQL | | EVQLVESGGGLVQPGGSLRLSCAASGIDFSSSGYMCWVRQAPGKGLEWVGCIYTYSSNTEY |
| 2005 | hIgG4P.ag/G1 | | AASVKGRFTISKTSSTTVYLQMNSLKTEDTAVYYCARGTYGYTGYTYTMGYFSLWGQGTLV |
| | VH-Y69E | | TVSSASTKGPSVFPLAPCSRSTSESTAALGCLVKDYFPEPVTVSWNSGALTSGVHTFPAVLQSS |
| | | | GLYSLSSVVTVPSSSLGTKTYTCNVDHKPSNTKVDKRVESKYGPPCPPCPAPEFLGGPSVFLFP |
| | | | PKPKDTLMISRTPEVTCVVVDVSQEDPEVQFNWYVDGVEVHNAKTKPREEQFQSTYRVVSV |
| | | | LTVLHQDWLNGKEYKCKVSNKGLPSSIEKTISKAKGQPREPQVYTLPPSRDELTKNQVSLTCL |
| | | | VKGFYPSDIAVEWESNGQPENNYKTTPPVLDSDGSFFLYSKLTVDKSRWQQGNVFSCSVMH |
| | | | EALHNHYTQKSLSLSPG |
| HC- | EVQL | | EVQLVESGGGLVQPGGSLRLSCAASGIDFSSSGYMCWVRQAPGKGLEWVGCIYTYSSETYY |
| 2006 | hIgG4P.ag/G1 | | AASVKGRFTISKTSSTTVYLQMNSLKTEDTAVYYCARGTYGYTGYTYTMGYFSLWGQGTLV |
| | VH-N67E | | TVSSASTKGPSVFPLAPCSRSTSESTAALGCLVKDYFPEPVTVSWNSGALTSGVHTFPAVLQSS |
| | | | GLYSLSSVVTVPSSSLGTKTYTCNVDHKPSNTKVDKRVESKYGPPCPPCPAPEFLGGPSVFLFP |
| | | | PKPKDTLMISRTPEVTCVVVDVSQEDPEVQFNWYVDGVEVHNAKTKPREEQFQSTYRVVSV |
| | | | LTVLHQDWLNGKEYKCKVSNKGLPSSIEKTISKAKGQPREPQVYTLPPSRDELTKNQVSLTCL |
| | | | VKGFYPSDIAVEWESNGQPENNYKTTPPVLDSDGSFFLYSKLTVDKSRWQQGNVFSCSVMH |
| | | | EALHNHYTQKSLSLSPG |
| HC- | EVQL | | EVQLVESGGGLVQPGGSLRLSCAASGIDFSSSGYMCWVRQAPGKGLEWVGCIATYSSNTYY |
| 2007 | hIgG4P.ag/G1 | | AASVKGRFTISKTSSTTVYLQMNSLKTEDTAVYYCARGTYGYTGYTYTMGYFSLWGQGTLV |
| | VH-Y59A | | TVSSASTKGPSVFPLAPCSRSTSESTAALGCLVKDYFPEPVTVSWNSGALTSGVHTFPAVLQSS |
| | | | GLYSLSSVVTVPSSSLGTKTYTCNVDHKPSNTKVDKRVESKYGPPCPPCPAPEFLGGPSVFLFP |
| | | | PKPKDTLMISRTPEVTCVVVDVSQEDPEVQFNWYVDGVEVHNAKTKPREEQFQSTYRVVSV |
| | | | LTVLHQDWLNGKEYKCKVSNKGLPSSIEKTISKAKGQPREPQVYTLPPSRDELTKNQVSLTCL |
| | | | VKGFYPSDIAVEWESNGQPENNYKTTPPVLDSDGSFFLYSKLTVDKSRWQQGNVFSCSVMH |
| | | | EALHNHYTQKSLSLSPG |
| HC- | EVQL | | EVQLVESGGGLVQPGGSLRLSCAASGIDFSSSGYMCWVRQAPGKGLEWVGCIDTYSSNTYY |
| 2008 | hIgG4P.ag/G1 | | AASVKGRFTISKTSSTTVYLQMNSLKTEDTAVYYCARGTYGYTGYTYTMGYFSLWGQGTLV |
| | VH-Y59D | | TVSSASTKGPSVFPLAPCSRSTSESTAALGCLVKDYFPEPVTVSWNSGALTSGVHTFPAVLQSS |
| | | | GLYSLSSVVTVPSSSLGTKTYTCNVDHKPSNTKVDKRVESKYGPPCPPCPAPEFLGGPSVFLFP |
| | | | PKPKDTLMISRTPEVTCVVVDVSQEDPEVQFNWYVDGVEVHNAKTKPREEQFQSTYRVVSV |
| | | | LTVLHQDWLNGKEYKCKVSNKGLPSSIEKTISKAKGQPREPQVYTLPPSRDELTKNQVSLTCL |
| | | | VKGFYPSDIAVEWESNGQPENNYKTTPPVLDSDGSFFLYSKLTVDKSRWQQGNVFSCSVMH |
| | | | EALHNHYTQKSLSLSPG |
| HC- | EVQL | | EVQLVESGGGLVQPGGSLRLSCAASGIDFSSSGYMCWVRQAPGKGLEWVGCIYTDSSETYY |
| 2009 | hIgG4P.ag/G1 | | AASVKGRFTISKTSSTTVYLQMNSLKTEDTAVYYCARGTYGYTGYTYTMGYFSLWGQGTLV |
| | VH- | | TVSSASTKGPSVFPLAPCSRSTSESTAALGCLVKDYFPEPVTVSWNSGALTSGVHTFPAVLQSS |
| | Y61D/N67E | | GLYSLSSVVTVPSSSLGTKTYTCNVDHKPSNTKVDKRVESKYGPPCPPCPAPEFLGGPSVFLFP |
| | | | PKPKDTLMISRTPEVTCVVVDVSQEDPEVQFNWYVDGVEVHNAKTKPREEQFQSTYRVVSV |
| | | | LTVLHQDWLNGKEYKCKVSNKGLPSSIEKTISKAKGQPREPQVYTLPPSRDELTKNQVSLTCL |
| | | | VKGFYPSDIAVEWESNGQPENNYKTTPPVLDSDGSFFLYSKLTVDKSRWQQGNVFSCSVMH |
| | | | EALHNHYTQKSLSLSPG |
| HC- | EVQL | | EVQLVESGGGLVQPGGSLRLSCAASGIDFSSSGYMCWVRQAPGKGLEWVGCIYTYSSETYY |
| 2010 | hIgG4P.ag/G1 | | AASVKGRFTISKTSSTTVYLQMNSLKTEDTAVYYCARGTYGYTGATYTMGYFSLWGQGTLV |
| | VH- | | TVSSASTKGPSVFPLAPCSRSTSESTAALGCLVKDYFPEPVTVSWNSGALTSGVHTFPAVLQSS |
| | N67E/Y116A | | GLYSLSSVVTVPSSSLGTKTYTCNVDHKPSNTKVDKRVESKYGPPCPPCPAPEFLGGPSVFLFP |
| | | | PKPKDTLMISRTPEVTCVVVDVSQEDPEVQFNWYVDGVEVHNAKTKPREEQFQSTYRVVSV |
| | | | LTVLHQDWLNGKEYKCKVSNKGLPSSIEKTISKAKGQPREPQVYTLPPSRDELTKNQVSLTCL |
| | | | VKGFYPSDIAVEWESNGQPENNYKTTPPVLDSDGSFFLYSKLTVDKSRWQQGNVFSCSVMH |
| | | | EALHNHYTQKSLSLSPG |
| HC- | EVQL | | EVQLVESGGGLVQPGGSLRLSCAASGIDFSSSGYMCWVRQAPGKGLEWVGCIYTYSSETEYA |
| 2011 | hIgG4P.ag/G1 | | ASVKGRFTISKTSSTTVYLQMNSLKTEDTAVYYCARGTYGYTGYTYTMGYFSLWGQGTLVT |
| | VH- | | VSSASTKGPSVFPLAPCSRSTSESTAALGCLVKDYFPEPVTVSWNSGALTSGVHTFPAVLQSS |
| | N67E/Y69E | | GLYSLSSVVTVPSSSLGTKTYTCNVDHKPSNTKVDKRVESKYGPPCPPCPAPEFLGGPSVFLFP |
| | | | PKPKDTLMISRTPEVTCVVVDVSQEDPEVQFNWYVDGVEVHNAKTKPREEQFQSTYRVVSV |
| | | | LTVLHQDWLNGKEYKCKVSNKGLPSSIEKTISKAKGQPREPQVYTLPPSRDELTKNQVSLTCL |
| | | | VKGFYPSDIAVEWESNGQPENNYKTTPPVLDSDGSFFLYSKLTVDKSRWQQGNVFSCSVMH |
| | | | EALHNHYTQKSLSLSPG |
| HC- | EVQL | | EVQLVESGGGLVQPGGSLRLSCAASGIDFSSSGYMCWVRQAPGKGLEWVGCIYTYSSNTYY |
| 2012 | hIgG4P.ag/ | | AASVKGRFTISKTSSTTVYLQMNSLKTEDTAVYYCARGTYGYTGYTYTMGYFSLWGQGTLV |
| | G1- | | TVSSASTKGPSVFPLAPCSRSTSESTAALGCLVKDYFPEPVTVSWNSGALTSGVHTFPAVLQSS |
| | T366W/H435R/ | | GLYSLSSVVTVPSSSLGTKTYTCNVDHKPSNTKVDKRVESKYGPPCPPCPAPEFLGGPSVFLFP |
| | Y436F HC | | PKPKDTLMISRTPEVTCVVVDVSQEDPEVQFNWYVDGVEVHNAKTKPREEQFQSTYRVVSV |
| | (parent) | | LTVLHQDWLNGKEYKCKVSNKGLPSSIEKTISKAKGQPREPQVYTLPPSRDELTKNQVSLWC |
| | | | LVKGFYPSDIAVEWESNGQPENNYKTTPPVLDSDGSFFLYSKLTVDKSRWQQGNVFSCSVM |
| | | | HEALHNRFTQKSLSLSPG |
| HC- | EVQL | | EVQLVESGGGLVQPGGSLRLSCAASGIDFSSDGYMCWVRQAPGKGLEWVGCIYTYSSNTYY |
| 2013 | hIgG4P.ag/ | | AASVKGRFTISKTSSTTVYLQMNSLKTEDTAVYYCARGTYGYTGYTYTMGYFSLWGQGTLV |
| | G1- | | TVSSASTKGPSVFPLAPCSRSTSESTAALGCLVKDYFPEPVTVSWNSGALTSGVHTFPAVLQSS |
| | T366W/H435R/ | | GLYSLSSVVTVPSSSLGTKTYTCNVDHKPSNTKVDKRVESKYGPPCPPCPAPEFLGGPSVFLFP |
| | Y436F VH- | | PKPKDTLMISRTPEVTCVVVDVSQEDPEVQFNWYVDGVEVHNAKTKPREEQFQSTYRVVSV |
| | S38D | | LTVLHQDWLNGKEYKCKVSNKGLPSSIEKTISKAKGQPREPQVYTLPPSRDELTKNQVSLWC |
| | | | LVKGFYPSDIAVEWESNGQPENNYKTTPPVLDSDGSFFLYSKLTVDKSRWQQGNVFSCSVM |
| | | | HEALHNRFTQKSLSLSPG |
| HC- | EVQL | | EVQLVESGGGLVQPGGSLRLSCAASGIDFSSSGYMCWVRQAPGKGLEWVGCIYTDSSNTYY |
| 2014 | hIgG4P.ag/ | | AASVKGRFTISKTSSTTVYLQMNSLKTEDTAVYYCARGTYGYTGYTYTMGYFSLWGQGTLV |
| | G1- | | TVSSASTKGPSVFPLAPCSRSTSESTAALGCLVKDYFPEPVTVSWNSGALTSGVHTFPAVLQSS |
| | T366W/H435R/ | | GLYSLSSVVTVPSSSLGTKTYTCNVDHKPSNTKVDKRVESKYGPPCPPCPAPEFLGGPSVFLFP |
| | Y436F VH- | | PKPKDTLMISRTPEVTCVVVDVSQEDPEVQFNWYVDGVEVHNAKTKPREEQFQSTYRVVSV |
| | Y61D | | LTVLHQDWLNGKEYKCKVSNKGLPSSIEKTISKAKGQPREPQVYTLPPSRDELTKNQVSLWC |
| | | | LVKGFYPSDIAVEWESNGQPENNYKTTPPVLDSDGSFFLYSKLTVDKSRWQQGNVFSCSVM |
| | | | HEALHNRFTQKSLSLSPG |
| HC- | EVQL | | EVQLVESGGGLVQPGGSLRLSCAASGIDFSSSGYMCWVRQAPGKGLEWVGCIYTYSSNTYY |
| 2015 | hIgG4P.ag/ | | AASVKGRFTISKTSSTTVYLQMNSLKTEDTAVYYCARGTYGYTGATYTMGYFSLWGQGTLV |
| | G1- | | TVSSASTKGPSVFPLAPCSRSTSESTAALGCLVKDYFPEPVTVSWNSGALTSGVHTFPAVLQSS |
| | T366W/H435R/ | | GLYSLSSVVTVPSSSLGTKTYTCNVDHKPSNTKVDKRVESKYGPPCPPCPAPEFLGGPSVFLFP |
| | Y436F VH- | | PKPKDTLMISRTPEVTCVVVDVSQEDPEVQFNWYVDGVEVHNAKTKPREEQFQSTYRVVSV |
| | Y116A | | LTVLHQDWLNGKEYKCKVSNKGLPSSIEKTISKAKGQPREPQVYTLPPSRDELTKNQVSLWC |
| | | | LVKGFYPSDIAVEWESNGQPENNYKTTPPVLDSDGSFFLYSKLTVDKSRWQQGNVFSCSVM |
| | | | HEALHNRFTQKSLSLSPG |
| HC- | EVQL | | EVQLVESGGGLVQPGGSLRLSCAASGIDFSSSGYMCWVRQAPGKGLEWVGCIYTYSSDTYY |
| 2016 | hIgG4P.ag/ | | AASVKGRFTISKTSSTTVYLQMNSLKTEDTAVYYCARGTYGYTGYTYTMGYFSLWGQGTLV |
| | G1- | | TVSSASTKGPSVFPLAPCSRSTSESTAALGCLVKDYFPEPVTVSWNSGALTSGVHTFPAVLQSS |
| | T366W/H435R/ | | GLYSLSSVVTVPSSSLGTKTYTCNVDHKPSNTKVDKRVESKYGPPCPPCPAPEFLGGPSVFLFP |
| | Y436F VH- | | PKPKDTLMISRTPEVTCVVVDVSQEDPEVQFNWYVDGVEVHNAKTKPREEQFQSTYRVVSV |
| | N67D | | LTVLHQDWLNGKEYKCKVSNKGLPSSIEKTISKAKGQPREPQVYTLPPSRDELTKNQVSLWC |
| | | | LVKGFYPSDIAVEWESNGQPENNYKTTPPVLDSDGSFFLYSKLTVDKSRWQQGNVFSCSVM |
| | | | HEALHNRFTQKSLSLSPG |
| HC- | EVQL | | EVQLVESGGGLVQPGGSLRLSCAASGIDFSSSGYMCWVRQAPGKGLEWVGCIYTYSSNTEY |
| 2017 | hIgG4P.ag/ | | AASVKGRFTISKTSSTTVYLQMNSLKTEDTAVYYCARGTYGYTGYTYTMGYFSLWGQGTLV |
| | G1- | | TVSSASTKGPSVFPLAPCSRSTSESTAALGCLVKDYFPEPVTVSWNSGALTSGVHTFPAVLQSS |
| | T366W/H435R/ | | GLYSLSSVVTVPSSSLGTKTYTCNVDHKPSNTKVDKRVESKYGPPCPPCPAPEFLGGPSVFLFP |
| | Y436F VH- | | PKPKDTLMISRTPEVTCVVVDVSQEDPEVQFNWYVDGVEVHNAKTKPREEQFQSTYRVVSV |
| | Y69E | | LTVLHQDWLNGKEYKCKVSNKGLPSSIEKTISKAKGQPREPQVYTLPPSRDELTKNQVSLWC |
| | | | LVKGFYPSDIAVEWESNGQPENNYKTTPPVLDSDGSFFLYSKLTVDKSRWQQGNVFSCSVM |
| | | | HEALHNRFTQKSLSLSPG |
| HC- | EVQL | | EVQLVESGGGLVQPGGSLRLSCAASGIDFSSSGYMCWVRQAPGKGLEWVGCIYTYSSETYY |
| 2018 | hIgG4P.ag/ | | AASVKGRFTISKTSSTTVYLQMNSLKTEDTAVYYCARGTYGYTGYTYTMGYFSLWGQGTLV |
| | G1- | | TVSSASTKGPSVFPLAPCSRSTSESTAALGCLVKDYFPEPVTVSWNSGALTSGVHTFPAVLQSS |
| | T366W/H435R/ | | GLYSLSSVVTVPSSSLGTKTYTCNVDHKPSNTKVDKRVESKYGPPCPPCPAPEFLGGPSVFLFP |
| | Y436F VH- | | PKPKDTLMISRTPEVTCVVVDVSQEDPEVQFNWYVDGVEVHNAKTKPREEQFQSTYRVVSV |
| | N67E | | LTVLHQDWLNGKEYKCKVSNKGLPSSIEKTISKAKGQPREPQVYTLPPSRDELTKNQVSLWC |
| | | | LVKGFYPSDIAVEWESNGQPENNYKTTPPVLDSDGSFFLYSKLTVDKSRWQQGNVFSCSVM |
| | | | HEALHNRFTQKSLSLSPG |
| HC- | EVQL | | EVQLVESGGGLVQPGGSLRLSCAASGIDFSSSGYMCWVRQAPGKGLEWVGCIATYSSNTYY |
| 2019 | hIgG4P.ag/ | | AASVKGRFTISKTSSTTVYLQMNSLKTEDTAVYYCARGTYGYTGYTYTMGYFSLWGQGTLV |
| | G1- | | TVSSASTKGPSVFPLAPCSRSTSESTAALGCLVKDYFPEPVTVSWNSGALTSGVHTFPAVLQSS |
| | T366W/H435R/ | | GLYSLSSVVTVPSSSLGTKTYTCNVDHKPSNTKVDKRVESKYGPPCPPCPAPEFLGGPSVFLFP |
| | Y436F VH- | | PKPKDTLMISRTPEVTCVVVDVSQEDPEVQFNWYVDGVEVHNAKTKPREEQFQSTYRVVSV |
| | Y59A | | LTVLHQDWLNGKEYKCKVSNKGLPSSIEKTISKAKGQPREPQVYTLPPSRDELTKNQVSLWC |
| | | | LVKGFYPSDIAVEWESNGQPENNYKTTPPVLDSDGSFFLYSKLTVDKSRWQQGNVFSCSVM |
| | | | HEALHNRFTQKSLSLSPG |
| HC- | EVQL | | EVQLVESGGGLVQPGGSLRLSCAASGIDFSSSGYMCWVRQAPGKGLEWVGCIDTYSSNTYY |
| 2020 | hIgG4P.ag/ | | AASVKGRFTISKTSSTTVYLQMNSLKTEDTAVYYCARGTYGYTGYTYTMGYFSLWGQGTLV |
| | G1- | | TVSSASTKGPSVFPLAPCSRSTSESTAALGCLVKDYFPEPVTVSWNSGALTSGVHTFPAVLQSS |
| | T366W/H435R/ | | GLYSLSSVVTVPSSSLGTKTYTCNVDHKPSNTKVDKRVESKYGPPCPPCPAPEFLGGPSVFLFP |
| | Y436F VH- | | PKPKDTLMISRTPEVTCVVVDVSQEDPEVQFNWYVDGVEVHNAKTKPREEQFQSTYRVVSV |
| | Y59D | | LTVLHQDWLNGKEYKCKVSNKGLPSSIEKTISKAKGQPREPQVYTLPPSRDELTKNQVSLWC |
| | | | LVKGFYPSDIAVEWESNGQPENNYKTTPPVLDSDGSFFLYSKLTVDKSRWQQGNVFSCSVM |
| | | | HEALHNRFTQKSLSLSPG |
| LC- | LC VL- | | DIQMTQSPSTLSASVGDRVTITCQASQSISSYLAWYQQKPGKAPKLLIYRASSLESGVPSRFSG |
| 2021 | N30S/N32S/ | | SGSGTEFTLTISSLQPDDFATYYCQSYYYSGDSNYNAFGGGTKVEIKRTVAAPSVFIFPPSDEQ |
| | S133D | | LKSGTASVVCLLNNFYPREAKVQWKVDNALQSGNSQESVTEQDSKDSTYSLSSTLTLSKADY |
| | | | EKHKVYACEVTHQGLSSPVTKSFNRGEC |
| Fc- | hIgG4P.ag/ | | SKYGPPCPPCPAPEFLGGPSVFLFPPKPKDTLMISRTPEVTCVVVDVSQEDPEVQFNWYVDGV |
| 2022 | G1- | | EVHNAKTKPREEQFQSTYRVVSVLTVLHQDWLNGKEYKCKVSNKGLPSSIEKTISKAKGQPR |
| | T366S/L368A/ | | EPQVYTLPPSRDELTKNQVSLSCAVKGFYPSDIAVEWESNGQPENNYKTTPPVLDSDGSFFLV |
| | Y407V Fc | | SKLTVDKSRWQQGNVFSCSVMHEALHNHYTQKSLSLSPG |

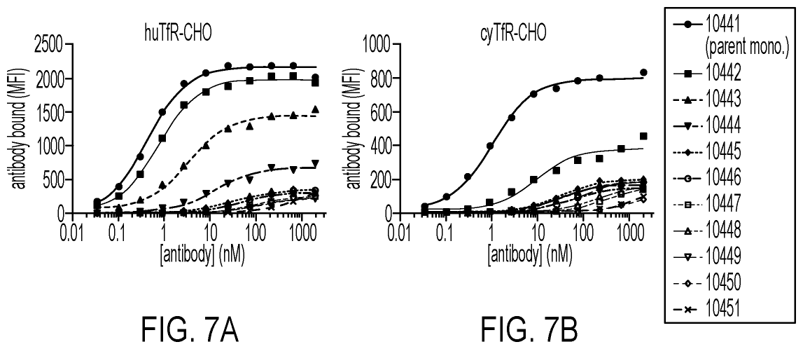

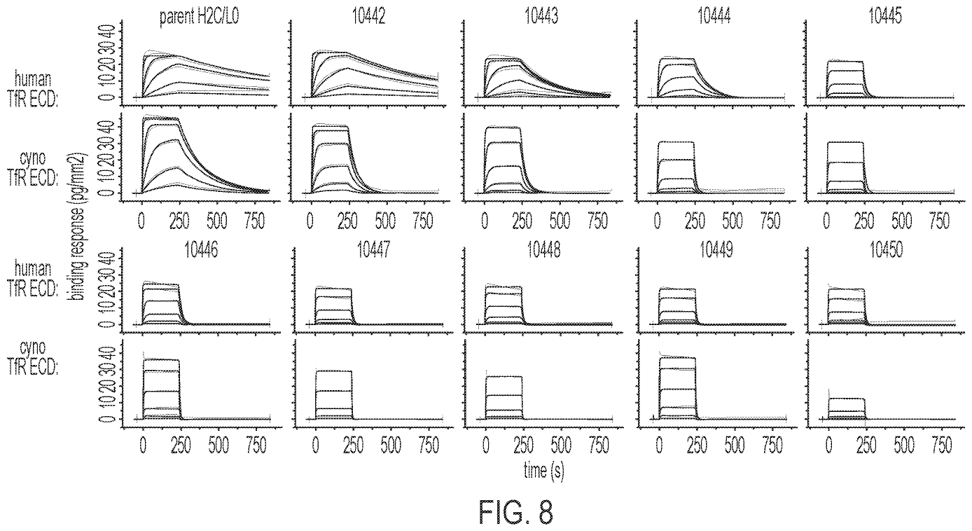

For cell binding studies, CHO cells expressing full length human TfR1 (huTfR-CHO) or cynomologus monkey TfR1 (cyTfR-CHO), without endogenous hamster TfR1 (TfR KO CHO background), were incubated with bivalent or monovalent antibodies at concentrations spanning 0.01 to 2000 nM for 1-2 hours on ice, then washed three times with isotonic buffer, incubated with fluorescent secondary reagent (PE conjugated) that binds human IgG, washed again, fixed in 1% paraformaldehyde and analyzed on a flow cytometer. Mean fluorescence intensity (MFI) of the PE fluorophore was calculated and represents binding of antibody to cells. Non-specific binding was assessed by binding of antibodies to TfR KO CHO cells. Binding data was fit to standard sigmoidal log (dose)-response curves, shown in FIGS. 6A and 6B and FIGS. 7A and 7B, with EC₅₀ and maximum MFI values reported in Table 24 and Table 25, for bivalent and monovalent antibodies, respectively. A wide range of EC₅₀ values, 0.2 to ~400 nM, were determined. In some cases, a precise EC₅₀ could not be determined due to weak binding (indicated as EC₅₀>500 nM), although all variants showed significant binding (MFI >3-fold higher on huTfR-CHO than TfR KO CHO at the highest concentration tested).

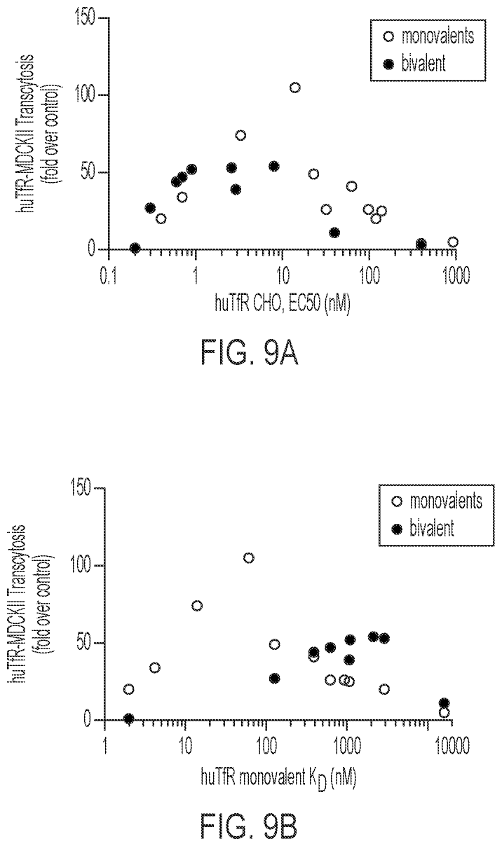

To evaluate the monovalent affinity and kinetics of H2C/L0 variants, monovalent antibodies were tested for binding to human and cynomolgus monkey TfR1 ectodomain by surface plasmon resonance. In cases where a H2C/L0 variant was not expressed as a monovalent antibody, a Fab fragment was generated from the bivalent form through papain digest (digested with papain, Roche 108014 at 10 ug per mg of antibody incubated at 37 degC for 4 hours). Histidine tagged ectodomain of human TfR1 (8×His-Gly-huTfR1 (aa89-760)) or cynomolgus monkey TfR1 (8×His-Gly-cyTfR1 (aa89-732)) was captured at 30-50 pg/mm² on a SPR chip (CM5 chip in a Biacore 8K+) coated with anti-His capture reagent (Cytiva). Monovalent antibodies were injected at concentrations ranging from 1 to 1000 nM for stronger-binding monovalent antibodies (those with EC₅₀<30 nM by flow cytometry) or 8 to 8000 nM for weaker monovalent antibodies and Fab fragments. Time-dependent binding and dissociation was analyzed with a 1:1 kinetics model in Biacore Insight Evaluation Software (FIG. 8). Affinity and kinetics parameter results are listed in Table 24 and Table 25. Monovalent dissociation kinetics varied from k_{d}~0.001/s for parental H2C/L0 antibody to >0.1/s for some of the weakest variants, and corresponding monovalent affinities were K_{D}~2 nM to >16,000 nM. The upper end of these ranges are outside of confidence for the measurements, with affinities of the weakest variants estimated by steady-state affinity analysis with a fixed saturation binding response (Rₘₐₓ).

TABLE 24
Binding properties of H2C/L0 mutant bivalent antibodies

| | | Flow Cytometry (cell surface binding) | | | | SPR (monovalent binding) | | | |
|---|---|---|---|---|---|---|---|---|---|
| | | Human TfR1-CHO | | Cyno TfR1-CHO | | Human TfR1 ECD | | Cyno TfR1 ECD | |
| ProtID | Description | EC₅₀ (nM) | Max MFI | EC₅₀ (nM) | Max MFI | k_{d} (/s) | K_{D} (nM) | k_{d} (/s) | K_{D} (nM) |
| 10423 | H2C/L0-EVLQ hIgG4P.ag/G1 | 0.2 | 608 | 0.2 | 506 | 0.0011 | 2.0 | 0.0069 | 6.3 |
| | (parent) | | | | | | | | |
| 10424 | H2C/L0-EVLQ hIgG4P.ag/G1 | 0.3 | 533 | 0.6 | 402 | 0.057 | 127 | 0.18 | 282 |
| | VH-Y116A | | | | | | | | |
| 10425 | H2C/L0-EVLQ hIgG4P.ag/G1 | 0.6 | 583 | 0.8 | 542 | 0.051 | 391 | 0.22 | 655 |
| | VH-N67D | | | | | | | | |
| 10426 | H2C/L0-EVLQ hIgG4P.ag/G1 | 0.9 | 576 | 1.0 | 541 | 0.054 | 1060 | >0.1 | 1420 |
| | VH-S32D/N67D | | | | | | | | |
| 10427 | H2C/L0-EVLQ hIgG4P.ag/G1 | 0.7 | 497 | 4.7 | 387 | >0.1 | 624 | >0.1 | 2810 |
| | VH-Y69E | | | | | | | | |
| 10428 | H2C/L0-EVLQ hIgG4P.ag/G1 | 2.9 | 454 | 0.6 | 589 | >0.1 | 1070 | >0.1 | 600 |
| | VH-N67E | | | | | | | | |
| 10429 | H2C/L0-EVLQ hIgG4P.ag/G1 | 2.6 | 367 | 43 | 177 | >0.1 | 2900 | >0.1 | ~16000 |
| | VH-Y59A | | | | | | | | |
| 10430 | H2C/L0-EVLQ hIgG4P.ag/G1 | 8.00 | 325 | 3.1 | 440 | >0.1 | 2130 | >0.1 | 2730 |
| | VH-N67E VL- | | | | | | | | |
| | N30S/N32S/S133D | | | | | | | | |
| 10431 | H2C/L0-EVLQ hIgG4P.ag/G1 | 40 | 238 | 94 | 134 | >0.1 | ~16000 | >0.1 | >16000 |
| | VH-Y59D | | | | | | | | |
| 10434 | H2C/L0-EVLQ hIgG4P.ag/G1 | ~400 | 72 | 160 | 85 | | | | |
| | VH-Y61D/N67E | | | | | | | | |
| 10435 | H2C/L0-EVLQ hIgG4P.ag/G1 | ~400 | 25 | 46 | 139 | | | | |
| | VH-N67E/Y116A | | | | | | | | |
| 10437 | H2C/L0-EVLQ hIgG4P.ag/G1 | >500 | 22 | >500 | 28 | | | | |
| | VH-N67E/Y69E | | | | | | | | |

TABLE 25
Binding properties of H2C/L0 mutant monovalent antibodies

| | | Flow Cytometry (cell surface binding) | | | | SPR (monovalent binding) | | | |
|---|---|---|---|---|---|---|---|---|---|
| | | Human TfR1-CHO | | Cyno TfR1-CHO | | Human TfR1 ECD | | Cyno TfR1 ECD | |
| ProtID | Description | EC₅₀ (nM) | Max MFI | EC₅₀ (nM) | Max MFI | k_{d} (/s) | K_{D} (nM) | k_{d} (/s) | K_{D} (nM) |
| 10441 | H2C/LO-EVQL hIgG4P.ag/G1 | 0.4 | 2023 | 1.0 | 835 | 0.0011 | 2.0 | 0.0069 | 6.3 |
| | one-armed (parent) | | | | | | | | |
| 10442 | H2C/LO-EVQL hIgG4P.ag/G1 | 0.7 | 1946 | 10 | 456 | 0.0015 | 4.2 | 0.018 | 23 |
| | one-armed VL-N30S/N32S/ | | | | | | | | |
| | S133D | | | | | | | | |
| 10443 | H2C/LO-EVQL hIgG4P.ag/G1 | 3.3 | 1547 | 38 | 209 | 0.0052 | 14.3 | 0.031 | 105 |
| | one-armed VH-S38D | | | | | | | | |
| 10444 | H2C/LO-EVQL hIgG4P.ag/G1 | 14 | 742 | 27 | 161 | 0.011 | 61 | 0.076 | 21. |
| | one-armed VH-Y61D | | | | | | | | |
| 10445 | H2C/LO-EVQL hIgG4P.ag/G1 | 23 | 285 | 31 | 187 | 0.057 | 127 | 0.18 | 282 |
| | one-armed VH-Y116A | | | | | | | | |
| 10446 | H2C/LO-EVQL hIgG4P.ag/G1 | 63 | 344 | 81 | 182 | 0.051 | 391 | 0.22 | 655 |
| | one-armed VH-N67D | | | | | | | | |
| 10447 | H2C/LO-EVQL hIgG4P.ag/G1 | 98 | 249 | 300 | 138 | 0.095 | 929 | 0.46 | 2470 |
| | one-armed VH-N67D | | | | | | | | |
| | VL-N30S/N32S/S133D | | | | | | | | |
| 10448 | H2C/LO-EVQL hIgG4P.ag/G1 | 32 | 306 | 200 | 159 | >0.1 | 624 | >0.1 | 2810 |
| | one-armed VH-Y69E | | | | | | | | |
| 10449 | H2C/LO-EVQL hIgG4P.ag/G1 | 140 | 228 | 49 | 143 | >0.1 | 1070 | >0.1 | 600 |
| | one-armed VH-N67E | | | | | | | | |
| 10450 | H2C/LO-EVQL hIgG4P.ag/G1 | 120 | 229 | >500 | 79 | >0.1 | 2900 | >0.1 | ~1600 |
| | one-armed VH-Y59A | | | | | | | | |
| 10451 | H2C/LO-EVQL hIgG4P.ag/G1 | >500 | 302 | >500 | 97 | >0.1 | ~16000 | >0.1 | >1600 |
| | one-armed VH-Y59D | | | | | | | | |

Cross-reactivity between human and cynomolgus monkey TfR1 was generally good (EC50 and K_{D}<3-fold different between species), but in some cases substantially weaker binding to one species was observed. For example, the VH-S38D, VH-Y59A, VH-Y69E, and VL-N30S/N32S/S133D mutations all weakened binding to cynomolgus monkey relative to human TfR1, whereas the VH-N67E mutations weakened binding to human more than to cynomolgus TfR1. Alternatively, the single mutation VH-Y59D, the double mutation VH-N67E/Y61D, or the combination of VH-N67E with VL-N30S/N32S/S133D all resulted in antibodies with a substantially weakened (but measurable) bivalent affinity for human TfR1 (EC₅₀~4-400 nM) with well-match affinity for cynomolgus TfR1. Similarly, the single mutations VH-Y61D, VH-N67D, VH-N67E, or VH-Y116A, or the combination of VH-N67D with VL-N30S/N32S/S133D all resulted in antibodies with monovalent affinity for human TfR1 spanning K_{D}~50-1000 nM with well-matched affinity for cynomolgus TfR1.

Thus, by refining the mutagenesis of H2C/L0, an expanded set of variants of anti-TfR1 H2C/L0 is found with differential binding properties compared to the unmodified parent H2C/L0, while maintaining species cross-reactivity between human and non-human primate.

### Example 11: Affinity Modulation of H2C/L0 Provides Optimization of Transcytosis for Both Monovalent and Bivalent Antibody Formats

To evaluate the impact of anti-TfR1 affinity modulation on TfR1-mediated transcellular trafficking for H2C/L0 variants in bivalent and monovalent format, transcytosis was measured in a hTfR1-expressing cell barrier model. Madin-Darby canine kidney II cells (MDCK II, ECACC 00062107) were stably transduced lentiviral particles encoding the human TfR1 gene and expression of human TfR1 was validated using anti-TfR antibodies in flow cytometry. hTfR-MDCKII cells were plated onto Corning 0.4 micron pore transwell inserts with 25,000 cells per insert in complete medium, cultured for five days, and treated with anti-TfR1 antibodies, or non-TfR1 targeting control hIgG, at 100 nM in the top well. After two days of treatment, samples were collected from top and bottom chambers and analyzed for concentration of the antibody using commonly available anti-human IgG reagents in a mesoscale discovery assay system.

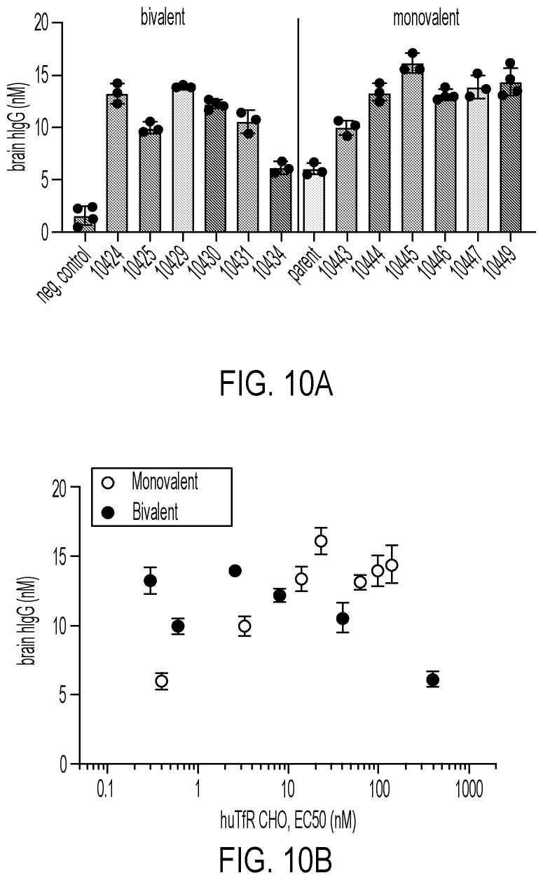

Very little transcytosis was observed for control hIgG accumulated in the transwell bottom chamber (~1 fmol). As expected, the bivalent parent H2C/L0 with high affinity (no mutations) had similar low level transcytosis as hIgG control. However, weaker affinity bivalent variants showed greatly improved transcytosis, up to ~50-fold over control (Table 26). Monovalent antibodies generally performed better in transcytosis, with the weak-affinity variants up to ~100-fold over non-targeted hIgG control and ~5-fold over parent H2C/L0 monovalent comparator (Table 27). Comparing the transcytosis function of these anti-TfR1 antibodies to their affinity/avidity for cell surface human TfR1 (EC₅₀ of binding by flow cytometry), both the bivalent and monovalent antibodies follow the same bell-shaped dependence on EC50, with an optimal function at EC₅₀~10 nM (FIG. 9A), although the monovalent antibodies achieve higher transcytosis overall (particularly the VH-Y61D mutant). On the other hand, comparing transcytosis function to the antibody monovalent affinity (K_{D} by SPR), the monovalent antibodies show optimal transport at K_{D}~20-100 nM, whereas bivalent antibodies have optimal transport at K_{D}~1000-5000 nM, in this assay (FIG. 9B).

Overall, the affinity de-modulation of anti-TfR1 H2C/L0 identified variants with optimal cellular trafficking in both bivalent and monovalent formats.

TABLE 26
In vitro transcytosis of H2C/L0 mutant bivalent antibodies

| | | Flow | | | |
|---|---|---|---|---|---|
| | | Cytometry | SPR | MDCKII-huTfR | |
| | | huTfR- | huTfR | Trancytosis | |
| | | CHO | ECD | | Fold / |
| ProtID | Description | EC₅₀ (nM) | K_{D} (nM) | fmol | Control |
| 10423 | H2C/L0-EVLQ hIgG4P.ag/G1 (parent) | 0.2 | 2.0 | 1.7 | 1.3 |
| 10424 | H2C/L0-EVLQ hIgG4P.ag/G1 VH-Y116A | 0.3 | 127 | 36 | 27 |
| 10425 | H2C/L0-EVLQ hIgG4P.ag/G1 VH-N67D | 0.6 | 391 | 58 | 44 |
| 10426 | H2C/L0-EVLQ hIgG4P.ag/G1 VH-S32D/N67D | 0.9 | 1060 | 70 | 52 |
| 10427 | H2C/L0-EVLQ hIgG4P.ag/G1 VH-Y69E | 0.7 | 624 | 63 | 47 |
| 10428 | H2C/L0-EVLQ hIgG4P.ag/G1 VH-N67E | 2.9 | 1070 | 52 | 39 |
| 10429 | H2C/L0-EVLQ hIgG4P.ag/G1 VH-Y59A | 2.6 | 2900 | 71 | 53 |
| 10430 | H2C/L0-EVLQ hIgG4P.ag/G1 VH-N67E | 8.00 | 2130 | 72 | 54 |
| | VL-N30S/N32S/S133D | | | | |
| 10431 | H2C/L0-EVLQ hIgG4P.ag/G1 VH-Y59D | 40 | ~16000 | 14 | 11 |
| 10434 | H2C/L0-EVLQ hIgG4P.ag/G1 VH-Y61D/N67E | ~400 | | 5.6 | 4.2 |
| 10435 | H2C/L0-EVLQ hIgG4P.ag/G1 VH-N67E/Y116A | ~400 | | 4.4 | 3.3 |
| 10437 | H2C/L0-EVLQ hIgG4P.ag/G1 VH-N67E/Y69E | >500 | | 9.3 | 6.9 |

TABLE 27
In vitro transcytosis of H2C/L0 mutant monovalent antibodies

| | | Flow | | | |
|---|---|---|---|---|---|
| | | Cytometry | SPR | MDCKII-huTfR | |
| | | huTfR- | huTfR | Trancytosis | |
| | | CHO | ECD | | Fold / |
| ProtID | Description | EC₅₀ (nM) | K_{D} (nM) | fmol | Control |
| parent | H2C/L0-EVQL hIgG4P.ag/G1 one-armed (parent) | 0.4 | 2.0 | 27 | 20 |
| 10442 | H2C/L0-EVQL hIgG4P.ag/G1 one-armed | 0.7 | 4.2 | 46 | 34 |
| | VL-N30S/N32S/S133D | | | | |
| 10443 | H2C/L0-EVQL hIgG4P.ag/G1 one-armed VH-S38D | 3.3 | 14.3 | 99 | 74 |
| 10444 | H2C/L0-EVQL hIgG4P.ag/G1 one-armed VH-Y61D | 14 | 61 | 142 | 105 |
| 10445 | H2C/L0-EVQL hIgG4P.ag/G1 one-armed VH-Y116A | 23 | 127 | 66 | 49 |
| 10446 | H2C/L0-EVQL hIgG4P.ag/G1 one-armed VH-N67D | 63 | 391 | 55 | 41 |
| 10447 | H2C/L0-EVQL hIgG4P.ag/G1 one-armed VH-N67D | 98 | 929 | 35 | 26 |
| | VL-N30S/N32S/S133D | | | | |
| 10448 | H2C/L0-EVQL hIgG4P.ag/G1 one-armed VH-Y69E | 32 | 624 | 34 | 26 |
| 10449 | H2C/L0-EVQL hIgG4P.ag/G1 one-armed VH-N67E | 140 | 1070 | 34 | 25 |
| 10450 | H2C/L0-EVQL hIgG4P.ag/G1 one-armed VH-Y59A | 120 | 2900 | 27 | 20 |
| 10451 | H2C/L0-EVQL hIgG4P.ag/G1 one-armed VH-Y59D | >500 | ~16000 | 6.3 | 4.7 |

### Example 12: Affinity Modulation of H2C/L0 Provides Optimization of Brain Biodistribution for Both Monovalent and Bivalent Antibody Formats

To compare the degree of brain biodistribution for anti-TfR1 H2C/L0 variants, across multiple affinities in both bivalent and monovalent formats, several exemplary variants were administered to mice expressing an engineered TfR (containing the H2C/L0 epitope), at 20 mg/kg molar IgG equivalent IV by tail-vein injection (four mice per test article group). At one or seven day(s) dosing, mice were anesthetized with ketamine/xylazine (100/10 mg/kg i.p.). Blood samples were collected via cardiac puncture. Then mice were perfused, through the left ventricle, with ice-cold PBS/Heparin (lu/mL) at 2 ml/minute for 10 minutes to clear the vasculature of blood. Brains were then removed and hemisected, with one hemisphere flash frozen in liquid nitrogen and the other hemisphere fixed in 10% neutral buffered formalin for 24 hours. Blood samples were analyzed for complete blood cell count, including reticulocytes, within 24 hours of collection (IDEXX). Additionally, serum was generated by allowing blood to clot for 15-30 minutes at room temperature, centrifugation at 2000 g for 10 min, freezing supernatants for further analysis.

Frozen brain hemispheres were homogenized in lysis buffer (50 mM Tris pH 7.5, 150 mM NaCl, 0.25% Na deoxycholate, 1 mM EDTA, 1% NP40, complete protease inhibitors) with zirconium oxide beads (ZROB05 and ZROB10) in a tissue homogenizer (NextAdvance Bullet Blender) for 10 minutes, then incubated at 4 degC, rotating for 1 hour. Lysates were then cleared of debris by centrifugation at 20,000 g for 20 minutes.

Human IgG levels were analyzed in serum and brain lysate by MSD immunoassay. Samples were incubated on MSD plates (MSD, Cat #L15XB-3/L11XB-3) coated with anti-hIgG Fc capture reagent (Jackson ImmunoResearch, Cat #709-006-098), detected with sulfo-tagged anti-hIgG (MSD, Cat #R32AJ-1), and quantified by interpolation on a standard curve generated for each test antibody.

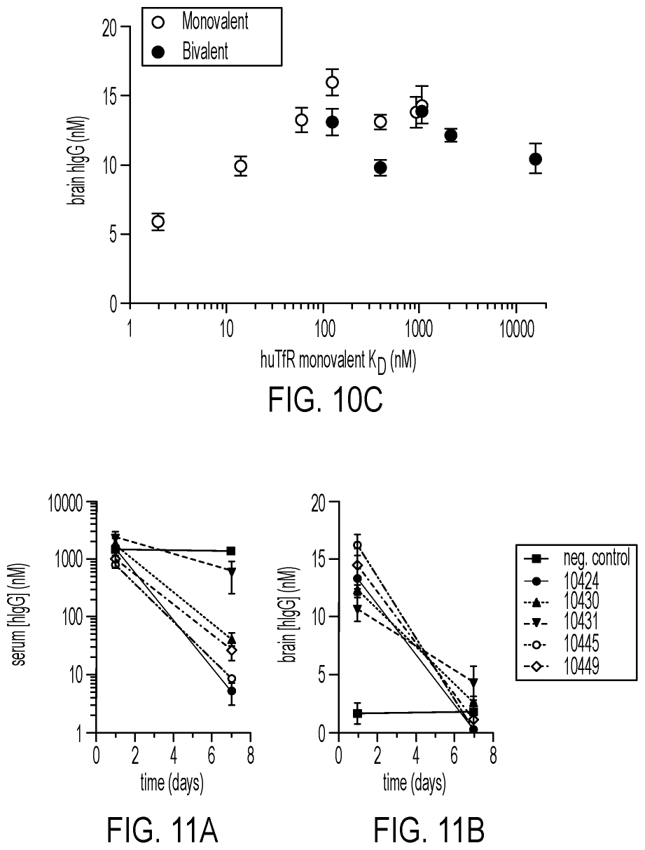

After 24 hours in 10% neutral buffered formalin, fixed brain hemispheres were transferred into phosphate buffered saline, paraffin embedded, separated into six 5 mm corronal segments, sectioned 3-5 mm thick, proteinase K treated for antigen retrieval, and stained for human IgG (Southern Biotech 6145-01, 2 mg/ml, then Leica Refine HRP polymer and DAB chromogen). Multiple anti-TfR1 H2C/L0 weakened affinity variants showed enhanced brain uptake relative to a non-targeted hIgG control antibody, one day after IV administration (FIG. 10A). Variants in both bivalent and monovalent format were detected at 10-16 nM in brain lysate, whereas control hIgG was at 1-2 nM. These antibodies had affinities/avidities for cell-surface TfR1 that spanned a wide range (EC₅₀~1-100 nM), however the optimal range for bivalent antibodies is seemingly lower (EC₅₀~1-10 nM) than for monovalent antibodies (EC₅₀~10-100 nM) (FIG. 10B). Strikingly, the degree of enhanced brain uptake seems to correspond more closely to monovalent affinity, regardless of whether the antibodies are monovalent or bivalent (FIG. 10C), suggesting that in vivo anti-TfR1 H2C/L0 bivalent antibodies engage TfR1 at the BBB primarily in a monovalent binding mode. The bell-shaped relationship between brain uptake and affinity indicates that antibodies that are either too high affinity (K_{D}<50 nM) or too weak affinity (K_{D}>~5000 nM) do not cross the BBB as efficiently as those with optimal affinity (K_{D}~50-5000 nM).

While anti-TfR1 antibodies can have elevated brain biodistribution, they also typically demonstrate increased affinity-dependent distribution to peripheral organs (where TfR1 is also expressed), leading to faster clearance from circulation and limiting long-term brain uptake. To investigate the affinity and valency dependence of sustained brain exposure of H2C/L0 variants, a subset of bivalent and monovalent variants with different affinities (all of which had elevated brain uptake at one day after dosing) were tested for serum and brain biodistribution, compared to a control hIgG, one week after IV administration at 20 mg/kg molar IgG equivalent in engineered TfR mice.

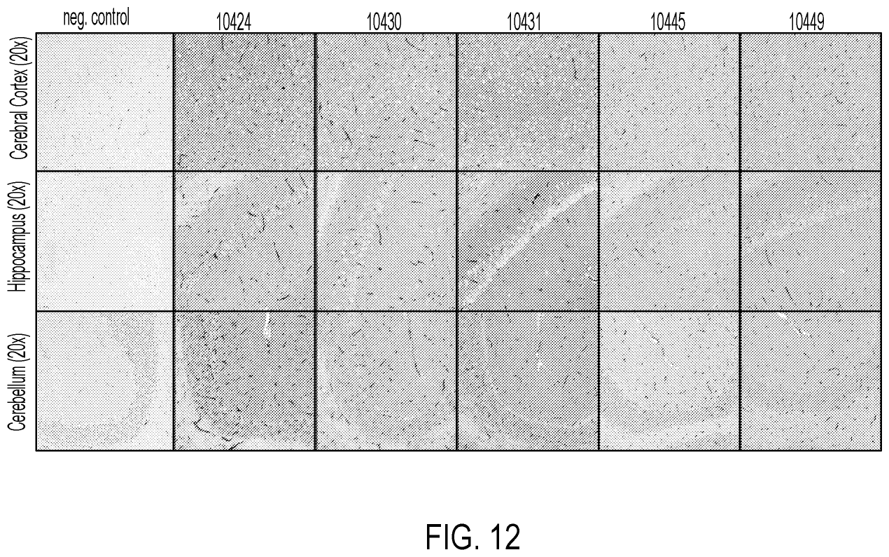

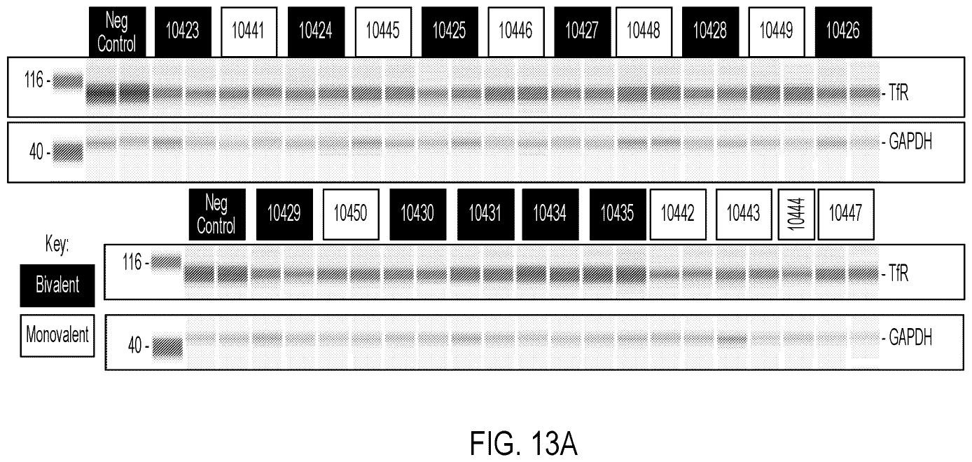

Most anti-TfR1 H2C/L0 variant antibodies had very low serum levels (~5-50 nM) compared to their levels observed one day after dosing (~800-2000 nM), whereas the control hIgG had roughly the same serum level at seven days as it did at one day after dosing (1000-2000 nM), (FIG. 11). However, the weakest-affinity bivalent antibody tested (VH-Y59D mutant; EC50~40 nM, K_{D}~16000 nM) maintained circulating serum levels of >500 nM at seven days post IV administration. Correspondingly, the levels of anti-TfR1 antibodies in mouse brain lysate dropped from well above control hIgG at one day to at or below control hIgG at seven days after IV dosing, except for the weak-affinity bivalent VH-Y59D mutant which was significantly higher than control hIgG at both one day (~7-fold, p<0.0005) and seven days (2-3 fold, p<0.05). Thus, for more sustained delivery of antibody into brain, the weak-affinity bivalent antibody profile is advantageous within this set of anti-TfR1 H2C/L0 variants, but for short-term brain delivery more modest affinity antibodies perform best (e.g. monovalent with K_{D}~100 nM). The distribution of antibody within the brain is another important consideration for CNS therapeutic delivery applications. To address the affinity and valency dependence of distribution of anti-TfR1 H2C/L0 variants within the brain, hIgG IHC was performed on brain sections from TfR-engineered mice at one day following IV administration. Representative regions of cerebral cortex, hippocampus and cerebellum were examined for three bivalent antibodies of various affinities (ProtID: 10424, 10430 and 10431; EC₅₀~0.3, 8, and 40 nM; K_{D}~130, 2100, and 16000 nM, respectively) and two monovalent antibodies (ProtID: 10445 and 10449; EC₅₀~20 and 140 nM; K_{D}~100 and 1000 nM, respectively), compared to the negative control hIgG. While some degree of vascular staining was observed for all antibodies, significantly more diffuse parenchymal staining was observed for anti-TfR1 H2C/L0 variants over control hIgG (FIG. 12), consistent with the overall brain lysate concentrations for these antibodies at the one day timepoint (FIG. 10A). While there is no striking difference in parenchymal exposure for these anti-TfR1 variants, there is a slight trend toward more parenchymal staining for weaker affinity antibodies. More obvious is that there is noticeably more staining of neuronal cell bodies (e.g. pyramidal neurons of the cortex and hippocampus, as well as Purkinje cells of the cerebellum) for the higher affinity antibodies than for the low affinity antibodies. This suggests that another differentiating factor for the choice of anti-TfR1 affinity for transporting therapeutic cargo into the brain is whether neuronal targeting is desired, and that both options are available within the range of H2C/L0 variants described here.

### Example 13: Affinity Modulation of H2C/L0 Mitigates Antibody-Induced TfR1 Degradation and Reticulocyte Depletion

To understand the role of affinity and valency in TfR1 down-modulation induced by anti-TfR1 H2C/L0 variants, bivalent and monovalent mutant antibodies were tested in an in vitro human brain endothelial cell model. hCMEC/D3 cells (Sigma, SCC066) were seeded at 30,000 cells/well in a 12-well plate, cultured for 3 days, then incubated with anti-TfR1 or control hIgG at 1000 nM for 24 hrs. Following antibody treatment, cells were lysed in RIPA buffer (Cell Signaling Technology 9806S) with phosphatase and protease inhibitors (PhosSTOP and complete; Sigma) on ice for 30 min, centrifuged at 13,000 rpm for 15 min, and supernatant was collected and analyzed for total protein by BCA (Pierce 23227). Lysates were then normalized to 100 ug/mL protein and analyzed in duplicate by capillary Western Blot (Jess, ProteinSimple), probing for total protein load (Bio-Techne DMTP01, RP-001), TfR1 (H68.4; Thermo 13-6800) and GAPDH (1D4; Novus NB300-221) with chemiluminescent secondary antibodies (Bio-Techne DM002).

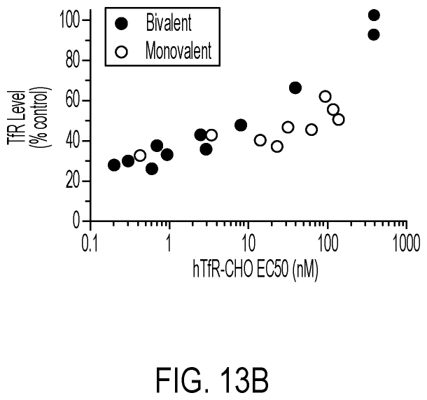

Treatment of hCMEC/D3 with most anti-TfR1 H2C/L0 mutant antibodies, in either bivalent or monovalent format, resulted in substantial reduction in TfR1 in total cell lysate (30-70% relative to control antibody treatment, FIG. 13A-13B). However, two bivalent mutant antibodies with weak affinity for TfR1 (ProtID: 10434, 10435; EC₅₀~400 nM) did not reduce TfR1, suggestive of an affinity-dependent effect. Plotting the TfR1 level versus antibody affinity/avidity for cell surface human TfR1 (FIG. 13B) confirms that there is a correlation between TfR1 loss and the strength of the mutant antibody binding. However, the monovalent mutant antibodies seem to have a stronger effect on TfR1 reduction across the full range of cell surface TfR affinities tested than the bivalent antibodies, likely because of the weaker monovalent affinity of the mutant bivalent antibodies.

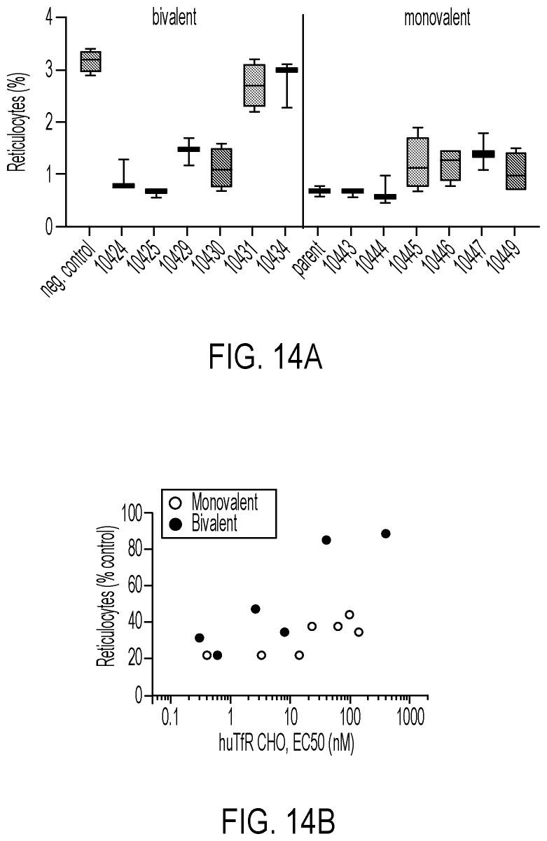

A similar analysis was conducted examining the in vivo impact of anti-TfR1 H2C/L0 mutant antibodies on circulating reticulocytes. One day after IV administration of antibodies at 20 mg/kg molar IgG equivalent, the percentage of reticulocytes in whole blood cell counts was recorded. Most anti-TfR1 H2C/L0 variants resulted in a substantial loss of reticulocytes: 50-80% loss relative to treatment with a control hIgG (FIG. 14A-14B). However, two weak affinity bivalent variants (ProtID: 10431 and 10434) did not reduce reticulocyte counts by more than 20% relative to control hIgG treatment. Reticulocyte depletion correlated with antibody affinity/avidity for cell surface TfR1 (EC₅₀ by flow cytometry of huTfR-CHO), but monovalent antibodies had a stronger effect on reticulocyte counts across the range of EC50 values tested than the bivalent antibodies (FIG. 14B).

Thus, weak affinity bivalent H2C/L0 mutants achieve effective engagement of cell surface TfR1 and transport across the BBB, with minimal impact on TfR1 expression or reticulocyte levels.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody that binds to human transferrin receptor, comprising a heavy chain variable region (VH) comprising VH complementarity determining region (CDR) 1, VH CDR2, and VH CDR3, and a light chain variable region (VL) comprising VL CDR1, VL CDR2, and VL CDR3,
wherein (a) the VH CDR1, VH CDR2, VH CDR3, VL CDR1, VL CDR2, and VL CDR3 contain collectively one to ten amino acid differences as compared to a parental antibody whose heavy chain CDRs 1, 2, and 3 comprise the amino acid sequences GIDFSSSGYMC (SEQ ID NO: XX), CIYTYSSNTYYAASVKG (SEQ ID NO:XX), and GTYGYTGYTYTMGYFSL (SEQ ID NO: XX), and whose light chain CDRs 1, 2, and 3 comprise the amino acid sequences QASQNINSYLA (SEQ ID NO:XX), RASSLES (SEQ ID NO:XX), and QSYYYSGSSNYNA (SEQ ID NO:XX), or (b) the VH CDR1, VH CDR2, VH CDR3, VL CDR1, VL CDR2, and VL CDR3 contain collectively one to ten amino acid differences as compared to a parental antibody whose heavy chain CDRs 1, 2, and 3 comprise the amino acid sequences GIDFSSSG (SEQ ID NO: XX), TYSS (SEQ ID NO:XX), and TYGYTGYTYTMGYFS (SEQ ID NO:XX), and whose light chain CDRs 1, 2, and 3 comprise the amino acid sequences SQNINSY (SEQ ID NO:XX), RAS (SEQ ID NO:XX), and YYYSGSSNYN (SEQ ID NO:XX), and
wherein the one to ten amino acid differences as compared to the parental antibody are (using AHo numbering) selected from the group consisting of:
VH-S32A, VH-S32D, or VH-S32H;
VH-S33A or VH-S33D;
VH-S33E or VH-S33H;
VH-S38A, VH-S38D, or VH-S38H;
VH-Y59A, VH-Y59D, VH-Y59H, VH-Y59E, or VH-Y59F;
VH-Y61A, VH-Y61D, or VH-Y61H;
VH-N67A, VH-N67D, VH-N67E, VH-N67H, VH-N67K, or VH-N67R;
VH-Y69A, VH-Y69D, VH-Y69E, or VH-Y69H;
VH-Y113A, VH-Y113D, or VH-Y113H;
VH-Y116A, VH-Y116D, or VH-Y116H;
VH-T130A, VH-T130D, or VH-T130H;
VH-Y131A, VH-Y131D, or VH-Y131H;
VL-N30S;
VL-N32S;
VL-R58D;
VL-Y111D, VL-Y111H, or VL-Y111S;
VL-S133A, VL-S133D, or VL-S133H;
VH-Y61D and VH-N67E;
VH-N67E and VH-Y69E; and
VL-N30S and VL-N32S.

2. The antibody of claim 1, wherein the VH CDR1, VH CDR2, VH CDR3, VL CDR1, VL CDR2, and VL CDR3 contain collectively two amino acid differences as compared to the parental antibody.

3. The antibody of claim 1, wherein the VH CDR1, VH CDR2, VH CDR3, VL CDR1, VL CDR2, and VL CDR3 contain collectively three amino acid differences as compared to the parental antibody.

4. The antibody of claim 1, wherein the VH CDR1, VH CDR2, VH CDR3, VL CDR1, VL CDR2, and VL CDR3 contain collectively four amino acid differences as compared to the parental antibody.

5. The antibody of claim 1, wherein the VH CDR1, VH CDR2, VH CDR3, VL CDR1, VL CDR2, and VL CDR3 contain collectively five amino acid differences as compared to the parental antibody.

6. The antibody of claim 1, wherein the VH CDR1, VH CDR2, VH CDR3, VL CDR1, VL CDR2, and VL CDR3 contain collectively six amino acid differences as compared to the parental antibody.

7. The antibody of claim 1, wherein:
(a) at least one of VH CDR1, VH CDR2, VH CDR3, VL CDR1, VL CDR2, and/or VL CDR3 are selected from the mutant CDRs depicted in Table 1, and any of VH CDR1, VH CDR2, VH CDR3, VL CDR1, VL CDR2, or VL CDR3 that are not selected from the mutant CDRs depicted in Table 1 are selected from the parental CDRs depicted in Table 1; or
(b) at least one of VH CDR1, VH CDR2, VH CDR3, VL CDR1, VL CDR2, and/or VL CDR3 are selected from the mutant CDRs depicted in Table 2, and any of VH CDR1, VH CDR2, VH CDR3, VL CDR1, VL CDR2, or VL CDR3 that are not selected from the mutant CDRs depicted in Table 2 are selected from the parental CDRs depicted in Table 2.

8. The antibody of claim 1, wherein:
(a) one of VH CDR1, VH CDR2, VH CDR3, VL CDR1, VL CDR2, or VL CDR3 are selected from the mutant CDRs depicted in Table 1 and five of VH CDR1, VH CDR2, VH CDR3, VL CDR1, VL CDR2, and/or VL CDR3 are selected from the parental CDRs depicted in Table 1; or
(b) one of VH CDR1, VH CDR2, VH CDR3, VL CDR1, VL CDR2, or VL CDR3 are selected from the mutant CDRs depicted in Table 2 and five of VH CDR1, VH CDR2, VH CDR3, VL CDR1, VL CDR2, and/or VL CDR3 are selected from the parental CDRs depicted in Table 2.

9. The antibody of claim 1, wherein:
(a) two of VH CDR1, VH CDR2, VH CDR3, VL CDR1, VL CDR2, and/or VL CDR3 are selected from the mutant CDRs depicted in Table 1 and four of VH CDR1, VH CDR2, VH CDR3, VL CDR1, VL CDR2, and/or VL CDR3 are selected from the parental CDRs depicted in Table 1; or
(b) two of VH CDR1, VH CDR2, VH CDR3, VL CDR1, VL CDR2, and/or VL CDR3 are selected from the mutant CDRs depicted in Table 2 and four of VH CDR1, VH CDR2, VH CDR3, VL CDR1, VL CDR2, and/or VL CDR3 are selected from the parental CDRs depicted in Table 2.

10. The antibody of claim 1, wherein:
(a) three of VH CDR1, VH CDR2, VH CDR3, VL CDR1, VL CDR2, and/or VL CDR3 are selected from the mutant CDRs depicted in Table 1 and three of VH CDR1, VH CDR2, VH CDR3, VL CDR1, VL CDR2, and/or VL CDR3 are selected from the parental CDRs depicted in Table 1; or
(b) three of VH CDR1, VH CDR2, VH CDR3, VL CDR1, VL CDR2, and/or VL CDR3 are selected from the mutant CDRs depicted in Table 2 and three of VH CDR1, VH CDR2, VH CDR3, VL CDR1, VL CDR2, and/or VL CDR3 are selected from the parental CDRs depicted in Table 2.

11. The antibody of claim 1, wherein:
(a) four of VH CDR1, VH CDR2, VH CDR3, VL CDR1, VL CDR2, and/or VL CDR3 are selected from the mutant CDRs depicted in Table 1 and two of VH CDR1, VH CDR2, VH CDR3, VL CDR1, VL CDR2, and/or VL CDR3 are selected from the parental CDRs depicted in Table 1; or
(b) four of VH CDR1, VH CDR2, VH CDR3, VL CDR1, VL CDR2, and/or VL CDR3 are selected from the mutant CDRs depicted in Table 2 and two of VH CDR1, VH CDR2, VH CDR3, VL CDR1, VL CDR2, and/or VL CDR3 are selected from the parental CDRs depicted in Table 2.

12. The antibody of claim 1, wherein:
(a) five of VH CDR1, VH CDR2, VH CDR3, VL CDR1, VL CDR2, and/or VL CDR3 are selected from the mutant CDRs depicted in Table 1 and one of VH CDR1, VH CDR2, VH CDR3, VL CDR1, VL CDR2, or VL CDR3 are selected from the parental CDRs depicted in Table 1; or
(b) five of VH CDR1, VH CDR2, VH CDR3, VL CDR1, VL CDR2, and/or VL CDR3 are selected from the mutant CDRs depicted in Table 2 and one of VH CDR1, VH CDR2, VH CDR3, VL CDR1, VL CDR2, or VL CDR3 are selected from the parental CDRs depicted in Table 2.

13. The antibody of claim 1, wherein:
(a) all of VH CDR1, VH CDR2, VH CDR3, VL CDR1, VL CDR2, and VL CDR3 are selected from the mutant CDRs depicted in Table 1; or
(b) all of VH CDR1, VH CDR2, VH CDR3, VL CDR1, VL CDR2, and VL CDR3 are selected from the mutant CDRs depicted in Table 2.

14. The antibody of claim 1, wherein:
the VH CDR1 comprises the amino acid sequence GIDFASSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFDSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFHSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSASGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSDSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSESGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSHSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSAGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSDGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSHGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIATYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIDTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIHTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIETYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIFTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTASSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTDSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIHTDSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTHSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIYTYSSATYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSETYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSHTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSKTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIYTYSSRTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTAYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTDYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTEYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTHYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGATGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGDTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGHTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGATYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGDTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGHTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYAYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYDYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYHYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTATMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTDTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTHTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQSINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNISSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence DASSLES (SEQ ID NO:XX); and
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYDSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYHSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYSSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGASNYNA (SEQ ID NO:XX);
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGHSNYNA (SEQ ID NO:XX);
the VH CDR1 comprises the amino acid sequence GIDFSSSGYMC (SEQ ID NO:XX); the VH CDR2 comprises the amino acid sequence CIYTDSSETYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
the VH CDR1 comprises the amino acid sequence GIDFSSSGYMC (SEQ ID NO:XX); the VH CDR2 comprises the amino acid sequence CIYTYSSETEYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX); or
the VH CDR1 comprises the amino acid sequence GIDFSSSGYMC (SEQ ID NO:XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO: XX); the VL CDR1 comprises the amino acid sequence QASQSISSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX).15. The antibody of claim 1, wherein:
(a) the VH CDR1 comprises the amino acid sequence GIDFDSSGYMC (SEQ ID NO: XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence DASSLES (SEQ ID NO:XX); and
(b) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence DASSLES (SEQ ID NO:XX); and the VL CDR3 comprises the amino acid sequence QSYYYSGSSNYNA (SEQ ID NO:XX);
(c) the VH CDR1 comprises the amino acid sequence GIDFDSSGYMC (SEQ ID NO: XX); the VH CDR2 comprises the amino acid sequence CIYTDSSNTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(d) the VH CDR1 comprises the amino acid sequence GIDFDSSGYMC (SEQ ID NO: XX); the VH CDR2 comprises the amino acid sequence CIYTYSSETYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(e) the VH CDR1 comprises the amino acid sequence GIDFDSSGYMC (SEQ ID NO: XX); the VH CDR2 comprises the amino acid sequence CIYTYSSNTDYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(f) the VH CDR1 comprises the amino acid sequence GIDFDSSGYMC (SEQ ID NO: XX); the VH CDR2 comprises the amino acid sequence CIYTYSSNTEYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and
(g) the VH CDR1 comprises the amino acid sequence GIDFDSSGYMC (SEQ ID NO: XX); the VH CDR2 comprises the amino acid sequence CIHTDSSNTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(h) the VH CDR1 comprises the amino acid sequence GIDFDSSGYMC (SEQ ID NO: XX); the VH CDR2 comprises the amino acid sequence CIYTYSSNTYYAASVKG (SEQ ID NO: XX); the VH CDR3 comprises the amino acid sequence GTYGYTGATYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(i) the VH CDR1 comprises the amino acid sequence GIDFSSSGYMC (SEQ ID NO: XX); the VH CDR2 comprises the amino acid sequence CIYTYSSNTAYAASVKG (SEQ ID NO: XX); the VH CDR3 comprises the amino acid sequence GTYGYTGATYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(j) the VH CDR1 comprises the amino acid sequence GIDFSSSGYMC (SEQ ID NO: XX); the VH CDR2 comprises the amino acid sequence CIYTYSSNTEYAASVKG (SEQ ID NO: XX); the VH CDR3 comprises the amino acid sequence GTYGYTGATYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(k) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence DASSLES (SEQ ID NO:XX); and
(l) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence RASSLES (SEQ ID NO:XX); and the VL CDR3 comprises the amino acid sequence QSYYSSGSSNYNA (SEQ ID NO:XX);
(m) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence RASSLES (SEQ ID NO:XX); and the VL CDR3 comprises the amino acid sequence QSYYDSGSSNYNA (SEQ ID NO:XX);
(n) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence RASSLES (SEQ ID NO:XX); and the VL CDR3 comprises the amino acid sequence QSYYYSGDSNYNA (SEQ ID NO:XX);
(o) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence DASSLES (SEQ ID NO:XX); and the VL CDR3 comprises the amino acid sequence QSYYSSGSSNYNA (SEQ ID NO:XX);
(p) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSINSYLA (SEQ ID NO: XX); the VL CDR2 comprises the amino acid sequence DASSLES (SEQ ID NO:XX); and
(q) the VH CDR1 comprises the amino acid sequence GIDFDSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(r) the VH CDR1 comprises the amino acid sequence GIDFSSSGYMC (SEQ ID NO: XX); the VH CDR2 comprises the amino acid sequence CIYTYSSETYYAASVKG (SEQ ID NO: XX); the VH CDR3 comprises the amino acid sequence GTYGYTGATYTMGYFSL (SEQ ID NO:XX); the VL CDR1 comprises the amino acid sequence QASQNINSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(s) the VH CDR1 comprises the amino acid sequence GIDFSSSGYMC (SEQ ID NO: XX); the VH CDR2 comprises the amino acid sequence CIYTYSSETYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSISSYLA (SEQ ID NO: XX); the VL CDR2 comprises the amino acid sequence RASSLES (SEQ ID NO:XX); and the VL CDR3 comprises the amino acid sequence QSYYYSGSSNYNA (SEQ ID NO:XX);
(t) the VH CDR1 comprises the amino acid sequence GIDFSSSGYMC (SEQ ID NO: XX); the VH CDR2 comprises the amino acid sequence CIYTYSSNTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSISSYLA (SEQ ID NO: XX); the VL CDR2 comprises the amino acid sequence RASSLES (SEQ ID NO:XX); and the VL CDR3 comprises the amino acid sequence QSYYYSGDSNYNA (SEQ ID NO:XX); or
(u) the VH CDR1 comprises the amino acid sequence GIDFSSSGYMC (SEQ ID NO: XX); the VH CDR2 comprises the amino acid sequence CIYTYSSDTYYAASVKG (SEQ ID NO: XX); the VH CDR3 comprises the amino acid sequence GTYGYTGYTYTMGYFSL (SEQ ID NO:XX); the VL CDR1 comprises the amino acid sequence QASQSISSYLA (SEQ ID NO: XX); the VL CDR2 comprises the amino acid sequence RASSLES (SEQ ID NO:XX); and the VL CDR3 comprises the amino acid sequence QSYYYSGDSNYNA (SEQ ID NO:XX).

16. The antibody of any one of the preceding claims, wherein:
(i) the VH is at least 80%, 85%, 90%, 95%, 99%, or 100% identical to any one of SEQ ID NOs: 100-160; and
(ii) the VL is at least 80%, 85%, 90%, 95%, 99%, or 100% identical to any one of SEQ ID NOs: 200-216.

17. The antibody of claim 1, wherein the VH comprises the amino acid sequence of any one of SEQ ID NOs: 100-160 and the VL comprises the amino acid sequence of any one of SEQ ID NOs: 200-216.

18. The antibody of any one of the preceding claims, wherein the antibody is (a) monovalent and has a monovalent affinity (K_{D}) for hTfR1 of >10 nM or is bivalent and has a monovalent affinity (K_{D}) for hTfR1 of >100 nM and/or (b) has an off rate (k_{d}) of >=0.01/s.

19. The antibody of claim 1, wherein:
the VH comprises the amino acid sequence of SEQ ID NO: 100 and the VL comprises the amino acid sequence of SEQ ID NO:301;
the VH comprises the amino acid sequence of SEQ ID NO: 101 and the VL comprises the amino acid sequence of SEQ ID NO:301;
the VH comprises the amino acid sequence of SEQ ID NO: 102 and the VL comprises the amino acid sequence of SEQ ID NO:301;
the VH comprises the amino acid sequence of SEQ ID NO:103 and the VL comprises the amino acid sequence of SEQ ID NO:301;
the VH comprises the amino acid sequence of SEQ ID NO: 104 and the VL comprises the amino acid sequence of SEQ ID NO:301;
the VH comprises the amino acid sequence of SEQ ID NO: 105 and the VL comprises the amino acid sequence of SEQ ID NO:301;
the VH comprises the amino acid sequence of SEQ ID NO: 106 and the VL comprises the amino acid sequence of SEQ ID NO:301;
the VH comprises the amino acid sequence of SEQ ID NO: 107 and the VL comprises the amino acid sequence of SEQ ID NO:301;
the VH comprises the amino acid sequence of SEQ ID NO: 108 and the VL comprises the amino acid sequence of SEQ ID NO:301;
the VH comprises the amino acid sequence of SEQ ID NO: 109 and the VL comprises the amino acid sequence of SEQ ID NO:301;
the VH comprises the amino acid sequence of SEQ ID NO:110 and the VL comprises the amino acid sequence of SEQ ID NO:301;
the VH comprises the amino acid sequence of SEQ ID NO: 111 and the VL comprises the amino acid sequence of SEQ ID NO:301;
the VH comprises the amino acid sequence of SEQ ID NO: 112 and the VL comprises the amino acid sequence of SEQ ID NO:301;
the VH comprises the amino acid sequence of SEQ ID NO: 113 and the VL comprises the amino acid sequence of SEQ ID NO:301;
the VH comprises the amino acid sequence of SEQ ID NO: 114 and the VL comprises the amino acid sequence of SEQ ID NO:301;
the VH comprises the amino acid sequence of SEQ ID NO:115 and the VL comprises the amino acid sequence of SEQ ID NO:301;
the VH comprises the amino acid sequence of SEQ ID NO: 116 and the VL comprises the amino acid sequence of SEQ ID NO:301;
the VH comprises the amino acid sequence of SEQ ID NO: 117 and the VL comprises the amino acid sequence of SEQ ID NO:301;
the VH comprises the amino acid sequence of SEQ ID NO: 118 and the VL comprises the amino acid sequence of SEQ ID NO:301;
the VH comprises the amino acid sequence of SEQ ID NO: 119 and the VL comprises the amino acid sequence of SEQ ID NO:301;
the VH comprises the amino acid sequence of SEQ ID NO: 120 and the VL comprises the amino acid sequence of SEQ ID NO:301;
the VH comprises the amino acid sequence of SEQ ID NO: 121 and the VL comprises the amino acid sequence of SEQ ID NO:301;
the VH comprises the amino acid sequence of SEQ ID NO: 122 and the VL comprises the amino acid sequence of SEQ ID NO:301;
the VH comprises the amino acid sequence of SEQ ID NO: 123 and the VL comprises the amino acid sequence of SEQ ID NO:301;
the VH comprises the amino acid sequence of SEQ ID NO: 124 and the VL comprises the amino acid sequence of SEQ ID NO:301;
the VH comprises the amino acid sequence of SEQ ID NO:125 and the VL comprises the amino acid sequence of SEQ ID NO:301;
the VH comprises the amino acid sequence of SEQ ID NO:126 and the VL comprises the amino acid sequence of SEQ ID NO:301;
the VH comprises the amino acid sequence of SEQ ID NO: 127 and the VL comprises the amino acid sequence of SEQ ID NO:301;
the VH comprises the amino acid sequence of SEQ ID NO: 128 and the VL comprises the amino acid sequence of SEQ ID NO:301;
the VH comprises the amino acid sequence of SEQ ID NO: 129 and the VL comprises the amino acid sequence of SEQ ID NO:301;
the VH comprises the amino acid sequence of SEQ ID NO:130 and the VL comprises the amino acid sequence of SEQ ID NO:301;
the VH comprises the amino acid sequence of SEQ ID NO: 131 and the VL comprises the amino acid sequence of SEQ ID NO:301;
the VH comprises the amino acid sequence of SEQ ID NO: 132 and the VL comprises the amino acid sequence of SEQ ID NO:301;
the VH comprises the amino acid sequence of SEQ ID NO: 133 and the VL comprises the amino acid sequence of SEQ ID NO:301;
the VH comprises the amino acid sequence of SEQ ID NO: 134 and the VL comprises the amino acid sequence of SEQ ID NO:301;
the VH comprises the amino acid sequence of SEQ ID NO:135 and the VL comprises the amino acid sequence of SEQ ID NO:301;
the VH comprises the amino acid sequence of SEQ ID NO: 136 and the VL comprises the amino acid sequence of SEQ ID NO:301;
the VH comprises the amino acid sequence of SEQ ID NO: 137 and the VL comprises the amino acid sequence of SEQ ID NO:301;
the VH comprises the amino acid sequence of SEQ ID NO: 138 and the VL comprises the amino acid sequence of SEQ ID NO:301;
the VH comprises the amino acid sequence of SEQ ID NO:139 and the VL comprises the amino acid sequence of SEQ ID NO:301;
the VH comprises the amino acid sequence of SEQ ID NO: 140 and the VL comprises the amino acid sequence of SEQ ID NO:301;
the VH comprises the amino acid sequence of SEQ ID NO: 141 and the VL comprises the amino acid sequence of SEQ ID NO:301;
the VH comprises the amino acid sequence of SEQ ID NO: 142 and the VL comprises the amino acid sequence of SEQ ID NO:301;
the VH comprises the amino acid sequence of SEQ ID NO: 143 and the VL comprises the amino acid sequence of SEQ ID NO:301;
the VH comprises the amino acid sequence of SEQ ID NO: 144 and the VL comprises the amino acid sequence of SEQ ID NO:301;
the VH comprises the amino acid sequence of SEQ ID NO: 145 and the VL comprises the amino acid sequence of SEQ ID NO:301;
the VH comprises the amino acid sequence of SEQ ID NO:146 and the VL comprises the amino acid sequence of SEQ ID NO:301;
the VH comprises the amino acid sequence of SEQ ID NO:147 and the VL comprises the amino acid sequence of SEQ ID NO:301;
the VH comprises the amino acid sequence of SEQ ID NO: 148 and the VL comprises the amino acid sequence of SEQ ID NO:301;
the VH comprises the amino acid sequence of SEQ ID NO: 149 and the VL comprises the amino acid sequence of SEQ ID NO:301;
the VH comprises the amino acid sequence of SEQ ID NO: 150 and the VL comprises the amino acid sequence of SEQ ID NO:301;
the VH comprises the amino acid sequence of SEQ ID NO: 151 and the VL comprises the amino acid sequence of SEQ ID NO:301;
the VH comprises the amino acid sequence of SEQ ID NO: 152 and the VL comprises the amino acid sequence of SEQ ID NO:301;
the VH comprises the amino acid sequence of SEQ ID NO: 153 and the VL comprises the amino acid sequence of SEQ ID NO:301;
the VH comprises the amino acid sequence of SEQ ID NO: 154 and the VL comprises the amino acid sequence of SEQ ID NO:301;
the VH comprises the amino acid sequence of SEQ ID NO: 155 and the VL comprises the amino acid sequence of SEQ ID NO:301;
the VH comprises the amino acid sequence of SEQ ID NO: 156 and the VL comprises the amino acid sequence of SEQ ID NO:301;
the VH comprises the amino acid sequence of SEQ ID NO: 157 and the VL comprises the amino acid sequence of SEQ ID NO:301;
the VH comprises the amino acid sequence of SEQ ID NO: 158 and the VL comprises the amino acid sequence of SEQ ID NO:301;
the VH comprises the amino acid sequence of SEQ ID NO: 159 and the VL comprises the amino acid sequence of SEQ ID NO:301;
the VH comprises the amino acid sequence of SEQ ID NO: 160 and the VL comprises the amino acid sequence of SEQ ID NO:301;
the VH comprises the amino acid sequence of SEQ ID NO:300 and the VL comprises the amino acid sequence of SEQ ID NO:200;
the VH comprises the amino acid sequence of SEQ ID NO:300 and the VL comprises the amino acid sequence of SEQ ID NO:201;
the VH comprises the amino acid sequence of SEQ ID NO:300 and the VL comprises the amino acid sequence of SEQ ID NO:202;
the VH comprises the amino acid sequence of SEQ ID NO:300 and the VL comprises the amino acid sequence of SEQ ID NO:203;
the VH comprises the amino acid sequence of SEQ ID NO:300 and the VL comprises the amino acid sequence of SEQ ID NO:204;
the VH comprises the amino acid sequence of SEQ ID NO:300 and the VL comprises the amino acid sequence of SEQ ID NO:205;
the VH comprises the amino acid sequence of SEQ ID NO:300 and the VL comprises the amino acid sequence of SEQ ID NO:206;
the VH comprises the amino acid sequence of SEQ ID NO:300 and the VL comprises the amino acid sequence of SEQ ID NO:207;
the VH comprises the amino acid sequence of SEQ ID NO:300 and the VL comprises the amino acid sequence of SEQ ID NO:208;
the VH comprises the amino acid sequence of SEQ ID NO:300 and the VL comprises the amino acid sequence of SEQ ID NO:209;
the VH comprises the amino acid sequence of SEQ ID NO:300 and the VL comprises the amino acid sequence of SEQ ID NO:210;
the VH comprises the amino acid sequence of SEQ ID NO:300 and the VL comprises the amino acid sequence of SEQ ID NO:211;
the VH comprises the amino acid sequence of SEQ ID NO:300 and the VL comprises the amino acid sequence of SEQ ID NO:212;
the VH comprises the amino acid sequence of SEQ ID NO:300 and the VL comprises the amino acid sequence of SEQ ID NO:213;
the VH comprises the amino acid sequence of SEQ ID NO:300 and the VL comprises the amino acid sequence of SEQ ID NO:214; or
the VH comprises the amino acid sequence of SEQ ID NO:300 and the VL comprises the amino acid sequence of SEQ ID NO:215;
the VH comprises the amino acid sequence of SEQ ID NO:300 and the VL comprises the amino acid sequence of SEQ ID NO:216.

20. The antibody of claim 1, wherein:
(a) the VH comprises the amino acid sequence of SEQ ID NO: 112 and the VL comprises the amino acid sequence of SEQ ID NO:202;
(b) the VH comprises the amino acid sequence of SEQ ID NO:128 and the VL comprises the amino acid sequence of SEQ ID NO:202;
(c) the VH comprises the amino acid sequence of SEQ ID NO:150 and the VL comprises the amino acid sequence of SEQ ID NO:301;
(d) the VH comprises the amino acid sequence of SEQ ID NO: 152 and the VL comprises the amino acid sequence of SEQ ID NO:301;
(e) the VH comprises the amino acid sequence of SEQ ID NO:153 and the VL comprises the amino acid sequence of SEQ ID NO:301;
(f) the VH comprises the amino acid sequence of SEQ ID NO: 154 and the VL comprises the amino acid sequence of SEQ ID NO:301;
(g) the VH comprises the amino acid sequence of SEQ ID NO:155 and the VL comprises the amino acid sequence of SEQ ID NO:301;
(h) the VH comprises the amino acid sequence of SEQ ID NO: 147 and the VL comprises the amino acid sequence of SEQ ID NO:301;
(i) the VH comprises the amino acid sequence of SEQ ID NO:148 and the VL comprises the amino acid sequence of SEQ ID NO:301;
(j) the VH comprises the amino acid sequence of SEQ ID NO:149 and the VL comprises the amino acid sequence of SEQ ID NO:301;
(k) the VH comprises the amino acid sequence of SEQ ID NO: 128 and the VL comprises the amino acid sequence of SEQ ID NO:209;
(l) the VH comprises the amino acid sequence of SEQ ID NO:128 and the VL comprises the amino acid sequence of SEQ ID NO:214;
(m) the VH comprises the amino acid sequence of SEQ ID NO: 128 and the VL comprises the amino acid sequence of SEQ ID NO:212;
(n) the VH comprises the amino acid sequence of SEQ ID NO: 128 and the VL comprises the amino acid sequence of SEQ ID NO:213;
(o) the VH comprises the amino acid sequence of SEQ ID NO: 128 and the VL comprises the amino acid sequence of SEQ ID NO:210;
(p) the VH comprises the amino acid sequence of SEQ ID NO:128 and the VL comprises the amino acid sequence of SEQ ID NO:211;
(q) the VH comprises the amino acid sequence of SEQ ID NO: 129 and the VL comprises the amino acid sequence of SEQ ID NO:216; or
(r) the VH comprises the amino acid sequence of SEQ ID NO:128 and the VL comprises the amino acid sequence of SEQ ID NO:216.

21. The antibody of any one of claims 1 to 20, which is a multispecific antibody, bispecific antibody, single chain antibody, an Fab fragment, an F(ab′)₂ fragment, an Fab′ fragment, an Fsc fragment, an Fv fragment, an scFv, an sc(Fv)₂, or a diabody.

22. The antibody of any one of claims 1 to 20, comprising a constant heavy chain (CH) domain and a constant light chain (CL) domain.

23. The antibody of any one of the preceding claims, wherein:
(i) the HC comprises an amino acid sequence that is at least 80%, 85%, 90%, 95%, 99%, or 100% identical to the amino acid sequence as set forth in any one of HC-1043 to HC-1094 (as shown in Table 10) and HC-2002 to HC-2020 (as shown in Table 22); and
(ii) the LC comprises an amino acid sequence that is at least 80%, 85%, 90%, 95%, 99%, or 100% identical to the amino acid sequence as set forth in any one of LC-1095 to LC-1114 (as shown in Table 10) and LC-2021 (as shown in Table 22).

24. The antibody of claim 1, wherein the antibody comprises:
a heavy chain comprising the amino acid sequence set forth in HC-1043, and a light chain comprising the amino acid sequence set forth in LC-1096;
a heavy chain comprising the amino acid sequence set forth in HC-1044, and a light chain comprising the amino acid sequence set forth in LC-1096;
a heavy chain comprising the amino acid sequence set forth in HC-1045, and a light chain comprising the amino acid sequence set forth in LC-1096;
a heavy chain comprising the amino acid sequence set forth in HC-1046, and a light chain comprising the amino acid sequence set forth in LC-1096;
a heavy chain comprising the amino acid sequence set forth in HC-1047, and a light chain comprising the amino acid sequence set forth in LC-1096;
a heavy chain comprising the amino acid sequence set forth in HC-1048, and a light chain comprising the amino acid sequence set forth in LC-1096;
a heavy chain comprising the amino acid sequence set forth in HC-1049, and a light chain comprising the amino acid sequence set forth in LC-1096;
a heavy chain comprising the amino acid sequence set forth in HC-1050, and a light chain comprising the amino acid sequence set forth in LC-1096;
a heavy chain comprising the amino acid sequence set forth in HC-1051, and a light chain comprising the amino acid sequence set forth in LC-1096;
a heavy chain comprising the amino acid sequence set forth in HC-1052, and a light chain comprising the amino acid sequence set forth in LC-1096;
a heavy chain comprising the amino acid sequence set forth in HC-1053, and a light chain comprising the amino acid sequence set forth in LC-1096;
a heavy chain comprising the amino acid sequence set forth in HC-1055, and a light chain comprising the amino acid sequence set forth in LC-1096;
a heavy chain comprising the amino acid sequence set forth in HC-1056, and a light chain comprising the amino acid sequence set forth in LC-1096;
a heavy chain comprising the amino acid sequence set forth in HC-1057, and a light chain comprising the amino acid sequence set forth in LC-1096;
a heavy chain comprising the amino acid sequence set forth in HC-1058, and a light chain comprising the amino acid sequence set forth in LC-1096;
a heavy chain comprising the amino acid sequence set forth in HC-1059, and a light chain comprising the amino acid sequence set forth in LC-1096;
a heavy chain comprising the amino acid sequence set forth in HC-1060, and a light chain comprising the amino acid sequence set forth in LC-1096;
a heavy chain comprising the amino acid sequence set forth in HC-1061, and a light chain comprising the amino acid sequence set forth in LC-1096;
a heavy chain comprising the amino acid sequence set forth in HC-1062, and a light chain comprising the amino acid sequence set forth in LC-1096;
a heavy chain comprising the amino acid sequence set forth in HC-1063, and a light chain comprising the amino acid sequence set forth in LC-1096;
a heavy chain comprising the amino acid sequence set forth in HC-1064, and a light chain comprising the amino acid sequence set forth in LC-1096;
a heavy chain comprising the amino acid sequence set forth in HC-1065, and a light chain comprising the amino acid sequence set forth in LC-1096;
a heavy chain comprising the amino acid sequence set forth in HC-1066, and a light chain comprising the amino acid sequence set forth in LC-1096;
a heavy chain comprising the amino acid sequence set forth in HC-1067, and a light chain comprising the amino acid sequence set forth in LC-1096;
a heavy chain comprising the amino acid sequence set forth in HC-1068, and a light chain comprising the amino acid sequence set forth in LC-1096;
a heavy chain comprising the amino acid sequence set forth in HC-1069, and a light chain comprising the amino acid sequence set forth in LC-1096;
a heavy chain comprising the amino acid sequence set forth in HC-1070, and a light chain comprising the amino acid sequence set forth in LC-1096;
a heavy chain comprising the amino acid sequence set forth in HC-1071, and a light chain comprising the amino acid sequence set forth in LC-1096;
a heavy chain comprising the amino acid sequence set forth in HC-1072, and a light chain comprising the amino acid sequence set forth in LC-1096;
a heavy chain comprising the amino acid sequence set forth in HC-1073, and a light chain comprising the amino acid sequence set forth in LC-1096;
a heavy chain comprising the amino acid sequence set forth in HC-1074, and a light chain comprising the amino acid sequence set forth in LC-1096;
a heavy chain comprising the amino acid sequence set forth in HC-1075, and a light chain comprising the amino acid sequence set forth in LC-1096;
a heavy chain comprising the amino acid sequence set forth in HC-1076, and a light chain comprising the amino acid sequence set forth in LC-1096;
a heavy chain comprising the amino acid sequence set forth in HC-1077, and a light chain comprising the amino acid sequence set forth in LC-1096;
a heavy chain comprising the amino acid sequence set forth in HC-1078, and a light chain comprising the amino acid sequence set forth in LC-1096;
a heavy chain comprising the amino acid sequence set forth in HC-1079, and a light chain comprising the amino acid sequence set forth in LC-1096;
a heavy chain comprising the amino acid sequence set forth in HC-1080, and a light chain comprising the amino acid sequence set forth in LC-1096;
a heavy chain comprising the amino acid sequence set forth in HC-1081, and a light chain comprising the amino acid sequence set forth in LC-1096;
a heavy chain comprising the amino acid sequence set forth in HC-1082, and a light chain comprising the amino acid sequence set forth in LC-1096;
a heavy chain comprising the amino acid sequence set forth in HC-1083, and a light chain comprising the amino acid sequence set forth in LC-1096;
a heavy chain comprising the amino acid sequence set forth in HC-1084, and a light chain comprising the amino acid sequence set forth in LC-1096;
a heavy chain comprising the amino acid sequence set forth in HC-1085, and a light chain comprising the amino acid sequence set forth in LC-1096;
a heavy chain comprising the amino acid sequence set forth in HC-1086, and a light chain comprising the amino acid sequence set forth in LC-1096;
a heavy chain comprising the amino acid sequence set forth in HC-1087, and a light chain comprising the amino acid sequence set forth in LC-1096;
a heavy chain comprising the amino acid sequence set forth in HC-1054, and a light chain comprising the amino acid sequence set forth in LC-1098;
a heavy chain comprising the amino acid sequence set forth in HC-1054, and a light chain comprising the amino acid sequence set forth in LC-1099;
a heavy chain comprising the amino acid sequence set forth in HC-1054, and a light chain comprising the amino acid sequence set forth in LC-1100;
a heavy chain comprising the amino acid sequence set forth in HC-1054, and a light chain comprising the amino acid sequence set forth in LC-1101;
a heavy chain comprising the amino acid sequence set forth in HC-1054, and a light chain comprising the amino acid sequence set forth in LC-1102;
a heavy chain comprising the amino acid sequence set forth in HC-1054, and a light chain comprising the amino acid sequence set forth in LC-1103;
a heavy chain comprising the amino acid sequence set forth in HC-1054, and a light chain comprising the amino acid sequence set forth in LC-1104;
a heavy chain comprising the amino acid sequence set forth in HC-1054, and a light chain comprising the amino acid sequence set forth in LC-1105;
a heavy chain comprising the amino acid sequence set forth in HC-1054, and a light chain comprising the amino acid sequence set forth in LC-1106;
a heavy chain comprising the amino acid sequence set forth in HC-1039, and a light chain comprising the amino acid sequence set forth in LC-1100;
a heavy chain comprising the amino acid sequence set forth in HC-1056, and a light chain comprising the amino acid sequence set forth in LC-1100;
a heavy chain comprising the amino acid sequence set forth in HC-1071, and a light chain comprising the amino acid sequence set forth in LC-1095;
a heavy chain comprising the amino acid sequence set forth in HC-1071, and a light chain comprising the amino acid sequence set forth in LC-1100;
a heavy chain comprising the amino acid sequence set forth in HC-1073, and a light chain comprising the amino acid sequence set forth in LC-1095;
a heavy chain comprising the amino acid sequence set forth in HC-1004, and a light chain comprising the amino acid sequence set forth in LC-1095;
a heavy chain comprising the amino acid sequence set forth in HC-1005, and a light chain comprising the amino acid sequence set forth in LC-1095;
a heavy chain comprising the amino acid sequence set forth in HC-1006, and a light chain comprising the amino acid sequence set forth in LC-1095;
a heavy chain comprising the amino acid sequence set forth in HC-1007, and a light chain comprising the amino acid sequence set forth in LC-1095;
a heavy chain comprising the amino acid sequence set forth in HC-1008, and a light chain comprising the amino acid sequence set forth in LC-1095;
a heavy chain comprising the amino acid sequence set forth in HC-1009, and a light chain comprising the amino acid sequence set forth in LC-1095;
a heavy chain comprising the amino acid sequence set forth in HC-1010, and a light chain comprising the amino acid sequence set forth in LC-1095;
a heavy chain comprising the amino acid sequence set forth in HC-1011, and a light chain comprising the amino acid sequence set forth in LC-1095;
a heavy chain comprising the amino acid sequence set forth in HC-1012, and a light chain comprising the amino acid sequence set forth in LC-1095;
a heavy chain comprising the amino acid sequence set forth in HC-1013, and a light chain comprising the amino acid sequence set forth in LC-1095;
a heavy chain comprising the amino acid sequence set forth in HC-1014, and a light chain comprising the amino acid sequence set forth in LC-1095;
a heavy chain comprising the amino acid sequence set forth in HC-1015, and a light chain comprising the amino acid sequence set forth in LC-1095;
a heavy chain comprising the amino acid sequence set forth in HC-1016, and a light chain comprising the amino acid sequence set forth in LC-1095;
a heavy chain comprising the amino acid sequence set forth in HC-1017, and a light chain comprising the amino acid sequence set forth in LC-1095;
a heavy chain comprising the amino acid sequence set forth in HC-1018, and a light chain comprising the amino acid sequence set forth in LC-1095;
a heavy chain comprising the amino acid sequence set forth in HC-1019, and a light chain comprising the amino acid sequence set forth in LC-1095;
a heavy chain comprising the amino acid sequence set forth in HC-1020, and a light chain comprising the amino acid sequence set forth in LC-1095;
a heavy chain comprising the amino acid sequence set forth in HC-1021, and a light chain comprising the amino acid sequence set forth in LC-1095;
a heavy chain comprising the amino acid sequence set forth in HC-1022, and a light chain comprising the amino acid sequence set forth in LC-1095;
a heavy chain comprising the amino acid sequence set forth in HC-1023, and a light chain comprising the amino acid sequence set forth in LC-1095;
a heavy chain comprising the amino acid sequence set forth in HC-1024, and a light chain comprising the amino acid sequence set forth in LC-1095;
a heavy chain comprising the amino acid sequence set forth in HC-1025, and a light chain comprising the amino acid sequence set forth in LC-1095;
a heavy chain comprising the amino acid sequence set forth in HC-1026, and a light chain comprising the amino acid sequence set forth in LC-1095;
a heavy chain comprising the amino acid sequence set forth in HC-1027, and a light chain comprising the amino acid sequence set forth in LC-1095;
a heavy chain comprising the amino acid sequence set forth in HC-1028, and a light chain comprising the amino acid sequence set forth in LC-1095;
a heavy chain comprising the amino acid sequence set forth in HC-1029, and a light chain comprising the amino acid sequence set forth in LC-1095;
a heavy chain comprising the amino acid sequence set forth in HC-1030, and a light chain comprising the amino acid sequence set forth in LC-1095;
a heavy chain comprising the amino acid sequence set forth in HC-1031, and a light chain comprising the amino acid sequence set forth in LC-1095;
a heavy chain comprising the amino acid sequence set forth in HC-1033, and a light chain comprising the amino acid sequence set forth in LC-1095;
a heavy chain comprising the amino acid sequence set forth in HC-1013, and a light chain comprising the amino acid sequence set forth in LC-1100;
a heavy chain comprising the amino acid sequence set forth in HC-1013, and a light chain comprising the amino acid sequence set forth in LC-1107;
a heavy chain comprising the amino acid sequence set forth in HC-1013, and a light chain comprising the amino acid sequence set forth in LC-1108;
a heavy chain comprising the amino acid sequence set forth in HC-1013, and a light chain comprising the amino acid sequence set forth in LC-1109;
a heavy chain comprising the amino acid sequence set forth in HC-1013, and a light chain comprising the amino acid sequence set forth in LC-1110;
a heavy chain comprising the amino acid sequence set forth in HC-1013, and a light chain comprising the amino acid sequence set forth in LC-1111;
a heavy chain comprising the amino acid sequence set forth in HC-1013, and a light chain comprising the amino acid sequence set forth in LC-1112;
a heavy chain comprising the amino acid sequence set forth in HC-1034, and a light chain comprising the amino acid sequence set forth in LC-1113;
a heavy chain comprising the amino acid sequence set forth in HC-1034, and a light chain comprising the amino acid sequence set forth in LC-1114;
a heavy chain comprising the amino acid sequence set forth in HC-2002, and a light chain comprising the amino acid sequence set forth in LC-1095;
a heavy chain comprising the amino acid sequence set forth in HC-2003, and a light chain comprising the amino acid sequence set forth in LC-1095;
a heavy chain comprising the amino acid sequence set forth in HC-2004, and a light chain comprising the amino acid sequence set forth in LC-1095;
a heavy chain comprising the amino acid sequence set forth in HC-2005, and a light chain comprising the amino acid sequence set forth in LC-1095;
a heavy chain comprising the amino acid sequence set forth in HC-2006, and a light chain comprising the amino acid sequence set forth in LC-1095;
a heavy chain comprising the amino acid sequence set forth in HC-2007, and a light chain comprising the amino acid sequence set forth in LC-1095;
a heavy chain comprising the amino acid sequence set forth in HC-2006, and a light chain comprising the amino acid sequence set forth in LC-2021;
a heavy chain comprising the amino acid sequence set forth in HC-2008, and a light chain comprising the amino acid sequence set forth in LC-1095;
a heavy chain comprising the amino acid sequence set forth in HC-2009, and a light chain comprising the amino acid sequence set forth in LC-1095;
a heavy chain comprising the amino acid sequence set forth in HC-2010, and a light chain comprising the amino acid sequence set forth in LC-1095;
a heavy chain comprising the amino acid sequence set forth in HC-2011, and a light chain comprising the amino acid sequence set forth in LC-1095;
a heavy chain comprising the amino acid sequence set forth in HC-2012, and a light chain comprising the amino acid sequence set forth in LC-2021;
a heavy chain comprising the amino acid sequence set forth in HC-2013, and a light chain comprising the amino acid sequence set forth in LC-1095;
a heavy chain comprising the amino acid sequence set forth in HC-2014, and a light chain comprising the amino acid sequence set forth in LC-1095;
a heavy chain comprising the amino acid sequence set forth in HC-2015, and a light chain comprising the amino acid sequence set forth in LC-1095;
a heavy chain comprising the amino acid sequence set forth in HC-2016, and a light chain comprising the amino acid sequence set forth in LC-1095;
a heavy chain comprising the amino acid sequence set forth in HC-2016, and a light chain comprising the amino acid sequence set forth in LC-2021;
a heavy chain comprising the amino acid sequence set forth in HC-2017, and a light chain comprising the amino acid sequence set forth in LC-1095;
a heavy chain comprising the amino acid sequence set forth in HC-2018, and a light chain comprising the amino acid sequence set forth in LC-1095;
a heavy chain comprising the amino acid sequence set forth in HC-2019, and a light chain comprising the amino acid sequence set forth in LC-1095;
a heavy chain comprising the amino acid sequence set forth in HC-2020, and a light chain comprising the amino acid sequence set forth in LC-1095;
the amino acid sequence set forth in HC-1088;
the amino acid sequence set forth in HC-1089;
the amino acid sequence set forth in HC-1090;
the amino acid sequence set forth in HC-1091;
the amino acid sequence set forth in HC-1092;
the amino acid sequence set forth in HC-1093; or
the amino acid sequence set forth in HC-1094,
wherein the HA tag is optionally excluded from a foregoing amino acid sequence that contains an HA tag.

25. The antibody of any one of claims 1-24, wherein the antibody is a monovalent antibody comprising one VH and one VL, or comprising one heavy chain and one light chain.

26. The monovalent antibody of claim 25, wherein:
the VH comprises a VH CDR1 comprising the amino acid sequence GIDFSSSGYMC (SEQ ID NO:XX), a VH CDR2 comprising the amino acid sequence CIYTYSSNTYYAASVKG (SEQ ID NO:XX), a VH CDR3 comprising the amino acid sequence GTYGYTGATYTMGYFSL (SEQ ID NO:XX); and wherein the VL comprises a VL CDR1 comprising the amino acid sequence QASQNINSYLA (SEQ ID NO:XX), a VL CDR2 comprising the amino acid sequence RASSLES (SEQ ID NO:XX), and a VL CDR3 comprising the amino acid sequence QSYYYSGSSNYNA (SEQ ID NO:XX);
the VH comprises the amino acid sequence of SEQ ID NO: 138 and the VL comprises the amino acid sequence of SEQ ID NO:301; or
the heavy chain comprises the amino acid sequence set forth in HC-2015, and the light chain comprises the amino acid sequence set forth in LC-1095.

27. The antibody of any one of claims 1-24, wherein the antibody is a bivalent antibody comprising two VHs and two VLs, or comprising two heavy chains and two light chains.

28. The bivalent antibody of claim 27, wherein:
each VH comprises a VH CDR1 comprising the amino acid sequence GIDFSSSGYMC (SEQ ID NO:XX), a VH CDR2 comprising the amino acid sequence CIDTYSSNTYYAASVKG (SEQ ID NO:XX), and a VH CDR3 comprising the amino acid sequence GTYGYTGYTYTMGYFSL (SEQ ID NO:XX); and each VL comprises a VL CDR1 comprising the amino acid sequence QASQNINSYLA (SEQ ID NO:XX); a VL CDR2 comprising the amino acid sequence RASSLES (SEQ ID NO:XX); and a VL CDR3 comprising the amino acid sequence QSYYYSGSSNYNA (SEQ ID NO:XX);
each VH comprises the amino acid sequence of SEQ ID NO: 122 and each VL comprises the amino acid sequence of SEQ ID NO:301; or
each heavy chain comprises the amino acid sequence set forth in HC-2008, and each light chain comprises the amino acid sequence set forth in LC-1095.

29. A nucleic acid or nucleic acids encoding the antibody of any one of claims 1 to 28.

30. An expression vector or expression vectors comprising the nucleic acid or nucleic acids of claim 29 operably linked to a promoter.

31. An isolated cell comprising the nucleic acid or nucleic acids of claim 29 or the expression vector or expression vectors of claim 30.

32. An isolated cell comprising a first expression vector comprising a first nucleic acid encoding a first polypeptide comprising the VH of the antibody of any one of claims 1 to 24 operably linked to a promoter, and a second expression vector comprising a second nucleic acid encoding a second polypeptide comprising the VL of the antibody of any one of claims 1 to 24 operably linked to a promoter.

33. A method of making the antibody of any one of claims 1 to 28, comprising culturing the cell of claim 31 or 32 and isolating the antibody.

34. A pharmaceutical composition comprising the antibody of any one of claims 1 to 28 and a pharmaceutically acceptable carrier.

35. A conjugate comprising the antibody of any one of claims 1 to 28 and an agent.

36. The conjugate of claim 35, wherein the agent is an antibody, protein, or peptide.

37. The conjugate of claim 35, wherein the agent is an anti-beta amyloid antibody.

38. The conjugate of claim 37, wherein anti-beta amyloid antibody is aducanumab, bapineuzumab, gantenerumab, solanezumab, donanemab, or lecanemab.

39. The conjugate of claim 35, wherein the agent is an anti-tau antibody, an anti-alpha synuclein antibody, an anti-TDP-43 antibody, an anti-LINGO-1 antibody, an anti-LINGO-2 antibody, an anti-LINGO-3 antibody, an anti-LINGO-4 antibody, an anti-TREM2 antibody, or an anti-C9orf72 dipeptide repeat poly-GA antibody.

40. The conjugate of claim 35, wherein the agent is protein.

41. The conjugate of claim 40, wherein the protein is progranulin.

42. The conjugate of claim 35, wherein the agent is an enzyme.

43. The conjugate of claim 42, wherein the enzyme is glucocerebrosidase.

44. The conjugate of any one of claims 35 to 43, wherein the conjugate is a recombinant fusion protein comprising the antibody and the agent.

45. The conjugate of claim 35, wherein the agent is a nucleic acid.

46. The conjugate of claim 45, wherein the nucleic acid is an mRNA, a siRNA, an antisense oligonucleotide, microRNA (miRNA), guide RNA (gRNA), or a phosphoroamidate morpholino oligomer (PMO).

47. The conjugate of claim 45 or 46, wherein the nucleic acid is linked to the antibody via a linker.

48. The conjugate of claim 35, wherein the agent is a nanoparticle, liposome, or viral vector.

49. A method of transporting an agent across the blood brain barrier via transcytosis, the method comprising administering to a human subject the conjugate of any one of claims 35 to 48.

50. A method of delivering an agent in vivo, the method comprising administering to a human subject the conjugate of any one of claims 35 to 48.

51. The method of claim 50, wherein the human subject has a neurological disorder and the method delivers the agent to brain tissue.

52. The method of claim 51, wherein the neurological disorder is Alzheimer's disease, Parkinson's disease, frontotemporal dementia, ALS, Huntington's disease, multiple sclerosis, spinal muscular atrophy, muscular dystrophy, spinal cord injury, stroke, an ophthalmological condition, acute or chronic optic neuritis, a psychiatric disorder, Tourette's disease brain injury, a brain tumor, or epilepsy.

53. A method of treating Alzheimer's disease in a human subject in need thereof, comprising administering to the subject a therapeutically effective amount of the conjugate of claim 36 or 37.
